(19) United States
(12) Patent Application Publication
(10) Pub. No.: US 2026/0345796 A1
(43) Pub. Date: Nov. 5, 2026

(54) COMPOSITIONS OF CONJUGATED AND UNCONJUGATED PROTEINS

(71) Applicant: KODIAK SCIENCES INC., Palo Alto, CA (US)

(72) Inventors: Daniel Victor Perlroth, Palo Alto, CA (US); Wah Yuen To, San Mateo, CA (US); Carrie Jiali Su, San Mateo, CA (US); Martin Georg Nussbaumer, Wabern (CH); Andreas Sebastian Zurbriggen, Termen (CH)

(21) Appl. No.: 19/490,291

(22) PCT Filed: Jun. 6, 2024
(86) PCT No.: PCT/US2024/032770

§ 371 Date: Dec. 4, 2025

Related U.S. Application Data
(60) Provisional application No. 63/506,781, filed on Jun. 7, 2023.

(51) Int. Cl. A61K 47/60; A61K 9/00; A61K 47/68; C07K 16/22

(52) U.S. Cl. CPC A61K 47/60; A61K 9/0048; A61K 47/6847; C07K 16/22

(57) ABSTRACT

Provided herein are pharmaceutical compositions and formulations of a conjugate comprising a first protein, such as an anti-VEGF antibody, conjugated to a phosphorylcholine-containing polymer, and a second protein that is not conjugated to a phosphorylcholine-containing polymer. The second protein can be the same as the first protein, or can be different. In some embodiments, the pH of the formulation is different from the isoelectric point (pI) of the second protein so as to prevent or reduce turbidity of the formulation. Also provided are methods of preparing the formulations, methods of use thereof, and kits with pre-filled syringes containing the formulations.

### REFERENCE TO RELATED APPLICATIONS

The present application is claims priority to U.S. Provisional Application No. 63/506,781, filed Jun. 7, 2023. The content of each of the aforementioned related application(s) is incorporated herein by reference in its entirety.

### REFERENCE TO SEQUENCE LISTING

The present application is being filed along with a Sequence Listing in electronic format. The Sequence Listing is provided as a file entitled KDIAK210WO_SEQLIST.xml, created May 29, 2024, which is 196,681 bytes in size. The information in the electronic format of the Sequence Listing is incorporated herein by reference in its entirety.

### FIELD

The present invention relates to a composition comprising a mixture of unconjugated and conjugated proteins (e.g., antibodies and conjugates thereof) and methods of using and manufacturing said composition.

### BACKGROUND

Diabetic retinopathy is a leading cause of blindness in people between the ages of about 20 to 64 years of age. Engelgau M, Geiss L, Saaddine J, Boyle J, et al. 2004. The Evolving Diabetes Burden in the United States. Ann of Int Med. 140 (11): 945-951. In the United States, diabetic retinopathy accounts for some 12% of new cases of blindness. Typically, in cases of diabetic retinopathy, retinal blood vessels will swell and leak fluid into the rear of the eye. Hyperglycemia induces intramural and thickening of the basement membrane, resulting in leaky or permeable blood vessels.

In diabetic retinopathy, changes in blood glucose level cause changes to retinal blood vessels. All people with diabetes mellitus are at risk. The longer a person has diabetes, the higher their risk of developing some ocular problem. Between 40 to 45 percent of Americans diagnosed with diabetes have some stage of diabetic retinopathy. Causes and Risk Factors. Diabetic Retinopathy. United States National Library of Medicine. 15 Sep. 2009.

Diabetic retinopathy is first exhibited in the development of microaneurysms in the retina. Microaneurysms occur when there is a swelling of capillaries (very small blood vessels) that feed the retina. The presence of relatively small numbers of microaneurysms will not usually cause problems with vision. However, if the retinopathy develops to later stages, there are significant chances of vision loss. Such early-stage retinopathy are referred to as background diabetic retinopathy or non-proliferative diabetic retinopathy (NPDR). While NPDR patients are generally asymptomatic, early detection of retinopathy is crucial because if the disease proceeds to later stages, significant vision loss is very likely.

In the next stage of diabetic retinopathy, neovascularization occurs in the back of the eye (proliferative diabetic retinopathy). The neovasculature is leaky and the vessels can burst, followed by bleeding and resulting in blurred or obscured vision. Due to lack of oxygen in the eye, still further neovascularization occurs. Blood vessels grow along the retina and in the vitreous humor. As these vessels burst, there is further bleeding and the retina can be badly damaged or destroyed. The accumulation of fluid in the macula due to leaking blood vessels is called diabetic macular edema. Many patients with diabetic retinopathy will develop diabetic macular edema.

There are generally three treatment pathways for patients with diabetic retinopathy: laser surgery, injection of corticosteroids and injection of biologics (e.g. AVASTIN®(bevacizumab), LUCENTIS®(ranibizumab), Eylea®(aflibercept), Beovu® (brocizumab), and Vabysmo™ (faricimab)). While laser surgery is generally effective in treating diabetic retinopathy, retinal damage induced by the laser is a frequent side effect. Steroid preparations such as triamcinolone acetonide have been administered via intravitreal injection to treat diabetic retinopathy. However, to treat diabetic retinopathy, the steroid solutions must be frequently administered. Moreover, intravitreal treatment with steroids has been associated with cataracts, steroid-induced glaucoma and endophthalmitis.

Another way to treat diabetic retinopathy is the intravitreal injection of anti-VEGF agents. In this regard, anti-VEGF therapies such as LUCENTIS®(ranibizumab) and EYLEA®(aflibercept) have been approved for treatment of diabetic retinopathy in patients with diabetic macular edema. VEGF-directed therapies are effective not just for diabetic retinopathy, but also for retinal vascular diseases such as Age-Related Macular Degeneration (AMD), neovascular (wet) AMD and Retinal Vein Occlusion (RVO).

In order to treat diseases such as retinal diseases, it can be useful to use biologics such as antibodies or antibody fragments. These proteins should be formulated in clear solutions. Such formulations can have a defined buffer system and some excipients added for further enhancement of protein stability. Proteins can be conjugated to other moieties to bring an enhanced property or set of properties to the protein. For example, the conjugation of the protein to a potent toxin to create an antibody drug conjugate that targets the toxin to particular receptor-containing cell type for enhanced potency or enhanced safety. The present disclosure is not limited to diabetic retinopathy, and can be applied to various indications as will be appreciated by those in the art.

### SUMMARY

Provided herein is a formulation comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein. Also provided is a therapeutically acceptable composition comprising: a first molar amount of a conjugate comprising a first protein conjugated to a phosphorylcholine-containing polymer; a second molar amount of a second protein that is not conjugated to a phosphorylcholine-containing polymer; and a therapeutically acceptable carrier, wherein the composition comprises the second protein at about 1% or more of a total molar amount of the conjugate and the second protein, wherein the total molar amount comprises a sum of the first molar amount and the second molar amount, wherein the composition has a pH that is about 0.5 pH units away or more from the isoelectric point (pI) of the second protein. Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more, wherein the composition has a pH that is about 0.5 pH units away or more from the isoelectric point (pI) of the second protein. Also provided is a formulation comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the formulation has a reduced viscosity and/or an enhanced injectability compared to a reference formulation comprising the conjugate at the total molar amount.

Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein is about 1% or more, with the remainder comprising the first protein, wherein the composition has a pH that is about 0.5 pH units away or more from the isoelectric point (pI) of the second protein, wherein the composition has a reduced viscosity and/or an enhanced injectability compared to a reference composition comprising the conjugate, wherein the first protein of the conjugate is present in the reference composition at the total mass weight concentration of the first and second proteins in the composition. Also provided is a low-viscosity formulation of a protein conjugate, comprising: a first molar amount of a conjugate comprising a protein conjugated to a phosphorylcholine-containing polymer; a second molar amount of the protein that is not conjugated to the phosphorylcholine-containing polymer; and a pharmaceutically acceptable carrier, wherein the formulation has a pH that is about 0.5 pH units away or more from the isoelectric point (pI) of the protein, wherein the formulation has reduced viscosity and/or an enhanced injectability compared to a reference formulation comprising the conjugate at a total molar amount that is the sum of the first and second molar amounts. Provided herein is a low-viscosity therapeutically acceptable composition of a protein conjugate, comprising a first molar amount of a conjugate comprising a protein conjugated to a phosphorylcholine-containing polymer; a second molar amount of the protein that is not conjugated to the phosphorylcholine-containing polymer; and a pharmaceutically acceptable carrier, wherein the composition has a pH that is about 0.5 pH units away or more from the isoelectric point (pI) of the protein, wherein the composition has reduced viscosity and/or an enhanced injectability compared to a reference composition comprising the conjugate at a total molar amount that is the sum of the first and second molar amounts.

Also provided is a formulation comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the second protein is present in the formulation at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the formulation has a reduced turbidity compared to a reference formulation comprising the first molar amount (e.g., the same first molar amount) of the conjugate and the second molar amount (e.g., the same second molar amount) of the second protein at a pH within 0.5 pH units of the pI of the second protein. In some embodiments, the formulation comprises: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the second protein is present in the formulation at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the formulation has a reduced turbidity compared to a reference formulation comprising the same first molar amount of the conjugate and the same second molar amount of the second protein at a pH about the same as (e.g., within 0.05, 0.1, 0.15, 0.2, 0.2, 0.3, 0.4, or 0.5 pH units of) the pI of the second protein. Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein is about 1% or more, with the remainder comprising the first protein, wherein the composition has a pH that is about 0.5 pH units away or more from the isoelectric point (pI) of the second protein, wherein the composition has a reduced turbidity compared to a reference composition comprising the second protein at the percent composition (e.g., the same percent composition), with the remainder comprising the first protein, at a pH within 0.5 pH units of the pI of the second protein. In some embodiments, the therapeutically acceptable composition comprises: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein is about 1% or more, with the remainder comprising the first protein, wherein the composition has a pH that is about 0.5 pH units away or more from the isoelectric point (pI) of the second protein, wherein the composition has a reduced turbidity compared to a reference composition comprising the second protein at the same percent composition, with the remainder comprising the first protein, at a pH about the same as (e.g., within 0.05, 0.1, 0.15, 0.2, 0.3, 0.4, or 0.5 pH units of) the pI of the second protein. Provided herein is a pharmaceutical formulation comprising: a first molar amount of a conjugate comprising a protein conjugated to a phosphorylcholine-containing polymer; a second molar amount the protein that is not conjugated to the phosphorylcholine-containing polymer; and a pharmaceutically acceptable carrier, wherein the formulation comprises the protein that is not conjugated to the phosphorylcholine-containing polymer at about 1% or more of a total molar amount of the conjugate and unconjugated proteins, wherein the total molar amount comprises a sum of the first molar amount and the second molar amount, wherein the formulation has a pH that is about 0.5 pH units away or more from the isoelectric point (pI) of the protein, wherein the formulation is substantially free of turbidity. Also provided is a formulation comprising: a phosphorylcholine-containing polymer present in the formulation at 100 mg/mL or higher; and a protein that is not conjugated to a phosphorylcholine-containing polymer, wherein the protein is present in the formulation at a second molar amount, wherein the protein is present in the formulation at about 1% or more of a total molar amount of the polymer and the protein, wherein the total molar amount comprises a sum of the first molar amount and the second molar amount, wherein the formulation has a pH that is about 0.5 pH units away or more from the isoelectric point (pI) of the protein.

Provided herein is a formulation comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the difference between the pI of the second protein and the pH of the formulation in the acidic or basic direction is selected to be greater than the minimum difference in the corresponding acidic or basic direction between the pI of the second protein and the pH for a reference formulation comprising: a third molar amount of the conjugate comprising the first protein conjugated to the phosphorylcholine-containing polymer; a fourth molar amount of the second protein that is not conjugated to the phosphorylcholine-containing polymer; and the pharmaceutically acceptable carrier, wherein a first total molar amount comprising a sum of the first molar amount and the second molar amount, and a second total molar amount comprising a sum of the third molar amount and the fourth molar amount are substantially the same, wherein the second molar amount is greater than the fourth molar amount, wherein the reference formulation is substantially free of turbidity. Also provided is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the difference between the pI of the second protein and the pH of the formulation is selected to be greater than the minimum difference between the pI of the second protein and the pH for a reference formulation comprising: the conjugate comprising the first protein conjugated to the phosphorylcholine-containing polymer; the second protein that is not conjugated to the phosphorylcholine-containing polymer; and the pharmaceutically acceptable carrier, wherein the percent composition of the second protein in the composition is higher than the percent composition of the second protein in the reference composition, wherein the reference composition is substantially free of turbidity.

Also provided is a formulation comprising: a first molar amount of a conjugate comprising a first protein conjugated to a polymer; and a second molar amount of a second protein that is not conjugated to a polymer, wherein the formulation comprises the second protein at about 1% or more of a total molar amount of the first protein and the second protein, wherein the total molar amount comprises a sum of the first molar amount and the second molar amount. Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein is about 1% or more, with the remainder comprising the first protein. Also provided is a therapeutically acceptable composition comprising: a first molar amount of a conjugate comprising a first protein conjugated to a polymer; and a second molar amount of a second protein that is not conjugated to a polymer, wherein the composition comprises the second protein at about 1% or more of a total molar amount of the first protein and the second protein, wherein the total molar amount comprises a sum of the first molar amount and the second molar amount. Further provided herein is a formulation comprising: a first molar amount of a first protein that is conjugated to a polymer; and a second molar amount of a second protein that is not conjugated to a polymer, the further improvement comprising: the formulation comprises the second protein at about 1% or more of a total molar amount of the conjugate and the second protein, wherein the total molar amount comprises a sum of the first molar amount and the second molar amount. Provided herein is a therapeutically acceptable composition comprising: a first protein that is conjugated to a polymer; and a second protein that is not conjugated to a polymer, the further improvement comprising: the percent composition of the second protein is about 1% or more, with the remainder comprising the first protein.

Also provided is a formulation comprising: a conjugate comprising a first protein conjugated to a polymer; and a second protein that is not conjugated to a polymer, wherein a first molar amount of the conjugate and a second molar amount of the second protein has been combined in the formulation such that the second molar amount is about 1% or more of a total molar amount of the conjugate and the second protein, wherein the total molar amount comprises a sum of the first molar amount and the second molar amount. Also provided is a therapeutically acceptable composition comprising: a conjugate comprising a first protein conjugated to a polymer; and a second protein that is not conjugated to a polymer, wherein a first molar amount of the conjugate and a second molar amount of the second protein has been combined in the composition such that the second molar amount is about 1% or more of a total molar amount of the conjugate and the second protein, wherein the total molar amount comprises a sum of the first molar amount and the second molar amount. Further provided is a therapeutically acceptable composition comprising: a conjugate comprising a first protein conjugated to a polymer; and a second protein that is not conjugated to a polymer, wherein the second protein at a percent composition relative to the total protein mass weight concentration of the first protein and the second protein in the composition of about 1% or more (e.g., about 5-90%, 15-25%, 25-35%, etc.) has been combined with the conjugate, wherein the remainder of the total protein mass weight concentration comprises the first protein.

Provided herein is a formulation comprising: a conjugate comprising a first protein conjugated to a phosphorylcholine-containing polymer, wherein the polymer has 9 arms and a molecular weight of between 600,000 and 1,000,000 Da, wherein the polymer is present in the formulation at about 100 mg/mL or more; and a second protein that is not conjugated to a polymer, wherein the second protein is present in the formulation at 5-15 mg/mL. Also provided is an intraocular therapeutic composition comprising an anti-VEGF-A antibody at about 50 mg/mL of protein, the anti-VEGF-A antibody comprising: a heavy chain comprising a complementarity determining region 1 (CDR_{H}1): GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11); and a light chain comprising CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14), wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody, wherein the antibody conjugate comprises the anti-VEGF-A antibody conjugated to a phosphorylcholine-containing polymer at a non-native cysteine outside a variable region of the antibody, wherein the phosphorylcholine-containing polymer is present in the composition at about 100 mg/mL or more, wherein the phosphorylcholine-containing polymer has 9 arms and a molecular weight of between 600,000 and 1,000,000 Da, wherein the pH of the composition is about 5.5 or lower.

Also provided is an intraocular therapeutic composition comprising an anti-VEGF-A antibody at about 50 mg/mL of protein, the anti-VEGF-A antibody comprising: a heavy chain comprising an amino acid sequence of SEQ ID NO: 1 (with or without the C-terminal lysine); and a light chain comprising an amino acid sequence of SEQ ID NO: 2, wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody, wherein the antibody conjugate comprises the following structure:

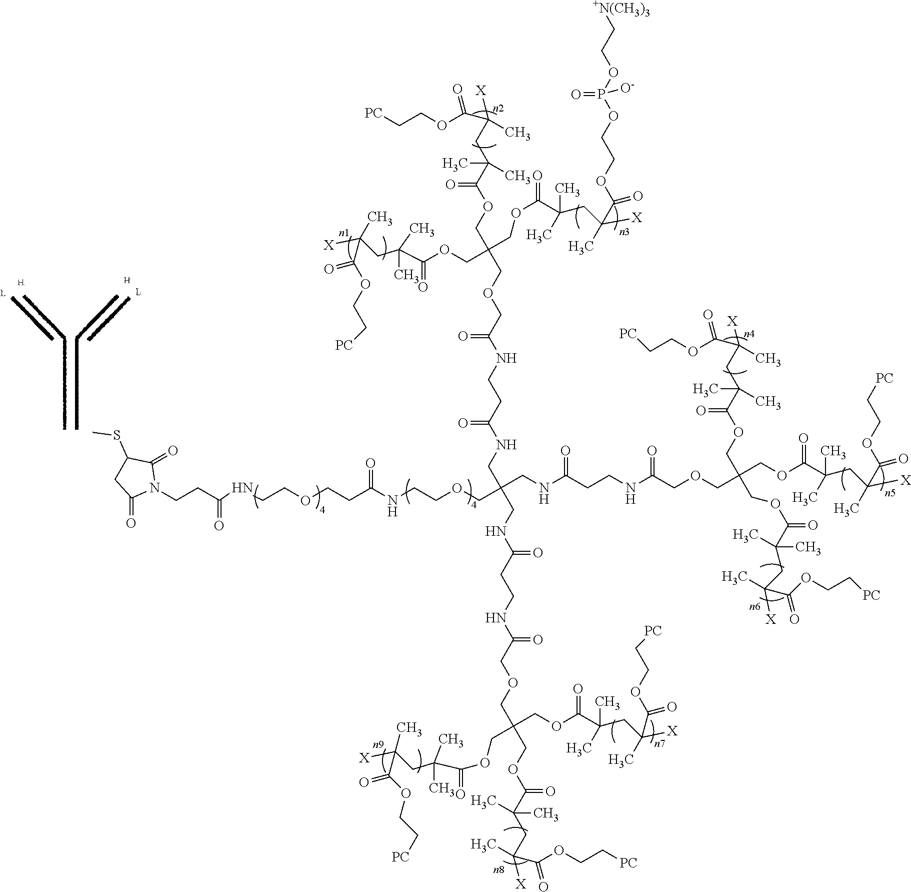

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted on one of the heavy chains; PC is

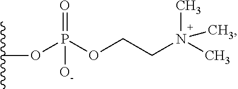

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5.5 or lower.

Also provided is an intraocular therapeutic composition comprising a fusion construct at about 53 mg/mL of protein, the fusion construct comprising a VEGF trap fused to an anti-IL-6 antibody, wherein the fusion construct comprises: a heavy chain comprising an amino acid sequence of SEQ ID NO:105 (with or without the C-terminal lysine); and a light chain comprising an amino acid sequence of SEQ ID NO:106, wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the fusion construct conjugated to a phosphorylcholine-containing polymer, wherein the phosphorylcholine-containing polymer is present in the composition at about 100 mg/mL or more, wherein the pH of the composition is about 5 or lower.

Provided herein is an intraocular therapeutic composition comprising a fusion construct at about 50 mg/mL of protein, the fusion construct comprising a VEGF trap fused to an anti-IL-6 antibody, wherein the fusion construct comprises: a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139), wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the following structure:

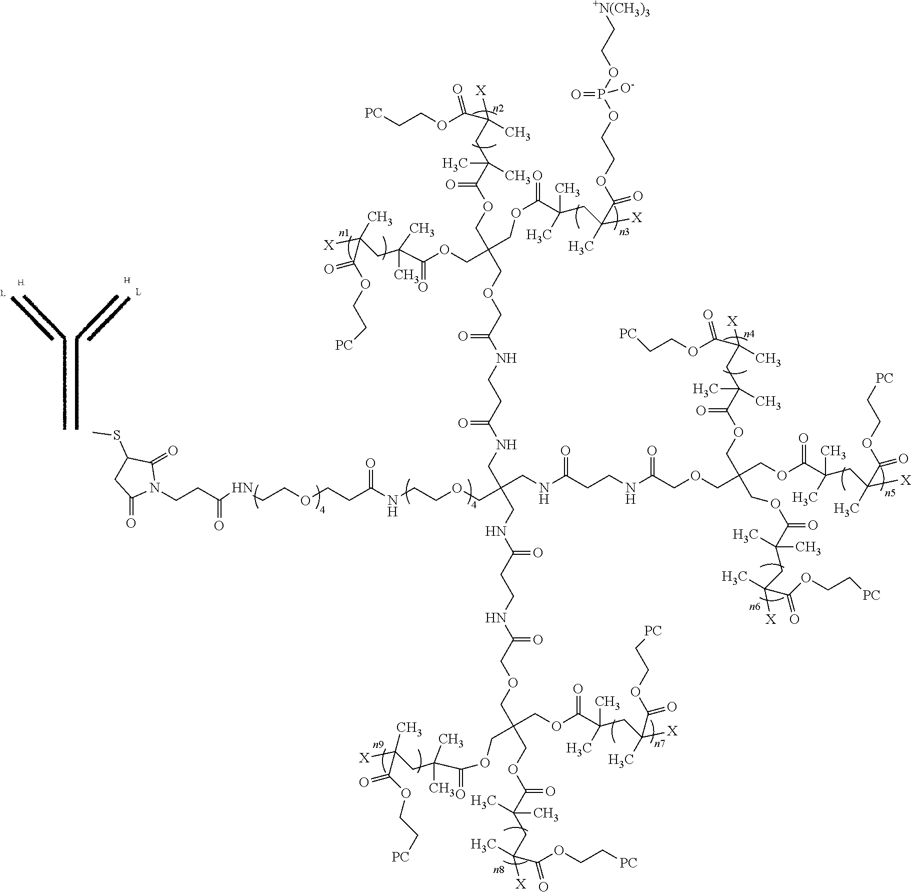

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted on one of the heavy chains; PC is

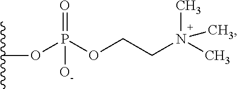

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5 or lower. In some embodiments, the sum of n1, n2, n3, n4, n5, n6, n7, n8 and n9 is about 1500 to about 3500 plus or minus about 10% to about 20%. Also provided is an intraocular therapeutic composition comprising a fusion construct at about 50 mg/mL of protein, the fusion construct comprising a VEGF trap fused to an anti-IL-6 antibody, wherein the fusion construct comprises: a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139), wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the following structure:

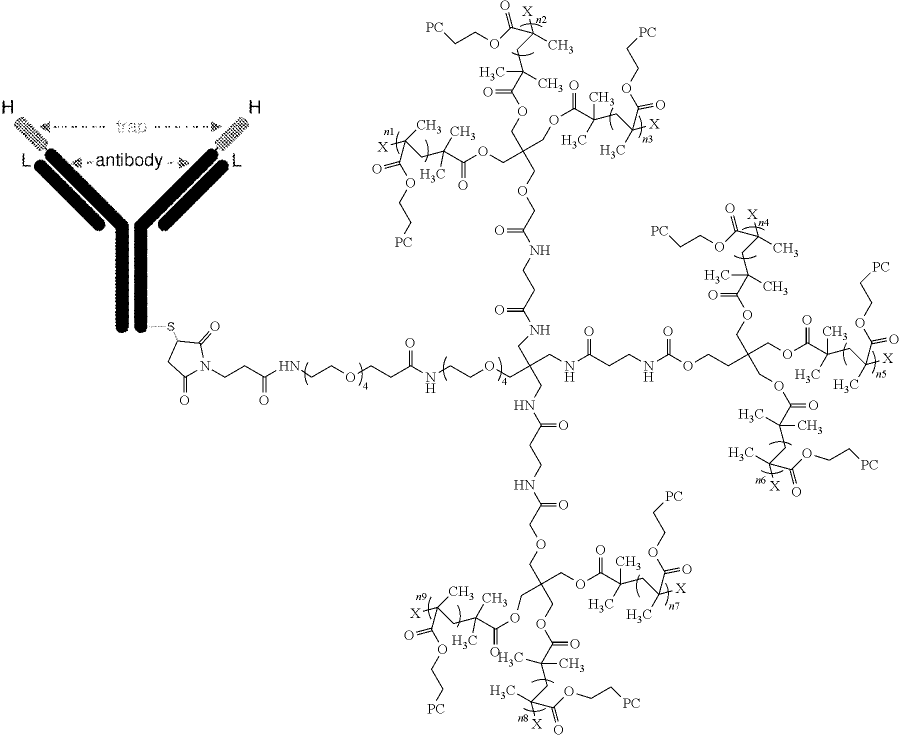

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted on one of the heavy chains; PC is

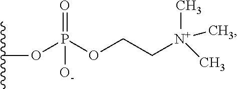

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5 or lower.

Provided herein is a method of preparing a formulation, comprising combining in a formulation: a first molar amount of a conjugate comprising a first protein conjugated to a phosphorylcholine-containing polymer; and a second molar amount of a second protein that is not conjugated to a phosphorylcholine-containing polymer, wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein. Also provided is a method of preparing a therapeutically acceptable composition, comprising combining in a therapeutically acceptable composition: a first molar amount of a conjugate comprising a first protein conjugated to a phosphorylcholine-containing polymer; and a second molar amount of a second protein that is not conjugated to a phosphorylcholine-containing polymer, wherein the composition comprises the second protein at about 1% or more of a total molar amount of the conjugate and the second protein, wherein the total molar amount comprises a sum of the first molar amount and the second molar amount, wherein the composition has a pH that is about 0.5 pH units away or more from the isoelectric point (pI) of the second protein. Further provided herein is a method of preparing a therapeutically acceptable composition, comprising combining in a therapeutically acceptable composition: a conjugate comprising a first protein conjugated to a phosphorylcholine-containing polymer; and a second protein that is not conjugated to a phosphorylcholine-containing polymer, wherein the percent composition of the second protein is about 1% or more, with the remainder comprising the first protein, wherein the composition has a pH that is about 0.5 pH units away or more from the isoelectric point (pI) of the second protein. Also provided is a method of preparing a formulation, comprising adjusting the pH of a formulation to be about 0.5 pH units away or more from the isoelectric point (pI) of an unconjugated protein comprised in the formulation, wherein the formulation comprises: a first molar amount of a conjugate comprising a first protein conjugated to a phosphorylcholine-containing polymer; and a second molar amount of the unconjugated protein, wherein the unconjugated protein is not conjugated to a phosphorylcholine-containing polymer, wherein the formulation comprises the unconjugated protein at about 1% or more of a total molar amount of the conjugate and the unconjugated protein, wherein the total molar amount comprises a sum of the first molar amount and the second molar amount.

Provided herein is a method of preparing a therapeutically acceptable composition, comprising adjusting the pH of a composition to be about 0.5 pH units away or more from the isoelectric point (pI) of an unconjugated protein comprised in the composition, wherein the composition comprises: a first molar amount of a conjugate comprising a first protein conjugated to a phosphorylcholine-containing polymer; and a second molar amount of the unconjugated protein, wherein the unconjugated protein is not conjugated to a phosphorylcholine-containing polymer, wherein the composition comprises the unconjugated protein at about 0.1% or more of a total molar amount of the conjugate and the unconjugated protein, wherein the total molar amount comprises a sum of the first molar amount and the second molar amount. Also provided is a method of preparing a therapeutically acceptable composition, comprising adjusting the pH of a therapeutically acceptable composition to be about 0.5 pH units away or more from the isoelectric point (pI) of an unconjugated protein comprised in the composition, wherein the composition comprises: a conjugate comprising a first protein conjugated to a phosphorylcholine-containing polymer; and a second protein that is not conjugated to a phosphorylcholine-containing polymer, wherein the percent composition of the second protein is about 1% or more, with the remainder comprising the first protein.

Provided herein is a method of preparing a low-viscosity formulation of a protein conjugated to a phosphorylcholine-containing polymer, comprising combining in a formulation: a first molar amount of a conjugate comprising a first protein conjugated to a phosphorylcholine-containing polymer; and a second molar amount of the protein that is not conjugated to a phosphorylcholine-containing polymer, wherein the formulation comprises the protein that is not conjugated to the phosphorylcholine-containing polymer at about 1% or more of a total molar amount of the conjugate unconjugated proteins, wherein the total molar amount comprises a sum of the first molar amount and the second molar amount, wherein the formulation has a pH that is about 0.5 pH units away or more from the isoelectric point (pI) of the second protein, wherein the formulation has reduced viscosity and/or an enhanced injectability compared to a reference formulation comprising the conjugate at the total molar amount. Also provided is a method of preparing a low-viscosity therapeutically acceptable composition of a protein conjugated to a phosphorylcholine-containing polymer, comprising combining in a therapeutically acceptable composition: a conjugate comprising a first protein conjugated to a phosphorylcholine-containing polymer; and a second protein that is not conjugated to a phosphorylcholine-containing polymer, wherein the percent composition of the second protein is about 1% or more, with the remainder comprising the first protein, wherein the composition has a pH that is about 0.5 pH units away or more from the isoelectric point (pI) of the second protein, wherein the composition has reduced viscosity and/or an enhanced injectability compared to a reference composition comprising the conjugate, wherein the first protein of the conjugate is present in the reference composition at the total mass weight concentration of the first and second proteins in the composition.

Also provided is a method of preparing a formulation, comprising combining in a formulation: a first molar amount of a conjugate comprising a first protein conjugated to a phosphorylcholine-containing polymer; and a second molar amount of a second protein that is not conjugated to a phosphorylcholine-containing polymer, wherein the formulation comprises the second protein at about 1% or more of a total molar amount of the conjugate and the second protein, wherein the total molar amount comprises a sum of the first molar amount and the second molar amount. Provided herein is a method of preparing a therapeutically acceptable composition, comprising combining in a therapeutically acceptable composition: a first molar amount of a conjugate comprising a first protein conjugated to a phosphorylcholine-containing polymer; and a second molar amount of a second protein that is not conjugated to a phosphorylcholine-containing polymer, wherein the composition comprises the second protein at about 1% or more of a total molar amount of the conjugate and the second protein, wherein the total molar amount comprises a sum of the first molar amount and the second molar amount. Further provided herein is a method of preparing a therapeutically acceptable composition, comprising combining in a therapeutically acceptable composition: a conjugate comprising a first protein conjugated to a phosphorylcholine-containing polymer; and a second protein that is not conjugated to a phosphorylcholine-containing polymer, wherein the percent composition of the second protein is about 1% or more, with the remainder comprising the first protein.

Provided herein is a formulation or composition made by any one of the methods described herein.

Also provided is a method of treating a subject, comprising: intraocularly administering a therapeutically effective amount of any one of the formulation or composition described herein to a subject in need thereof. Also provided is a method of treating a subject, comprising: intraocularly administering a therapeutically effective amount of a low-viscosity formulation to a subject in need thereof, wherein the formulation comprises: a first concentration of a conjugate comprising a first anti-VEGF antibody conjugated to a phosphorylcholine-containing polymer; a second concentration of an anti-VEGF agent that is not conjugated to a phosphorylcholine-containing polymer; and a pharmaceutically acceptable carrier, wherein the formulation has a pH that is about 0.5 pH units away or more from the isoelectric point (pI) of the anti-VEGF agent.

Provided herein is a kit comprising: a pre-filled syringe comprising a low-viscosity formulation comprising: a conjugate comprising a first protein conjugated to a phosphorylcholine-containing polymer; and a second protein that is not conjugated to a phosphorylcholine-containing polymer; and a syringe needle for injection of the low-viscosity formulation, wherein the gauge of the needle is 27 or higher.

Also provided is a formulation comprising about 40 to about 60 mM sodium acetate, about 0.01% to about 0.04% polysorbate 20, about 40 to about 60 mg/mL (total protein concentration) of a mixture of OG1950 and OG1953, the mixture containing about 15% to about 25% OG1950 and about 75% to about 85% OG1953 by molar amount, at pH about 4.5 to about 5.5. Provided herein is a formulation comprising, consisting of, or consisting essentially of, about 50 mM sodium acetate, about 0.025% polysorbate 20, about 50 mg/mL (total protein concentration) of a mixture of OG1950 and OG1953, the mixture containing about 20% OG1950 and about 80% OG1953 by molar amount, at about pH 5.

Provided herein is a method of storing a protein, comprising maintaining a protein in a formulation for at least 2 months and up to 2 years, the formulation comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the protein comprises an antibody or a fusion construct.

### BRIEF DESCRIPTION OF THE DRAWINGS

FIG. 1 shows Compound L.

FIG. 2 shows Compound K.

FIG. 3 shows the synthesis of OG1802 from R3707.

FIG. 4 shows OG1786.

FIG. 5 shows the synthesis of OG1546 from OG1550.

FIG. 6 shows the synthesis of OG1784 from OG1546 and OG1563.

FIG. 7 shows the synthesis of OG1405 from OG1784.

FIG. 8 shows the synthesis of OG 1785 from OG1405.

FIG. 9 shows the synthesis of OG1786 from OG1785.

FIG. 10 shows OG1802.

FIG. 11 shows Compound E.

FIG. 12 depicts some embodiments of anti-VEGF-A heavy chain with certain effector function mutations and L443C (EU numbering, which is position 449 in SEQ ID NO. 1).

FIG. 13 depicts some embodiments of an anti-VEGF-A light chain (SEQ ID NO. 2).

FIG. 14 depicts some embodiments of a bevacizumab heavy chain (SEQ ID NO. 3).

FIG. 15 depicts some embodiments of a bevacizumab light chain (SEQ ID NO. 4).

FIG. 16 depicts some embodiments of a ranibizumab heavy chain (SEQ ID NO. 5).

FIG. 17 depicts some embodiments of a ranibizumab light chain (SEQ ID NO. 6).

FIG. 18 depicts some embodiments of a method for preparing an antibody conjugate.

FIG. 19 depicts Ion Exchanger analysis (A280 absorbance) of reactions A through G.

FIG. 20 depicts the effect of various anti-VEGF molecules on binding of biotin-VEGF to plate bound VEGFR ECD-Fc protein, and their IC50 values.

FIG. 21 depicts the OG1950 binding affinity to VEGF measured by BIAcore single cycle kinetics.

FIG. 22 depicts binding of the OG1950 to Fc gamma receptor I.

FIG. 23 depicts binding of the OG1950 to Fc gamma receptor IIIa.

FIG. 24 depicts binding of QG 1950 to human complement protein C1q.

FIG. 25 depicts the results of a proliferation assay (including IC50 values).

FIG. 26 depicts the results of single cycle kinetics of VEGF binding to anti-VEGF agents.

FIG. 27 depicts some embodiments of nucleic acid sequences encoding heavy and light chain variable regions.

FIG. 28 is a collection of images and a table depicting results of mixing the free antibody with either polymer or OG1953 composition.

FIG. 29 is a collection of images showing visual appearance of 30 different formulation conditions in two design of experiment (DOE) screenings.

FIG. 30 is a graph depicting turbidity measurements of formulations antibody with OG1801 polymer (samples #1-#30) in the presence of different excipients.

FIG. 31 is a plot produced using JMP SAS software depicting the turbidity changes over time for one embodiment of a DOE experiment with the OG1950 antibody mixed with OG1801 polymer in the presence of different excipients.

FIG. 32 is an image and a table showing the effect of Histidine and pH on turbidity of the formulation.

FIG. 33 is an image showing some embodiments of formulations at differing pHs and corresponding turbidity results.

FIG. 34 is a graph showing some embodiments of formulations at differing pHs and corresponding turbidity results.

FIG. 35A is a collection of images showing the appearances of four different preparations at 50 mg/ml of conjugated antibody supplemented with unconjugated antibody at different pH setpoints. From left to right: A, preparation at pH 6.5 results in a cloudy mixture; B-D, preparations at 5.0-5.2 result in a clear solution irrespective of the formulation buffer and percent unconjugated antibody. The mixtures are first prepared by concentrating the purified conjugated antibody to approx. 6 mg/ml, adding a desired amount of unconjugated antibody, buffer exchange the antibody solution into the formulation buffer followed by a concentration to 20 mg/ml. The protein solution is supplemented with polysorbate 20 and then concentrated to the final target combined concentration of 50 mg/ml.

FIGS. 35B and 35C are a collection of chromatograms and tables showing cation exchanger chromatography (CEX) analysis and analytical size exclusion chromatography (SEC) analysis of several embodiments of formulations of OG1950/OG1953.

FIG. 36 is a table showing the turbidity result for an embodiment of mixing the OG2072 fusion protein with its conjugated form OG2074.

FIGS. 37A and 37B are a collection of a table, images, and a graph showing the screening result of mixing various proteins other than OG1950 antibody with a fixed concentration of OG1801 polymer. This is a control in order to evaluate if the turbidity is a more general phenomenon related to the presence of polymer.

FIGS. 38A and 38B are a collection of images and a plot showing the effect of pH adjustment on the turbidity of formulations. The result demonstrated that the turbidity is reversible or reproducible dependent on the pI of the protein.

FIG. 39 is a table showing the sample setup for formulations with different percentages of free protein relative to conjugated protein.

FIG. 40A is a collection of images showing the appearance of formulations having different percentages of free protein relative to conjugated protein.

FIG. 40B is a collection of traces showing analysis of different partitions during the tandem method analysis of a formulation.

FIG. 40C is a collection of plots showing analysis of different partitions during the tandem method analysis of a formulation.

FIG. 40D is a table showing peak area comparison of CEX bound (free protein) to unbound fraction (conjugate).

FIG. 40E is a collection of traces showing analysis of different partitions during the tandem method analysis of a formulation.

FIG. 40F is a collection of plots showing analysis of different partitions during the tandem method analysis of a formulation.

FIG. 40G is a table showing peak area comparison of CEX bound (free protein) to unbound fraction (conjugate).

FIG. 41 is a table showing the sample setup for a long-term stability plan for various formulations of OG1953 conjugate containing free proteins from 7.5-20% with a total combined protein concentrations of 50-65 mg/ml.

FIGS. 42 and 43 are a collection of tables and plots showing the results of ELISA assays measuring the potency of various formulations of OG1953 conjugate containing free proteins from 7.5-20% with a total combined protein concentrations of 50-65 mg/ml after storage.

FIG. 44 is a collection of a table and a graph showing protein concentration of formulations measured by SoloVPE OD280 nm method.

FIG. 45 is a data table showing a summary of the results of the stability testing of formulations.

FIG. 46A is a collection of SEC-HPLC traces of formulation #4 (20% OG1950, 80% OG1953, 50 mM Na-acetate, 0.025% Tween 20, pH 5.0) after 6 months at the temperatures highlighted. FIG. 46B provides a table and graphs showing size exclusion chromatography analysis of formulations for the aggregation and degradation level of the OG1953 conjugate.

FIG. 47A shows a schematic diagram showing an overview of the Tandem HPLC method, which combines the CEX-HPLC in tandem with a SEC-HPLC column.

FIG. 47B is a collection of traces, a table, and graphs showing tandem method analysis of the OG1950 free protein and its aggregated forms (P1 and P2).

FIGS. 47C and 47D are a collection of traces and a table showing tandem method analysis of formulations.

FIGS. 48A-48C are a collection of a table, image and graph showing the design matrix and visual result of the formulations.

FIGS. 49A and 49B are a collection of plots showing analysis of the turbidity results against the concentration of polymer, free protein and pH.

FIGS. 49C and 49D are a collection of plots and schematic representations showing turbidity of formulations at various pH for the different levels of OG1801 polymer and free protein.

FIG. 49E is a plot showing turbidity measurements results against the concentration of polymer, free protein and pH. FIGS. 49F-49I are a collection of plots showing an overlay of the pH boundary curve plot with other OG1801 polymer with free protein only experimental data.

FIGS. 49J-49M are a collection of plots showing an overlay of the pH boundary curve plot with other OG1953 conjugate solution and free protein only experimental data. FIG. 49N is a plot showing analysis of turbidity measurements in OG1801 polymer solution with free protein against the concentration of polymer, free protein and pH.

FIG. 49O is a plot showing analysis of turbidity measurements in OG1953 conjugate solution with free protein against the concentration of polymer, free protein and pH.

FIG. 50A is a collection of graphs and schematic diagram showing formulation viscosity at 25° C.

FIGS. 50B and 50C are a collection of graphs and a table showing impact of increasing free protein percent composition on viscosity of formulations at 25° C.

FIG. 50D is a collection of plots and tables showing the viscosity of formulations with various concentrations of OG1801 polymer.

FIG. 51 is a collection of images showing air movement in syringes filled with different formulations.

FIGS. 52A-52C are a collection of plots and tables showing potency comparison of various OG1953 formulations using ELISA or cell-based assay.

FIGS. 53A-53D are a collection of graphs, charts, and tables showing improved vision in wet AMD patients administered with OG1953, aflibercept, or other anti-VEGF agents.

FIG. 54 is a collection of sequences showing some non-limiting embodiments of a light chain and heavy chain of a fusion construct.

FIGS. 55A and 55B are a collection of sequences showing some non-limiting embodiments of a heavy chain and light chain, respectively, of a fusion construct.

FIGS. 56A-56C are a collection of plots showing the level of impurities (e.g., aggregation and/or degradation level) in OG1953 conjugate formulations over time.

FIGS. 57A and 57B are a collection of plots and tables showing potency comparison of various OG1953 formulations using ELISA or cell-based assay.

FIGS. 58A-58C are a collection of schematic diagrams showing components of non-limiting examples of formulations of the present disclosure.

FIGS. 59A-59D depict continuous 80 min tandem method separation with PhotoDiol Array (PDA) detection set at 200-350 nm. FIG. 59A depicts the 2D contour view of elution time versus wavelength; FIG. 59B depicts the extracted wavelength profile at 280 nm and the peak identification of the various eluted fractions collected for further characterization using SDS-PAGE analysis followed by Silver Staining, and results were shown in FIG. 59C as a non-reducing gel and FIG. 59D as a reducing gel.

FIGS. 60A-60C depict KSI-301 stability data up to 9 months under different temperature conditions; −20±5° C. (FIG. 60A), 5±3° C. (FIG. 60B), and 25±2° C./60±5% RH (relative humidity) (FIG. 60C).

FIG. 61 depicts KSI-501DS Batches 1-3 Lot Release Data.

FIG. 62 depicts an injection force comparison of (Panel A) OG1953 (100%) conjugate versus the KSI-301 mix formulation using a 27G or 29G dosing needle; (Panel B) OG2074 (100%) conjugate versus the KSI-501_batch 2 mix formulation using a 27G or 29G dosing needle.

FIG. 63 depicts a viscosity comparison at ambient temperature of (Panel A) OG1953 (100%) conjugate versus the KSI-301 mix formulation; (Panel B) OG2074 (100%) conjugate versus various batches of the KSI-501 mix formulation.

### DETAILED DESCRIPTION

Provided herein are formulations and compositions comprising a mixture of an unconjugated protein (e.g., an unconjugated anti-VEGF-A antibody) and conjugates thereof. In some embodiments, the conjugates can include a protein (which may or may not be the same as the unconjugated protein) conjugated to a phosphorylcholine-containing polymer. In some embodiments, the pH of the formulation can be different from the isoelectric point (pI) of the unconjugated protein in the formulation such that the formulation is not or is less turbid. In some embodiments, the formulation has reduced viscosity compared to a reference formulation of the conjugated protein (without the unconjugated protein). The reduced viscosity can improve injectability of the formulation (e.g. by a syringe) and/or handling of the formulation during manufacture. In some embodiments, the composition can be used for the treatment of certain conditions, such as eye disorders, including retinal vascular disorders. The formulations, compositions, and methods of the present disclosure can be provided for treating any disease or disorder as described herein, and is not necessarily limited to diabetic retinopathy or diabetic macular edema.

Provided herein in some embodiments is a drug composition (a formulation) which is a mixture of a protein and a conjugate of that protein and which is a stable solution. Without being limited by theory, lowering the end concentration of biopolymer while keeping the amount of the antibody bioactive the same can result in (i) a decrease in solution viscosity which has many advantages for pharmaceutical manufacturing of drug substance and drug product, and (ii) better handling of the drug for dose preparation and handling in the physician office, and (iii) easier injectability into the patient for example in retina, where a needle is inserted into the vitreous of the eye and the syringe plunger is pushed with a maximum force of the hand, including shorter injection time, reduced force required to drive the plunger down to express out the drug, and a narrower needle such as 30G or 29G, a higher dose level with smaller volume. These properties (i), (ii), and (iii) in turn may lower the chances of unwanted side effects of intravitreal injection, such as but not limited to contamination of bacteria into the eye. In some cases, the side effect includes a cataract. Without being limited by theory, a mixture of unconjugated and conjugated protein can benefit from the direct activity of the protein and a modified activity of the protein as modified by conjugation to a polymer. For example, an immediacy of effect of the unconjugated protein at a defined ratio (for example, 20% of the administered antibody) as well as a modified durability of effect of the conjugated protein at a defined ratio (for example, the remaining 80% of the antibody is in a conjugated form) which modified effect can in combination provide a basal effect driven by the conjugated protein and a bolus effect driven by the unconjugated antibody. In some embodiments, the formulation is a clear solution containing the free (unconjugated) antibody, the antibody conjugated to the phosphorylcholine polymer, the buffer system and at a particular pH, where the formulation is stable and suitable for drug development, manufacturing and/or storage.

In some embodiments, an anti-VEGF antibody (OG1950) conjugated to a phosphorylcholine containing biopolymer (OG1802) which conjugate is called OG1953 can be formulated at 50 mg/mL by weight of antibody and formulated in sodium phosphate pH 6.5 that is clear and stable, and adding a desired amount of the free unconjugated anti-VEGF antibody (OG1950) to create a solution of 40 mg/mL of conjugate (OG1953) and 10 mg/mL of OG1950 without adjusting the formulation system as provided herein can result in turbidity. In some embodiments, formulations and methods provided herein can provide a clear solution of the unconjugated and conjugated antibody coformulation.

Provided herein in some embodiments is a clear and stable ‘formulation system’ of a protein with a phosphorylcholine-biopolymer-conjugated version of that protein, in which the protein can be soluble and clear and stable and avoids turbidity formation. Also provided, in some embodiments, is a method to reduce the turbidity.

In some embodiments, a solubility switch can be applied to OG1953 to create OG1953 80%+OG1950 20% at a pH 5.0, thereby preventing turbidity that forms at a pH of 7.5, which is at or close to the pI of the protein. In some embodiments, the solubility switch involves the application of pH to control this switch. In some embodiments, this solubility switch is applied to OG2074 to create OG2074 70%+OG2072 30% at pH 5.0, for ophthalmology (intravitreal) injection or for systemic diseases.

In some embodiments, in ophthalmology where dose volume is about 100 microliters, the concentration of the dose formulation, for OG1953 is for example 50 mg/mL (as measured by the antibody portion), and the bioconjugate has durability due to the conjugated biopolymer, but to improve manufacturability the same amount of protein bioactive (5.0 mg in 100 microliter dose, i.e. 50 mg/mL in the formulation by weight of antibody) is kept while increasing the relative amount of unconjugated protein. For example, in some embodiments, OG1953 is 80% and OG1950 is 20%; or OG1953 is 75% and OG1950 is 25%; or OG1953 is 70% and OG1950 is 30%; or OG1953 is 65% and OG1950 is 35%; or OG1953 is 60% and OG1950 is 40%; or OG1953 is 50% and OG1950 is 50%. In some embodiments, the pH is at 5.0 (without the addition of histidine or sucrose or trehalose) by shifting the pH to 5.0 and adjusting the formulation buffering constituents to acetate from phosphate. In some embodiments, a high dose formulation with improved manufacturability is achieved because of decreased viscosity for drug substance and drug product (vials, prefilled syringes); improved usability and dose administration (because of decreased viscosity); improved clinical immediacy (so improved balance of clinical immediacy of the for example 20% mAb while retaining clinical durability of the for example 80% mAb-conjugate). In some embodiments, the composition can be tuned to achieve an optimal viscosity to enable large scale manufacturing (of drug substance, of drug product, of pre-filled syringes) and to enable safer dose handling and preparation (for example by physicians and patients) and to enable safer dose administration for example injectability requiring lower injection force by the doctor. In some embodiments, the injection force is less than 10 Newtons, or less than 5 Newtons, injection time is 10 seconds or less, or 5 seconds or less, and the injection needle has a bore size of between 30 gauge and 27 gauge, including 28 gauge or 29 gauge. In some embodiments, the composition can be tuned to achieve an optimal balance of durability of clinical effect (a basal activity) and immediacy of clinical effect (a bolus activity). In some embodiments, the therapeutics are formulated into a clear solution with long-term stability.

Further provided herein are methods for preparing conjugate compositions of antibodies (of any type of antibody and/or protein). In some embodiments, these methods allow for lower aggregate formation or higher efficiency of formation of the desired composition of antibody and antibody conjugate.

These and additional embodiments are provided below, following the definition section.

### Terms

All terms can have their customary and ordinary meaning to one of ordinary skill int the art, in view of the present disclosure. A “neovascular disorder” is a disorder or disease state characterized by altered, dysregulated or unregulated angiogenesis. Examples of neovascular disorders include neoplastic transformation (e.g., cancer) and ocular neovascular disorders including diabetic retinopathy and age-related macular degeneration.

An “ocular neovascular” disorder is a disorder characterized by altered, dysregulated or unregulated angiogenesis in the eye of a patient. Such disorders include optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

The term “percent composition” refers to the percent amount (in mass or concentration units) of a component present in a composition. Percent composition is calculated by determining the amount of a component in mass units (e.g., μg) or in concentration units (e.g., mg/mL), dividing that amount by the total amount of all components in the composition in the corresponding unit, and multiplying by 100. For compositions and formulations of a conjugate and an unconjugated protein described herein, the amount of the unconjugated protein can be divided by the total amount of the protein component in the solution (excluding the contribution from the polymer component of the conjugate to the mass of the conjugate) to obtain a percent composition.

As used herein, “% total molar amount” denotes the proportion (in percent) of the amount (in moles or a molar concentration) of one component of a composition relative to the amount(s) (in moles or a molar concentration) of one or more other component of the composition, that together make up the whole (100%). It is understood that percent composition and % total molar amount can be converted between each other where the molecular weight of all of the relevant components is known.

The term antibody includes intact antibodies and binding fragments thereof. A binding fragment refers to a molecule other than an intact antibody that comprises a portion of an intact antibody that binds the antigen to which the intact antibody binds. Examples of binding fragments include Fv, Fab′, Fab′-SH, F(ab′)2; diabodies; linear antibodies; single-chain antibody molecules (e.g., scFv); and multispecific antibodies formed from antibody fragments. scFv antibodies are described in Houston J S. 1991. Methods in Enzymol. 203:46-96. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies.

Specific binding of an antibody to its target antigen(s) means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, bispecific antibodies can be made in which the two binding sites are different (see, e.g., Songsivilai S, Lachmann P C. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148: 1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. For convenience, the variable heavy CDRs can be referred to as CDR_{H}1, CDR_{H}2 and CDR_{H}3; the variable light chain CDRs can be referred to as CDR_{L}1, CDR_{L}2 and CDR_{L}3. The assignment of amino acids to each domain is in accordance with the definitions of Kabat E A, et al. 1987 and 1991. Sequences of Proteins of Immunological Interest (National Institutes of Health, Bethesda, MD) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, may be missing or derivatized in a proportion or all of the molecules.

The term “epitope” refers to a site on an antigen to which an antibody or extracellular trap segment binds. An epitope on a protein can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody (or Fab fragment) bound to its antigen to identify contact residues.

Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50: 1495, 1990). A test antibody competes with a reference antibody if an excess of a test antibody (e.g., at least 2×, 5×, 10×, 20× or 100×) inhibits binding of the reference antibody by at least 50%. In some embodiments the test antibody inhibits binding of the reference antibody by 75%, 90%, or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “conjugate” refers to a protein covalently linked to a polymer. In some embodiments, the protein is an antibody.

As used herein “unconjugated” and “free” with reference to a protein or antibody are used interchangeably to denote the protein or antibody that is not conjugated to a polymer (e.g., not conjugated to a phosphorylcholine-containing polymer).

The term “isotype” refers to a distinct class of antibody identifiable by the structure of its heavy chain, with each class differing in the (1) structure of the antibody's hinge, (2) sequence (and thus domains), and (3) valency.

As used herein, “VEGF Trap” or similar term denotes the VEGF binding domains (e.g., VEGFR1 domain 2, VEGFR2 domain 3). This fragment allows for the protein to work as a VEGF trap, preventing VEGF from binding to cellularly expressed VEGF receptors. An example of this sequence can be found in Table 0.5. In some embodiments, the VEGF Trap only includes VEGFR1 domain 2, VEGFR2 domain 3. Various embodiments of Trap proteins are known in the art and can be found, for example in U.S. Pub. No. 20150376271, the entirety of which, with respect to various VEGF Trap embodiments (which are VEGFR proteins or fragments thereof) and fusions thereof, is incorporated herein by reference. In some embodiments, the term “VEGF Trap” or similar term refers to a full length extracellular region or any portion thereof, or combination of portions from different VEGF receptors that can antagonize signaling between at least one VEGF and VEGFR.

The term “patient” includes human and other mammalian subjects that receive either prophylactic or therapeutic treatment. In some embodiments, the patient is a human patient.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, WI, using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

Compositions or methods “comprising” one or more recited elements may include other elements not specifically recited. For example, a composition that comprises an antibody may contain the antibody alone or in combination with other ingredients, such as an antibody conjugate. Compositions can comprise a conjugated antibody and an unconjugated antibody.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcy receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component Clq which in turn activates the complement cascade leading to target cell death. Activation of complement may also result in deposition of complement components on the target cell surface that facilitate ADCC by binding complement receptors (e.g., CR3) on leukocytes.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (which can be as defined by Kabat) from a mouse antibody, they can also be made with less than all CDRs (e.g., at least 3, 4, or 5 CDRs from a mouse antibody) (e.g., De Pascalis R, Iwahashi M, Tamura M, et al. 2002. Grafting “Abbreviated” Complementary-Determining Regions Containing Specificity-Determining Residues Essential for Ligand Contact to Engineer a Less Immunogenic Humanized Monoclonal Antibody. J Immunol. 169:3076-3084; Vajdos F F, Adams C W, Breece T N, Presta L G, de Vos A M, Sidhu, S S. 2002. Comprehensive functional maps of the antigen-binding site of an anti-ErbB2 antibody obtained with shotgun scanning mutagenesis. J Mol Biol. 320: 415-428; Iwahashi M, Milenic D E, Padlan E A, et al. 1999. CDR substitutions of a humanized monoclonal antibody (CC49): Contributions of individual CDRs to antigen binding and immunogenicity. Mol Immunol. 36:1079-1091; Tamura M, Milenic D E, Iwahashi M, et al. 2000. Structural correlates of an anticarcinoma antibody: Identification of specificity-determining regions (SDRs) and development of a minimally immunogenic antibody variant by retention of SDRs only. J Immunol. 164:1432-1441).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan E A. 1991. A possible procedure for reducing the immunogenicity of antibody variable domains while preserving their ligand-binding properties. Mol Immunol. 28:489-98) with residues from the corresponding positions of a human antibody sequence. The result is an antibody in which the CDRs are entirely or substantially from a non-human antibody and the variable region frameworks of the non-human antibody are made more human-like by the substitutions. A human antibody can be isolated from a human, or otherwise result from expression of human immunoglobulin genes (e.g., in a transgenic mouse, in vitro or by phage display). Methods for producing human antibodies include the trioma method of Ostberg L, Pursch E. 1983. Human×(mouse×human) hybridomas stably producing human antibodies. Hybridoma 2:361-367; Ostberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, .e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinylpyridine, vinylpyrrolidone and vinyl esters such as vinyl acetate. Additional monomers are useful in high MW polymers. When two different monomers are used, the two monomers are called “comonomers,” meaning that the different monomers are copolymerized to form a single polymer. In some embodiments, one monomer is a phosphorylcholine-containing monomer, and a second comonomer is a different comonomer with a different pendant group chemistry (for example a click chemistry to be a reactive group/recipient of a chemical reaction to be conjugated, for example, to a small molecule bioactive or to a chemical linker). The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

“Initiator” refers to a compound capable of initiating a polymerization using monomers or comonomers. The polymerization can be a conventional free radical polymerization or a controlled/“living” radical polymerization, such as Atom Transfer Radical Polymerization (ATRP), Reversible Addition-Fragmentation-Termination (RAFT) polymerization or nitroxide mediated polymerization (NMP). The polymerization can be a “pseudo” controlled polymerization, such as degenerative transfer. When the initiator is suitable for ATRP, it contains a labile bond which can be homolytically cleaved to form an initiator fragment, I, being a radical capable of initiating a radical polymerization, and a radical scavenger, I′, which reacts with the radical of the growing polymer chain to reversibly terminate the polymerization. The radical scavenger I′ is typically a halogen, but can also be an organic moiety, such as a nitrile. In some embodiments, the initiator contains one of more 2-bromoisobutyrate groups as sites for polymerization via ATRP.

A “chemical linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage consisting of one or more bonds to a bioactive agent. Non-limiting examples include those illustrated in Table 1 of WO2013059137 (incorporated by reference).

The term “reactive group” refers to a group that is capable of reacting with another chemical group to form a covalent bond, i.e. is covalently reactive under suitable reaction conditions, and generally represents a point of attachment for another substance. The reactive group is a moiety, such as maleimide or succinimidyl ester, is capable of chemically reacting with a functional group on a different moiety to form a covalent linkage. Reactive groups generally include nucleophiles, electrophiles and photoactivatable groups.

“Phosphorylcholine,” also denoted as “PC,” refers to the following:

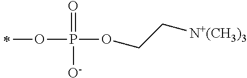

where * denotes the point of attachment. The phosphorylcholine is a zwitterionic group and includes salts (such as inner salts), and protonated and deprotonated forms thereof.

“Phosphorylcholine containing polymer” is a polymer that contains phosphorylcholine. “Zwitterion containing polymer” refers to a polymer that contains a zwitterion.

Poly(acryloyloxyethyl phosphorylcholine) containing polymer refers to a polymer containing 2-(acryloyloxy)ethyl-2-(trimethylammonium)ethyl phosphate (HEA-PC shown below in Example 6) as monomer.

Poly(methacryloyloxyethyl phosphorylcholine) containing polymer refers to a polymer containing 2-(methacryloyloxy)ethyl-2-(trimethylammonium)ethyl phosphate (HEMA-PC or MPC) as monomer (see below):

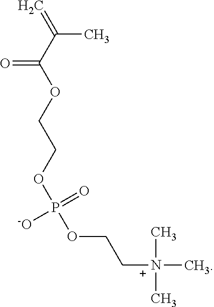

As used herein, “MPC” and “HEMA-PC” are interchangeable.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of endgroup analysis or the measurement of colligative properties (e.g., freezing point depression, boiling point elevation, or osmotic pressure) to determine number average molecular weight, or the use of light scattering techniques, ultracentrifugation or viscometry to determine weight average molecular weight. In some embodiments, the molecular weight is measured by SEC-MALS (size exclusion chromatography—multi angle light scattering). In some embodiments, the multi-angle light scattering includes 18-angle MALS. In some embodiments, the multi-angle light scattering includes 3-angle and 18-angle MALS. In some embodiments, the polymeric reagents are typically polydisperse (i.e., number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

The phrase “a” or “an” entity refers to one or more of that entity; for example, a compound refers to one or more compounds or at least one compound. As such, the terms “a” (or “an”), “one or more”, and “at least one” can be used interchangeably herein.

“About” means variation one might see in measurements taken among different instruments, samples, and sample preparations.

“Protected,” “protected form,” “protecting group” and “protective group” refer to the presence of a group (i.e., the protecting group) that prevents or blocks reaction of a particular chemically reactive functional group in a molecule under certain reaction conditions. Protecting groups vary depending upon the type of chemically reactive group being protected as well as the reaction conditions to be employed and the presence of additional reactive or protecting groups in the molecule, if any. Suitable protecting groups include those such as found in the treatise by Greene et al., “Protective Groups In Organic Synthesis,” 3^{rd} Edition, John Wiley and Sons, Inc., New York, 1999.

“Alkyl” refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together.

The term “lower” referred to above and hereinafter in connection with organic radicals or compounds respectively defines a compound or radical which can be branched or unbranched with up to and including 7 or up to and including 4 and (as unbranched) one or two carbon atoms.

“Alkylene” refers to an alkyl group, as defined above, linking at least two other groups, i.e., a divalent hydrocarbon radical. The two moieties linked to the alkylene can be linked to the same atom or different atoms of the alkylene. For instance, a straight chain alkylene can be the bivalent radical of —(CH₂)ₙ, where n is 1, 2, 3, 4, 5 or 6. Alkylene groups include, but are not limited to, methylene, ethylene, propylene, isopropylene, butylene, isobutylene, sec-butylene, pentylene and hexylene.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO2 in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. The term “alkyl” includes groups such as haloalkyl (e.g., —CF₃ and —CH₂CF₃) and acyl (e.g., —C(O)CH₃, —C(O)CF₃, —C(O)CH₂OCH₃, and the like). In some embodiments, the substituted alkyl and heteroalkyl groups have from 1 to 4 substituents. In some embodiments, the substituted alkyl and heteroalkyl groups have 1, 2 or 3 substituents. Exceptions are those perhalo alkyl groups (e.g., pentafluoroethyl and the like).

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —CONR′R″, —O C(O)NR′R″, —NR″C(O)R′, —NR′—C(O)NR″R″′, —NR″C(O)₂R′, —NR—C(NR′R″R″′)═NR″ ″, —N R—C(NR′R″)═NR″′, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

“Alkoxy” refers to an alkyl group having an oxygen atom that either connects the alkoxy group to the point of attachment or is linked to two carbons of the alkoxy group. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

“Carboxyalkyl” means an alkyl group (as defined herein) substituted with a carboxy group. The term “carboxycycloalkyl” means a cycloalkyl group (as defined herein) substituted with a carboxy group. The term alkoxyalkyl means an alkyl group (as defined herein) substituted with an alkoxy group. The term “carboxy” employed herein refers to carboxylic acids and their esters.

“Haloalkyl” refers to alkyl as defined above where some or all of the hydrogen atoms are substituted with halogen atoms. Halogen (halo) represents chloro or fluoro but may also be bromo or iodo. For example, haloalkyl includes trifluoromethyl, fluoromethyl, 1,2,3,4,5-pentafluoro-phenyl, etc. The term “perfluoro” defines a compound or radical which has all available hydrogens that are replaced with fluorine. For example, perfluorophenyl refers to 1,2,3,4,5-pentafluorophenyl, perfluoromethyl refers to 1,1,1-trifluoromethyl, and perfluoromethoxy refers to 1,1,1-trifluoromethoxy.

“Fluoro-substituted alkyl” refers to an alkyl group where one, some, or all hydrogen atoms have been replaced by fluorine.

“Cytokine” is a member of a group of protein signaling molecules that may participate in cell-cell communication in immune and inflammatory responses. Cytokines are typically small, water-soluble glycoproteins that have a mass of about 8-35 kDa.

“Cycloalkyl” refers to a cyclic hydrocarbon group that contains from about 3 to 12, from 3 to 10, or from 3 to 7 endocyclic carbon atoms. Cycloalkyl groups include fused, bridged and spiro ring structures.

“Endocyclic” refers to an atom or group of atoms which comprise part of a cyclic ring structure.

“Exocyclic” refers to an atom or group of atoms which are attached but do not define the cyclic ring structure.

“Cyclic alkyl ether” refers to a 4 or 5 member cyclic alkyl group having 3 or 4 endocyclic carbon atoms and 1 endocyclic oxygen or sulfur atom (e.g., oxetane, thietane, tetrahydrofuran, tetrahydrothiophene); or a 6 to 7 member cyclic alkyl group having 1 or 2 endocyclic oxygen or sulfur atoms (e.g., tetrahydropyran, 1,3-dioxane, 1,4-dioxane, tetrahydrothiopyran, 1,3-dithiane, 1,4-dithiane, 1,4-oxathiane).

“Alkenyl” refers to either a straight chain or branched hydrocarbon of 2 to 6 carbon atoms, having at least one double bond. Examples of alkenyl groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can also have from 2 to 3, 2 to 4, 2 to 5, 3 to 4, 3 to 5, 3 to 6, 4 to 5, 4 to 6 and 5 to 6 carbons. The alkenyl group is typically monovalent, but can be divalent, such as when the alkenyl group links two moieties together.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene.

“Alkynyl” refers to either a straight chain or branched hydrocarbon of 2 to 6 carbon atoms, having at least one triple bond. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can also have from 2 to 3, 2 to 4, 2 to 5, 3 to 4, 3 to 5, 3 to 6, 4 to 5, 4 to 6 and 5 to 6 carbons. The alkynyl group is typically monovalent, but can be divalent, such as when the alkynyl group links two moieties together.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, butynylene, sec-butynylene, pentynylene and hexynylene.

“Cycloalkyl” refers to a saturated or partially unsaturated, monocyclic, fused bicyclic or bridged polycyclic ring assembly containing from 3 to 12 ring atoms, or the number of atoms indicated. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane.

“Cycloalkylene” refers to a cycloalkyl group, as defined above, linking at least two other groups, i.e., a divalent hydrocarbon radical. The two moieties linked to the cycloalkylene can be linked to the same atom or different atoms of the cycloalkylene. Cycloalkylene groups include, but are not limited to, cyclopropylene, cyclobutylene, cyclopentylene, cyclohexylene, and cyclooctylene.

“Heterocycloalkyl” refers to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

“Heterocycloalkylene” refers to a heterocyclalkyl group, as defined above, linking at least two other groups. The two moieties linked to the heterocycloalkylene can be linked to the same atom or different atoms of the heterocycloalkylene.

“Aryl” refers to a monocyclic or fused bicyclic, tricyclic or greater, aromatic ring assembly containing 6 to 16 ring carbon atoms. For example, aryl may be phenyl, benzyl or naphthyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy- C₂-C₃-alkylene-phenyl is 2,3-dihydrobenzofuran-5-yl.

In some embodiments the aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular phenyl.

Examples of substituted phenyl groups as R are, e.g. 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl-(methyl)-aminomethylphen-1-yl, 3-aminomethylphen-1-yl, 4-N-acetylaminomethylphen-1-yl, 4-aminophen-1-yl, 3-aminophen-1-yl, 2-aminophen-1-yl, 4-phenyl-phen-1-yl, 4-(imidazol-1-yl)-phenyl, 4-(imidazol-1-ylmethyl)-phen-1-yl, 4-(morpholin-1-yl)-phen-1-yl, 4-(morpholin-1-ylmethyl)-phen-1-yl, 4-(2-methoxyethylaminomethyl)-phen-1-yl and 4-(pyrrolidin-1-ylmethyl)-phen-1-yl, 4-(thiophenyl)-phen-1-yl, 4-(3-thiophenyl)-phen-1-yl, 4-(4-methylpiperazin-1-yl)-phen-1-yl, and 4-(piperidinyl)-phenyl and 4-(pyridinyl)-phenyl optionally substituted in the heterocyclic ring.

“Arylene” refers to an aryl group, as defined above, linking at least two other groups. The two moieties linked to the arylene are linked to different atoms of the arylene. Arylene groups include, but are not limited to, phenylene.

“Arylene-oxy” refers to an arylene group, as defined above, where one of the moieties linked to the arylene is linked through an oxygen atom. Arylene-oxy groups include, but are not limited to, phenylene-oxy.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)R′, —S(O)₂R′, —S(O)₂NR′R″, —N3, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

“Heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. In some embodiments, quinolinyl represents 2-, 3- or 4-quinolinyl. In some embodiments, isoquinolinyl represents 1-, 3- or 4-isoquinolinyl. In some embodiments, benzopyranyl, benzothiopyranyl can represent 3-benzopyranyl or 3-benzothiopyranyl, respectively. In some embodiments, thiazolyl can represent 2- or 4-thiazolyl. In some embodiments, triazolyl can be 1-, 2- or 5-(1,2,4-triazolyl). In some embodiments, tetrazolyl can be 5-tetrazolyl.

In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

The term “heteroalkyl” refers to an alkyl group having from 1 to 3 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers, alkyl-amines and alkyl-thiols.

The term “heteroalkylene” refers to a heteroalkyl group, as defined above, linking at least two other groups. The two moieties linked to the heteroalkylene can be linked to the same atom or different atoms of the heteroalkylene.

“Electrophile” refers to an ion or atom or collection of atoms, which may be ionic, having an electrophilic center, i.e., a center that is electron seeking, capable of reacting with a nucleophile. An electrophile (or electrophilic reagent) is a reagent that forms a bond to its reaction partner (the nucleophile) by accepting both bonding electrons from that reaction partner.

“Nucleophile” refers to an ion or atom or collection of atoms, which may be ionic, having a nucleophilic center, i.e., a center that is seeking an electrophilic center or capable of reacting with an electrophile. A nucleophile (or nucleophilic reagent) is a reagent that forms a bond to its reaction partner (the electrophile) by donating both bonding electrons. A “nucleophilic group” refers to a nucleophile after it has reacted with a reactive group. Non limiting examples include amino, hydroxyl, alkoxy, haloalkoxy and the like.

“Maleimido” refers to a pyrrole-2,5-dione-1-yl group having the structure:

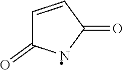

which upon reaction with a sulfhydryl (e.g., a thio alkyl) forms an —S-maleimido group having the structure

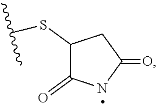 [SMILES: *SC1CC(=O)NC1=O]

where “⋅” indicates the point of attachment for the maleimido group and [symbol] indicates the point of attachment of the sulfur atom the thiol to the remainder of the original sulfhydryl bearing group.

For the purpose of this disclosure, “naturally occurring amino acids” found in proteins and polypeptides are L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine, and or L-valine. “Non-naturally occurring amino acids” found in proteins are any amino acid other than those recited as naturally occurring amino acids. Non-naturally occurring amino acids include, without limitation, the D isomers of the naturally occurring amino acids, and mixtures of D and L isomers of the naturally occurring amino acids. Other amino acids, such as N-alpha- methyl amino acids (e.g. sarcosine), 4-hydroxyproline, desmosine, isodesmosine, 5-hydroxylysine, epsilon-N-methyllysine, 3-methylhistidine, although found in naturally occurring proteins, are considered to be non-naturally occurring amino acids found in proteins for the purpose of this disclosure as they are generally introduced by means other than ribosomal translation of mRNA.

“Linear” in reference to the geometry, architecture, or overall structure of a polymer, refers to polymer having a single polymer arm.

“Branched,” in reference to the geometry, architecture, or overall structure of a polymer, refers to a polymer having 2 or more polymer “arms” extending from a core structure contained within an initiator. The initiator may be employed in an atom transfer radical polymerization (ATRP) reaction. A branched polymer may possess 2 polymer chains (arms), 3 polymer arms, 4 polymer arms, 5 polymer arms, 6 polymer arms, 7 polymer arms, 8 polymer arms, 9 polymer arms or more. Each polymer arm extends from a polymer initiation site. Each polymer initiation site is capable of being a site for the growth of a polymer chain by the addition of monomers. For example and not by way of limitation, using ATRP, the site of polymer initiation on an initiator is typically an organic halide undergoing a reversible redox process catalyzed by a transition metal compound such as cuprous halide. In some embodiments, the halide is a bromine.

“Pharmaceutically acceptable excipient” or “pharmaceutically acceptable carrier” refers to an excipient that can be included in compositions and that causes no significant adverse toxicological effect on the patient and is approved or approvable by the FDA for therapeutic use, particularly in humans. Nonlimiting examples of pharmaceutically acceptable excipients or carriers include water, NaCl, normal saline solutions, lactated Ringer's, normal sucrose, normal glucose and the like. In any embodiment, a pharmaceutically acceptable carrier can be acceptable for administering directly into the eye of a patient (e.g., acceptable for intravitreal administration).

Therapeutic proteins are administered in an effective regime meaning a dosage, route of administration and frequency of administration that delays the onset, reduces the severity, inhibits further deterioration, and/or ameliorates at least one sign or symptom of a disorder. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

The “biological half-life” of a substance is a pharmacokinetic parameter which specifies the time required for one half of the substance to be removed from a tissue or an organism following introduction of the substance.

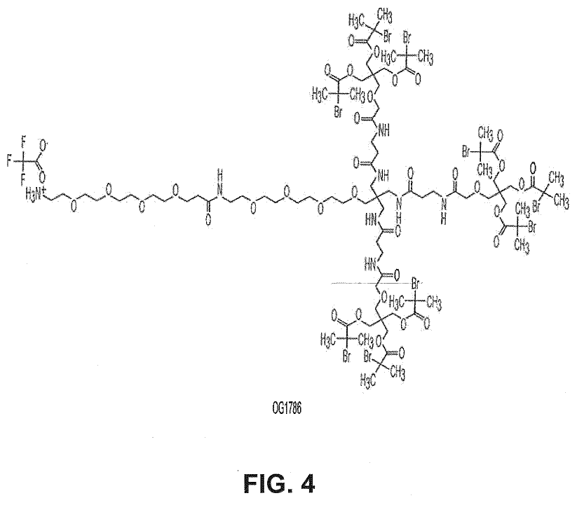

“OG1786” is a 9-arm initiator used for polymer synthesis with the structure shown in FIG. 4, which depicts that salt form of OG1786 with trifluororacetic acid. OG1786 may be used as other salts are used or as the free base.

“OG1801” is an approximately (+/−25%) 800 kDa polymer (either by Mn or Mp) made using OG1786 as an initiator for ATRP synthesis using the monomer HEMA-PC.

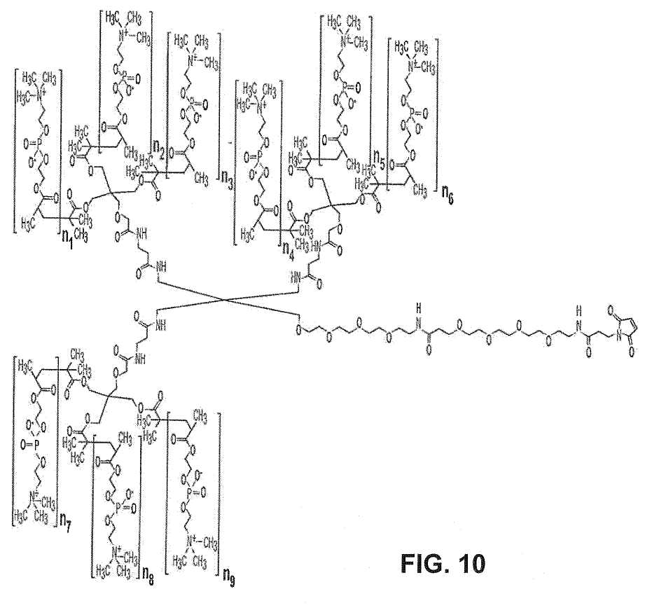

“OG1802” is OG1801 with a maleimide functionality added and is shown in FIG. 10 wherein each of n₁, n₂, n₃, n₄, n₅, n₆, n₇, n₈ and n₉ is an integer (positive) (from 0 up to about 3000) such that the total molecular weight of the polymer is (Mw) 800,000±20% Daltons.

Multi-angle light scattering (MALS) is a technique of analyzing macromolecules where the laser light impinges on the molecule, the oscillating electric field of the light induces an oscillating dipole within it. This oscillating dipole will re-radiate light and can be measured using a MALS detector such as Wyatt miniDawn TREOS. The intensity of the radiated light depends on the magnitude of the dipole induced in the macromolecule which in turn is proportional to the polarizability of the macromolecule, the larger the induced dipole, and hence, the greater the intensity of the scattered light. Therefore, in order to analyze the scattering from a solution of such macromolecules, one should know their polarizability relative to the surrounding medium (e.g., the solvent). This may be determined from a measurement of the change, Δn, of the solution's refractive index n with the molecular concentration change, Δc, by measuring the dn/dc (=Δn/Δc) value using a Wyatt Optilab T-rEX differential refractometer. Two molar weight parameters that MALS determination employ are number average molecular weight (Mn) and weight average molecular weight (Mw) where the polydispersity index (PDI) equals Mw divided by Mn. SEC also allows another average molecular weight determination of the peak molecular weight Mp which is defined as the molecular weight of the highest peak at the SEC.

The PDI is used as a measure of the broadness of a molecular weight distribution of a polymer and bioconjugate which is derived from conjugation of a discrete protein (e.g. OG1950) to a polydisperse biopolymer (e.g., OG1802). For a protein sample, its polydispersity is close to 1.0 due to the fact that it is a product of translation where every protein molecule in a solution is expected to have almost the same length and molar mass. In contrast, due to the polydisperse nature of the biopolymer where the various length of polymer chains are synthesized during the polymerization process, it is very important to determine the PDI of the sample as one of its quality attribute for narrow distribution of molecular weight.

Size exclusion chromatography (SEC) is a chromatography technique in which molecules in solution are separated by their size. Typically an aqueous solution is applied to transport the sample through the column which is packed with resins of various pore sizes. The resin is expected to be inert to the analyte when passing through the column and the analytes separate from each other based on their unique size and the pore size characteristics of the selected column.

Coupling the SEC with MALS or SEC/MALS provides accurate distribution of molar mass and size (root mean square radius) as opposed to relying on a set of SEC calibration standards. This type of arrangement has many advantages over traditional column calibration methods. Since the light scattering and concentration are measured for each eluting fraction, the molar mass and size can be determined independently of the elution position. This is particularly relevant for species with non-globular shaped macromolecules such as the biopolymers (OG1802) or bioconjugates (OG1953); such species typically do not elute in a manner that might be described by a set of column calibration standards.

In some embodiments, a SEC/MALS analysis includes a Waters HPLC system with Alliance 2695 solvent delivery module and Waters 2996 Photodiole Array Detector equipped with a Shodex SEC-HPLC column (7.8×300 mm). This is connected online with a Wyatt miniDawn TREOS and Wyatt Optilab T-rEX differential refractometer. The Empower software from Waters can be used to control the Waters HPLC system and the ASTRA V 6.1.7.16 software from Wyatt can be used to acquire the MALS data from the Wyatt miniDawn TREOS, dn/dc data from the T-rEX detector and the mass recovery data using the A280 absorbance signal from the Waters 2996 Photodiole Array detector. SEC can be carried out at 1 ml/min in 1×PBS pH 7.4, upon sample injection, the MALS and RI signals can be analyzed by the ASTRA software for determination of absolute molar mass (Mp, Mw, Mn) and polydisperse index (PDI). In addition, the calculation also involves the input dn/dc values for polymer and protein as 0.142 and 0.183, respectively. For OG1953 bioconjugates dn/dc value, the dn/dc is calculated based on the weighted MW of the polymer and the protein to be about 0.148 using the formula below:

Conjugate dn/dc=0.142×[MWpolymer/(MWpolymer+MWprotein)]+0.183×[MWprotein/(MWpolymer+MWprotein)]

where MWpolymer for OG1802 is 800 kDa and the MWprotein for OG1950 is 150 kDa.

### General

### Protein and Antibody Compositions, and Methods of Preparing Same

In some embodiments, a formulation or composition (e.g., therapeutically acceptable composition) that includes a first protein that is conjugated to a polymer (e.g., a phosphorylcholine-containing polymer) and a second protein that is unconjugated is provided. In some embodiments, the first protein is an antibody and the second protein is an antibody. Both antibodies can be therapeutic antibodies. In some embodiments, the composition is for treating an eye disorder in a subject. In some embodiments, a formulation includes at least a polymer (e.g., a phosphorylcholine-containing polymer) and an unconjugated protein (e.g., an unconjugated antibody). As used herein, formulation and composition (e.g., therapeutically acceptable composition, pharmaceutical composition, or therapeutic composition) can be used interchangeably. In some embodiments, a formulation or therapeutically acceptable composition is safe for human use (e.g., administering to a human). In some embodiments, a formulation or therapeutically acceptable composition is not an intermediate product generated during manufacture of a final product (e.g., that may be suitable for use in a human).

In some embodiments, provided herein is a composition (e.g., therapeutically acceptable composition) comprising any two proteins that can be the same or different in function, wherein one is conjugated to a polymer and the other is not conjugated to the polymer (or conjugated to any polymer or conjugated to any effective amount of a polymer). The composition can be for the treatment of an eye disorder. In some embodiments, both of the proteins are therapeutics proteins for the treatment of an eye disorder. In some embodiments, one or both of the proteins are therapeutics, antibodies and/or therapeutic antibodies. In some embodiments, one antibody is conjugated to a polymer and the other antibody is not conjugated to a polymer. In some embodiments, the antibody may be synthesized. In some embodiments, the antibody may be a native sequence antibody. In some embodiments, the antibody may be a Fab fragment. In some embodiments, the antibody may be a Trap fragment. In some embodiments, the antibody may be a fusion protein such as a Trap-antibody fusion protein. In some embodiments, the antibody may be a peptide fragment. In some embodiments, a non-antibody scaffold protein can be used instead of an antibody.

Provided herein is a formulation (or therapeutically acceptable composition) comprising: a first molar amount of a conjugate comprising a first protein conjugated to a phosphorylcholine-containing polymer (the “conjugated protein”); a second molar amount of a second protein that is not conjugated to a phosphorylcholine-containing polymer (the “unconjugated protein”); and a pharmaceutically acceptable carrier, wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away (e.g., above or below) or more from the isoelectric point (pI) of the second protein. As used herein, “molar amount” denotes a measure of the molar quantity of a molecule. In some embodiments, molar amount is a molar concentration (e.g., M, mM, PM, nM, etc.). In some embodiments, molar amount is expressed in units of moles (e.g., moles, millimoles, micromoles, etc.). As used herein, the isoelectric point (pI) of a protein has its customary and ordinary meaning to one of ordinary skill int the art, in view of the present disclosure. The pI denotes the pH at which the protein carries no net charge. The pI can be a previously known value for the same or similar protein, or be determined based on a model, or empirically. In some embodiments, the pI is a theoretically determined pI. In some embodiments, the pI is an empirically determined pI.

Low-viscosity formulations of a protein conjugate (e.g., a protein conjugated to a phosphorylcholine-containing polymer) are also provided. A high concentration of the protein conjugate in the formulation can raise the viscosity of the formulation. In some embodiments, lowering the viscosity of the formulation (while maintaining the total amount of active protein) improves one or more of manufacturability, handling, storage, and injectability, for example when delivering the formulation with a syringe to a site of treatment (e.g., intraocular administration).

Formulations (or therapeutically acceptable compositions) of the present disclosure (e.g., having a polymer or polymer-conjugated protein and unconjugated protein) can include the unconjugated protein (e.g., protein that is not conjugated to the phosphorylcholine-containing polymer) at any suitable % molar amount of the total molar amount of the polymer/polymer conjugate and the unconjugated protein. In some embodiments, the formulation (or therapeutically acceptable composition) comprises the second protein (the unconjugated protein) at about 1% or more of a total molar amount of the conjugate and the second protein, wherein the total molar amount comprises a sum of the first molar amount of the conjugate and the second molar amount of the second protein. For example, where the combined concentration of the conjugate and the unconjugated protein is 100 μM, the unconjugated protein at 1% of the total molar amount is at 1 μM, and the conjugate is at 99 μM. In some embodiments, the formulation (or therapeutically acceptable compositions) comprises the second protein (the unconjugated protein) at about 1% or more of a total molar amount of the conjugate and the second protein, wherein the total molar amount comprises a sum of the first molar amount of the conjugate and the second molar amount of the second protein. In some embodiments, the formulation comprises the second protein (the unconjugated protein) at, or at about 1% or more, e.g., about 2% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, or about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less of a total molar amount of the conjugate and the second protein, or optionally, the formulation includes the second protein at a percentage in a range defined by any two of the preceding values (e.g., about 1-95%, 5-90%, 10-80%, 5-50%, 10-40%, 15-35%, 15-25%, 25-35%, 25-40%, 40-95%, 50-80%, etc.) of a total molar amount of the conjugate and the second protein, wherein the total molar amount comprises a sum of the first molar amount of the conjugate and the second molar amount of the second protein. In some embodiments, the formulation comprises the second protein (the unconjugated protein) at between about 5% and about 50%, or between about 15% and about 30% of a total molar amount of the conjugate and the second protein, wherein the total molar amount comprises a sum of the first molar amount of the conjugate and the second molar amount of the second protein. In some embodiments, the formulation comprises the second protein (the unconjugated protein) at between about 15% and about 25% of a total molar amount of the conjugate and the second protein, wherein the total molar amount comprises a sum of the first molar amount of the conjugate and the second molar amount of the second protein. In some embodiments, the formulation comprises the second protein (the unconjugated protein) at between about 25% and about 35% of a total molar amount of the conjugate and the second protein, wherein the total molar amount comprises a sum of the first molar amount of the conjugate and the second molar amount of the second protein. In some embodiments, the formulation comprises the second protein (the unconjugated protein) at about 20% of a total molar amount of the conjugate and the second protein, wherein the total molar amount comprises a sum of the first molar amount of the conjugate and the second molar amount of the second protein. In some embodiments, the formulation comprises the second protein (the unconjugated protein) at about 30% of a total molar amount of the conjugate and the second protein, wherein the total molar amount comprises a sum of the first molar amount of the conjugate and the second molar amount of the second protein. In some embodiments, any formulation or composition provided herein comprises the second protein (the unconjugated protein) at more than 5% of a total molar amount of the conjugate and the second protein, wherein the total molar amount comprises a sum of the first molar amount of the conjugate and the second molar amount of the second protein. In some embodiments, any formulation or composition provided herein comprises the second protein (the unconjugated protein) at more than 10% of a total molar amount of the conjugate and the second protein, wherein the total molar amount comprises a sum of the first molar amount of the conjugate and the second molar amount of the second protein. In some embodiments, any composition or formulation herein includes two or more (e.g., 2, 3, 4, 5 or more) different second proteins (or unconjugated proteins), where the second molar amount is the sum of the molar amounts of the two or more different second proteins.

Provided herein is a formulation (or therapeutically acceptable compositions) comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the formulation (or therapeutically acceptable compositions) comprises the second protein at about 1% or more (e.g., about 5-90%, 15-25%, 25-35%, 25-40%, etc.) of a total molar amount of the conjugate and the second protein, wherein the total molar amount comprises a sum of the first molar amount and the second molar amount, wherein the formulation (or therapeutically acceptable compositions) has a pH that is about 0.5 pH units (e.g., above or below) or more from the isoelectric point (pI) of the second protein, wherein the formulation (or therapeutically acceptable compositions) has a reduced viscosity and/or an enhanced injectability compared to a reference formulation (or reference composition) comprising the conjugate at the total molar amount. In some embodiments, the reference formulation or composition is one that includes the conjugate at the total molar amount and effectively does not include the second protein (or includes the second protein at less than 1% of the total molar amount), but is otherwise the same as the formulation or composition for which it serves as a reference. In some embodiments, the reference formulation or composition is a therapeutically acceptable reference composition.

Also provided is a low-viscosity formulation (or therapeutically acceptable compositions) of a protein conjugate, comprising: a first molar amount of a conjugate comprising a protein conjugated to a phosphorylcholine-containing polymer; a second molar amount of the protein that is not conjugated to the phosphorylcholine-containing polymer; and a pharmaceutically acceptable carrier, wherein the formulation (or therapeutically acceptable compositions) has a pH that is about 0.5 pH units away (e.g., above or below) or more from the isoelectric point (pI) of the protein, wherein the formulation (or therapeutically acceptable compositions) has reduced viscosity and/or an enhanced injectability compared to a reference formulation (or reference composition) comprising the conjugate at a total molar amount that is the sum of the first and second molar amounts. In some embodiments, the reference formulation or composition is one that includes the conjugate at the total molar amount and effectively does not include the second protein (or includes the second protein at less than 1% of the total molar amount), but is otherwise the same as the formulation or composition for which it serves as a reference. In some embodiments, the reference formulation or composition is a therapeutically acceptable reference composition.

The formulation (or therapeutically acceptable compositions) of the present disclosure can have any suitable viscosity. In some embodiments, the formulation has a viscosity of, or of about 1000 mPas·s or less, e.g., about 900 mPas·s or less, about 800 mPas·s or less, about 700 mPas·s or less, about 600 mPas·s or less, about 500 mPas·s or less, about 400 mPas·s or less, about 300 mPas·s or less, about 200 mPas·s or less, about 100 mPas·s or less, or about 200 mPas·s or more, 300 mPas·s or more, about 400 mPas·s or more, about 500 mPas·s or more, about 600 mPas·s or more, about 700 mPas·s or more, about 800 mPas·s or more, about 900 mPas·s or more, or a viscosity in a range defined by any two of the preceding values (e.g., 100-1000 mPas·s, 200-1000 mPas·s, 200-500 mPas·s, 300-800 mPas·s, 300-600 mPas·s, 200-300 mPas·s, etc.). In some embodiments, the formulation has a viscosity of about 100 to about 300 mPas·s or about 200 to about 500 mPas·s. In some embodiments, the formulation has a viscosity of about 300 to about 400 mPas·s. The viscosity can be measured using any suitable option, e.g., a rotational rheometer such as a TA Instruments DHR-20.

In some embodiments, the formulation (or therapeutically acceptable composition) has a viscosity that is reduced compared to a reference formulation having a molar amount of the conjugate that is the same as the total amount of the conjugate and unconjugated protein. In some embodiments, the viscosity is reduced by about 10% or more, e.g., about 20% or more, about 30% or more, about 40% or more, about 50% or more, about 60% or more, about 70% or more, about 80% or more, about 90% or more, or by about 90% or less, about 80% or more, about 70% or less, about 60% or less, about 50% or less, about 40% or less, about 30% or less, or by a percentage in a range defined by any two of the preceding values (e.g., by about 10-90%, by about 20-80%, by about 50-80%, by about 30-70%, by about 40-90%, etc.). In some embodiments, the viscosity is reduced by about 50-80%. In some embodiments, the viscosity is reduced by about 70-80%.

In some embodiments, the reference formulation or composition (having the conjugate form only at the total molar amount) may include the conjugate (e.g., the polymer portion of the conjugate) at a sufficiently high molar amount to result in a formulation with high viscosity. In some embodiments, the reference formulation has a viscosity of, or of about 700 mPas·s or more, e.g., about 800 mPas·s or more, about 900 mPas·s or more, about 1000 mPas·s or more, about 1000 mPas·s or more, or about 1500 mPas·s or less, about 1400 mPas·s or less, about 1300 mPas·s or less, about 1200 mPas·s or less, about 1100 mPas·s or less, about 1000 mPas·s or less, or a viscosity in a range defined by any two of the preceding values (e.g., about 700-1500 mPas·s, about 800-1300 mPas·s, about 900-1200, mPas·s, about 1000-1300 mPas·s, etc.). In some embodiments, where the first protein and the second protein are the same or substantially the same protein (having substantially the same activity), replacing a portion of the conjugated protein with the unconjugated protein, while keeping the total molar amount the same, provides a formulation having lower viscosity and the same total amount of the protein as the reference formulation.

Formulations (or therapeutically acceptable compositions) of the present disclosure can have low turbidity (e.g., a visually clear solution). In some embodiments, adding an unconjugated protein to a formulation of a conjugate (e.g., a protein conjugated to a phosphorylcholine-containing polymer as provided herein) results in a mixed formulation that is turbid (e.g., cloudy visual appearance). In some embodiments, the turbidity of a formulation with a mixture of unconjugated and conjugated proteins is reduced when the pH of the formulation is not at or is not around (e.g., at least 0.5 pH units away from) the isoelectric point (pI) of the unconjugated protein. Where it is desirable for the formulation to be clear (e.g., when the formulation is for intraocular administration), a low-turbidity formulation of a mixture of unconjugated and conjugated proteins is obtained when the pH of the formulation is different from (e.g., at least 0.5 pH units above or below) the pI of the unconjugated protein.

Provided is a formulation (or therapeutically acceptable composition) comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the second protein is present in the formulation at about 1% or more (e.g., about 5-90%, 15-25%, 25-35%, 25-40%, etc.) of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the formulation has a reduced turbidity compared to a reference formulation comprising the first molar amount of the conjugate and the second molar amount of the second protein at a pH about the same as (e.g., within 0.05, 0.1, 0.15, 0.2, 0.3, 0.4, or 0.5 pH units of) the pI of the second protein. In some embodiments, the formulation has a reduced turbidity compared to a reference formulation comprising the first molar amount of the conjugate and the second molar amount of the second protein at a pH within 0.5 pH units of the pI of the second protein. The turbidity can be measured using any suitable option. For example, turbidity can be measured at OD 600 nm using a plate reader and calibrating the OD values against a suitable standard (e.g., a 4000 NTU Formazin calibration standard). In some embodiments, the turbidity is reduced by about 10% or more, e.g., about 20% or more, about 30% or more, about 40% or more about 50% or more about 60% or more about 70% or more, about 80% or more, about 90% or more, or about 100%, or by a percentage in a range defined by any two of the preceding values (e.g., 10-100%, 10-50%, 30-70%, 50-100%, 80-100%, etc.).

In some embodiments, the formulation (or therapeutically acceptable composition) is substantially free of turbidity. In some embodiments, the formulation is free of turbidity based on visual inspection. In some embodiments, a formulation or composition that is substantially free of turbidity is free of turbidity based on visual inspection. In some embodiments, the prepared formulation has a turbidity that, when expressed as NTU values as measured at OD 600 nm and calibrated against a 4000 NTU Formazin calibration standard, is (or corresponds to) about 500 Nephelometric Turbidity Units (NTU) or less, e.g., about 400 NTU or less, about 300 NTU or less, about 200 NTU or less, about 100 NTU or less, or about −50 NTU or more, about 0 NTU or more, about 100 NTU or more, about 200 NTU or more, about 300 NTU or more, about 400 NTU or more, or a turbidity measure in a range defined by any two of the preceding values (e.g., about −50-500 NTU, about 0-400 NTU, about 100-300 NTU, or about 50-400 NTU). In some embodiments, the prepared formulation has a turbidity that, when expressed as NTU values as measured at OD 600 nm and calibrated against a 4000 NTU Formazin calibration standard, is (or corresponds to) about 300 NTU or less. In some embodiments, the prepared formulation has a turbidity that, when expressed as NTU values as measured at OD 600 nm and calibrated against a 4000 NTU Formazin calibration standard, is (or corresponds to) about 200 NTU or less. In some embodiments, the prepared formulation has a turbidity that, when expressed as NTU values as measured at OD 600 nm and calibrated against a 4000 NTU Formazin calibration standard, is (or corresponds to) about 100 NTU or less. In some embodiments, the formulation or composition is free of turbidity based on visual inspection when the turbidity expressed as NTU values as measured at OD 600 nm and calibrated against a 4000 NTU Formazin calibration standard is, is about, or is at most 500, 450, 400, 350, 300, 250, 200, 150, 100, or 50 NTU.

Also provided herein is a pharmaceutical formulation that includes: a first molar amount of a conjugate comprising a protein conjugated to a phosphorylcholine-containing polymer; a second molar amount the protein that is not conjugated to the phosphorylcholine-containing polymer; and a pharmaceutically acceptable carrier, wherein the formulation comprises the protein that is not conjugated to the phosphorylcholine-containing polymer at about 1% or more (e.g., about 5-90%, 15-25%, 25-35%, 25-40%, etc.) of a total molar amount of the conjugate and unconjugated proteins, wherein the total molar amount comprises a sum of the first molar amount and the second molar amount, wherein the formulation has a pH that is about 0.5 pH units away or more from the isoelectric point (pI) of the protein, wherein the formulation is substantially free of turbidity.

Provided herein is a formulation that includes: a phosphorylcholine-containing polymer present in the formulation at about 100 mg/mL or higher; and a protein that is not conjugated to a phosphorylcholine-containing polymer, wherein the protein is present in the formulation at a second molar amount, wherein the protein is present in the formulation at about 1% or more (e.g., about 5-90%, 15-25%, 25-35%, 25-40%, etc.) of a total molar amount of the polymer and the protein, wherein the total molar amount comprises a sum of the first molar amount and the second molar amount, wherein the formulation has a pH that is about 0.5 pH units away or more from the isoelectric point (pI) of the protein. In some embodiments, the polymer is conjugated to a protein (e.g., an antibody, fusion construct, etc.).

Also provided is a formulation (or therapeutically acceptable composition) comprising: a first molar amount of a conjugate comprising a first protein conjugated to a polymer; and a second molar amount of a second protein that is not conjugated to a polymer, wherein the formulation comprises the second protein at about 1% or more (e.g., about 5-90%, 15-25%, 25-35%, 25-40%, etc.) of a total molar amount of the first protein and the second protein, wherein the total molar amount comprises a sum of the first molar amount and the second molar amount. In some embodiments, the formulation comprises the second protein at about 1-90%, about 5-80%, about 10-95%, about 15-30%, about 5-50%, or about 10-40%, of the total molar amount of the conjugate and the second protein. In some embodiments, the polymer is a phosphorylcholine-containing polymer. In some embodiments, the formulation comprises the second protein at about 5-50%, or about 15-30% of the total molar amount of the conjugate and the second protein. In some embodiments, the polymer is a phosphorylcholine-containing polymer.

Also provided is a formulation (or therapeutically acceptable composition) comprising: a conjugate comprising a first protein conjugated to a polymer; and a second protein that is not conjugated to a polymer, wherein a first molar amount of the conjugate and a second molar amount of the second protein has been combined in the formulation such that the second molar amount is about 1% or more (e.g., about 5-90%, 15-25%, 25-35%, 25-40%, etc.) of a total molar amount of the conjugate and the second protein, wherein the total molar amount comprises a sum of the first molar amount and the second molar amount. In some embodiments, the formulation is prepared by combining the first molar amount of the conjugate with the second molar amount of the second protein that is not conjugated to a polymer, such that the second molar amount is at the specified percentage of the sum of the first molar amount and the second molar amount (e.g., specified percentage of the total molar amount). In some embodiments, the second molar amount is about 1-90%, about 5-90%, about 5-80%, about 10-95%, about 15-30%, about 5-50%, or about 10-40%, of the total molar amount of the conjugate and the second protein. In some embodiments, the second molar amount is about 5-50% of the total molar amount of the conjugate and the second protein. In some embodiments, the second molar amount is about 15-30% of the total molar amount of the conjugate and the second protein. In some embodiments, the polymer is a phosphorylcholine-containing polymer.

In some embodiments, the formulation or composition has been prepared by combining the conjugate at a percent composition of about 1% or more (e.g., about 5-93%, 15-25%, 25-35%, 25-40%, etc.) of the second protein relative to the total protein mass weight concentration of the first protein and the second protein, where the remainder of the total protein mass weight concentration includes the first protein. For example, for a total mass weight concentration of 50 mg/mL, the therapeutically acceptable composition can be prepared by combining an amount of the second protein that corresponds to 10 mg/mL in the final composition (at percent composition of 20%) with an amount of the conjugate that corresponds to 40 mg/mL of the first protein (as the conjugate, excluding any contribution of the polymer to the mass weight concentration calculation) in the final composition.

In some embodiments, the conjugate includes a first protein conjugated to a polymer, wherein the polymer includes one or more of: polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(1-hydroxymethyethylene hydroxymethylformal) (PHF), a zwitterionic polymer, a phosphorylcholine containing polymer and a polymer comprising MPC, Poly (Glyₓ-Sery), Hyaluronic acid (HA), Heparosan polymers (HEP), Fleximers, Dextran, and Poly-sialic acids (PSA).

Also provided is a formulation (or therapeutically acceptable composition) comprising: a conjugate comprising a first protein conjugated to a phosphorylcholine-containing polymer, wherein the polymer has 9 arms and a molecular weight of between 600,000 and 1,000,000 Da, wherein the polymer is present in the formulation at about 100 mg/mL or more; and a second protein that is not conjugated to a polymer, wherein the second protein is present in the formulation at about 5-15 mg/mL. In some embodiments, the first and second proteins are a therapeutic protein. In some embodiments, the first protein and second protein are the same (e.g., are at least, or at least about 85%, 90%, 95%, 97%, 98%, 99%, or are about 100% identical in amino acid sequence). In some embodiments, the first protein and second protein are different proteins.

In some embodiments, the phosphorylcholine-containing polymer is present in the formulation at about 100 mg/mL or more, e.g., about 150 mg/mL or more, about 200 mg/mL or more, about 250 mg/mL or more, about 300 mg/mL or more, about 350 mg/mL or more, about 400 mg/mL or more, about 450 mg/mL or more, or a concentration in range defined by any two of the preceding values (e.g., 100-450 mg/mL, 150-400 mg/mL, 200-400 mg/mL, 250-450 mg/mL, 300-450 mg/mL, etc.). In some embodiments, the phosphorylcholine-containing polymer is present in the formulation at between about 150-400 mg/mL. In some embodiments, the phosphorylcholine-containing polymer is present in the formulation at between about 200-300 mg/mL.

In some embodiments, the polymer has a molecular weight of about 100,000 Da or more, e.g., about 150,000 Da or more, about 200,000 Da or more, about 350,000 Da or more, about 400,000 Da or more, about 450,000 Da or more, about 500,000 Da or more, about 550,000 Da or more, about 600,000 Da or more, about 650,000 Da or more, about 700,000 or more, about 750,000 Da or more, about 800,000 Da or more, about 850,000 Da or more, about 900,000 Da or more, about 950,000 Da or more, about 1,000,000 Da or more, or a molecular weight in a range defined by any two of the preceding values (e.g., 100,000-1,000,000 Da, 300,000-950,000 Da, 400,000-800,000 Da, 500,000-750.000 Da, 600,000-700,000 Da, 600,000-1,000,000 Da, etc.). In some embodiments, the polymer has a molecular weight in the range of about 700,000 to about 800,000 Da. In some embodiments, the polymer is any of the polymers disclosed herein. In some embodiments, the polymer is OG1801 or OG1802.

In some embodiments, formulations (or therapeutically acceptable compositions) of the present disclosure (e.g., having a polymer or polymer-conjugated protein and unconjugated protein) have a pH that is different (e.g., higher or lower) from the pI of the unconjugated protein (e.g., protein that is not conjugated to a phosphorylcholine-containing polymer). In some embodiments, the pH of the formulation is about 0.5 pH units away or more from the pI of the unconjugated protein (e.g., protein that is not conjugated to the phosphorylcholine-containing polymer). In some embodiments, the pH of the formulation is about 0.5 pH units away or more, e.g., about 0.6 pH units away or more, about 0.7 pH units away or more, about 0.8 pH units away or more, about 0.9 pH units away or more, about 1.0 pH units away or more, about 1.1 pH units away or more, about 1.2 pH units away or more, about 1.3 pH units away or more, about 1.4 pH units away or more, about 1.5 pH units away or more, about 1.6 pH units away or more, about 1.7 pH units away or more, about 1.8 pH units away or more, about 1.9 pH units away or more, about 2.0 pH units away or more, about 2.1 pH units away or more, about 2.2 pH units away or more, about 2.3 pH units away or more, about 2.4 pH units away or more, about 2.5 pH units away or more, about 2.6 pH units away or more, about 2.7 pH units away or more, about 2.8 pH units away or more, about 2.9 pH units away or more, about 3.0 pH units away or more, about 3.2 pH units away or more, about 3.4 pH units away or more, about 3.6 pH units away or more, about 3.8 pH units away or more, about 4.0 pH units away or more, about 4.5 pH units away or more, about 5.0 pH unit away or more, from the pI of the unconjugated protein (e.g., protein that is not conjugated to a phosphorylcholine-containing polymer), or away from the pI of the unconjugated protein by a pH unit in a range defined by any two of the preceding values (e.g., 0.5-5.0 pH units away, 1.0-4.0 pH units away, 1.5-3.0 pH units away, 2.0-3.0 pH units away, 1.5-3.6 pH units away, etc.). In some embodiments, the pH of the formulation is between about 2.0-3.0 pH units away from the pI of the unconjugated protein (e.g., protein that is not conjugated to the phosphorylcholine-containing polymer).

In some embodiments, formulations (or therapeutically acceptable compositions) of the present disclosure (e.g., having a polymer or polymer-conjugated protein and unconjugated protein) have a pH that is more acidic than the pI of the unconjugated protein (e.g., protein that is not conjugated to a phosphorylcholine-containing polymer). In some embodiments, the pH of the formulation is about 0.5 pH units less or lower, e.g., about 0.6 pH units less or lower, about 0.7 pH units less or lower, about 0.8 pH units less or lower, about 0.9 pH units less or lower, about 1.0 pH units less or lower, about 1.1 pH units less or lower, about 1.2 pH units less or lower, about 1.3 pH units less or lower, about 1.4 pH units less or lower, about 1.5 pH units less or lower, about 1.6 pH units less or lower, about 1.7 pH units less or lower, about 1.8 pH units less or lower, about 1.9 pH units less or lower, about 2.0 pH units less or lower, about 2.1 pH units less or lower, about 2.2 pH units less or lower, about 2.3 pH units less or lower, about 2.4 pH units less or lower, about 2.5 pH units less or lower, about 2.6 pH units less or lower, about 2.7 pH units less or lower, about 2.8 pH units less or lower, about 2.9 pH units less or lower, about 3.0 pH units less or lower, about 3.2 pH units less or lower, about 3.4 pH units less or lower, about 3.6 pH units less or lower, about 3.8 pH units less or lower, about 4.0 pH units less or lower, about 4.5 pH units less or lower, about 5.0 pH units less or lower from the pI of the unconjugated protein (e.g., protein that is not conjugated to the phosphorylcholine-containing polymer), or lower than the pI of the unconjugated protein by a pH unit in a range defined by any two of the preceding values (e.g., 0.5-5.0 pH units lower, 1.0-4.0 pH units lower, 1.5-3.0 pH units lower, 2.0-3.0 pH units lower, 1.5-3.6 pH units lower, etc.). In some embodiments, the pH of the formulation is between about 2.0-3.0 pH units lower than the pI of the unconjugated protein (e.g., protein that is not conjugated to the phosphorylcholine-containing polymer).

In some embodiments, formulations (or therapeutically acceptable compositions) of the present disclosure (e.g., having a polymer or polymer-conjugated protein and unconjugated protein) have a pH that is more basic than the pI of the unconjugated protein (e.g., protein that is not conjugated to the phosphorylcholine-containing polymer). In some embodiments, the pH of the formulation is about 0.5 pH units more or higher, e.g., about 0.6 pH units more or higher, about 0.7 pH units more or higher, about 0.8 pH units more or higher, about 0.9 pH units more or higher, about 1.0 pH units more or higher, about 1.1 pH units more or higher, about 1.2 pH units more or higher, about 1.3 pH units more or higher, about 1.4 pH units more or higher, about 1.5 pH units more or higher, about 1.6 pH units more or higher, about 1.7 pH units more or higher, about 1.8 pH units more or higher, about 1.9 pH units more or higher, about 2.0 pH units more or higher, about 2.1 pH units more or higher, about 2.2 pH units more or higher, about 2.3 pH units more or higher, about 2.4 pH units more or higher, about 2.5 pH units more or higher, about 2.6 pH units more or higher, about 2.7 pH units more or higher, about 2.8 pH units more or higher, about 2.9 pH units more or higher, about 3.0 pH units more or higher, about 3.2 pH units more or higher, about 3.4 pH units more or higher, about 3.6 pH units more or higher, about 3.8 pH units more or higher, about 4.0 pH units more or higher, about 4.5 pH units more or higher, about 5.0 pH units more or higher, than the pI of the unconjugated protein (e.g., protein that is not conjugated to the phosphorylcholine-containing polymer), or greater than the pI of the unconjugated protein by a pH unit in a range defined by any two of the preceding values (e.g., 0.5-4.0 pH units greater, 1.0-3.0 pH units greater, 1.5-3.0 pH units greater, 2.0-3.0 pH units greater, 1.5-3.6 pH units greater, etc.). In some embodiments, the pH of the formulation is between about 2.0-3.0 pH units greater than the pI of the unconjugated protein (e.g., protein that is not conjugated to the phosphorylcholine-containing polymer).

In some embodiments, the difference between the pH of a formulation (or therapeutically acceptable composition) containing a mixture of a polymer or polymer-conjugated protein and an unconjugated protein (e.g., protein that is not conjugated to a phosphorylcholine-containing polymer), as provided herein, and the pI of the unconjugated protein depends on the relative amounts (e.g., molar amounts) of the polymer/polymer-conjugate and the unconjugated protein in the formulation. Without being bound by theory, in general, the higher the relative amount (e.g., molar amount) of the unconjugated protein compared to the polymer/polymer-conjugate, the further away the pH will be relative to the pI of the unconjugated protein (for a given concentration (e.g., molar amount) of the polymer or conjugate above a threshold in the formulation). Without being bound by theory, when the pH is too close to the pI of the unconjugated protein, the formulation can turn turbid. In some embodiments, a formulation having a pH at about 4.0 or lower is substantially free of turbidity regardless of the concentration of polymer/polymer conjugate, or the concentration of the unconjugated protein.

Also provided herein is a formulation (or therapeutically acceptable composition) that includes: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the difference between the pI of the second protein and the pH of the formulation in the acidic or basic direction is selected to be greater than the minimum difference in the corresponding acidic or basic direction between the pI of the second protein and the pH for a reference formulation comprising: a third molar amount of the conjugate comprising the first protein conjugated to the phosphorylcholine-containing polymer; a fourth molar amount of the second protein that is not conjugated to the phosphorylcholine-containing polymer; and the pharmaceutically acceptable carrier, wherein a first total molar amount comprising a sum of the first molar amount and the second molar amount, and a second total molar amount comprising a sum of the third molar amount and the fourth molar amount are substantially the same, wherein the second molar amount is greater than the fourth molar amount, wherein the reference formulation is substantially free of turbidity. Without being bound by theory, between two formulations each having an unconjugated protein (e.g., antibody or fusion construct) and a polymer conjugate, and the same total molar amount of the protein (unconjugated and conjugated), and when the concentration of polymer is sufficiently high, the difference between the pI of the unconjugated protein and the pH of the formulation having a higher percentage of the total molar amount of the unconjugated protein (in either the acidic or basic direction) will generally be greater than the minimum difference between the pI of the unconjugated protein and the pH of the formulation having the lower percentage of the total molar amount of the unconjugated protein (in the corresponding acidic or basic direction) to maintain a clear formulation that is substantially free of turbidity. For example and without limitation, for two formulations of the same unconjugated/conjugated protein pair (where the protein component of the conjugate may or may not be the same as the unconjugated protein), the same total molar amount of unconjugated and conjugated protein and a sufficiently high concentration of polymer from the conjugate, the pH of the formulation may be lower in the formulation having a higher proportion of unconjugated to conjugated protein if the pH is below the pI of the unconjugated protein, to maintain a clear solution. For example and without limitation, a formulation containing the unconjugated protein (having a pI of around 7.4) at about 5% of the total molar amount of unconjugated protein and conjugate, and at least about 200 mg/mL polymer, may be clear up to around pH 6, and another formulation having the unconjugated protein at 10% or 15% of the total molar amount, and at least about 200 mg/mL polymer, may be clear up to around pH 5, but turbid at around pH 6. In some embodiments, the pH of the formulation and the reference formulation is each at least about 4.5 for this trend. In some embodiments, the pH of the formulation is selected to be lower than the maximum pH for the reference formulation (e.g., for maintaining a clear formulation), wherein the pH of the formulation and a maximum pH for the reference formulation are lower than the pI of the second protein. In some embodiments, the pH of the formulation is selected to be higher than a minimum pH for the reference formulation (e.g., for maintaining a clear formulation), wherein the pH of the formulation and the minimum pH for the reference formulation are higher than the pI of the second protein. In some embodiments, the pH of the formulation and the reference formulation is each at most about 8.5 for this trend. In some embodiments, the pH of the formulation and the reference formulation is between about 4.5 and 8.5 for this trend. In some embodiments, the concentration of the polymer component of the conjugate in the formulation and the reference formulation is each at least about 100 mg/mL (e.g., about 150 mg/mL, about 200 mg/mL, about 250 mg/mL, or about 300 mg/mL, or in a range of 150-300 mg/mL, e.g., 200-300 mg/mL, or 250-300 mg/mL).

The formulation (or therapeutically acceptable composition) can have any suitable pH. In some embodiments, the pH of the formulation is about 3.0 or higher, about 3.5 or higher, about 4.0 or higher, about 4.5 or higher, about 5.0 or higher, about 5.5 or higher, about 6.0 or higher, about 6.5 or higher, about 7.0 or higher, about 7.5 or higher, about 8.0 or higher, about 8.5 or higher, about 9.0 or higher, about 9.5 or higher, about 10.0 or higher, or about 10.5 or higher, or about 12.0 or lower, about 11.5 or lower, about 11.0 or lower, about 10.5 or lower, about 10.0 or lower, about 9.5 or lower, about 9.0 or lower, about 8.5 or lower, about 8.0 or lower, about 7.5 or lower, about 7.0 or lower, about 6.5 or lower, about 6.0 or lower, about 5.5 or lower, about 5.0 or lower, about 4.5 or lower, about 4.0 or lower, or a pH in a range defined by any two of the preceding values (e.g., 3.0-12.0, 3.5-10.0, 4.0-6.0, 8-12, 5.0-8.0, 9-10, etc.). In some embodiments, the pH of the formulation is in the range of about pH 4 to about pH 5.5. In some embodiments, the pH of the formulation is in the range of about pH 4.5 to about pH 5.3. In some embodiments, the pH of the formulation is about 4.0, 4.1, 4.2, 4.3, 4.4, 4.5, 4.6, 4.7, 4.8, 4.9, 5.0, 5.1, 5.2, 5.3, 5.4, or about 5.5. In some embodiments, the pH of the formulation is about 5.0. In some embodiments, the pH of the formulation is about 4.5.

In some embodiments, the unconjugated protein (e.g., the protein that is not conjugated to a phosphorylcholine-containing polymer) in the formulation has a pH of, or of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.7, 8.8, 8.9, 9.0, 9.1, 9.2, 9.3, 9.4, or about 9.5 or higher. In some embodiments, the pI of the unconjugated protein (e.g., the protein that is not conjugated to a phosphorylcholine-containing polymer) in the formulation is between about 4.0 and about 9.5, between about 5.0 and about 9.5, between about 5.5 and about 8.5, between about 6 and about 8.5, between about 7.0 and about 9.5, or between about 5.0 and about 8.0. In some embodiments, the pI of the unconjugated protein (e.g., the protein that is not conjugated to a phosphorylcholine-containing polymer) in the formulation is between about 7.0 and about 8.5, the pH of the formulation is between about 4.0 and about 5.5, and the phosphorylcholine-containing polymer is present in the formulation (e.g., as a conjugate of the protein) at a concentration of between about 200 and about 300 mg/mL. In some embodiments, the pI of the unconjugated protein (e.g., the protein that is not conjugated to a phosphorylcholine-containing polymer) in the formulation is between about 7.0 and about 8.5, the pH of the formulation is between about 4.8 and about 5.2, and the phosphorylcholine-containing polymer is present in the formulation (e.g., as a conjugate of the protein) at a concentration of between about 200 and about 300 mg/mL.

In some embodiments, a formulation (or therapeutically acceptable composition, pharmaceutical composition, or therapeutic composition) provided herein is storage stable. In some embodiments, a formulation (or therapeutically acceptable composition, pharmaceutical composition, or therapeutic composition) provided herein shows long-term stability (e.g., when stored under standard storage conditions). In some embodiments, components of the formulation (the polymer/polymer conjugate and unconjugated protein) are soluble, and the formulation is a clear solution. In some embodiments, a storage stable formulation remains substantially free of turbidity. In some embodiments, the formulation is stable (e.g., clarity of the solution, structurally in terms of the protein and polymer components, and/or functionally with respect to the protein activity) after extended periods of time after being formulated at standard storage temperatures. The formulation (or therapeutic composition, pharmaceutical composition, or therapeutically acceptable composition) can be stored at any suitable temperature. In some embodiments, the formulation (or therapeutic composition, pharmaceutical composition, or therapeutically acceptable composition) is stored at, at about, or at most at 0, 1, 2, 3, 4, 5, 6, 7, 8, 9, 10, 11, 12, 13, 14, 15, 16, 17, 18, 19, 20, 21, 22, 23, 24, 25, 30, 35, 40° C., or optionally it is stored at a temperature in a range defined by any two of the preceding values (e.g., 0-40° C., 0-10° C., 10-20° C., 20-30° C., 30-40° C., etc.). In some embodiments, the formulation (or therapeutic composition, pharmaceutical composition, or therapeutically acceptable composition) is stored at a temperature in the range of 0-10° C. In some embodiments, the formulation (or therapeutic composition, pharmaceutical composition, or therapeutically acceptable composition) is stored at a temperature in the range of 10-20° C. In some embodiments, the formulation (or therapeutic composition or therapeutically acceptable composition) is stored at or at about 5° C. In some embodiments, the formulation (or therapeutic composition, pharmaceutical composition, or therapeutically acceptable composition) is stored at or at about 25° C. In some embodiments, the formulation (or therapeutic composition, pharmaceutical composition, or therapeutically acceptable composition) is stored at room temperature. In some embodiments, the formulation (or therapeutically acceptable composition) is stored under ambient atmospheric pressure. In some embodiments, the formulation (or therapeutic composition, pharmaceutical composition, or therapeutically acceptable composition) is stored at, at about, or at least at −5° C., −10° C., −15° C., −20° C., or −25° C.

In some embodiments, the formulation is stable when a measured feature of the formulation (e.g., turbidity, structural integrity and concentration of the protein and polymer components, activity of the protein) remains within at least about 20%, at least about 15%, at least about 10%, or at least about 5% of the originally measured level when the formulation was initially prepared. In some embodiments, the formulation is stable when a measured feature of the formulation (e.g., turbidity, structural integrity and concentration of the protein and polymer components, activity of the protein) remains within at least about 20%, at least about 15%, at least about 10%, or at least about 5% of the originally measured level at an initial time point. In some embodiments, the formulation (or therapeutically acceptable composition) is storage stable with respect to at least one of the following functional properties: turbidity, percent impurities, concentration of intact protein, concentration of intact polymer, activity of protein (e.g., inhibition of VEGF-A binding to VEGFR). In some embodiments, “impurities” refer to product related impurities such as degraded, aggregated, unconjugated (e.g., where the product of interest is conjugated), or modified proteins. In some embodiments, product unrelated impurities such as Host Cell Protein, endotoxin, Host cell DNA, are not considered “impurities” as defined herein. In some embodiments, the formulation is stable (e.g., less than about 5% or about 10% total impurities, and/or IC₅₀ for competitive binding to VEGF bound to a VEGFR that is no more than about 10% or about 15% lower than a control formulation of the conjugate only) for at least about 1 month, at least about 2 months, at least about 3 months, at least about 4 months, at least about 5 months, at least about 6 months, at least about 9 months, at least about 12 months, at least about 16 months, at least about 20 months, at least about 24 months, at least about 36 months, at least about 48 months, or longer, or optionally, it is stable for a period of time in a range defined by any two of the preceding values (e.g., 1-48 months, 1-24 months, 1-12 months, 1-6 months, 3-9 months, 4-12 months, 12-24 months, 12-36 months, etc.), when stored at about 5° C. In some embodiments, the formulation is stable (e.g., less than about 5% or about 10% total impurities, and/or IC₅₀ for competitive binding to VEGF bound to a VEGFR that is no more than about 10% or about 15% lower than a control formulation of the conjugate only) for at least about 1 month, at least about 2 months, at least about 3 months, at least about 4 months, at least about 5 months, at least about 6 months, at least about 9 months, at least about 12 months, at least about 16 months, at least about 20 months, at least about 24 months, or longer, or optionally, it is stable for a period of time in a range defined by any two of the preceding values (e.g., 1-24 months, 1-20 months, 1-12 months, 1-6 months, 3-9 months, 4-12 months, 12-24 months, etc.), when stored at about 25° C. In some embodiments, the formulation is stable (e.g., less than about 5% or about 10% total impurities, and/or IC₅₀ for competitive binding to VEGF bound to a VEGFR that is no more than about 10% or about 15% lower than a control formulation of the conjugate only) for at least about 1 month, at least about 2 months, at least about 3 months, at least about 4 months, at least about 5 months, at least about 6 months, at least about 9 months, at least about 12 months, at least about 16 months, at least about 20 months, at least about 24 months, at least about 36 months, or longer, or optionally, it is stable for a period of time in a range defined by any two of the preceding values (e.g., 1-12 months, 1-6 months, 3-9 months, 4-12 months, etc.), when stored at room temperature. In some embodiments, the formulation is stable for, for about, or for at least 3 months when stored at about 5° C. In some embodiments, the formulation is stable for, for about, or for at least 6 months when stored at about 5° C. In some embodiments, the formulation is stable for, for about, or for at least 9 months when stored at about 5° C. In some embodiments, the formulation is stable for, for about, or for at least 12 months when stored at about 5° C. In some embodiments, the formulation is stable for, for about, or for at least 24 months when stored at about 5° C. In some embodiments, the formulation is stable for, for about, or for at least 3 months when stored at about 25° C. In some embodiments, the formulation is stable for, for about, or for at least 6 months when stored at about 25° C. In some embodiments, the formulation is stable for, for about, or for at least 9 months when stored at about 25° C. In some embodiments, the formulation is stable for, for about, or for at least 12 months when stored at about 25° C.

In some embodiments, the formulation (or therapeutically acceptable composition) is storage stable with respect to at least one of the following measures: color and clarity by visual inspection; aggregation or degradation of the unconjugated protein and/or the conjugated protein at different temperature as measured by size exclusion chromatography (SEC-HPLC), ion-exchanger chromatography (IEX-HPLC), SEC with Multi-angle light scattering (MALS) detector, or a Tandem HPLC method that allows monitoring of the stability of either the conjugate or free protein population separately; potency; and maintenance of the percent unconjugated protein (e.g., percent composition or % of the total molar amount) in the formulation.

In some embodiments, the formulation or composition comprises a mixture, by mass weight concentration, of 5% to 10% unconjugated protein (e.g. unconjugated antibody or unconjugated fusion construct) with the remainder comprising the conjugated protein (e.g., conjugated antibody or conjugated fusion construct). In some embodiments, the amount of the unconjugated protein relative to the total amount of unconjugated and conjugated protein in the formulation is expressed as a percentage by mass weight concentration when the unconjugated protein and the protein portion of the conjugated protein has the same or similar (e.g., within about 10% of each other) molecular weight. In some embodiments, the composition comprises a mixture, by mass weight concentration of 5% to 15% unconjugated protein (e.g. unconjugated antibody) with the remainder comprising the conjugated protein. In some embodiments, the composition comprises a mixture of the two proteins (e.g., antibodies and/or fusion constructs) of the same molecular weight, of which 15% to 25% unconjugated protein (e.g. unconjugated antibody) by mass weight concentration with the remainder comprising the conjugated protein. In some embodiments, the composition comprises a mixture of two antibodies of the same molecular weight, of which 15-25% of unconjugated protein (e.g. unconjugated antibody) concentration in mass weight concentration (e.g. Gram/liter) of the total protein concentration in the mixture. In some embodiments, the composition comprises a mixture of the two proteins (e.g., antibodies and/or fusion constructs) by mass weight concentration of 25% to 35% unconjugated protein (e.g. unconjugated antibody) with the remainder comprising the conjugated protein. In some embodiments, the composition comprises a mixture of the two proteins (e.g., antibodies and/or fusion constructs) by mass weight concentration of 35% to 45% unconjugated protein (e.g. unconjugated antibody) with the remainder comprising the conjugated protein. In some embodiments, the composition comprises a mixture of the two proteins (e.g., two antibodies) by mass weight concentration of 5% to 20% unconjugated protein (e.g. unconjugated antibody) with the remainder comprising the conjugated protein. In some embodiments, the composition comprises a mixture of the two proteins (e.g., two antibodies) by mass weight concentration of 5% to 25% unconjugated protein (e.g. unconjugated antibody) with the remainder comprising the conjugated protein. In some embodiments, the composition comprises a mixture of the two proteins (e.g., antibodies and/or fusion constructs) by mass weight concentration of 5% to 30% unconjugated protein (e.g. unconjugated antibody) with the remainder comprising the conjugated protein. In some embodiments, the composition comprises a mixture of the two proteins (e.g., two antibodies) by mass weight concentration of 5% to 35% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 40% unconjugated protein (e.g., unconjugated antibody) with the remainder comprising the conjugated protein. In some embodiments, the composition comprises a mixture of the two proteins (e.g., two antibodies) by mass weight concentration of 5% to 50% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 55% unconjugated protein (e.g., unconjugated antibody) with the remainder comprising the conjugated protein.

In some embodiments, the composition or formulation comprises a mixture of the two proteins (e.g., antibodies and/or fusion constructs) by mass weight concentration of 5% to 55% unconjugated protein (e.g., unconjugated antibody or unconjugated fusion construct) with the remainder comprising the conjugated protein (e.g., the conjugated antibody or conjugated fusion construct). In some embodiments, the composition comprises a mixture of the two proteins (e.g., two antibodies) by mass weight concentration of 5% to 60% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 65% unconjugated protein (e.g., unconjugated antibody) with the remainder comprising the conjugated protein. In some embodiments, the composition comprises a mixture of the two proteins (e.g., two antibodies) by mass weight concentration of 5% to 70% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 75% unconjugated protein (e.g., unconjugated antibody) with the remainder comprising the conjugated protein. In some embodiments, the composition comprises a mixture of the two proteins (e.g., two antibodies) by mass weight concentration of 5% to 80% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 85% unconjugated protein (e.g., unconjugated antibody) with the remainder comprising the conjugated protein. In some embodiments, the composition comprises a mixture of the two proteins (e.g., antibodies and/or fusion constructs) by mass weight concentration of 5% to 90% unconjugated protein (e.g., unconjugated antibody) with the remainder comprising the conjugated protein.

In some embodiments, the percent of the conjugated to unconjugated protein (e.g., % total molar amount) is calculated by (1) measuring the conjugated protein and unconjugated protein in mg/mL; (2) converting the mg/mL values of the conjugated protein and unconjugated protein into a molecular weight measured in kDa; and (3) dividing the molecular weight of each of the conjugated protein and unconjugated protein by the total molecular weight of the conjugated protein and unconjugated protein in the composition, and multiplied by 100 to achieve a percent of the total molar amount for each.

Any of the formulations and compositions provided herein, in some embodiments, can be defined as a percent composition (e.g., in mass weight concentration) of one component relative to the total mass weight concentration of the proteins (e.g., excluding any contribution of a polymer that may be conjugated thereto) in the composition. In some embodiments, a formulation or composition defined in % total molar amount of the second protein (e.g., the unconjugated protein) can be defined in percent composition (e.g., in mass weight concentration) of the second protein relative to the total mass weight concentration of the first and second proteins, given the relevant molecular weight of each protein. In some embodiments, percent composition is measured in mass weight concentration (in other words, gram per liter or milligram per milliliter) of the free protein relative to the total mass weight concentration of the proteins (e.g., excluding any contribution of a polymer that may be conjugated thereto) in the mixture.

Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more (e.g., about 5-93%, 15-25%, 25-35%, 25-40%, etc.), wherein the composition has a pH that is about 0.5 pH units away or more from the isoelectric point (pI) of the second protein.

Also provided is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more (e.g., about 5-93%, 15-25%, 25-35%, 25-40%, etc.), wherein the composition has a pH that is about 0.5 pH units away or more from the isoelectric point (pI) of the second protein, wherein the composition has a reduced viscosity and/or an enhanced injectability compared to a reference composition comprising the conjugate, wherein the first protein of the conjugate is present in the reference composition at the total mass weight concentration of the first and second proteins in the composition. In some embodiments, the reference composition is one that includes the first protein of the conjugate at the total mass weight concentration and effectively does not include the second protein (or includes the second protein at a percent composition relative to the total mass weight concentration of the first protein and the second protein in the reference composition of less than 1%), but is otherwise the same as the composition for which it serves as a reference.

Also provided is a therapeutically acceptable composition comprising a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more (e.g., about 5-93%, 15-25%, 25-35%, 25-40%, etc.), wherein the composition has a pH that is about 0.5 pH units away or more from the isoelectric point (pI) of the second protein, wherein the composition has a reduced turbidity compared to a reference composition comprising the second protein at the percent composition relative to the total protein mass weight concentration of the first protein and the second protein in the composition, at a pH about the same as (e.g., within 0.1, 0.15, 0.2, 0.3, 0.4, or 0.5 pH units of) the pI of the second protein. In some embodiments, the composition has a reduced turbidity compared to a reference composition comprising the second protein at the percent composition relative to the total protein mass weight concentration of the first protein and the second protein in the composition. In some embodiments, the reference composition includes the first protein conjugated to a phosphorylcholine-containing polymer and the second protein that is not conjugated to a phosphorylcholine-containing polymer; and the pharmaceutically acceptable carrier, wherein the percent composition of the second protein relative to the total protein mass weight concentration of the first protein and the second protein in the reference composition is the same as the percent composition of the second protein in the therapeutically acceptable composition, and the pH is about the same as (e.g., within 0.05, 0.1, 0.15, 0.2, 0.3, 0.4, or 0.5 pH units of) the pI of the second protein. In some embodiments, the composition has a reduced turbidity compared to a reference composition comprising the second protein at the percent composition relative to the total protein mass weight concentration of the first protein and the second protein in the composition.

Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the difference between the pI of the second protein and the pH of the formulation is selected to be greater than the minimum difference between the pI of the second protein and the pH for a reference formulation comprising: the conjugate comprising the first protein conjugated to the phosphorylcholine-containing polymer; the second protein that is not conjugated to the phosphorylcholine-containing polymer; and the pharmaceutically acceptable carrier, wherein the percent composition of the second protein relative to the total protein mass weight concentration of the first protein and the second protein in the composition in the composition is higher than the percent composition of the second protein relative to the total protein mass weight concentration of the first protein and the second protein in the composition in the reference composition, wherein the reference composition is substantially free of turbidity.

Provided herein is a therapeutically acceptable composition comprising: a conjugate comprising a first protein conjugated to a phosphorylcholine-containing polymer; a second protein that is not conjugated to a phosphorylcholine-containing polymer; and a pharmaceutically acceptable carrier, wherein the percent composition of the second protein relative to the total protein mass weight concentration of the first protein and the second protein in the composition is about 1% or more (e.g., about 5-90%, 15-25%, 25-35%, 25-40%, etc.).

Further provided is a therapeutically acceptable composition comprising: a conjugate comprising a first protein conjugated to a polymer; and a second protein that is not conjugated to a polymer, wherein the second protein at a percent composition relative to the total protein mass weight concentration of the first protein and the second protein in the composition of about 1% or more (e.g., about 5-93%, 15-25%, 25-35%, 25-40%, etc.) has been combined with the conjugate, wherein the remainder of the total protein mass weight concentration comprises the first protein. In some embodiments, the composition has been prepared by combining the second protein at a percent composition of about 1% or more (e.g., about 5-93%, 15-25%, 25-35%, 25-40%, etc.) relative to the total protein mass weight concentration of the first protein and the second protein, with the conjugate such that the first protein at a percent composition of at a remainder of the total protein mass weight concentration. For example, for a total mass weight concentration of 50 mg/mL, the therapeutically acceptable composition can be prepared by combining an amount of the second protein that corresponds to 10 mg/mL in the final composition (percent composition of 20%) with an amount of the conjugate that corresponds to 40 mg/mL of the first protein (as the conjugate, excluding any contribution of the polymer to the mass weight concentration calculation) in the final composition.

In any embodiment herein, the second protein that is not conjugated to a polymer (e.g., a phosphorylcholine-containing polymer) can be referred to as the unconjugated protein, and the first protein that is conjugated to a polymer (e.g., a phosphorylcholine-containing polymer) can be referred to as the conjugated protein. In some embodiments, the percent composition of the unconjugated protein (e.g., unconjugated antibody) relative to the total protein mass weight concentration of the first protein and the second protein in the composition is between 5% and 6%, with the remainder comprising the conjugated protein. As used herein, “the remainder” denotes the portion of the total protein mass weight concentration of the composition (excluding any contribution of a polymer conjugated to the first protein to the mass weight concentration) that is not the unconjugated protein (e.g., the second protein), where the percent composition of the unconjugated protein (e.g., the second protein) and the remainder adds up to 100% of the total protein mass weight concentration. For example, where the percent composition of the unconjugated protein is between 5% and 6%, with the remainder comprising the conjugated protein, between 5% and 6% of the total mass weight concentration of the total protein concentration in the mixture is the unconjugated protein, and between 94% and 95% of the total mass weight concentration of the proteins in the mixture is the conjugated protein, where the percentages add up to 100%. In some embodiments, the percent composition of the unconjugated protein (e.g., unconjugated antibody) is between 1% and 2%, with the remainder comprising the conjugated protein. In some embodiments, the percent composition of the unconjugated protein (e.g., unconjugated antibody) is between 3% and 4%, with the remainder comprising the conjugated protein. In some embodiments, the percent composition of the unconjugated protein (e.g., unconjugated antibody) is between 6% and 7%, with the remainder comprising the conjugated protein. In some embodiments, the percent composition of the unconjugated protein (e.g., unconjugated antibody) is between 7% and 8%, with the remainder comprising the conjugated protein. In some embodiments, the percent composition of the unconjugated protein (e.g., unconjugated antibody) is between 8% and 9%, with the remainder comprising the conjugated protein. In some embodiments, the percent composition of the unconjugated protein (e.g., unconjugated antibody) is between 9% and 10%, with the remainder comprising the conjugated protein. In some embodiments, the percent composition of the unconjugated protein (e.g., unconjugated antibody) is between 10% and 11%, with the remainder comprising the conjugated protein. In some embodiments, the percent composition of the unconjugated protein (e.g., unconjugated antibody) is between 11% and 12%, with the remainder comprising the conjugated protein. In some embodiments, the percent composition of the unconjugated protein (e.g., unconjugated antibody) is between 12% and 13%, with the remainder comprising the conjugated protein. In some embodiments, the percent composition of the unconjugated protein (e.g., unconjugated antibody) is between 13% and 14%, with the remainder comprising the conjugated protein. In some embodiments, the percent composition of the unconjugated protein (e.g., unconjugated antibody) is between 14% and 15%, with the remainder comprising the conjugated protein. In some embodiments, the percent composition of the unconjugated protein (e.g., unconjugated antibody) is between 15% and 16%, with the remainder comprising the conjugated protein. In some embodiments, the percent composition of the unconjugated protein (e.g., unconjugated antibody) is between 16% and 17%, with the remainder comprising the conjugated protein. In some embodiments, the percent composition of the unconjugated protein (e.g., unconjugated antibody) is between 17% and 18%, with the remainder comprising the conjugated protein. In some embodiments, the percent composition of the unconjugated protein (e.g., unconjugated antibody) is between 18% and 19%, with the remainder comprising the conjugated protein. In some embodiments, the percent composition of the unconjugated protein (e.g., unconjugated antibody) is between 19% and 20%, with the remainder comprising the conjugated protein. In some embodiments, the percent composition of the unconjugated protein (e.g., unconjugated antibody) is between 20% and 21%, with the remainder comprising the conjugated protein. In some embodiments, the percent composition of the unconjugated protein (e.g., unconjugated antibody) is between 21% and 22%, with the remainder comprising the conjugated protein. In some embodiments, the percent composition of the unconjugated protein (e.g., unconjugated antibody) is between 22% and 23%, with the remainder comprising the conjugated protein. In some embodiments, the percent composition of the unconjugated protein (e.g., unconjugated antibody) is between 23% and 24%, with the remainder comprising the conjugated protein. In some embodiments, the percent composition of the unconjugated protein (e.g., unconjugated antibody) is between 24% and 25%.

In some embodiments, the percent composition of the unconjugated protein (e.g., unconjugated antibody or unconjugated fusion construct) is between 25% and 26%, with the remainder comprising the conjugated protein (e.g., conjugated antibody or conjugated fusion construct). In some embodiments, the percent composition of the unconjugated protein (e.g., unconjugated antibody) is between 26% and 27%, with the remainder comprising the conjugated protein. In some embodiments, the percent composition of the unconjugated protein (e.g., unconjugated antibody) is between 27% and 28%, with the remainder comprising the conjugated protein. In some embodiments, the percent composition of the unconjugated protein (e.g., unconjugated antibody) is between 28% and 29%, with the remainder comprising the conjugated protein. In some embodiments, the percent composition of the unconjugated protein (e.g., unconjugated antibody) is between 29% and 30%, with the remainder comprising the conjugated protein. In some embodiments, the percent composition of the unconjugated protein (e.g., unconjugated antibody) is between 30% and 31%, with the remainder comprising the conjugated protein. In some embodiments, the percent composition of the unconjugated protein (e.g., unconjugated antibody) is between 31% and 32%, with the remainder comprising the conjugated protein. In some embodiments, the percent composition of the unconjugated protein (e.g., unconjugated antibody) is between 32% and 33%, with the remainder comprising the conjugated protein. In some embodiments, the percent composition of the unconjugated protein (e.g., unconjugated antibody) is between 33% and 34%, with the remainder comprising the conjugated protein. In some embodiments, the percent composition of the unconjugated protein (e.g., unconjugated antibody) is between 34% and 35%, with the remainder comprising the conjugated protein. In some embodiments, the percent composition of the unconjugated protein (e.g., unconjugated antibody) is between 35% and 36%, with the remainder comprising the conjugated protein. In some embodiments, the percent composition of the unconjugated protein (e.g., unconjugated antibody) is between 36% and 37%, with the remainder comprising the conjugated protein. In some embodiments, the percent composition of the unconjugated protein (e.g., unconjugated antibody) is between 37% and 38%, with the remainder comprising the conjugated protein. In some embodiments, the percent composition of the unconjugated protein (e.g., unconjugated antibody) is between 38% and 39%, with the remainder comprising the conjugated protein. In some embodiments, the percent composition of the unconjugated protein (e.g., unconjugated antibody) is between 39% and 40%, with the remainder comprising the conjugated protein. In some embodiments, the percent composition of the unconjugated protein (e.g., unconjugated antibody) is between 40% and 41%, with the remainder comprising the conjugated protein. In some embodiments, the percent composition of the unconjugated protein (e.g., unconjugated antibody) is between 41% and 42%, with the remainder comprising the conjugated protein. In some embodiments, the percent composition of the unconjugated protein (e.g., unconjugated antibody) is between 42% and 43%, with the remainder comprising the conjugated protein. In some embodiments, the percent composition of the unconjugated protein (e.g., unconjugated antibody) is between 43% and 44%, with the remainder comprising the conjugated protein. In some embodiments, the percent composition of the unconjugated protein (e.g., unconjugated antibody) is between 44% and 45%.

In some embodiments, the percent composition of the unconjugated protein (e.g., unconjugated antibody or fusion construct) is between 45% and 46%, with the remainder comprising the conjugated protein (e.g., conjugated antibody or unconjugated fusion construct). In some embodiments, the percent composition of the unconjugated protein (e.g., unconjugated antibody) is between 46% and 47%, with the remainder comprising the conjugated protein. In some embodiments, the percent composition of the unconjugated protein (e.g., unconjugated antibody) is between 47% and 48%, with the remainder comprising the conjugated protein. In some embodiments, the percent composition of the unconjugated protein (e.g., unconjugated antibody) is between 48% and 49%, with the remainder comprising the conjugated protein. In some embodiments, the percent composition of the unconjugated protein (e.g., unconjugated antibody) is between 49% and 50%, with the remainder comprising the conjugated protein. In some embodiments, the percent composition of the unconjugated protein (e.g., unconjugated antibody) is between 50% and 51%, with the remainder comprising the conjugated protein. In some embodiments, the percent composition of the unconjugated protein (e.g., unconjugated antibody) is between 51% and 52%, with the remainder comprising the conjugated protein. In some embodiments, the percent composition of the unconjugated protein (e.g., unconjugated antibody) is between 52% and 53%, with the remainder comprising the conjugated protein. In some embodiments, the percent composition of the unconjugated protein (e.g., unconjugated antibody) is between 53% and 54%, with the remainder comprising the conjugated protein. In some embodiments, the percent composition of the unconjugated protein (e.g., unconjugated antibody) is between 54% and 55%, with the remainder comprising the conjugated protein. In some embodiments, the percent composition of the unconjugated protein (e.g., unconjugated antibody) is between 55% and 56%, with the remainder comprising the conjugated protein. In some embodiments, the percent composition of the unconjugated protein (e.g., unconjugated antibody) is between 56% and 57%, with the remainder comprising the conjugated protein. In some embodiments, the percent composition of the unconjugated protein (e.g., unconjugated antibody) is between 57% and 58%, with the remainder comprising the conjugated protein. In some embodiments, the percent composition of the unconjugated protein (e.g., unconjugated antibody) is between 58% and 59%, with the remainder comprising the conjugated protein. In some embodiments, the percent composition of the unconjugated protein (e.g., unconjugated antibody) is between 59% and 60%, with the remainder comprising the conjugated protein. In some embodiments, the percent composition of the unconjugated protein (e.g., unconjugated antibody) is between 60% and 61%, with the remainder comprising the conjugated protein. In some embodiments, the percent composition of the unconjugated protein (e.g., unconjugated antibody) is between 61% and 62%, with the remainder comprising the conjugated protein. In some embodiments, the percent composition of the unconjugated protein (e.g., unconjugated antibody) is between 62% and 63%, with the remainder comprising the conjugated protein. In some embodiments, the percent composition of the unconjugated protein (e.g., unconjugated antibody) is between 63% and 64%, with the remainder comprising the conjugated protein. In some embodiments, the percent composition of the unconjugated protein (e.g., unconjugated antibody) is between 64% and 65%.

In some embodiments, the percent composition of the unconjugated protein (e.g., unconjugated antibody or unconjugated fusion construct) is between 65% and 66%, with the remainder comprising the conjugated protein (e.g., conjugated antibody or conjugate fusion construct). In some embodiments, the percent composition of the unconjugated protein (e.g., unconjugated antibody) is between 66% and 67%, with the remainder comprising the conjugated protein. In some embodiments, the percent composition of the unconjugated protein (e.g., unconjugated antibody) is between 67% and 68%, with the remainder comprising the conjugated protein. In some embodiments, the percent composition of the unconjugated protein (e.g., unconjugated antibody) is between 68% and 69%, with the remainder comprising the conjugated protein. In some embodiments, the percent composition of the unconjugated protein (e.g., unconjugated antibody) is between 69% and 70%, with the remainder comprising the conjugated protein. In some embodiments, the percent composition of the unconjugated protein (e.g., unconjugated antibody) is between 70% and 71%, with the remainder comprising the conjugated protein. In some embodiments, the percent composition of the unconjugated protein (e.g., unconjugated antibody) is between 71% and 72%, with the remainder comprising the conjugated protein. In some embodiments, the percent composition of the unconjugated protein (e.g., unconjugated antibody) is between 72% and 73%, with the remainder comprising the conjugated protein. In some embodiments, the percent composition of the unconjugated protein (e.g., unconjugated antibody) is between 73% and 74%, with the remainder comprising the conjugated protein. In some embodiments, the percent composition of the unconjugated protein (e.g., unconjugated antibody) is between 74% and 75%, with the remainder comprising the conjugated protein. In some embodiments, the percent composition of the unconjugated protein (e.g., unconjugated antibody) is between 75% and 76%, with the remainder comprising the conjugated protein. In some embodiments, the percent composition of the unconjugated protein (e.g., unconjugated antibody) is between 76% and 77%, with the remainder comprising the conjugated protein. In some embodiments, the percent composition of the unconjugated protein (e.g., unconjugated antibody) is between 77% and 78%, with the remainder comprising the conjugated protein. In some embodiments, the percent composition of the unconjugated protein (e.g., unconjugated antibody) is between 78% and 79%, with the remainder comprising the conjugated protein. In some embodiments, the percent composition of the unconjugated protein (e.g., unconjugated antibody) is between 79% and 80%, with the remainder comprising the conjugated protein. In some embodiments, the percent composition of the unconjugated protein (e.g., unconjugated antibody) is between 80% and 81%, with the remainder comprising the conjugated protein. In some embodiments, the percent composition of the unconjugated protein (e.g., unconjugated antibody) is between 81% and 82%, with the remainder comprising the conjugated protein. In some embodiments, the percent composition of the unconjugated protein (e.g., unconjugated antibody) is between 82% and 83%, with the remainder comprising the conjugated protein. In some embodiments, the percent composition of the unconjugated protein (e.g., unconjugated antibody) is between 83% and 84%, with the remainder comprising the conjugated protein. In some embodiments, the percent composition of the unconjugated protein (e.g., unconjugated antibody) is between 84% and 85%.

In some embodiments, the percent composition of the unconjugated protein (e.g., unconjugated antibody or unconjugated fusion construct) is between 85% and 86%, with the remainder comprising the conjugated protein (e.g., conjugated antibody or conjugated fusion construct). In some embodiments, the percent composition of the unconjugated protein (e.g., unconjugated antibody) is between 86% and 87%, with the remainder comprising the conjugated protein. In some embodiments, the percent composition of the unconjugated protein (e.g., unconjugated antibody) is between 87% and 88%, with the remainder comprising the conjugated protein. In some embodiments, the percent composition of the unconjugated protein (e.g., unconjugated antibody) is between 88% and 89%, with the remainder comprising the conjugated protein. In some embodiments, the percent composition of the unconjugated protein (e.g., unconjugated antibody) is between 89% and 90%, with the remainder comprising the conjugated protein.

In some embodiments, the percent composition of the unconjugated protein (e.g., unconjugated antibody or unconjugated fusion construct) is between 5% and 25%, with the remainder comprising the conjugated protein (e.g., conjugated antibody or conjugated fusion construct). In some embodiments, the percent composition of the unconjugated protein (e.g., unconjugated antibody) is between 1% and 5%, with the remainder comprising the conjugated protein. In some embodiments, the percent composition of the unconjugated protein (e.g., unconjugated antibody) is between 5% and 12.5%, with the remainder comprising the conjugated protein. In some embodiments, the percent composition of the unconjugated protein (e.g., unconjugated antibody) is between 5% and 15%, with the remainder comprising the conjugated protein. In some embodiments, the percent composition of the unconjugated protein (e.g., unconjugated antibody) is between 5% and 17.5%. In some embodiments, the percent composition of the unconjugated protein (e.g., unconjugated antibody) is between 5% and 20%, with the remainder comprising the conjugated protein. In some embodiments, the percent composition of the unconjugated protein (e.g., unconjugated antibody) is between 5% and 25%, with the remainder comprising the conjugated protein. In some embodiments, the percent composition of the unconjugated protein (e.g., unconjugated antibody) is between 5% and 30%, with the remainder comprising the conjugated protein. In some embodiments, the percent composition of the unconjugated protein (e.g., unconjugated antibody) is between 5% and 35%, with the remainder comprising the conjugated protein. In some embodiments, the percent composition of the unconjugated protein (e.g., unconjugated antibody) is between 5% and 45%, with the remainder comprising the conjugated protein. In some embodiments, the percent composition of the unconjugated protein (e.g., unconjugated antibody) is between 5% and 50%, with the remainder comprising the conjugated protein. In some embodiments, the percent composition of the unconjugated protein (e.g., unconjugated antibody) is between 5% and 55%, with the remainder comprising the conjugated protein. In some embodiments, the percent composition of the unconjugated protein (e.g., unconjugated antibody) is between 5% and 60%, with the remainder comprising the conjugated protein. In some embodiments, the percent composition of the unconjugated protein (e.g., unconjugated antibody) is between 5% and 65%, with the remainder comprising the conjugated protein. In some embodiments, the percent composition of the unconjugated protein (e.g., unconjugated antibody) is between 5% and 70%, with the remainder comprising the conjugated protein. In some embodiments, the percent composition of the unconjugated protein (e.g., unconjugated antibody) is between 5% and 75%, with the remainder comprising the conjugated protein. In some embodiments, the percent composition of the unconjugated protein (e.g., unconjugated antibody) is between 5% and 80%, with the remainder comprising the conjugated protein. In some embodiments, the percent composition of the unconjugated protein (e.g., unconjugated antibody) is between 5% and 85%, with the remainder comprising the conjugated protein. In some embodiments, the percent composition of the unconjugated protein (e.g., unconjugated antibody) is between 5% and 90%.

In some embodiments, the percent composition of the unconjugated protein (e.g., unconjugated antibody or unconjugated fusion construct) is between 10% and 12.5%, with the remainder comprising the conjugated protein (e.g., conjugated antibody or conjugated fusion construct). In some embodiments, the percent composition of the unconjugated protein (e.g., unconjugated antibody) is between 10% and 15%, with the remainder comprising the conjugated protein. In some embodiments, the percent composition of the unconjugated protein (e.g., unconjugated antibody) is between 10% and 17.5%, with the remainder comprising the conjugated protein. In some embodiments, the percent composition of the unconjugated protein (e.g., unconjugated antibody) is between 10% and 20%, with the remainder comprising the conjugated protein. In some embodiments, the percent composition of the unconjugated protein (e.g., unconjugated antibody) is between 15% and 25%, with the remainder comprising the conjugated protein. In some embodiments, the percent composition of the unconjugated protein (e.g., unconjugated antibody) is between 10% and 25%, with the remainder comprising the conjugated protein. In some embodiments, the percent composition of the unconjugated protein (e.g., unconjugated antibody) is between 10% and 30%, with the remainder comprising the conjugated protein. In some embodiments, the percent composition of the unconjugated protein (e.g., unconjugated antibody) is between 10% and 35%, with the remainder comprising the conjugated protein. In some embodiments, the percent composition of the unconjugated protein (e.g., unconjugated antibody) is between 10% and 45%, with the remainder comprising the conjugated protein. In some embodiments, the percent composition of the unconjugated protein (e.g., unconjugated antibody) is between 10% and 50%, with the remainder comprising the conjugated protein. In some embodiments, the percent composition of the unconjugated protein (e.g., unconjugated antibody) is between 10% and 55%, with the remainder comprising the conjugated protein. In some embodiments, the percent composition of the unconjugated protein (e.g., unconjugated antibody) is between 10% and 60%, with the remainder comprising the conjugated protein. In some embodiments, the percent composition of the unconjugated protein (e.g., unconjugated antibody) is between 10% and 65%, with the remainder comprising the conjugated protein. In some embodiments, the percent composition of the unconjugated protein (e.g., unconjugated antibody) is between 10% and 70%, with the remainder comprising the conjugated protein. In some embodiments, the percent composition of the unconjugated protein (e.g., unconjugated antibody) is between 10% and 75%, with the remainder comprising the conjugated protein. In some embodiments, the percent composition of the unconjugated protein (e.g., unconjugated antibody) is between 10% and 80%, with the remainder comprising the conjugated protein. In some embodiments, the percent composition of the unconjugated protein (e.g., unconjugated antibody) is between 10% and 85%, with the remainder comprising the conjugated protein. In some embodiments, the percent composition of the unconjugated protein (e.g., unconjugated antibody) is between 10% and 90%.

In some embodiments, the percent composition of the unconjugated protein (e.g., unconjugated antibody or unconjugated fusion construct) is between 12.5% and 15%, with the remainder comprising the conjugated protein (e.g., the conjugated antibody or conjugated fusion construct). In some embodiments, the percent composition of the unconjugated protein (e.g., unconjugated antibody) is between 12.5% and 17.5%. In some embodiments, the percent composition of the unconjugated protein (e.g., unconjugated antibody) is between 17.5% and 22.5%. In some embodiments, the percent composition of the unconjugated protein (e.g., unconjugated antibody) is between 12.5% and 20%, with the remainder comprising the conjugated protein. In some embodiments, the percent composition of the unconjugated protein (e.g., unconjugated antibody) is between 12.5% and 25%, with the remainder comprising the conjugated protein. In some embodiments, the percent composition of the unconjugated protein (e.g., unconjugated antibody) is between 12.5% and 30%, with the remainder comprising the conjugated protein. In some embodiments, the percent composition of the unconjugated protein (e.g., unconjugated antibody) is between 12.5% and 35%, with the remainder comprising the conjugated protein. In some embodiments, the percent composition of the unconjugated protein (e.g., unconjugated antibody) is between 12.5% and 45%, with the remainder comprising the conjugated protein. In some embodiments, the percent composition of the unconjugated protein (e.g., unconjugated antibody) is between 12.5% and 50%, with the remainder comprising the conjugated protein. In some embodiments, the percent composition of the unconjugated protein (e.g., unconjugated antibody) is between 12.5% and 55%, with the remainder comprising the conjugated protein. In some embodiments, the percent composition of the unconjugated protein (e.g., unconjugated antibody) is between 12.5% and 60%, with the remainder comprising the conjugated protein. In some embodiments, the percent composition of the unconjugated protein (e.g., unconjugated antibody) is between 12.5% and 65%, with the remainder comprising the conjugated protein. In some embodiments, the percent composition of the unconjugated protein (e.g., unconjugated antibody) is between 12.5% and 70%, with the remainder comprising the conjugated protein. In some embodiments, the percent composition of the unconjugated protein (e.g., unconjugated antibody) is between 12.5% and 75%, with the remainder comprising the conjugated protein. In some embodiments, the percent composition of the unconjugated protein (e.g., unconjugated antibody) is between 12.5% and 80%, with the remainder comprising the conjugated protein. In some embodiments, the percent composition of the unconjugated protein (e.g., unconjugated antibody) is between 12.5% and 85%, with the remainder comprising the conjugated protein. In some embodiments, the percent composition of the unconjugated protein (e.g., unconjugated antibody) is between 12.5% and 90%.

In some embodiments, the percent composition of the unconjugated protein (e.g., unconjugated antibody or unconjugated fusion construct) is between 15% and 17.5%, with the remainder comprising the conjugated protein (e.g., conjugated antibody or fusion construct). In some embodiments, the percent composition of the unconjugated protein (e.g., unconjugated antibody) is between 15% and 20%, with the remainder comprising the conjugated protein. In some embodiments, the percent composition of the unconjugated protein (e.g., unconjugated antibody) is between 15% and 25%, with the remainder comprising the conjugated protein. In some embodiments, the percent composition of the unconjugated protein (e.g., unconjugated antibody) is between 15% and 30%, with the remainder comprising the conjugated protein. In some embodiments, the percent composition of the unconjugated protein (e.g., unconjugated antibody) is between 15% and 35%, with the remainder comprising the conjugated protein. In some embodiments, the percent composition of the unconjugated protein (e.g., unconjugated antibody) is between 15% and 45%, with the remainder comprising the conjugated protein. In some embodiments, the percent composition of the unconjugated protein (e.g., unconjugated antibody) is between 15% and 50%, with the remainder comprising the conjugated protein. In some embodiments, the percent composition of the unconjugated protein (e.g., unconjugated antibody) is between 15% and 55%, with the remainder comprising the conjugated protein. In some embodiments, the percent composition of the unconjugated protein (e.g., unconjugated antibody) is between 15% and 60%, with the remainder comprising the conjugated protein. In some embodiments, the percent composition of the unconjugated protein (e.g., unconjugated antibody) is between 15% and 65%, with the remainder comprising the conjugated protein. In some embodiments, the percent composition of the unconjugated protein (e.g., unconjugated antibody) is between 15% and 70%, with the remainder comprising the conjugated protein. In some embodiments, the percent composition of the unconjugated protein (e.g., unconjugated antibody) is between 15% and 75%, with the remainder comprising the conjugated protein. In some embodiments, the percent composition of the unconjugated protein (e.g., unconjugated antibody) is between 15% and 80%, with the remainder comprising the conjugated protein. In some embodiments, the percent composition of the unconjugated protein (e.g., unconjugated antibody) is between 15% and 85%, with the remainder comprising the conjugated protein. In some embodiments, the percent composition of the unconjugated protein (e.g., unconjugated antibody) is between 15% and 90%.

In some embodiments, the percent composition of the unconjugated protein (e.g., unconjugated antibody or unconjugated fusion construct) is between 20% and 25%, with the remainder comprising the conjugated protein (e.g., conjugated antibody or fusion construct). In some embodiments, the percent composition of the unconjugated protein (e.g., unconjugated antibody) is between 25% and 30%, with the remainder comprising the conjugated protein. In some embodiments, the percent composition of the unconjugated protein (e.g., unconjugated antibody) is between 25% and 35%, with the remainder comprising the conjugated protein. In some embodiments, the percent composition of the unconjugated protein (e.g., unconjugated antibody) is between 25% and 45%, with the remainder comprising the conjugated protein. In some embodiments, the percent composition of the unconjugated protein (e.g., unconjugated antibody) is between 25% and 50%, with the remainder comprising the conjugated protein. In some embodiments, the percent composition of the unconjugated protein (e.g., unconjugated antibody) is between 25% and 55%, with the remainder comprising the conjugated protein. In some embodiments, the percent composition of the unconjugated protein (e.g., unconjugated antibody) is between 25% and 60%, with the remainder comprising the conjugated protein. In some embodiments, the percent composition of the unconjugated protein (e.g., unconjugated antibody) is between 25% and 65%, with the remainder comprising the conjugated protein. In some embodiments, the percent composition of the unconjugated protein (e.g., unconjugated antibody) is between 25% and 70%, with the remainder comprising the conjugated protein. In some embodiments, the percent composition of the unconjugated protein (e.g., unconjugated antibody) is between 25% and 75%, with the remainder comprising the conjugated protein. In some embodiments, the percent composition of the unconjugated protein (e.g., unconjugated antibody) is between 25% and 80%, with the remainder comprising the conjugated protein. In some embodiments, the percent composition of the unconjugated protein (e.g., unconjugated antibody) is between 25% and 85%, with the remainder comprising the conjugated protein. In some embodiments, the percent composition of the unconjugated protein (e.g., unconjugated antibody) is between 25% and 90%, with the remainder comprising the conjugated protein.

In some embodiments, the percent composition of the unconjugated protein (e.g., unconjugated antibody or unconjugated fusion construct) is 1%, with the remainder comprising the conjugated protein (e.g., conjugated antibody or conjugated fusion construct). In some embodiments, the percent composition of the unconjugated protein (e.g., unconjugated antibody or unconjugated fusion construct) is 5%, with the remainder comprising the conjugated protein (e.g., conjugated antibody or conjugated fusion construct). In some embodiments, the percent composition of the unconjugated protein (e.g., unconjugated antibody) is 6%, with the remainder comprising the conjugated protein. In some embodiments, the percent composition of the unconjugated protein (e.g., unconjugated antibody) is 7%, with the remainder comprising the conjugated protein. In some embodiments, the percent composition of the unconjugated protein (e.g., unconjugated antibody) is 8%, with the remainder comprising the conjugated protein. In some embodiments, the percent composition of the unconjugated protein (e.g., unconjugated antibody) is 9%, with the remainder comprising the conjugated protein. In some embodiments, the percent composition of the unconjugated protein (e.g., unconjugated antibody) is 10%, with the remainder comprising the conjugated protein. In some embodiments, the percent composition of the unconjugated protein (e.g., unconjugated antibody) is 11%, with the remainder comprising the conjugated protein. In some embodiments, the percent composition of the unconjugated protein (e.g., unconjugated antibody) is 12%, with the remainder comprising the conjugated protein. In some embodiments, the percent composition of the unconjugated protein (e.g., unconjugated antibody) is 13%, with the remainder comprising the conjugated protein. In some embodiments, the percent composition of the unconjugated protein (e.g., unconjugated antibody) is 14%, with the remainder comprising the conjugated protein. In some embodiments, the percent composition of the unconjugated protein (e.g., unconjugated antibody) is 15%, with the remainder comprising the conjugated protein. In some embodiments, the percent composition of the unconjugated protein (e.g., unconjugated antibody) is 16%, with the remainder comprising the conjugated protein. In some embodiments, the percent composition of the unconjugated protein (e.g., unconjugated antibody) is 17%, with the remainder comprising the conjugated protein. In some embodiments, the percent composition of the unconjugated protein (e.g., unconjugated antibody) is 18%, with the remainder comprising the conjugated protein. In some embodiments, the percent composition of the unconjugated protein (e.g., unconjugated antibody) is 19%, with the remainder comprising the conjugated protein. In some embodiments, the percent composition of the unconjugated protein (e.g., unconjugated antibody) is 20%, with the remainder comprising the conjugated protein. In some embodiments, the percent composition of the unconjugated protein (e.g., unconjugated antibody) is 21%, with the remainder comprising the conjugated protein. In some embodiments, the percent composition of the unconjugated protein (e.g., unconjugated antibody) is 22%, with the remainder comprising the conjugated protein. In some embodiments, the percent composition of the unconjugated protein (e.g., unconjugated antibody) is 23%, with the remainder comprising the conjugated protein. In some embodiments, the percent composition of the unconjugated protein (e.g., unconjugated antibody) is 24%, with the remainder comprising the conjugated protein. In some embodiments, the percent composition of the unconjugated protein (e.g., unconjugated antibody) is 25%.

In some embodiments, the percent composition of the unconjugated protein (e.g., unconjugated antibody or unconjugated fusion construct) is 26%, with the remainder comprising the conjugated protein (e.g., conjugated antibody or conjugated fusion construct). In some embodiments, the percent composition of the unconjugated protein (e.g., unconjugated antibody) is 27%, with the remainder comprising the conjugated protein. In some embodiments, the percent composition of the unconjugated protein (e.g., unconjugated antibody) is 28%, with the remainder comprising the conjugated protein. In some embodiments, the percent composition of the unconjugated protein (e.g., unconjugated antibody) is 29%, with the remainder comprising the conjugated protein. In some embodiments, the percent composition of the unconjugated protein (e.g., unconjugated antibody) is 30%, with the remainder comprising the conjugated protein. In some embodiments, the percent composition of the unconjugated protein (e.g., unconjugated antibody) is 31%, with the remainder comprising the conjugated protein. In some embodiments, the percent composition of the unconjugated protein (e.g., unconjugated antibody) is 32%, with the remainder comprising the conjugated protein. In some embodiments, the percent composition of the unconjugated protein (e.g., unconjugated antibody) is 33%, with the remainder comprising the conjugated protein. In some embodiments, the percent composition of the unconjugated protein (e.g., unconjugated antibody) is 34%, with the remainder comprising the conjugated protein. In some embodiments, the percent composition of the unconjugated protein (e.g., unconjugated antibody) is 35%.

In some embodiments, the percent composition of the unconjugated protein (e.g., unconjugated antibody or unconjugated fusion construct) is 36%, with the remainder comprising the conjugated protein (e.g., conjugated antibody or fusion construct). In some embodiments, the percent composition of the unconjugated protein (e.g., unconjugated antibody) is 37%, with the remainder comprising the conjugated protein. In some embodiments, the percent composition of the unconjugated protein (e.g., unconjugated antibody) is 38%, with the remainder comprising the conjugated protein. In some embodiments, the percent composition of the unconjugated protein (e.g., unconjugated antibody) is 39%, with the remainder comprising the conjugated protein. In some embodiments, the percent composition of the unconjugated protein (e.g., unconjugated antibody) is 40%, with the remainder comprising the conjugated protein. In some embodiments, the percent composition of the unconjugated protein (e.g., unconjugated antibody) is 41%, with the remainder comprising the conjugated protein. In some embodiments, the percent composition of the unconjugated protein (e.g., unconjugated antibody) is 42%, with the remainder comprising the conjugated protein. In some embodiments, the percent composition of the unconjugated protein (e.g., unconjugated antibody) is 43%, with the remainder comprising the conjugated protein. In some embodiments, the percent composition of the unconjugated protein (e.g., unconjugated antibody) is 44%, with the remainder comprising the conjugated protein. In some embodiments, the percent composition of the unconjugated protein (e.g., unconjugated antibody) is 45%.

In some embodiments, the percent composition of the unconjugated protein (e.g., unconjugated antibody or unconjugated fusion construct) is 46%, with the remainder comprising the conjugated protein (e.g., conjugated antibody or conjugated fusion construct). In some embodiments, the percent composition of the unconjugated protein (e.g., unconjugated antibody) is 47%, with the remainder comprising the conjugated protein. In some embodiments, the percent composition of the unconjugated protein (e.g., unconjugated antibody) is 48%, with the remainder comprising the conjugated protein. In some embodiments, the percent composition of the unconjugated protein (e.g., unconjugated antibody) is 49%, with the remainder comprising the conjugated protein. In some embodiments, the percent composition of the unconjugated protein (e.g., unconjugated antibody) is 50%, with the remainder comprising the conjugated protein. In some embodiments, the percent composition of the unconjugated protein (e.g., unconjugated antibody) is 51%, with the remainder comprising the conjugated protein. In some embodiments, the percent composition of the unconjugated protein (e.g., unconjugated antibody) is 52%, with the remainder comprising the conjugated protein. In some embodiments, the percent composition of the unconjugated protein (e.g., unconjugated antibody) is 53%, with the remainder comprising the conjugated protein. In some embodiments, the percent composition of the unconjugated protein (e.g., unconjugated antibody) is 54%, with the remainder comprising the conjugated protein. In some embodiments, the percent composition of the unconjugated protein (e.g., unconjugated antibody) is 55%.

In some embodiments, the percent composition of the unconjugated protein (e.g., unconjugated antibody or unconjugated fusion construct) is 56%, with the remainder comprising the conjugated protein (e.g., conjugated antibody or conjugated fusion construct). In some embodiments, the percent composition of the unconjugated protein (e.g., unconjugated antibody) is 57%, with the remainder comprising the conjugated protein. In some embodiments, the percent composition of the unconjugated protein (e.g., unconjugated antibody) is 58%, with the remainder comprising the conjugated protein. In some embodiments, the percent composition of the unconjugated protein (e.g., unconjugated antibody) is 59%, with the remainder comprising the conjugated protein. In some embodiments, the percent composition of the unconjugated protein (e.g., unconjugated antibody) is 60%, with the remainder comprising the conjugated protein. In some embodiments, the percent composition of the unconjugated protein (e.g., unconjugated antibody) is 51%, with the remainder comprising the conjugated protein. In some embodiments, the percent composition of the unconjugated protein (e.g., unconjugated antibody) is 62%, with the remainder comprising the conjugated protein. In some embodiments, the percent composition of the unconjugated protein (e.g., unconjugated antibody) is 63%, with the remainder comprising the conjugated protein. In some embodiments, the percent composition of the unconjugated protein (e.g., unconjugated antibody) is 64%, with the remainder comprising the conjugated protein. In some embodiments, the percent composition of the unconjugated protein (e.g., unconjugated antibody) is 65%.

In some embodiments, the percent composition of the unconjugated protein (e.g., unconjugated antibody or unconjguated fusion construct) is 66%, with the remainder comprising the conjugated protein (e.g., conjugated antibody or conjugated fusion construct). In some embodiments, the percent composition of the unconjugated protein (e.g., unconjugated antibody) is 67%, with the remainder comprising the conjugated protein. In some embodiments, the percent composition of the unconjugated protein (e.g., unconjugated antibody) is 68%, with the remainder comprising the conjugated protein. In some embodiments, the percent composition of the unconjugated protein (e.g., unconjugated antibody) is 69%, with the remainder comprising the conjugated protein. In some embodiments, the percent composition of the unconjugated protein (e.g., unconjugated antibody) is 70%, with the remainder comprising the conjugated protein. In some embodiments, the percent composition of the unconjugated protein (e.g., unconjugated antibody) is 11%, with the remainder comprising the conjugated protein. In some embodiments, the percent composition of the unconjugated protein (e.g., unconjugated antibody) is 72%, with the remainder comprising the conjugated protein. In some embodiments, the percent composition of the unconjugated protein (e.g., unconjugated antibody) is 73%, with the remainder comprising the conjugated protein. In some embodiments, the percent composition of the unconjugated protein (e.g., unconjugated antibody) is 74%, with the remainder comprising the conjugated protein. In some embodiments, the percent composition of the unconjugated protein (e.g., unconjugated antibody) is 75%.

In some embodiments, the percent composition of the unconjugated protein (e.g., unconjugated antibody or unconjugated) is 76%, with the remainder comprising the conjugated protein (e.g., conjugated antibody or conjugated fusion construct). In some embodiments, the percent composition of the unconjugated protein (e.g., unconjugated antibody) is 77%, with the remainder comprising the conjugated protein. In some embodiments, the percent composition of the unconjugated protein (e.g., unconjugated antibody) is 78%, with the remainder comprising the conjugated protein. In some embodiments, the percent composition of the unconjugated protein (e.g., unconjugated antibody) is 79%, with the remainder comprising the conjugated protein. In some embodiments, the percent composition of the unconjugated protein (e.g., unconjugated antibody) is 80%, with the remainder comprising the conjugated protein. In some embodiments, the percent composition of the unconjugated protein (e.g., unconjugated antibody) is 81%, with the remainder comprising the conjugated protein. In some embodiments, the percent composition of the unconjugated protein (e.g., unconjugated antibody) is 82%, with the remainder comprising the conjugated protein. In some embodiments, the percent composition of the unconjugated protein (e.g., unconjugated antibody) is 83%, with the remainder comprising the conjugated protein. In some embodiments, the percent composition of the unconjugated protein (e.g., unconjugated antibody) is 84%, with the remainder comprising the conjugated protein. In some embodiments, the percent composition of the unconjugated protein (e.g., unconjugated antibody) is 85%.

In some embodiments, the percent composition of the unconjugated protein (e.g., unconjugated antibody or unconjugated fusion construct) is 86%, with the remainder comprising the conjugated protein. In some embodiments, the percent composition of the unconjugated protein (e.g., unconjugated antibody) is 87%, with the remainder comprising the conjugated protein. In some embodiments, the percent composition of the unconjugated protein (e.g., unconjugated antibody) is 88%, with the remainder comprising the conjugated protein. In some embodiments, the percent composition of the unconjugated protein (e.g., unconjugated antibody) is 89%, with the remainder comprising the conjugated protein. In some embodiments, the percent composition of the unconjugated protein (e.g., unconjugated antibody) is 90%.

In some embodiments, the ratio of the molecular weight of the unconjugated protein (e.g., second protein that is not conjugated to a phosphorylcholine-containing polymer) to the polymer in the formulation can be any suitable ratio. In some embodiments, the ratio is at most about 1:2, e.g., at most about 1:3, at most about 1:4, at most about 1:5, at most about 1:6, at most about 1:7, at most about 1:8, at most about 1:9 or at most about 1:10, or a ratio in a range defined by any two of the preceding values (e.g., 1:2-1:10, 1:3-1:8, 1:4-1:6). In some embodiments, the ratio is between about 1:4 and 1:6. In some embodiments, the ratio is about 1:5.33.

The protein conjugated to the phosphorylcholine-containing polymer and the unconjugated protein can each be any suitable protein. In some embodiments, the first protein (e.g., the protein conjugated to the phosphorylcholine-containing polymer) and the second protein (e.g., the protein that is not conjugated to a phosphorylcholine-containing polymer) have the same activity or function (e.g., bind the same epitope, inhibit the same target, catalyze the same reaction, etc.). In some embodiments, the first protein (e.g., the protein conjugated to the phosphorylcholine-containing polymer) and the second protein (e.g., the protein that is not conjugated to a phosphorylcholine-containing polymer) are the same protein. In some embodiments, the first protein (e.g., the protein conjugated to the phosphorylcholine-containing polymer) and the second protein (e.g., the protein that is not conjugated to a phosphorylcholine-containing polymer) are at least about 85%, at least about 90%, at least about 95%, at least about 97%, at least about 98%, at least about 99%, or about 100%, or by a percentage in a range defined by any two of the preceding values (e.g., 85-100%, 90-99%, 90-95%, etc.) identical to each other in amino acid sequence. In some embodiments, where the first and second proteins each include two or more polypeptide chains, each of the corresponding polypeptide chains can independently have any of the noted sequence identity. In some embodiments, the second protein is or comprises a derivative of the first protein (e.g., the protein portion without the polymer). In some embodiments, the second protein is alkylated form of the first protein (e.g., the protein portion without the polymer). For example, the second protein is an iodoacetamide (IAM)- or N-ethylmaleimide (NEM)-treated form of the first protein (e.g., the protein portion without the polymer).

In some embodiments, the first protein (e.g., the protein conjugated to the phosphorylcholine-containing polymer) has a molecular weight (based on the protein portion) of about 5 kDa or more, about 10 kDa or more, about 15 kDa or more, about 25 kDa or more, about 50 kDa or more, about 75 kDa or more, about 100 kDa or more, about 125 kDa or more, about 150 kDa or more, about 175 kDa or more, about 200 kDa or more, about 250 kDa or more, about 300 kDa or more, or a molecular weight in a range defined by any two of the preceding values (e.g., 50-300 kDa, 100-300 kDa, 100-200 kDa, 150-250 kDa, etc.). In some embodiments, the first protein (e.g., the protein conjugated to the phosphorylcholine-containing polymer) has a molecular weight (based on the protein portion) of about 150 kDa. In some embodiments, the first protein (e.g., the protein conjugated to the phosphorylcholine-containing polymer) has a molecular weight of about 200 kDa. In some embodiments, the second protein (e.g., the unconjugated protein or protein that is not conjugated to a phosphorylcholine-containing polymer) has a molecular weight of about 50 kDa or more, e.g., about 75 kDa or more, about 100 kDa or more, about 125 kDa or more, about 150 kDa or more, about 175 kDa or more, about 200 kDa or more, about 250 kDa or more, about 300 kDa or more, or a molecular weight in a range defined by any two of the preceding values (e.g., 50-300 kDa, 100-300 kDa, 100-200 kDa, 150-250 kDa, etc.). In some embodiments, the second protein (e.g., the unconjugated protein or protein that is not conjugated to a phosphorylcholine-containing polymer) has a molecular weight of about 150 kDa. In some embodiments, the second protein (e.g., the unconjugated protein or protein that is not conjugated to a phosphorylcholine-containing polymer) has a molecular weight of about 200 kDa.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a therapeutic protein. In some embodiments, at least one of the first protein (e.g., the protein conjugated to the phosphorylcholine-containing polymer) and the second protein (e.g., the unconjugated protein or protein that is not conjugated to a phosphorylcholine-containing polymer) is a therapeutic protein that is FDA approved as of May 2023. In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an antibody (e.g., therapeutic antibody). Any suitable antibody can be used in the formulations. In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a fusion construct. Any suitable fusion construct can be used in the formulations. The antibody or fusion construct of any of the formulation or composition herein may or may not include a C-terminal lysine. “Fusion protein” and “fusion construct” are used interchangeably herein.

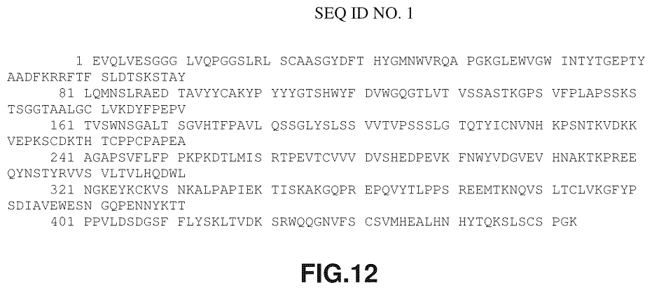

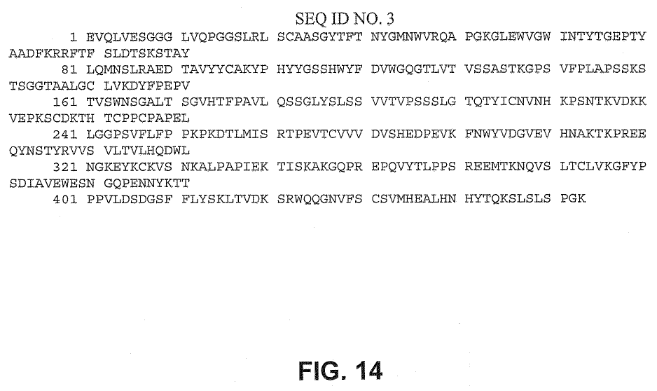

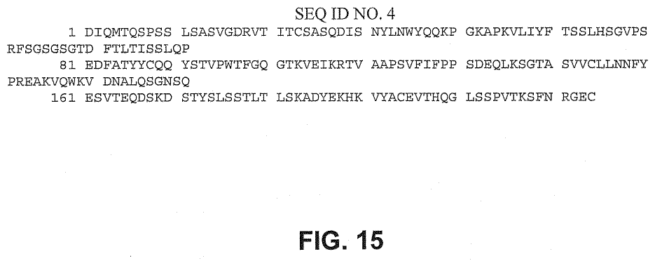

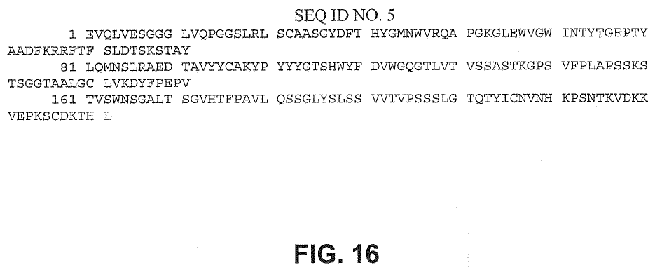

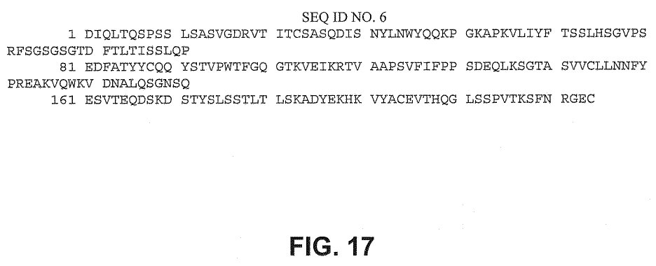

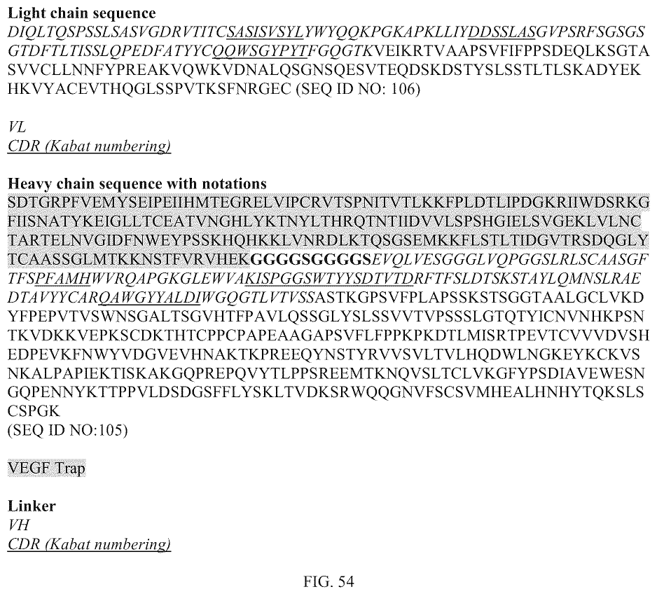

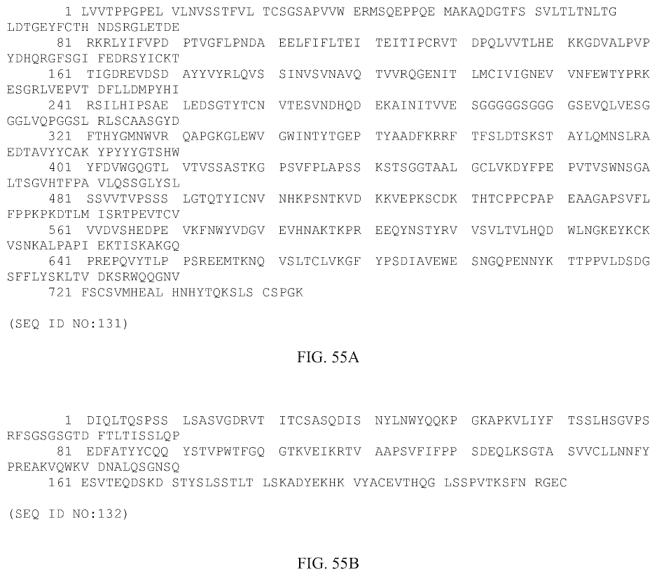

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-VEGF-A antibody, a fusion construct comprising a VEGF Trap fused to the heavy chain of an anti-IL-6 antibody, an anti-IL-6 antibody, a fusion construct comprising a PDGFR extracellular trap fused to the heavy chain of an anti-VEGF-A antibody, a VEGF trap-Fc fusion protein, an anti-HTRA1 antibody, or an anti-complement factor D (CFD) antibody. In some embodiments, both of the first protein (e.g., the protein conjugated to the phosphorylcholine-containing polymer) and the second protein (e.g., the unconjugated protein or protein that is not conjugated to a phosphorylcholine-containing polymer) are an anti-VEGF-A antibody. In some embodiments, both of the first protein (e.g., the protein conjugated to the phosphorylcholine-containing polymer) and the second protein (e.g., the unconjugated protein or protein that is not conjugated to a phosphorylcholine-containing polymer) are a fusion construct comprising a VEGF Trap fused to the heavy chain of an anti-IL-6 antibody. In some embodiments, the anti-VEGF-A antibody is a full-length antibody. In some embodiments, the anti-VEGF-A antibody is or includes an anti-VEGF-A Fab fragment. In some embodiments, the anti-VEGF-A antibody is OG1950, e.g., as described herein and in US patent publication no. 2017/0190766, the entirety of which is incorporated herein by reference. In some embodiments, the anti-VEGF-A antibody is selected from bevacizumab, ranibizumab, brolucizumab, or faricimab. In some embodiments, the VEGF trap-Fc fusion protein is aflibercept. In some embodiments, the fusion construct is OG2072, e.g., as described herein and in US patent publication no. 2019/0270806, the entirety of which is incorporated herein by reference. In some embodiments, the first protein comprises any one of the amino sequences or a combination thereof set forth in Tables 0.1, 0.2, 0.3, 0.4, 0.5, 0.6 and 0.7, and FIGS. 12-17, 54, 55A, and 55B. In some embodiments, the second protein comprises any one of the amino sequences or a combination thereof set forth in Tables 0.1, 0.2, 0.3, 0.4, 0.5, 0.6 and 0.7, and FIGS. 12-17, 54, 55A, and 55B. In some embodiments, the first protein comprises any one of the amino sequences or a combination thereof set forth in Tables 0.1, 0.2, 0.3, 0.4, 0.5, 0.6 and 0.7, and FIGS. 12-17, 54, 55A, and 55B, and the second protein comprises any one of the amino sequences or a combination thereof set forth in Tables 0.1, 0.2, 0.3, 0.4, 0.5, 0.6 and 0.7, and FIGS. 12-17, 54, 55A, and 55B. In some embodiments, the first protein comprises any one of the amino sequences or a combination thereof set forth in SEQ ID NOs: 1-6, 15-133, and 156-161. In some embodiments, the first protein comprises any one of the amino sequences or a combination thereof set forth in SEQ ID NOs: 1-6, 15-133, and 156-161. In some embodiments, the first protein comprises any one of the amino sequences or a combination thereof set forth in SEQ ID NOs: 1-6, 15-133, and 156-161, and the second protein comprises any one of the amino sequences or a combination thereof set forth in SEQ ID NOs: 1-6, 15-133, and 156-161. In some embodiments, the amino acid sequences of the first protein and second protein are paired as they are arranged in Tables 0.1, 0.2, 0.3, 0.4, 0.5, 0.6 and 0.7, and FIGS. 12-17, 54, 55A, and 55B).

In some embodiments, the conjugated and unconjugated proteins are antibodies, and one or both antibodies are therapeutic. In some embodiments, one or both antibodies are the same or variants of each other, wherein one antibody is conjugated to a polymer at a cysteine outside a variable region of the antibody and the other protein is unconjugated. In some embodiments, both antibodies are therapeutic antibodies. In some embodiments, at least one of the antibodies is a therapeutic antibody that is FDA approved as of May 2023. In some embodiments, both antibodies are therapeutic for the treatment of an eye disorder. In some embodiments, both antibodies (conjugated and unconjugated) share at least the same CDR sequences in the heavy and light chains. In some embodiments, both antibodies (conjugated and unconjugated) have at least one (e.g., 1, 2, 3, 4, 5, or 6) CDR in the heavy or light chains that is different from each other. In some embodiments, the composition comprises (a) an antibody conjugate comprising an anti-VEGF-A antibody and a phosphorylcholine-containing polymer wherein the polymer is covalently bonded to the antibody at a cysteine outside a variable region of the antibody; and (b) an unconjugated anti-VEGF-A antibody.

In some embodiments, the anti-VEGF-A antibody of the antibody conjugate comprises a light chain and a heavy chain, said heavy chain comprising an Fc region. In some embodiments, a cysteine of the antibody conjugate is in the Fc region of the heavy chain. In some embodiments, the anti-VEGF-A antibody of the antibody conjugate is an immunoglobulin G (IgG).

In some embodiments, the anti-VEGF-A antibody heavy chain of the antibody conjugate or the unconjugated antibody comprises: CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11), and the anti-VEGF-A light chain of the antibody conjugate or the unconjugated antibody comprises CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14). In some embodiments, the anti-VEGFA heavy chain isotype of the antibody conjugate or the unconjugated antibody is human IgG1. In some embodiments, the heavy chain constant domain of the anti-VEGF-A antibody of the antibody conjugate or the unconjugated antibody has one or more mutations relative to the constant domain of human IgG1 to modulate effector function. T

In some embodiments, mutations of the antibody conjugate (and/or unconjugated antibody) may be to one or more of the following amino acid positions (EU numbering): E233X, L234X, L235X, G236X, G237X, A327X, A330X, and P331X wherein X is any natural or unnatural amino acid. In some embodiments, mutations of the antibody conjugate (and/or unconjugated antibody) are selected from the group consisting of (EU numbering): E233P, L234V, L234A, L235A, G237A, A327G, A330S, and P331S. In some embodiments, mutations of the antibody conjugate (and/or unconjugated antibody) comprise: L234A, L235A, and G237A (EU numbering). In some embodiments, the cysteine of the antibody conjugate (and/or unconjugated antibody) is in the anti-VEGF-A antibody heavy chain and is Q347C (EU numbering) or L443C (EU numbering).

In some embodiments, the sequence of the anti-VEGF-A antibody heavy chain of the antibody conjugate or the unconjugated antibody is SEQ ID NO: 1 (with or without the C-terminal lysine) and the sequence of the anti-VEGF-A light chain of the antibody conjugate or the unconjugated antibody is SEQ ID NO: 2. In some embodiments, the sequence of the anti-VEGF-A antibody heavy chain of the antibody conjugate or the unconjugated antibody is at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 1, and the sequence of the anti-VEGF-A light chain of the antibody conjugate or the unconjugated antibody is at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 2. In some embodiments, the cysteine of the antibody conjugate or the unconjugated antibody is L443C (EU numbering).

In some embodiments, the solution pH of the composition depends on the isoelectric point of a desired protein, wherein the solution pH is at least 1-2 pH units away from the desired protein's isoelectric point.

In some embodiments, a composition comprising a first antibody and a second antibody is provided. The first antibody is conjugated to a phosphorylcholine-containing polymer, wherein the polymer is covalently bonded to the first antibody at a cysteine outside a variable region of the first antibody; wherein the second antibody is not conjugated to the phosphorylcholine-containing polymer.

In some embodiments, the first antibody comprises CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11), CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14). In some embodiments, mutations of the first antibody are to one or more of the following amino acid positions (EU numbering): E233X, L234X, L235X, G236X, G237X, A327X, A330X, and P331X wherein X is any natural or unnatural amino acid. In some embodiments, mutations of the first antibody are selected from the group consisting of (EU numbering): E233P, L234V, L234A, L235A, G237A, A327G, A330S, and P331S. In some embodiments, the following mutations of the first antibody: L234A, L235A, and G237A (EU numbering). In some embodiments, the cysteine of the first antibody is in the anti-VEGF-A antibody heavy chain and is Q347C (EU numbering) or L443C (EU numbering). In some embodiments, the sequence of the anti-VEGF-A antibody heavy chain of the first antibody is SEQ ID NO: 1 (with or without the C-terminal lysine) and the sequence of the anti-VEGF-A light chain of the first antibody is SEQ ID NO: 2. Any or all the foregoing embodiments in this paragraph could apply to the second antibody. OG1950 denotes an anti-VEGF-A antibody having a heavy chain of SEQ ID NO:1 (with or without the C-terminal lysine) and a light chain of SEQ ID NO: 2.

In some embodiments, the cysteine of the first antibody is L443C (EU numbering).

In some embodiments, the first and second antibody have the same CDRs (1, 2, 3, 4, 5, or all 6), same VH, VL, same VH and same VL, and/or same HC and LC, with the only difference being one is conjugated to a polymer and one is not conjugated to the polymer.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-complement factor D (CFD) antibody. In some embodiments, the anti-CFD antibody includes a heavy chain having any 3 of the heavy chain CDRs in any one of SEQ ID NOs: 15-47, and a light chain having any 3 of the light chain CDRs in any one of SEQ ID NOs: 48-80. In some embodiments, the anti-CFD antibody includes a heavy chain having any 3 of the heavy chain CDRs in any one of SEQ ID NOs: 15-47, and a light chain having any 3 of the light chain CDRs in any one of SEQ ID NOs: 48-80, as they are paired in Table 0.1. In some embodiments, the anti-CFD antibody includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 15-47, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 48-80. In some embodiments, the anti-CFD antibody includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 15-47, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 48-80, as they are paired in Table 0.1.

TABLE 0.1

| mAb | Heavy Chain | Light Chain |
|---|---|---|
| KCD002 | EVKLVESEGGLVQPGSSMKLSCTASGFTFS | DIQMTQSPASLSASVGETVTITCRASENIHSY |
| | DYYMAWVRQVPEKGLEWVGNINYDGSSTY | LAWYQQKQGKSPQLIVYNTKTLAEGVPSRF |
| | YLDSLKSRFIISRDSAKNILYLQMSSLKSEDT | SGSGSGTQFSLKINSLQPEDFGSYYCQHHYG |
| | ATYFCARGEDFYLYAMDYWGQGTSVTVSS | IPPTFGGGTKLEIK (SEQ ID NO: 48) |
| | (SEQ ID NO: 15) | |
| KCD003 | EVQLQQSRPELVKPGASVKIFCKASGYTFT | DVLMTQTPLSLPVSLGEQASISCRSSQTIVHS |
| | DYYMNWMRQRHGETLEWIGDINPNNGDPS | NGDTYLEWYLQKPGQSPNLLIYKVSNRFSG |
| | YNQKFKDKATLTVDKSSSTASMELRSLTSD | VPDRFSGSGSGTDFTLKISRVEAEDLGVYYC |
| | DSAVYYCAREGPSFAYWGQGTLVTVSA | FQGSHVPPTFGGGTKLEIK (SEQ ID NO: 49) |
| | (SEQ ID NO: 16) | |
| KCD005 | EVQLQQSGPELVKPGASVKISCKASGYTFT | DVLMTQTPLSLPVSLGDQASISCRSSQSIVHS |
| | DHYMNWVKQSHGKSLEWIGDINPNNGGTS | NGDTYLEWYLQKPGQSPKLLIYKVSNRFSG |
| | CNQKFKGKATLTVDKSSSTAYMELRSLTSG | VPDRFSGSGSGTDFTLKISRVEAEDLGVYYC |
| | DSAVYYCTREGASFAFWGQGTLVTVSA | FQGSHVPVTFGAGTKLELK (SEQ ID NO: 50) |
| | (SEQ ID NO: 17) | |
| KCD009 | QIQLVQSGPELKKPGETVKISCKASGYIFRN | DVLMTQTPLSLPVSLGDQASISCRSSLIIEHS |
| | YGMNWVKQGPGKGLKWMGWINTYTGEP | DGNTYLEWYLQKPGQSPKLLIYKVSNRFSG |
| | TYADDFKGRFAFSLETSASTAYLQISNLKNE | VPDRFSGSGSGTDFTLKISR VEADDLGVYYC |
| | DTATYFCVRDGPGFAYWGQGTLVTVSA | FQGSHVPVTFGAGTNLELK (SEQ ID NO: 51) |
| | (SEQ ID NO: 18) | |
| KCD010 | EVLLQQSGPELVKPGASVKIPCKASGYTFTD | QIVLTQSPAIMSVSPGEKVTLTCSASSSVSSS |
| | YDMDWVKQSHGKSLEWIGHINPNNGGTIY | YLYWYQKKPGSSPKLWIYSTSNLASGVPAR |
| | NQKFKGKATLTVDKSSSTAYMELRSLTSED | FSGSGSGTSYSLTISSMEAEDAASYFCHQWS |
| | TAVYYCGTGDFAYWGHGTLVTVSA (SEQ ID | SYPPTFGAGTKLELK (SEQ ID NO: 52) |
| | NO: 19) | |
| KCD023 | EVLLQQSGPELVKPGASVKIPCKASGYTFTD | QIVLTQSPAFMSASPGEKVTLTCSASSSVSSS |
| | YNIDWVKQSHGKSLEWIGDINPNNGGINYN | YLYWYQQKPGSSPKLWIYSTSNLASGVPGR |
| | QKFKGKATLTVDKSSSTAYMELRSLTSEDT | FSGSGSGTSYSLTISSMEAEDAASYFCHQWT |
| | AVYYCGTGDYAYWGQGTLVTVSA (SEQ ID | SYPPTFGAGTKLELK (SEQ ID NO: 53) |
| | NO: 20) | |
| KCD036 | EVQLQQSGPELVKPGASMKISCKASGYSFT | DIKLTQSPSSMYASLGERVTITCKASQDINT |
| | GYTMTWVKQSHGKNLEWIGLINPYNGGTN | YLSWFQQKPGKSPKTLIYRANRLVDGVPSR |
| | YNQKFKGKATFTVDKSSSTAYMELLSLTSE | FSGSGSGQDYSLTISSLEYEEMGIYYCLQYD |
| | DSAVYYCARRHYGSSWDYWGQGTTLTVSS | EFPYTFGGGTKLEIK (SEQ ID NO: 54) |
| | (SEQ ID NO: 21) | |
| KCD040 | EVQLQQSGAELVKPGASVKLSCTASDFNIK | QIVLTQSPAIMSASPGEKVTMTCSAGSSVSY |
| | DTYMHWVMQRPEQGLEWIGKIDPANGNTE | MYWYQQKPGSSPRVLIYDTSNLASGVPVRF |
| | FDPKFQGKATITADTSSNTAYLQLTSLTSED | SGSGSGTSYSLTISRMEAEDAATYYCQQWS |
| | TAVYYCTRAMDYWGQGTSVTVSS (SEQ ID | NYPYTFGGGTKLEIK (SEQ ID NO: 55) |
| | NO: 22) | |
| KCD042 | EVQLQQSGAELVKPGASVRLSCTASGFNIK | QSVLTQSPAIMSASPGEKVTMTCSANSSVSD |
| | HTYIHWVSQRPEQGLEWIGKIDPANGNTKY | MYWFQQRPGSSPRLLIYDTSNLASGVPVRFS |
| | DPKFQGKATITADTSSNTAYLQLSSLTSEDT | GSGSGTSYSLTISRMEAEDAATYYCQQWST |
| | AVYYCVNAMEYWGQGTSVTVSS (SEQ ID | YPWTFGGGTKLEIK (SEQ ID NO: 56) |
| | NO: 23) | |
| KCD044 | EVQLQQSGAELVKPGASVRLSCTASGFNIK | QSVLTQSPAIMSASPGEKVTMTCSANSSVSD |
| | HTYMHWVSQRPERGLEWIGKIDPANGNTK | MYWYQQRPGSSPRLLIYDTSNLASGVPVRF |
| | YDPKFQGKATITADTSSNTVYLQLSSLTSED | SGSGSGTSYSLTISRMEAEDAATYYCQQWS |
| | TAVYYCLNAMEYWGQGTSVTVSS (SEQ ID | TYPWTFGGGTKLEIK (SEQ ID NO: 57) |
| | NO: 24) | |
| KCD047 | EVQLQQSGAEFVKPGASVRLSCTASGFNIK | QIVLTQSPAVMSASPGEKVAMTCSASSSVTY |
| | DTYMHWVKQRPEQGLEWIGRIDPANGYTK | MYWYQQKPGSSPRLLIYDTSNLASGVPVRF |
| | DDPKFQGKATITADTSSNTAYLQLSSLTSED | SGSGSGTSYSLTISRMEAEDAATYYCQQWS |
| | TAVYYCASAMDYWGQGTSVTVSS (SEQ ID | TYPFPFGSGTKLEIK (SEQ ID NO: 58) |
| | NO: 25) | |
| KCD048 | EVQLQQSGADLVKPGASVKLSCTASGFNIK | QIVLTQSPAIMSASPGEKVTLTCSATSSVSY |
| | ATYMHWVRQRPEKGLEWIGRIDPANGHTI | MYWYQQKPGSSPRLLIYDTSNLASGVPVRF |
| | YDPQFQGKATITSDTSSNTAYLQLNSLTSED | SGSGSGTSYSLTISRMEAEDDATYYCQQWS |
| | TAVYYCAEAMDYWGQGTSVTVSS (SEQ ID | NYPFTFGGGTKLEIK (SEQ ID NO: 59) |
| | NO: 26) | |
| KCD070 | EIQLQQTGPELVKPGASVKISCKASGYSFTD | DIVMTQSHKFMSTSVGDRVSITCKASQDVG |
| | YIILWVKQSHGKSLEWIGNINPYYDYTSYNL | TAVAWYQQKPGQSPKLLIYWASTRHTGVP |
| | KFKGKATLTVDKSSSTAYMQLNSLTSEDSA | DRFTGSGSGTDFTLTINNVQSEDLADYFCQQ |
| | VYYCARSDGYYGGDYWGQGTSVTVSS (SEQ | YSSYPWTFGGGTTLEIK (SEQ ID NO: 60) |
| | ID NO: 27) | |
| KCD101 | QVQLQQPGAELVRPGTSVKLSCKASGYTFT | DIQMTQTTSSLSASLGDRVTISCRASQDISNY |
| | SYWMHWVKQRPGQGLEWIGVIDPSDSYTN | LNWYQQKPDGTVKLLIYYPSRLHSGVPSRF |
| | YNQKFKGKATLTVDTSSSTAYMQLSSLTSE | SGSGSGTDYSLTISNLEQEDFATYFCQQGNT |
| | DSAVYYCGRNGYDGSMDYWGQGTSVTVSS | LPYTFGGGTKLEIK (SEQ ID NO: 61) |
| | (SEQ ID NO: 28) | |
| KCD102 | QVQLQQPGAELVRPGTSVKLSCKASGYTFT | EIQMTQTTSSLSASLGDRVTISCRASQDISNY |
| | SYWMHWVKQRPGQGLEWIGVIDPSDSYTN | LNWYQQKPDGTVKLLIYYPSRLHSGVPSRF |
| | YNQKFKGKATLTVDTSSSTAYMQLSSLTSE | SGSGSGTDYSLTISNLEQEDFATYFCQQGNT |
| | DSAVYYCARNGYDGSMDYWGQGTSVTVSS | LPYTFGGGTKLEIK (SEQ ID NO: 62) |
| | (SEQ ID NO: 29) | |
| KCD103 | QVQLQQPGAELVRPGTSVKLSCKASGYTFT | DIQMTQTTSSLSASLGDRVTISCRASQDISNS |
| | SYWMHWVKQRPGQGLEWIGVIDPSDSYTK | LNWYQQKPDGTVKLLIYYTSRLHSRVPSRF |
| | YNQKFKDKATLTVETSSSTAYMQLSSLTSE | SGSGSGTDYSLTISNLDQEDIATYFCQQANT |
| | DSAVYYCAGNGYDGSMDYWGQGTSVTVSS | LPYTFGGGTKLEIK (SEQ ID NO: 63) |
| | (SEQ ID NO: 30) | |
| KCD104 | QVQLQQPGAELVRPGTSVKLSCKASGYTFT | EIQMTQTTSSLSASLGDRVTISCRASQDISNY |
| | SYWMHWVKQRPGQGLEWIGVIDPSDSYTY | LNWYQQKPDGTVKLLIYYPSRLHSGVPSRF |
| | YNQKFKGKATLTVDTSSSTAYMQLSSLTSE | SGSGSGTDYSLTISNLEQEDFATYFCQQGNT |
| | DSAVYYCARNGYDGAMDYWGQGTSVTVSS | LPYTFGGGTKLEIK (SEQ ID NO: 64) |
| | (SEQ ID NO: 31) | |
| KCD118 | EVQLQQSGPELVKPGASVKISCKAFGYTFT | DVLMTQTPLSLPVSLGDQASISCRSSQTIVHS |
| | DYYKNWMRQRHGESLEWIGDINPNSGDAN | NGDTYLEWYLQKPGQSPNLLIYKVSNRFSG |
| | YNQKFKGKATLTVDKSSSTAYMELRSLTSE | VPDRFSGSGSGTDFTLKISR VEAEDLGIYYC |
| | DSAVYYCAREGPSFAYWGHGTLVTVSA | FQGSHVPPTFGGGTKLEIK (SEQ ID NO: 65) |
| | (SEQ ID NO: 32) | |
| KCD119 | EVQLQQSGPELVKPGASVKISCKASGYTFT | DVLMTQTPLSLPVSLGDQASISCRSSQTIVHS |
| | DYYTNWMRQRHGESLEWIGDINPNTGDTS | NGDTYLEWYLQKPGQSPNLLIYKVSNRFSG |
| | YNQKFRVKATLTVDKSSGTAYMGLRSLTSE | VPDRFSGSGSGTDFTLKISRVEAEDLGVYYC |
| | DSAVYYCTREGPSFAYWGQGTLVTVSA | FQGSHVPPTFGGGTTLEIK (SEQ ID NO: 66) |
| | (SEQ ID NO: 33) | |
| KCD121 | EVQLQQSGPELVKPGASVKISCKASGYTFT | DVLMTQTPLSLPVSLGDQASISCRSNQTIVH |
| | DYYKNWMRQRHGESLEWIGDINPNNGDTS | SNGDTYLEWYLQKPGQSPNLLIYKVSNRFS |
| | YNQKFRGKATLTVDKSSSTAFMELRSLTSE | GVPDRFSGSGSGTDFTLRISRVEAEDLGVYY |
| | DSAVYYCAREGPSFAYWGQGTLVTVSA | CFQGSHVPPTFGGGTKLEIK (SEQ ID NO: 67) |
| | (SEQ ID NO: 34) | |
| KCD122 | EVQLQQSGPELVKPGASVKISCKASGYTFT | DVLMTQTPLSLPVSLGDQASISCRSSQTIVHS |
| | DYYKNWMRQRHGESLEWIGDINPNNGDAN | NGDTYLEWYLQKPGQSPNLLIYKVSNRFSG |
| | YNQKFKGKATLTVDKSSSTAYMELRSLTSE | VPDRFSGSGSGTDFTLKISR VEAEDLGVYYC |
| | DSAVYFCAREGPSFAYWGHGTLVTVSA | FQGSHVPPTFGGGTKLEIK (SEQ ID NO: 68) |
| | (SEQ ID NO: 35) | |
| KCD123 | EVHLQQSGPELVKPGASVKISCKASGYTFT | DVLMTQTPLSLPVSLGDQASISCRSSQTIVHS |
| | DFYKNWMRQRHGESLEWIGDINPNNGGTN | NGDTYLEWYLQKPGQSPNLLIYKVSNRFSG |
| | YNQKFKGKATLTVDKSSSTAYMELRSLTSE | VPDRFSGSGSGTDFTLKISRVEAEDLGVYYC |
| | DSAVYYCAREGPSFAYWGQGTLVTVSA | FQGSHVPPTFGGGTKLEIK (SEQ ID NO: 69) |
| | (SEQ ID NO: 36) | |
| KCD124 | EVQLQQSGPELVKPGASVKISCKASGYTFT | DVLMTQTPLSLPVSLGDQASISCRSSQSIVHS |
| | DHYMNWVKQSHGKSLEWIGDINPNNGGTS | NGDTYLEWYLQKPGQSPKLLIYKVSNRFSG |
| | YNQKFKGKATLTVDKSSSTAYMELRSLTSG | VPDRFSGSGSGTDFTLKISRVEAEDLGVYYC |
| | DSAVYYCTREGASFAFWGQGTLVTVSA | FQGSHVPLTFGAGTKLELK (SEQ ID NO: 70) |
| | (SEQ ID NO: 37) | |
| KCD131 | QVQLQQSGPELVKPGASVKISCKASAYTFT | DIQMTQSPASLSVSVGETVTITCRASENIYSH |
| | DYYINWVKQRPGQGPEWIGWIFPGSNSTYS | LAWFQQKQGKSPRLLVYSATNLPDGVPSRF |
| | NEKFEVKATLTVDESSSTAYMLLSSLTSEDS | SGSGSGTQYSLKINILQSEDFGSYYCQHFWG |
| | AVYFCARLGYFGSSYHALDYWGQGTSVTVS | TPWTFGGGTKLEIK (SEQ ID NO: 71) |
| | S (SEQ ID NO: 38) | |
| KCD136 | EVQLQQSVAELVRPGASVKLSCSASGFNIKN | QIVLTQSPAIMSASPGEKVTMTCSASSSVSY |
| | TYMHWVNQRPEQGLEWIGRIDPANGITKY | MYWYQQKPGSSPRLLIYDISNLASGVPVRES |
| | APNFQGKATITADTSSNTAYLQLSNLTSEDT | GSGSGTSYSLTISRMEAEDAATYYCQQWDT |
| | AIYYCTRAMDYWGQGTSVTVSS (SEQ ID NO: | YPWTFGGGTKLEIK (SEQ ID NO: 72) |
| | 39) | |
| KCD200 | EVQLQQSGPELVKPGASVKISCKASGYTFTS | DVLMTQTPLSLPVSLGDQVSISCRSSQTIVHS |
| | YYKNWMRQRHGESLEWIGDINPNSGDTAY | NGDTYLEWYLQKPGQSPNLLIYKVSNRFSG |
| | NQKFKGKATLTVDRSSSTAYMELRSLTSED | VPDRFSGSGSGTDFTLKISR VEAEDLGVYYC |
| | SAVYYCAREGPSFAYWGQGTLVTVSA (SEQ | FQGSHVPPTFGGGTKLEIK (SEQ ID NO: 73) |
| | ID NO: 40) | |
| KCD208 | EVQLQQSVAELVRPGASVKLSCTVSGFNIK | EIVLTQSPALMAASPGEKVTITCSVSSSISSSS |
| | NTYMHWVKQRPEQGLEWIGRIDPANGDTT | LHWYRQKSGTSPKPWIYGTSHLASGVPVRF |
| | YAPKFQGKATITADTSSNSAYLHLSRLTSED | SGSGSGTSYSLTISSMEAEDAATYYCQQWD |
| | TAIYYCSLYDYDGYWGQGTTLTVSS (SEQ ID | TYPWTFGGGTKLEIK (SEQ ID NO: 74) |
| | NO: 41) | |
| KCD214 | EVQLQQSVAEFVRPGASVKLSCTASGFNIKN | EIVLTQSPALMAASPGEKVTITCRVSSSISSSS |
| | TYMHWVKQRPEQGLEWIGRIDPANGNTEY | LHWYQQKSGTSPKPWIYGTSNLASGVPVRF |
| | APKFQGKATITADTSSNTAYLQLSSLTSEDT | SGSRSGTSYSLTISSMEAEDAATYYCQQWSD |
| | AIYYCALYDYDGYWGQGTTLTVSS (SEQ ID | YPWTFGGGTKLEIK (SEQ ID NO: 75) |
| | NO: 42) | |
| KCD220 | QVQLQQSGAELMEPGASVKLSCKATGYTF | DIQMTQTTSSLSASLGDRVTISCRASQDISNY |
| | TGYWIEWVKQRPGHGLEWIGETLPGSDSN | LNWYQQKPDGTVKLLIYYTSNLHSGVPSRF |
| | NYNEKFKGKATFTADTSSNTAYMQLSSLTT | SGSGSGTDYSLTISNLEQEDIATYFCQQDSK |
| | EDSAIYYCARDYSNYWYFDVWGTGTTVTVS | HRTFGGGTKLEIK (SEQ ID NO: 76) |
| | S (SEQ ID NO: 43) | |
| KCD224 | QVQLKESGPGLVAPSQSLSITCTVSGFSLTS | DIQMTQTTSSLSASLGDRVTISCRASQVISNY |
| | YGVDWIRQSPGKGLEWLGVIWGVGSTNYN | LNWYQQKPDGTVKLLIYYTSRLHSGVPSRF |
| | SALKSRLSISKDNSKSQVFLKMNSLQTDDTA | SGSGSGTDYSLTISNLEPEDIATYYCQQYSK |
| | MYYCARSYDGSYWYFDVWGTGTTVTVSS | LPYTFGSGTKLEIK (SEQ ID NO: 77) |
| | (SEQ ID NO: 44) | |
| 119_TAF | EVQLVESGGGLVQPGGSLRLSCAASGYTFT | DIQLTQSPSSLSASVGDRVTITCRSSQTIVHS |
| | DYYMNWVRQAPGKGLEWIGDINPNTGDTS | NGDTYLEWYQQKPGKAPNLLIYKVSNRFSG |
| | YNADFKRRFTFSLDTSKSTAYLQMNSLRAE | VPSRFSGSGSGTDFTLTISSLQPEDFATYYCF |
| | DTAVYYCTREGPSFAYWGQGTLVTVSS | QGSHVPPTFGQGTKVEIK (SEQ ID NO: 78) |
| | (SEQ ID NO: 45) | |
| 119_TAF | EVQLVESGGGLVQPGGSLRLSCAASGYTFT | DIQMTQSPSSLSASVGDRVTITCRSSQTIVHS |
| Germ | DYYMSWVRQAPGKGLEWIGDINPNTGDTS | NGDTYLEWYQQKPGKAPNLLIYKVSNRFSG |
| | YNADSVKGRFTISRDNSKNTLYLQMNSLRA | VPSRFSGSGSGTDFTLTISSLQPEDFATYYCF |
| | EDTAVYYCTREGPSFAYWGQGTLVTVSS | QGSHVPPTFGQGTKVEIK (SEQ ID NO: 79) |
| | (SEQ ID NO: 46) | |
| 119_Human | EVKKPGASVKVSCKASGYTFTDYYMHWVR | DVVMTQSPLSLPVTLGQPASISCRSSQTIVHS |
| Germ | QAPGQGLEWIGDINPNTGDTSYNQKFQGRV | NGDTYLEWFQQRPGQSPNLLIYKVSNRFSG |
| | TSTRDTSISTAYMELSRLRSDDTVVYYCTRE | VPDRFSGSGSGTDFTLKISRVEAEDVGVYYC |
| | GPSFAYWGQGTLVTVSS (SEQ ID NO: 47) | FQGSHVPPTFGGGTKVEIK (SEQ ID NO: 80) |

In some embodiments, the anti-CFD antibody includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 129 (or a heavy chain variable region thereof), and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 130 (or a light chain variable region thereof). In some embodiments, the anti-CFD antibody heavy chain includes the sequence of SEQ ID NO: 129 (with or without the C-terminal lysine). In some embodiments, the CFD antibody light chain can comprise the sequence of SEQ ID NO: 130.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-IL-6 antibody. In some embodiments, the anti-IL-6 antibody includes a heavy chain having any 3 of the heavy chain CDRs in any one of SEQ ID NOs: 81-89, and a light chain having any 3 of the light chain CDRs in any one of ID NOs: 90-92. In some embodiments, the anti-IL-6 antibody includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 81-89, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 90-92.

TABLE 0.2

| ID | Sequence |
|---|---|
| I | EVQLVESGGGLVQPGGSLRLSCAASGFTFSPFAISWVRQAPGKGLEWVAKISPGGSWTYYSDTVTDRFTFSLDTSKSTAYLQMNS |
| | LRAEDTAVYYCARQLWGYYALDVWGQGTLVTVSS (SEQ ID NO: 81) |
| II | EVQLVESGGGLVQPGGSLRLSCAASGFTFSPFAWSWVRQAPGKGLEWVAKISPGGSWTYYSDTVTDRFTFSLDTSKSTAYLQM |
| | NSLRAEDTAVYYCARQLWGYYALDVWGQGTLVTVSS (SEQ ID NO: 82) |
| Iia | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQAWGYYALDIWGQGTLVTVSS (SEQ ID NO: 83) |
| Iib | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQSWGYYALDIWGQGTLVTVSS (SEQ ID NO: 84) |
| Iic | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQGWGYYALDIWGQGTLVTVSS (SEQ ID NO: 85) |
| Iid | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQTWGYYALDIWGQGTLVTVSS (SEQ ID NO: 86) |
| Iie | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQVWGYYALDIWGQGTLVTVSS (SEQ ID NO: 87) |
| Iif | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQQWGYYALDIWGQGTLVTVSS (SEQ ID NO: 88) |
| Iig | EVQLVESGGGLVQPGGSLRLSCAASGFTFSPFAMHWVRQAPGKGLEWVAKISPGGSWTYYSDTVTDRFTFSLDTSKSTAYLQM |
| | NSLRAEDTAVYYCARQKWGYYALDIWGQGTLVTVSS (SEQ ID NO: 89) |

TABLE 0.3

| ID | Sequence |
|---|---|
| III | DIQLTQSPSSLSASVGDRVTITCSASISVSYLYWYQQKPGKAPKLLIYDDSSLASGVPSRFSGSGSGTDFTLTISSLQPEDFATYYCQQ |
| | WSGYPYTFGQGTKVEIK (SEQ ID NO: 90) |
| IV | DIQLTQSPSSLSASVGDRVTITCSASISVSYLYWYQQKPGKAPKLLIYDASSLASGVPSRFSGSGSGTDFTLTISSLQPEDFATYYCQQ |
| | WSGYPYTFGQGTKVEIK (SEQ ID NO: 91) |
| V | DIQLTQSPSSLSASVGDRVTITCSASISVSYLYWYQQKPGKAPKLLIYDDSNLASGVPSRFSGSGSGTDFTLTISSLQPEDFATYYCQQ |
| | WSGYPYTFGQGTKVEIK (SEQ ID NO: 92) |

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a fusion construct comprising a VEGF Trap fused to the heavy chain of an anti-IL-6 antibody. In some embodiments, the fusion construct includes a heavy chain variable region having any 3 of the heavy chain CDRs (or all 3 heavy chain CDRs) in any one of SEQ ID NOs: 93, 95, 97, 99, 101, 103, 105, 107, 109, 111, 113, 115, 117, 119, 121, 123, 125, and 127, and a light chain variable region having any 3 of the light chain CDRs (or all 3 light chain CDRs) in any one of ID NOs: 94, 96, 98, 100, 102, 104, 106, 108, 110, 112, 114, 116, 118, 120, 122, 124, 126, and 128. In some embodiments, the fusion construct includes a heavy chain variable region having any 3 of the heavy chain CDRs (or all 3 heavy chain CDRs) in any one of SEQ ID NOs: 93, 95, 97, 99, 101, 103, 105, 107, 109, 111, 113, 115, 117, 119, 121, 123, 125, and 127, and a light chain variable region having any 3 of the light chain CDRs (or all 3 light chain CDRs) in any one of SEQ ID NOs: 94, 96, 98, 100, 102, 104, 106, 108, 110, 112, 114, 116, 118, 120, 122, 124, 126, and 128, as they are paired in Table 0.4. In some embodiments, the fusion construct includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a heavy chain variable region in any one of SEQ ID NOs: 93, 95, 97, 99, 101, 103, 105, 107, 109, 111, 113, 115, 117, 119, 121, 123, 125, and 127, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a light chain variable region in any one of SEQ ID NOs: 94, 96, 98, 100, 102, 104, 106, 108, 110, 112, 114, 116, 118, 120, 122, 124, 126, and 128. In some embodiments, the fusion construct includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a heavy chain variable region in any one of SEQ ID NOs: 93, 95, 97, 99, 101, 103, 105, 107, 109, 111, 113, 115, 117, 119, 121, 123, 125, and 127, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a light chain variable region in any one of SEQ ID NOs: 94, 96, 98, 100, 102, 104, 106, 108, 110, 112, 114, 116, 118, 120, 122, 124, 126, and 128, as they are paired in Table 0.4. In some embodiments, the fusion construct includes a heavy chain having any 3 of the heavy chain CDRs in any one of SEQ ID NOs: 93, 95, 97, 99, 101, 103, 105, 107, 109, 111, 113, 115, 117, 119, 121, 123, 125, and 127, and a light chain having any 3 of the light chain CDRs in any one of ID NOs: 94, 96, 98, 100, 102, 104, 106, 108, 110, 112, 114, 116, 118, 120, 122, 124, 126, and 128. In some embodiments, the fusion construct includes a heavy chain having any 3 of the heavy chain CDRs in any one of SEQ ID NOs: 93, 95, 97, 99, 101, 103, 105, 107, 109, 111, 113, 115, 117, 119, 121, 123, 125, and 127, and a light chain having any 3 of the light chain CDRs in any one of SEQ ID NOs: 94, 96, 98, 100, 102, 104, 106, 108, 110, 112, 114, 116, 118, 120, 122, 124, 126, and 128, as they are paired in Table 0.4. In some embodiments, the fusion construct includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 93, 95, 97, 99, 101, 103, 105, 107, 109, 111, 113, 115, 117, 119, 121, 123, 125, and 127, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 94, 96, 98, 100, 102, 104, 106, 108, 110, 112, 114, 116, 118, 120, 122, 124, 126, and 128. In some embodiments, the fusion construct includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 93, 95, 97, 99, 101, 103, 105, 107, 109, 111, 113, 115, 117, 119, 121, 123, 125, and 127, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 94, 96, 98, 100, 102, 104, 106, 108, 110, 112, 114, 116, 118, 120, 122, 124, 126, and 128, as they are paired in Table 0.4. In some embodiments, the fusion construct includes: a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a heavy chain variable region in any one of SEQ ID NOs: 93, 95, 97, 99, 101, 103, 105, 107, 109, 111, 113, 115, 117, 119, 121, 123, 125, and 127, and heavy chain CDRs of the 3 heavy chain CDRs in the any one of SEQ ID NOs: 93, 95, 97, 99, 101, 103, 105, 107, 109, 111, 113, 115, 117, 119, 121, 123, 125, and 127; and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to a light chain variable region in any one of SEQ ID NOs: 94, 96, 98, 100, 102, 104, 106, 108, 110, 112, 114, 116, 118, 120, 122, 124, 126, and 128, and light chain CDRs of the 3 light chain CDRs in the any one of ID NOs: 94, 96, 98, 100, 102, 104, 106, 108, 110, 112, 114, 116, 118, 120, 122, 124, 126, and 128, where the SEQ ID NOs of the light chain and heavy chain are paired as they are in Table 0.4. In some embodiments, the fusion construct includes a heavy chain having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 93, 95, 97, 99, 101, 103, 105, 107, 109, 111, 113, 115, 117, 119, 121, 123, 125, and 127 (each with or without the C-terminal lysine), and a light chain having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of SEQ ID NOs: 94, 96, 98, 100, 102, 104, 106, 108, 110, 112, 114, 116, 118, 120, 122, 124, 126, and 128, as they are paired in Table 0.4. In some embodiments, the fusion construct includes a heavy chain having an amino acid sequence of any one of SEQ ID NOs: 93, 95, 97, 99, 101, 103, 105, 107, 109, 111, 113, 115, 117, 119, 121, 123, 125, and 127 (each with or without the C-terminal lysine), and a light chain having an amino acid sequence of any one of SEQ ID NOs: 94, 96, 98, 100, 102, 104, 106, 108, 110, 112, 114, 116, 118, 120, 122, 124, 126, and 128, as they are paired in Table 0.4.

TABLE 0.4

| ID | Heavy chain | Light chain |
|---|---|---|
| A | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAIS | EC (SEQ ID NO: 94) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 93) | |
| B | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDASSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAIS | EC (SEQ ID NO: 96) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 95) | |
| C | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSNLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAIS | EC (SEQ ID NO: 98) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 97) | |
| D | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAWS | EC (SEQ ID NO: 100) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 99) | |
| E | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDASSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAWS | EC (SEQ ID NO: 102) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 101) | |
| F | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSNLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAWS | EC (SEQ ID NO: 104) |
| | WVRQAPGKGLEWVAKISPGGSWTYYSDTVTDRFTFSLDTS | |
| | KSTAYLQMNSLRAEDTAVYYCARQLWGYYALDVWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 103) | |
| G | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAM | EC (SEQ ID NO: 106) |
| | HWVRQAPGKGLEWVAKISPGGSWTYYSDTVTDRFTFSLDT | |
| | SKSTAYLQMNSLRAEDTAVYYCARQAWGYYALDIWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 105) | |
| H | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDASSLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAM | EC (SEQ ID NO: 108) |
| | HWVRQAPGKGLEWVAKISPGGSWTYYSDTVTDRFTFSLDT | |
| | SKSTAYLQMNSLRAEDTAVYYCARQAWGYYALDIWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 107) | |
| I | SDTGRPFVEMYSEIPEIIHMTEGRELVIPCRVTSPNITVTLKK | DIQLTQSPSSLSASVGDRVTITCSASISVSYLYWYQQKPGKAP |
| | FPLDTLIPDGKRIIWDSRKGFIISNATYKEIGLLTCEATVNGH | KLLIYDDSNLASGVPSRFSGSGSGTDFTLTISSLQPEDFATYYC |
| | LYKTNYLTHRQTNTIIDVVLSPSHGIELSVGEKLVLNCTARTE | QQWSGYPYTFGQGTKVEIKRTVAAPSVFIFPPSDEQLKSGT |
| | LNVGIDFNWEYPSSKHQHKKLVNRDLKTQSGSEMKKFLST | ASVVCLLNNFYPREAKVQWKVDNALQSGNSQESVTEQDSK |
| | LTIDGVTRSDQGLYTCAASSGLMTKKNSTFVRVHEKGGGG | DSTYSLSSTLTLSKADYEKHKVYACEVTHQGLSSPVTKSFNRG |
| | SGGGGSEVQLVESGGGLVQPGGSLRLSCAASGFTFSPFAM | EC (SEQ ID NO: 110) |
| | HWVRQAPGKGLEWVAKISPGGSWTYYSDTVTDRFTFSLDT | |
| | SKSTAYLQMNSLRAEDTAVYYCARQAWGYYALDIWGQGTL | |
| | VTVSSASTKGPSVFPLAPSSKSTSGGTAALGCLVKDYFPEPVT | |
| | VSWNSGALTSGVHTFPAVLQSSGLYSLSSVVTVPSSSLGTQT | |
| | YICNVNHKPSNTKVDKKVEPKSCDKTHTCPPCPAPEAAGAP | |
| | SVFLFPPKPKDTLMISRTPEVTCVVVDVSHEDPEVKFNWYV | |
| | DGVEVHNAKTKPREEQYNSTYRVVSVLTVLHQDWLNGKEY | |
| | KCKVSNKALPAPIEKTISKAKGQPREPQVYTLPPSREEMTKN | |
| | QVSLTCLVKGFYPSDIAVEWESNGQPENNYKTTPPVLDSDG | |
| | SFFLYSKLTVDKSRWQQGNVFSCSVMHEALHNHYTQKSLSC | |
| | SPGK (SEQ ID NO: 109) | |
| J | EVQLVESGGGLVQPGGSLRLSCAASGFTFSPFAISWVRQAP | DIQLTQSPSSLSASVGDRVTITCSASISVSYLYWYQQKPGKAP |
| | GKGLEWVAKISPGGSWTYYSDTVTDRFTFSLDTSKSTAYLQ | KLLIYDDSSLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 112) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 111) | |
| K | EVQLVESGGGLVQPGGSLRLSCAASGFTFSPFAISWVRQAP | DIQLTQSPSSLSASVGDRVTITCSASISVSYLYWYQQKPGKAP |
| | GKGLEWVAKISPGGSWTYYSDTVTDRFTFSLDTSKSTAYLQ | KLLIYDASSLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 114) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 113) | |
| L | EVQLVESGGGLVQPGGSLRLSCAASGFTFSPFAISWVRQAP | DIQLTQSPSSLSASVGDRVTITCSASISVSYLYWYQQKPGKAP |
| | GKGLEWVAKISPGGSWTYYSDTVTDRFTFSLDTSKSTAYLQ | KLLIYDDSNLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 116) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDFNWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 115) | |
| M | EVQLVESGGGLVQPGGSLRLSCAASGFTFSPFAWSWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDDSSLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 118) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDFNWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 117) | |
| N | EVQLVESGGGLVQPGGSLRLSCAASGFTFSPFAWSWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDASSLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 120) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDFNWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 119) | |
| O | EVQLVESGGGLVQPGGSLRLSCAASGFTFSPFAWSWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDDSNLASGVPSRFSGSGSGTDFTLTISSLQPEDFATYYC |
| | MNSLRAEDTAVYYCARQLWGYYALDVWGQGTLVTVSSAS | QQWSGYPYTFGQGTKVEIKRTVAAPSVFIFPPSDEQLKSGT |
| | TKGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 122) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 121) | |
| P | EVQLVESGGGLVQPGGSLRLSCAASGFTFSPFAMHWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDDSSLASGVPSRFSGSGSGTDFTLTISSLQPEDFATYYC |
| | MNSLRAEDTAVYYCARQAWGYYALDIWGQGTLVTVSSAST | QQWSGYPYTFGQGTKVEIKRTVAAPSVFIFPPSDEQLKSGT |
| | KGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 124) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 123) | |
| Q | EVQLVESGGGLVQPGGSLRLSCAASGFTFSPFAMHWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDASSLASGVPSRFSGSGSGTDFTLTISSLQPEDFATYYC |
| | MNSLRAEDTAVYYCARQAWGYYALDIWGQGTLVTVSSAST | QQWSGYPYTFGQGTKVEIKRTVAAPSVFIFPPSDEQLKSGT |
| | KGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 126) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 125) | |
| R | EVQLVESGGGLVQPGGSLRLSCAASGFTFSPFAMHWVRQA | DIQLTQSPSSLSASVGDRVTITCSASISVSYLYWYQQKPGKAP |
| | PGKGLEWVAKISPGGSWTYYSDTVTDRFTFSLDTSKSTAYLQ | KLLIYDDSNLASGVPSRFSGSGSGTDFTLTISSLQPEDFATYYC |
| | MNSLRAEDTAVYYCARQAWGYYALDIWGQGTLVTVSSAST | QQWSGYPYTFGQGTKVEIKRTVAAPSVFIFPPSDEQLKSGT |
| | KGPSVFPLAPSSKSTSGGTAALGCLVKDYFPEPVTVSWNSG | ASVVCLLNNFYPREAKVQWKVDNALQSGNSQESVTEQDSK |
| | ALTSGVHTFPAVLQSSGLYSLSSVVTVPSSSLGTQTYICNVNH | DSTYSLSSTLTLSKADYEKHKVYACEVTHQGLSSPVTKSFNRG |
| | KPSNTKVDKKVEPKSCGGGGSGGGGSSDTGRPFVEMYSEI | EC (SEQ ID NO: 128) |
| | PEIIHMTEGRELVIPCRVTSPNITVTLKKFPLDTLIPDGKRIIW | |
| | DSRKGFIISNATYKEIGLLTCEATVNGHLYKTNYLTHRQTNT | |
| | IIDVVLSPSHGIELSVGEKLVLNCTARTELNVGIDENWEYPS | |
| | SKHQHKKLVNRDLKTQSGSEMKKFLSTLTIDGVTRSDQGL | |
| | YTCAASSGLMTKKNSTFVRVHEKDKTHTCPPCPAPEAAGA | |
| | PSVFLFPPKPKDTLMISRTPEVTCVVVDVSHEDPEVKFNWY | |
| | VDGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVLDSD | |
| | GSFFLYSKLTVDKSRWQQGNVFSCSVMHEALHNHYTQKSL | |
| | SCSPGK (SEQ ID NO: 127) | |

In some embodiments, the VEGF Trap comprises human VEGFR1 domain 2 and human VEGFR2 domain 3. In some embodiments, the VEGF Trap includes the amino acid sequence of SEQ ID NO: 133. In some embodiments, the VEGF Trap comprises an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 133.

TABLE 0.5

| | SEQ ID |
|---|---|
| Sequence | NO: |
| SDTGRPFVEMYSEIPEIIHMTEGRELVIPCRVTSPNITVT | 133 |
| LKKFPLDTLIPDGKRIIWDSRKGFIISNATYKEIGLLTCE | |
| ATVNGHLYKTNYLTHRQTNTIIDVVLSPSHGIELSVGEKL | |
| VLNCTARTELNVGIDENWEYPSSKHQHKKLVNRDLKTQSG | |
| SEMKKFLSTLTIDGVTRSDQGLYTCAASSGLMTKKNSTFV | |
| RVHEK | |

In some embodiments a fusion construct includes a heavy chain that includes a CDR_{H}1 that is the CDR_{H}1 in SEQ ID NO: 105; a CDR_{H}2 that is the CDR_{H}2 in SEQ ID NO: 105; a CDR_{H}3 that is the CDR_{H}3 in SEQ ID NO: 105; a CDR_{L}1 that is the CDR_{L}1 in SEQ ID NO: 106; a CDR_{L}2 that is the CDR_{L}2 in SEQ ID NO: 106; and a CDR_{L}3 that is the CDR_{L}3 in SEQ ID NO: 106. In some embodiments, the fusion construct includes a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139). In some embodiments, the heavy chain comprises an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO:105, and the light chain comprises an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO:106. In some embodiments, the heavy chain comprises the amino acid sequence of SEQ ID NO:105 (with or without the C-terminal lysine), and the light chain comprises the amino acid sequence of SEQ ID NO:106. OG2072 denotes a fusion construct that includes an anti-IL-6 antibody fused to a VEGF trap, and that includes a heavy chain of SEQ ID NO:105 (with or without the C-terminal lysine) and a light chain of SEQ ID NO:106.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is a fusion construct comprising a PDGFR extracellular trap fused to the heavy chain of an anti-VEGF-A antibody. In some embodiments, the fusion construct includes a heavy chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 131, and a light chain variable region having an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to SEQ ID NO: 132. In some embodiments, the heavy chain comprises the amino acid sequence of SEQ ID NO:131 (with or without the C-terminal lysine), and the light chain comprises the amino acid sequence of SEQ ID NO:132.

In some embodiments, one or both of the first protein (e.g., the protein conjugated to the phosphorylcholine-containing polymer) and the second protein (e.g., the unconjugated protein or protein that is not conjugated to a phosphorylcholine-containing polymer) is an anti-HTRA1 antibody. In some embodiments, the anti-HTRA1 antibody includes a heavy chain comprising a complementarity determining region 1 CDR_{H}1: FYHVH (SEQ ID NO: SEQ ID NO:140), CDR_{H}2: SIYTSGYTEYASALES (SEQ ID NO:141), and CDR_{H}3: EGLQRVGVLDA (SEQ ID NO:142) or EGLQRVGVFDA (SEQ ID NO:143) or EGLQRVGVMDA (SEQ ID NO:144), and a light chain comprising a CDR_{L}1: RSSQSLLDEAGETYLA (SEQ ID NO:145), CDR_{L}2: EVSLLES (SEQ ID NO:146), and CDR_{L}3: QQATYFPYT (SEQ ID NO:147). In some embodiments, the anti-HTRA1 antibody includes a heavy chain comprising a complementarity determining region 1 CDR_{H}1: GFSLTFYH (SEQ ID NO: SEQ ID NO:148), CDR_{H}2: IYTSGYT (SEQ ID NO:149), and CDR_{H}3: AREGLQRVGVFDA (SEQ ID NO:150) or AREGLQRVGVMDA (SEQ ID NO:151) or AREGLQRVGVLDA (SEQ ID NO:152), and a light chain comprising a CDR_{L}1: QSLLDEAGETY (SEQ ID NO:153), CDR_{L}2: EV, and CDR_{L}3: QQATYFPYT (SEQ ID NO:147). In some embodiments, the anti-HTRA1 antibody includes a heavy chain comprising a complementarity determining region 1 CDR_{H}1: GFSLTFY (SEQ ID NO: SEQ ID NO:154), CDR_{H}2: YTSGY (SEQ ID NO:155), and CDR_{H}3: EGLQRVGVLDA (SEQ ID NO:142) or EGLQRVGVFDA (SEQ ID NO:143) or EGLQRVGVMDA (SEQ ID NO:144), and a light chain comprising a CDR_{L}1: RSSQSLLDEAGETYLA (SEQ ID NO:145), CDR_{L}2: EVSLLES (SEQ ID NO:146), and CDR_{L}3: QQATYFPYT (SEQ ID NO:147). In some embodiments, the heavy chain comprises an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to any one of the VH sequences set forth in Table 0.6, and the light chain comprises an amino acid sequence at least 80%, at least 85%, at least 90%, at least 95%, at least 97%, at least 98%, at least 99%, or about 100%, or a percentage in a range defined by any two of the preceding values (e.g., 80-100%, 85-95%, 90-97%, etc.) identical to the VL sequence set forth in Table 0.7. In some embodiments, the heavy chain comprises any one of the VH sequences set forth in Table 0.6, and the light chain comprises the VL sequence set forth in Table 0.7.

TABLE 0.6

| ID | Sequence |
|---|---|
| HC17 | QVQLVESGGGVVQPGGSLRLSCAASGFSLTFYHVHWVRQAPGKGLEWVASIYTSGYTEY |
| | ASALESRFTISRDTSKSTLYLQMNSLRAEDTAVYYCAREGLQRVGVFDAWGQGTLVTVSS |
| | (SEQ ID NO: 156) |
| HC37 | QVQLVESGGGVVQPGGSLRLSCAASGFSLTFYHVHWVRQAPGKGLEWVASIYTSGYTEY |
| | ASALESRFTISRDTSKSTLYLQMNSLRAEDTAVYYCAREGLQRVGVMDAWGQGTLVTVSS |
| | (SEQ ID NO: 157) |
| HC39 | QVQLVESGGGVVQPGGSLRLSCAASGFSLTFYHVHWVRQAPGKGLEWVASIYTSGYTEY |
| | ASALESRFTISRDTSKSTLYLQMNSLRAEDTAVYYCAREGLQRVGVLDAWGQGTLVTVSS |
| | (SEQ ID NO: 158) |
| HC40 | EVQLVESGGGVVQPGGSLRLSCAASGFSLTFYHVHWVRQAPGKGLEWVASIYTSGYTEY |
| | ASALESRFTISRDTSKSTLYLQMNSLRAEDTAVYYCAREGLQRVGVMDAWGQGTLVTVSS |
| | (SEQ ID NO: 159) |
| HC41 | EVQLVESGGGVVQPGGSLRLSCAASGFSLTFYHVHWVRQAPGKGLEWVASIYTSGYTEY |
| | ASALESRFTISRDTSKSTLYLQMNSLRAEDTAVYYCAREGLQRVGVLDAWGQGTLVTVSS |
| | (SEQ ID NO: 160) |

TABLE 0.7

| ID | Sequence |
|---|---|
| LC | DIQLTQSPSSLSASVGDRVTITCRSSQSLLDEAGETYLAW |
| 21 | FQQKPGKAPKLLIYEVSLLESGVPSRFSGSGSGTDFTLTI |
| | SSLQPEDFATYYCQQATYFPYTFGQGTKVEIK |
| | (SEQ ID NO: 161) |

In some embodiments, the unconjugated protein (e.g., unconjugated antibody or unconjugated fusion construct) can have a conjugation cysteine or an engineered cysteine or a cysteine for conjugation that is blocked-thereby preventing polymer conjugation. In some embodiments, the unconjugated species within the composition is separate from the conjugated species by the fact that the conjugated species was subjected to a conjugation reaction and then unconjugated protein (e.g., unconjugated antibody or unconjugated fusion construct) was then added to the mix to create the composition. In some embodiments, the unconjugated protein does not have a non-native cysteine that can be conjugated to a polymer. In some embodiments, the conjugate includes a polymer conjugated to a cysteine, or free amino groups of the protein, e.g., using N-hydroxysuccinimide (NHS) esters. In some embodiments, the conjugate includes a polymer conjugated to ϵ-amine group of lysines, α-amine group of N-terminal amino acids, and/or δ-amine group of histidines.

In some embodiments, the formulation or therapeutically acceptable composition comprises KSI-301. In some embodiments, KSI-301 comprises OG1950 and its conjugated form with OG1802, OG1953. OG1950 denotes an anti-VEGF-A antibody having a heavy chain of SEQ ID NO:1 (with or without the C-terminal lysine) and a light chain of SEQ ID NO: 2. OG1802 may be bonded to the heavy chain of OG1950 at C443 (EU numbering) to form OG1953. In some embodiments, the first protein (e.g., the protein conjugated to the phosphorylcholine-containing polymer) is or comprises OG1950 conjugated with OG1802 (to form OG1953), and the second protein (e.g., the unconjugated protein or protein that is not conjugated to a phosphorylcholine-containing polymer) is or comprises OG1950. In some embodiments, the formulation or therapeutically acceptable composition comprises about 40 to about 60 mM sodium acetate, about 0.01% to about 0.04% polysorbate 20, about 40 to about 60 mg/mL (total protein concentration) of a mixture of OG1950 and OG1953, the mixture containing about 15% to about 25% OG1950 and about 75% to about 85% OG1953 (e.g., by molar amount or percent composition by total protein mass weight concentration), at pH about 4.5 to about 5.5. In some embodiments, the formulation or therapeutically acceptable composition comprises about 50 mM sodium acetate, about 0.025% polysorbate 20, about 50 mg/mL (total protein concentration) of a mixture of OG1950 and OG1953, the mixture containing about 20% OG1950 and about 80% OG1953 (e.g., by molar amount or percent composition by total protein mass weight concentration), at about pH 5. In some embodiments, the formulation or therapeutically acceptable composition consists of, or consists essentially of, about 50 mM sodium acetate, about 0.025% polysorbate 20, about 50 mg/mL (total protein concentration) of a mixture of OG1950 and OG1953, the mixture containing about 20% OG1950 and about 80% OG1953 (e.g., by molar amount or percent composition by total protein mass weight concentration), at about pH 5. In some embodiments, the second protein is or comprises OG1950IAM (e.g., OG1950 treated with iodoacetamide). FIG. 58A depicts components of a non-limiting example of a formulation of the present disclosure, that can include, among other components, a mixture of an anti-VEGF-A antibody and an anti-VEGF-A antibody conjugate, as described herein.

In some embodiments, the formulation or therapeutically acceptable composition comprises KSI-501. In some embodiments, KSI-501 comprises fusion protein OG2072 and its conjugated form with OG1802, OG2074. OG2072 denotes a fusion construct that includes an anti-IL-6 antibody fused to a VEGF trap, and that includes a heavy chain of SEQ ID NO:105 (with or without the C-terminal lysine) and a light chain of SEQ ID NO:106. OG1802 may be bonded to the heavy chain of OG2072 at C443 (EU numbering) to form OG2074. In some embodiments, the first protein (e.g., the protein conjugated to the phosphorylcholine-containing polymer) is or comprises OG2072 conjugated with OG1802 (to form OG2074), and the second protein (e.g., the unconjugated protein or protein that is not conjugated to a phosphorylcholine-containing polymer) is or comprises OG2072. In some embodiments, the formulation or therapeutically acceptable composition comprises about 50 to about 60 mM sodium acetate, polysorbate 20 (e.g., 0.025% polysorbate 20), sucrose (e.g., 4% sucrose), about 50 mg/mL (total protein concentration) of a mixture of OG2072 and OG2074, the mixture containing 20-40% OG2072 and 60-80% OG2074 (e.g., by molar amount or percent composition by total protein mass weight concentration), at pH about 4.5 to about 5.5. In some embodiments, the formulation or therapeutically acceptable composition comprises about 50 to about 60 mM sodium acetate, about 0.025% polysorbate 20, about 4% sucrose, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5 (e.g., about pH 4.8 to about pH 5). In some embodiments, the formulation or therapeutically acceptable composition consists of, or consists essentially of, about 50 to about 60 mM mM sodium acetate, about 0.025% polysorbate 20, about 4% sucrose, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5 (e.g., about pH 4.8 to about pH 5). In some embodiments, the formulation or therapeutically acceptable composition comprises about 50 mM sodium acetate, about 0.025% polysorbate 20, about 4% sucrose, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5. In some embodiments, the formulation or therapeutically acceptable composition consists of, or consists essentially of, about 50 mM sodium acetate, about 0.025% polysorbate 20, about 4% sucrose, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5. In some embodiments, the formulation or therapeutically acceptable composition does not include sucrose. In some embodiments, the formulation or therapeutically acceptable composition does not include a sugar. In some embodiments, the formulation or therapeutically acceptable composition comprises about 50 to about 60 mM sodium acetate, about 0.025% polysorbate 20, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5 (e.g., about pH 4.8 to about pH 5), optionally where the formulation or therapeutically acceptable composition does not include sucrose. In some embodiments, the formulation or therapeutically acceptable composition consists of, or consists essentially of, 50-60 mM sodium acetate, about 0.025% polysorbate 20, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5 (e.g., about pH 4.8 to about pH 5), optionally where the formulation or therapeutically acceptable composition does not include sucrose. In some embodiments, the formulation or therapeutically acceptable composition includes a histidine acetate buffer, about 0.025% polysorbate 20, about 4% sucrose, about 50 mg/mL (total protein concentration) of a mixture of OG2072 and OG2074, the mixture containing 30% OG2072 and 70% OG2074 (e.g., by molar amount or percent composition by total protein mass weight concentration), at about pH 5.2 to about pH 6.2. FIGS. 58B and 58C depict components of a non-limiting examples of a formulation of the present disclosure, that can include, among other components, a mixture of a fusion construct and a fusion construct conjugate, as described herein.

In some embodiments, the polymer component of the conjugated protein has a molecular weight of about 100,000 Da or more, e.g., about 150,000 Da or more, about 200,000 Da or more, about 350,000 Da or more, about 400,000 Da or more, about 450,000 Da or more, about 500,000 Da or more, about 550,000 Da or more, about 600,000 Da or more, about 650,000 Da or more, about 700,000 or more, about 750,000 Da or more, about 800,000 Da or more, about 850,000 Da or more, about 900,000 Da or more, about 950,000 Da or more, about 1,000,000 Da or more, or a molecular weight in a range defined by any two of the preceding values (e.g., 100,000-1,000,000 Da, 300,000-950,000 Da, 400,000-800,000 Da, 500,000-750.000 Da, 600,000-700,000 Da, 600,000-1,000,000 Da, etc.). In some embodiments, the polymer has a molecular weight of between about 700,000 to about 800,000 Da. In some embodiments, the polymer component of the conjugated protein is any of the polymers disclosed herein. In some embodiments, the polymer component of the conjugated protein is OG1801.

In some embodiments, the phosphorylcholine-containing polymer of the conjugate comprises 2-(methacryloyloxyethyl)-2′-(trimethylammonium)ethyl phosphate (MPC) monomers as set forth below:

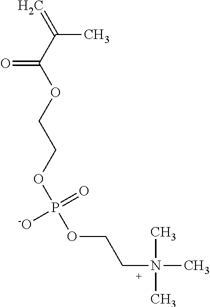

In some embodiments, the polymer of the conjugate has three or more arms or is synthesized with an initiator comprising 3 or more polymer initiation sites. In some embodiments, the polymer of the conjugate has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites. In some embodiments, the polymer of the conjugate has 9 arms or is synthesized with an initiator comprising 9 polymer initiation sites. In some embodiments, the polymer of the conjugate has a polydispersity value (PDI) of less than about 1.2.

In some embodiments, the polymer of the first protein that is conjugated (e.g., first antibody and/or conjugated antibody) has three or more arms or is synthesized with an initiator comprising 3 or more polymer initiation sites. In some embodiments, the polymer of the first protein that is conjugated (e.g., antibody) has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites. In some embodiments, the polymer of the first antibody has a polydispersity index (PDI) of less than about 1.2. In some embodiments, the polymer comprises MPC monomers, wherein first antibody comprises: an amino acid sequence of SEQ ID NO: 1 (with or without the C-terminal lysine); an amino acid sequence of SEQ ID NO: 2, and wherein the antibody is bonded at C449 in SEQ ID NO: 1 to the polymer. In some embodiments, the polymer has 9 arms, and wherein the polymer has a molecular weight of between about 600,000 to about 900,000 Da.

In some embodiments, the first protein or the conjugated protein (e.g., the antibody conjugated to the polymer) is an antibody or a fusion construct, and has the following structure:

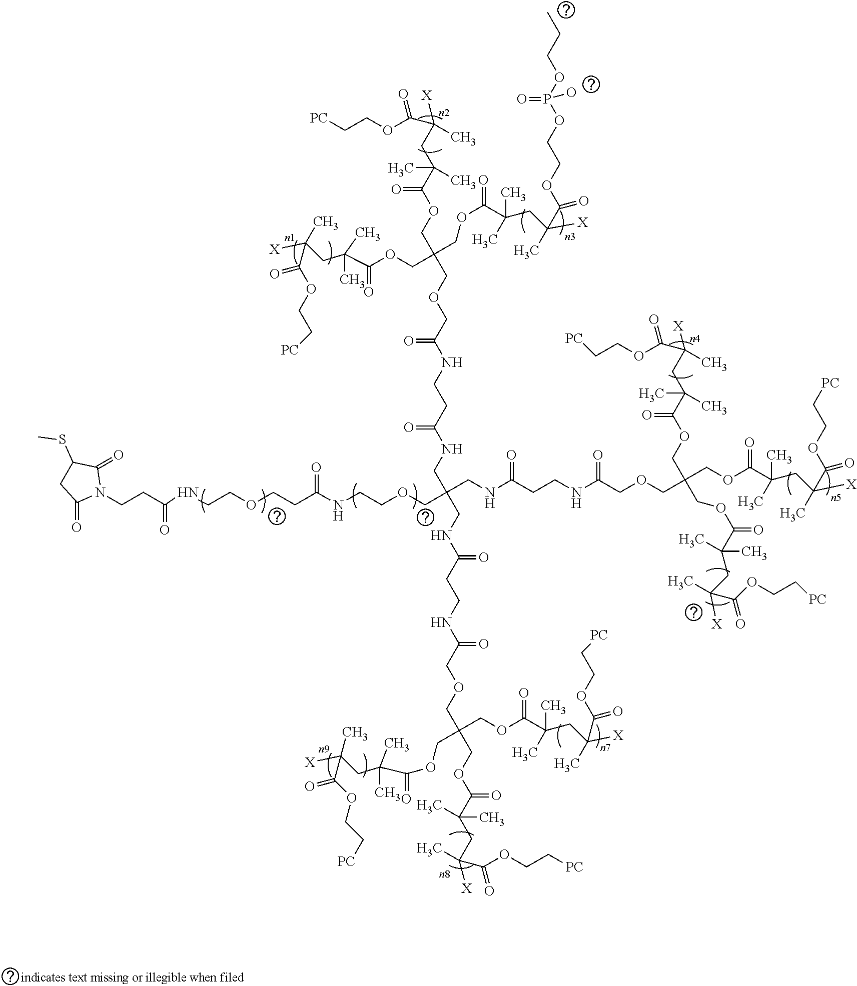

wherein each heavy chain of the antibody or fusion construct is denoted by the letter H, and each light chain of the antibody or fusion construct is denoted by the letter L; the polymer is bonded to the antibody or fusion construct through the sulfhydryl of C443 (EU numbering), which bond is depicted on one of the heavy chains; PC is

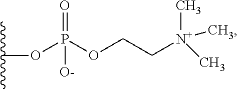 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%. In some embodiments, the sum of n1, n2, n3, n4, n5, n6, n7, n8 and n9 is about 1500 to about 3500 plus or minus about 10% to about 20%. In some embodiments, the first protein or the conjugated protein (e.g., the antibody conjugated to the polymer) is an antibody or a fusion construct, and has the following structure:

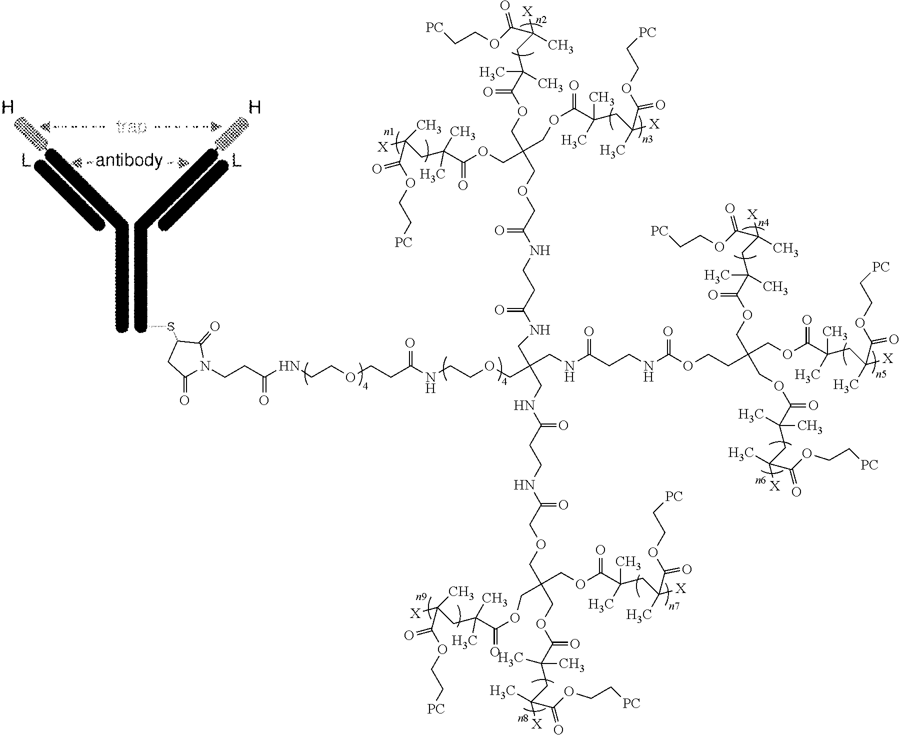

wherein each heavy chain of the antibody or fusion construct is denoted by the letter H, and each light chain of the antibody or fusion construct is denoted by the letter L; the polymer is bonded to the antibody or fusion construct through the sulfhydryl of C443 (EU numbering), which bond is depicted on one of the heavy chains; PC is

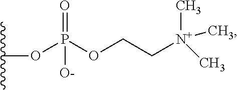 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%.

In some embodiments, the second (and/or the unconjugated) antibody comprises: a CDR_{H}1 that is the CDR_{H}1 in SEQ ID NO: 1; a CDR_{H}2 that is the CDR_{H}2 in SEQ ID NO: 1; a CDR_{H}3 that is the CDR_{H}3 in SEQ ID NO: 1; a CDR_{L}1 that is the CDR_{L}1 in SEQ ID NO: 2; a CDR_{L}2 that is the CDR_{L}2 in SEQ ID NO: 2; a CDR_{L}3 that is the CDR_{L}3 in SEQ ID NO: 2; at least one of the following mutations (EU numbering): L234A, L235A, and G237A; and at least one of the following mutations (EU numbering): Q347C or L443C.

Formulations and compositions provided herein can include a suitable pharmaceutically acceptable carrier. In some embodiments, the pharmaceutically acceptable carrier includes water or a buffer. In some embodiments, the pharmaceutically acceptable carrier comprises a buffer comprising a buffering agent having a pKa that is within, or within about 2, 1, or 0.5 pH units of the pH of the formulation. In some embodiment, the formulation includes as a pharmaceutically acceptable carrier a buffer selected from: acetate, phosphate, citrate, glycine, histidine, HEPES, and Tris buffers. In some embodiments, the formulation includes a sodium acetate buffer. In some embodiments, the formulation includes, includes about, or includes at least 10, 15, 20, 25, 30, 35, 40, 45, 50, 55, 60, 65, 70, 75, 80, 90, 100 mM or more sodium acetate, or optionally, the formulation includes sodium acetate at a concentration in a range defined by any two of the preceding values (e.g., 10-100 mM, 30-80 mM, 40-60 mM, 40-70 mM, etc.). In some embodiments, the formulation includes sodium acetate at a concentration in a range of 40-60 mM. In some embodiments, the formulation includes sodium acetate at a concentration in a range of 30-80 mM. In some embodiments, the formulation includes a histidine acetate buffer. In some embodiments, the formulation includes, includes about, or includes at least 5, 10, 15, 20, 25, 30, 35, 40, 45, 50, 55, 60, 65, 70, 75, 80, 90, 100 mM or more histidine acetate, or optionally, the formulation includes histidine acetate at a concentration in a range defined by any two of the preceding values (e.g., 5-100 mM, 20-80 mM, 10-60 mM, 15-70 mM, 10-30 mM, 15-25 mM, etc.). In some embodiments, the formulation includes histidine acetate at a concentration in a range of 10-60 mM. In some embodiments, the formulation includes histidine acetate at a concentration in a range of 10-30 mM. In some embodiments, the formulation includes histidine acetate at a concentration in a range of 15-25 mM.

In any of the composition or formulation described herein, in some embodiments, the composition or formulation includes a surfactant, such as but not limited to a polysorbate (e.g., polysorbate 20). The composition or formulation can include any suitable amount of the surfactant. In some embodiments, the composition or formulation includes, includes about, or includes at least 0.005, 0.01, 0.015, 0.02, 0.025, 0.03, 0.035, 0.04, 0.05, 0.0.6, 0.07, 0.08, 0.09, 0.1% (w/w) or more of the surfactant, or optionally, the composition or formulation includes a percentage (w/w) of the surfactant in a range defined by any two of the preceding values (e.g., 0.005-0.1%, 0.01-0.05%, 0.02-0.03%, 0.015-0.08%, etc.). In some embodiments, the composition or formulation includes the surfactant at about 0.01% to about 0.04% (w/w). In some embodiments, the composition or formulation includes the surfactant at about 0.02% to about 0.03% (w/w). In some embodiments, the composition or formulation includes about 0.025% (w/w) surfactant. In some embodiments, the surfactant is or comprises polysorbate 20 or polysorbate 80 or poloxamer 188. In some embodiments, the surfactant is polysorbate 20. In some embodiments, the composition or formulation includes about 0.01-0.05% (w/w) polysorbate 20. In some embodiments, the composition or formulation includes about 0.02-0.03% (w/w) polysorbate 20. In some embodiments, the composition or formulation includes about 0.025% (w/w) polysorbate 20. In some embodiments, the surfactant is poloxamer 188.

In any of the composition or formulation described herein, in some embodiments, the composition or formulation includes a tonicity agent. In some embodiments, the tonicity agent is or includes a sugar. In some embodiments, the tonicity agent is or includes sucrose or trehalose. The composition or formulation can include any suitable amount of the tonicity agent. In some embodiments, the composition or formulation includes, includes about, or includes at least 2.0%, 2.5%, 3.0%, 3.5%, 4.0%, 4.5%, 5.0%, 5.5%, 6.0%, 6.5%, 7.5%, 8.0%, 8.5%, 9.0%, 10.0% (w/v) or more of the tonicity agent, or optionally, the composition or formulation includes a percentage (w/v) of the tonicity agent in a range defined by any two of the preceding values (e.g., 2-10%, 2.5-6%, 3-5%, 3.5-8.5%, etc.). In some embodiments, the composition or formulation includes the tonicity agent at about 2% to about 10% (w/v). In some embodiments, the composition or formulation includes the tonicity agent at about 3% to about 5% (w/v). In some embodiments, the composition or formulation includes about 4% (w/v) tonicity agent. In some embodiments, the composition or formulation includes about 2% to about 10% (w/v) sucrose. In some embodiments, the composition or formulation includes about 3% to about 5% (w/v) sucrose. In some embodiments, the composition or formulation includes about 4% (w/v) sucrose.

Also provided is an intraocular therapeutic composition comprising an anti-VEGF-A antibody at about 50 mg/mL of protein, the anti-VEGF-A antibody comprising: a heavy chain comprising a complementarity determining region 1 (CDR_{H}1): GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11); and a light chain comprising CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14), wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody, wherein the antibody conjugate comprises the anti-VEGF-A antibody conjugated to a phosphorylcholine-containing polymer at a non-native cysteine outside a variable region of the antibody, wherein the phosphorylcholine-containing polymer is present in the composition at about 100 mg/mL or more, wherein the phosphorylcholine-containing polymer has a molecular weight of between 300,000 and 1,200,000 Da, wherein the pH of the composition is about 5.5 or lower.

Provided herein is an intraocular therapeutic composition comprising an anti-VEGF-A antibody at about 50 mg/mL of protein, the anti-VEGF-A antibody comprising: a heavy chain comprising an amino acid sequence of SEQ ID NO: 1 (with or without the C-terminal lysine); and a light chain comprising an amino acid sequence of SEQ ID NO: 2, wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody, wherein the antibody conjugate comprises the following structure:

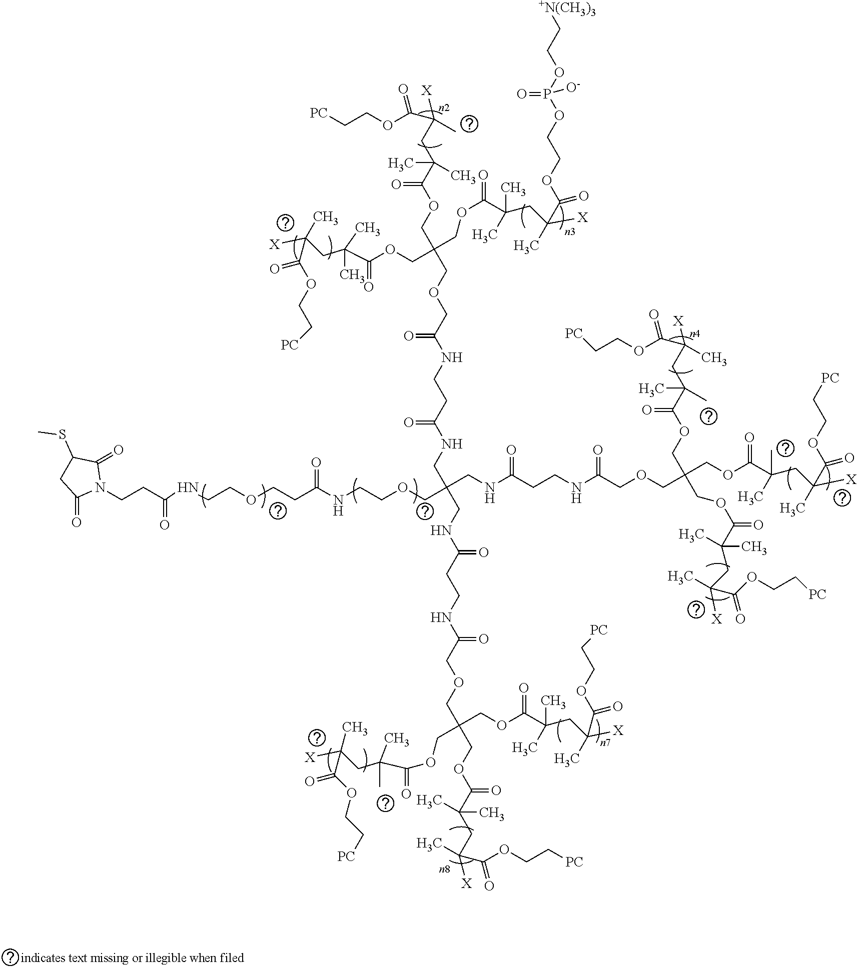

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted on one

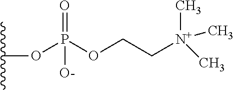 [SMILES: *OP(=O)([O-])OCC[N+](C)(C)C]

of the heavy chains; PC is, where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5.5 or lower.

Also provided is an intraocular therapeutic composition comprising a fusion construct at about 53 mg/mL of protein, the fusion construct comprising a VEGF trap fused to an anti-IL-6 antibody, wherein the fusion construct comprises: a heavy chain comprising an amino acid sequence of SEQ ID NO:105 (with or without the C-terminal lysine), and a light chain comprising an amino acid sequence of SEQ ID NO:106, wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the fusion construct conjugated to a phosphorylcholine-containing polymer, wherein the phosphorylcholine-containing polymer is present in the composition at about 100 mg/mL or more, wherein the pH of the composition is about 5 or lower.

Provided herein is an intraocular therapeutic composition comprising a fusion construct at about 53 mg/mL of protein, the fusion construct comprising a VEGF trap fused to an anti-IL-6 antibody, wherein the fusion construct comprises: a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139), wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the following structure:

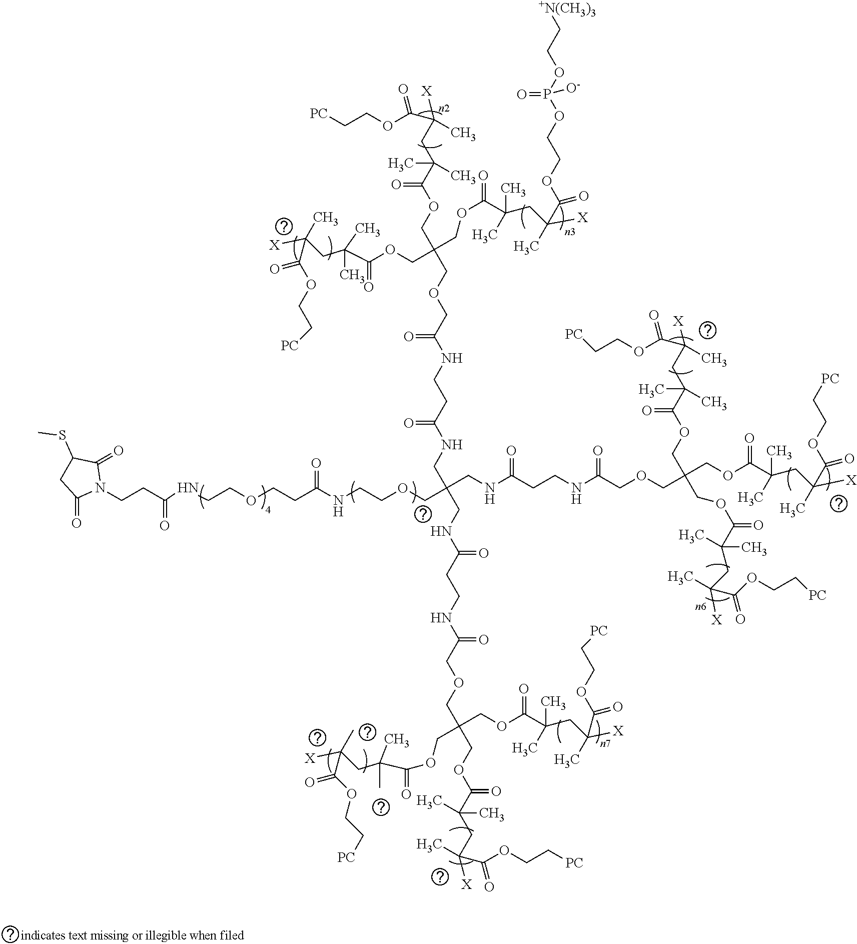

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted

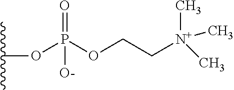 [SMILES: *OP(=O)([O-])OCC[N+](C)(C)C]

on one of the heavy chains; PC is, where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5 or lower. Also provided is an intraocular therapeutic composition comprising a fusion construct at about 53 mg/mL of protein, the fusion construct comprising a VEGF trap fused to an anti-IL-6 antibody, wherein the fusion construct comprises: a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139), wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct, wherein the conjugate comprises the following structure:

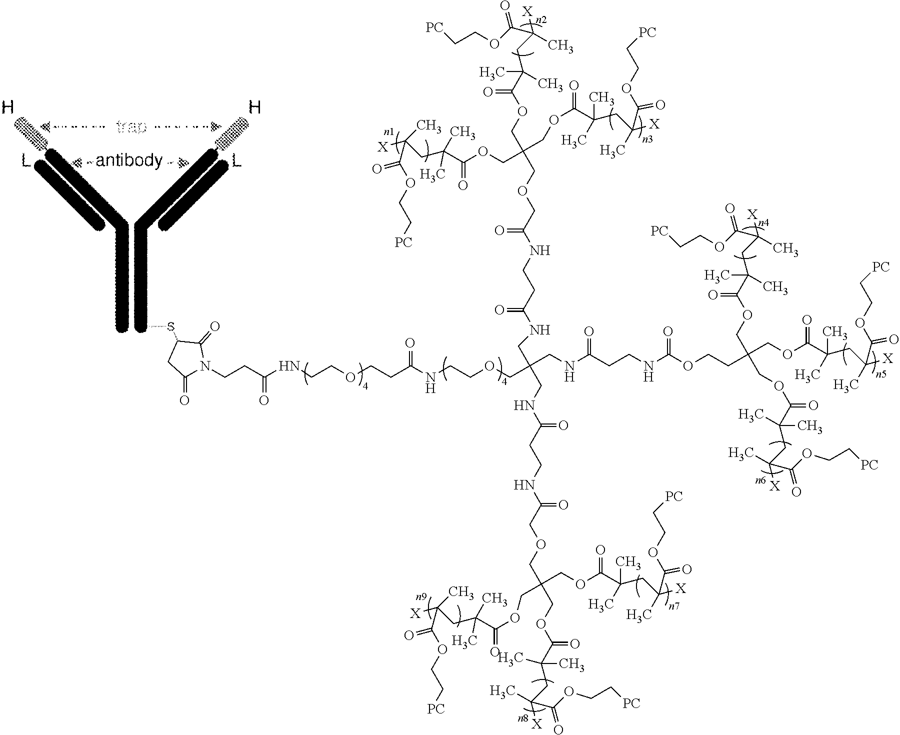

wherein: each heavy chain of the conjugate is denoted by the letter H, and each light chain of the conjugate is denoted by the letter L; the polymer is bonded to the heavy chain of the conjugate through the sulfhydryl of C443 (EU numbering), which bond is depicted on one of the heavy chains; PC is, where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and n1, n2, n3, n4, n5, n6, n7,

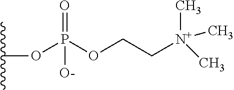 [SMILES: *OP(=O)([O-])OCC[N+](C)(C)C]

n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5 or lower.

In some embodiments, a method of making a composition comprising an anti-VEGF-A antibody conjugated to a phosphorylcholine-containing polymer is provided. The method comprises: conjugating a first antibody to a phosphorylcholine-containing polymer, wherein the polymer is covalently bonded to the first antibody at a cysteine outside a variable region of the first antibody; combining a second antibody that is not conjugated to the phosphorylcholine-containing polymer with the first antibody. In some embodiments, the second antibody is any anti-VEGF Fab. In some embodiments, the second antibody is a Lucentis antibody (ranibizumab). In some embodiments, the second antibody is present in the composition at a percent composition of 5% to 93% relative to the total protein mass weight concentration of the first antibody and the second antibody in the composition. In some embodiments, the second antibody is present in the composition at a percent composition of 5% to 50% relative to the total protein mass weight concentration of the first antibody and the second antibody in the composition. In some embodiments, the second antibody is present in the composition at a percent composition of 5% to 25% relative to the total protein mass weight concentration of the first antibody and the second antibody in the composition. In some embodiments, the purified antibody conjugate has at least a 1.5-fold increase in half-life relative to an unconjugated anti-VEGF-A antibody.

In some embodiments, unconjugated biologics have rapid efficacy in wAMD. In some embodiments, unconjugated biologics have deep efficacy in wAMD. In some embodiments, for any of the compositions herein, clinical durability of the composition resulting in little to no disease recurrence is desirable.

In some embodiments, ranibizumab port delivery reservoir implant delivery efficacy in wAMD is desirable. In some embodiments, any of the compositions herein can result in longer durability without sacrificing immediate efficacy in wAMD by combining a conjugated version of the antibody in combination with an unconjugated version of the antibody. In some embodiments, the combination of the conjugated and unconjugated antibody provides a desired balance of immediacy (or bolus activity), and of durability (or basal activity). In some embodiments, any of the formulations and compositions herein can act to slow progress of retinal disease. In some embodiments, any of the formulations and compositions herein can act to stop progress of retinal disease. In some embodiments, any of the formulations and compositions herein are prepared with, or with about a 10% loading, or a 15% loading, or a 20% loading or a 25% loading, or a 30% loading, or a 35% loading of OG1950 mAb.

In some embodiments, the composition comprises a first antibody and a second antibody: wherein the first antibody is a conjugate; wherein the first antibody and second antibody have the same molecular weight, but the first antibody and second antibody can be the same or different proteins. In some embodiments, the composition comprises a first antibody and a second antibody: wherein the first antibody is a conjugate; wherein the first antibody and second antibody have a similar molecular weight, but the first antibody and second antibody can be the same or different proteins. In some embodiments, the composition comprises a first antibody and a second antibody: wherein the first antibody is a conjugate; wherein the first antibody and second antibody have different molecular weights, but the first antibody and second antibody can be the same or different proteins. In some embodiments, this composition comprises a first active moiety and a second active moiety that can be any protein. In some embodiments, this composition comprises a first active moiety and a second active moiety in which the first active moiety is a full antibody and a second active moiety is an antibody fragment. In some embodiments, this composition comprises a first active moiety and a second active moiety in which the first active moiety is a full antibody and a second active moiety is an antibody fragment of the first active moiety.

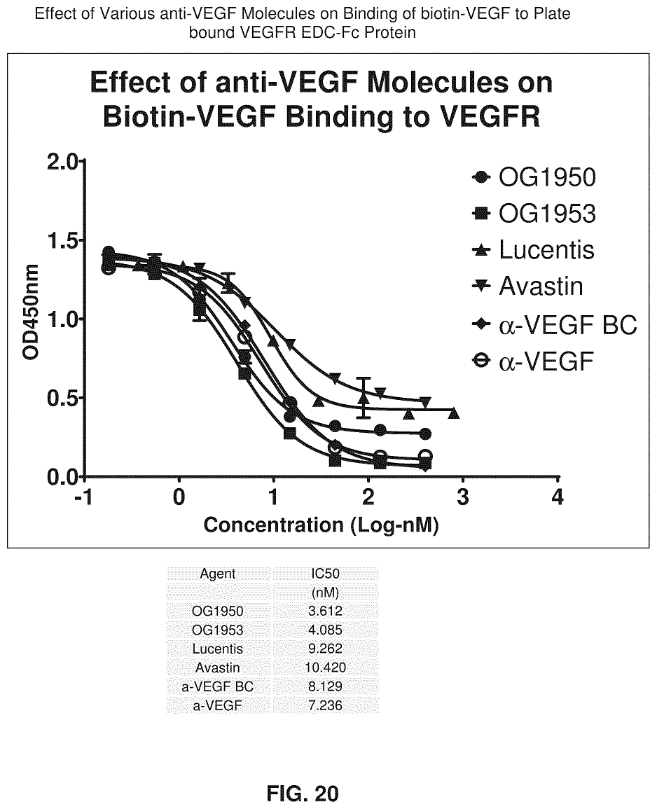

In some embodiments, an anti-VEGF antibody conjugate, in a mixed antibody and antibody-conjugate composition, is provided that is capable of blocking at least 90% of an interaction between a VEGF ligand (“VEGFL”) and a VEGF-receptor (“VEGFR”). For example, it can block at least 90, 91, 92, 93, 94, 95, 96, 97, 98, 99%, or effectively all of the interaction between VEGFR and VEGFL. In some embodiments, the noted blocking occurs at saturating concentrations. In some embodiments, an anti-VEGF antibody conjugate (with an additional unconjugated protein) blocks at least 95% of an interaction between a VEGF ligand and a VEGF-receptor. As an example of such superiority of blocking of the conjugate itself, see FIG. 20, regarding the ability of OG1953 (and antibody conjugate provided herein) to block to a higher degree than Lucentis®(ranibizumab) or Avastin®(bevacizumab) or even the antibody OG1950 (unconjugated).

In some embodiments, the antibodies mixed with the conjugates thereof, of the composition thereof inhibit at least 70, 80, 85, 86, 87, 88, 89, 90, 91, 92, 93, 94, 95, 96, 97, 98, 99, or 100% of the activity and/or interaction between VEGFR and VEGFL. In some embodiments, the IC50 value can be 0.1, 1, 2, 3, 4, 5, 6, 7, 8, 9, 10, 20, 30, 40, 50, 100 nM or less than any one or more of the preceding values. In some embodiments, the KD can be 2*10\hat{ }-13, 1*10\hat{ }-13, 1*10\hat{ }-12, 1*10\hat{ }-11, 1*10\hat{ }-10M or less than any one of the preceding values. In some embodiments, the IC₅₀ value can be 1, 5, 10, 20, 30, 40, 50, 60, 70 80, 90, 100, 200, 300, 400, 500, 600, 700, 800, 900, 1,000, 1,100, 1,200, 1,300, or less than any one of the preceding values. In some embodiments, this functionality can be in the antibody, the antibody conjugate, or both.

In some embodiments, an anti-VEGF antibody of the composition is provided that blocks at least 90% of an interaction between a VEGF ligand and a VEGF-receptor. For example, it can block at least 91, 92, 93, 94, 95, 96, 97, 98, 99%, or effectively all of the interaction between VEGFR and VEGFL. As an example of such superiority of blocking, see FIG. 20, regarding the ability of OG1950 (and antibody provided herein) to block to a higher degree than Lucentis®(ranibizumab) or Avastin®(bevacizumab). In some embodiments, this functionality can be in the antibody, the antibody conjugate, or both.

In some embodiments, the composition can comprise other antibodies, such as Lucentis®(ranibizumab) or Avastin®(bevacizumab), and those antibodies can be conjugated to one or more of the polymers as described herein, by one or more of the processes described herein. In some embodiments, any antibody, or fragment thereof, can be conjugated to one or more of the polymers as described herein, by one or more of the processes described herein. In some embodiments, these can be the antibody, the antibody conjugate, or both.

In some embodiments the antibody of the composition comprises a heavy chain amino acid variable region that comprises SEQ ID NO: 1 (with or without the C-terminal lysine), and a light chain amino acid variable region that comprises SEQ ID NO: 2. In some embodiments, the antibody of the composition is conjugated to one or more of the polymers provided herein. In some embodiments, the conjugated antibody of the composition is at least 90% identical to SEQ ID NO: 1 and/or 2. In some embodiments, the antibody of the composition contains the 6 CDRs within SEQ ID NO:1 and SEQ ID NO: 2, as well as a point mutation of L443C (EU numbering, or 449C in SEQ ID NO: 1). In some embodiments, the conjugated antibody of the composition is at least 90% identical to SEQ ID NO: 1 and/or 2 and includes the following mutations: L234A, L235A, and G237A (EU numbering), and at least one of the following mutations: Q347C (EU numbering) or L443C (EU numbering).

In some embodiments an antibody that binds to VEGF-A is provided in the composition. The antibody comprises: a CDR_{H}1 that is the CDR_{H}1 in SEQ ID NO: 1, a CDR_{H}2 that is the CDR_{H}2 in SEQ ID NO: 1, a CDR_{H}3 that is the CDR_{H}3 in SEQ ID NO: 1, a CDR_{L}1 that is the CDR_{L}1 in SEQ ID NO: 2, a CDR_{L}2 that is the CDR_{L}2 in SEQ ID NO: 2, a CDR_{L}3 that is the CDR_{L}3 in SEQ ID NO: 2, at least one of the following mutations: L234A, L235A, and G237A (EU numbering), and at least one of the following mutations: Q347C (EU numbering) or L443C (EU numbering).

As will be appreciated by one of skill in the art, in light of the present specification, any of the antibodies provided herein can be conjugated to any of the polymers provided herein and/or any antibody provided herein can have a cysteine added such that it allows for site specific conjugation to a polymer. In some embodiments, these can be the antibody, the antibody conjugate, or both.

“VEGF” or “vascular endothelial growth factor” is a human vascular endothelial growth factor that affects angiogenesis or an angiogenic process. In particular, the term VEGF means any member of the class of growth factors that (i) bind to a VEGF receptor such as VEGFR-1 (Flt-1), VEGFR-2 (KDR/Flk-1), or VEGFR-3 (FLT-4); (ii) activates a tyrosine kinase activity associated with the VEGF receptor; and (iii) thereby affects angiogenesis or an angiogenic process.

The VEGF family of factors is made up of five related glycoproteins: VEGF-A (also known as VPE), -B, -C, -D and PGF (placental growth factor). Of these, VEGF-A is the most well studied and is the target of anti-angiogenic therapy. Ferrara et al, (2003) Nat. Med. 9:669-676. VEGF-A exists as a number of different isotypes which are generated both by alternative splicing and proteolysis: VEGF-A₂₀₆, VEGF-Ais₉, VEGF-A₁₆s, and VEGF-A₁₂₁. The isoforms differ in their ability to bind heparin and non-signaling binding proteins called neuropilins. The isoforms are all biologically active as dimers.

The various effects of VEGF are mediated by the binding of a VEGF, e.g., VEGF-A (P15692), —B (P49766), —C(P49767) and -D (Q43915), to receptor tyrosine kinases (RTKs). The VEGF family receptors belong to class V RTKs and each carry seven Ig-like domains in the extracellular domain (ECD). In humans, VEGF binds to three types of RTKs: VEGFR-1 (Flt-1) (P17948), VEGFR-2 (KDR, Flk-1) (P935968) and VEGFR-3 (Flt-4) (P35916). Unless otherwise apparent from the context reference to a VEGF means any of VEGF-A, -B, -C, -D, and PGF, in any of the natural isoforms or natural variants or induced variants having at least 90, 95, 98 or 99% or 100% sequence identity to a natural form. In some embodiments, such VEGFs are human VEGFs. Likewise reference to a VEGFR means any of VEGR-1, R-2 or R-3, including any natural isoform or natural variant, or an induced variant having at least 90, 95, 98 or 99% or 100% sequence identity to a natural sequence.

VEGF antagonist therapies have been approved for the treatment of certain cancers and wet AMD. Bevacizumab (AVASTIN, Genentech/Roche) is a humanized mouse monoclonal antibody that binds to and neutralizes human VEGF, in particular to all isoforms of VEGF-A and to bioactive proteolytic fragments of VEGF-A. See, e.g., Ferrara N, Hillan K J, Gerber H P, Novotny W. 2004. Discovery and development of bevacizumab, an anti-VEGF antibody for treating cancer. Nat Rev Drug Discov. 3(5):391-400. Bevacizumab has been approved for the treatment of certain cancers. The protein sequence of the heavy and light chains of bevacizumab (DrugBank DB00112) are set forth in SEQ ID NO. 3 (heavy) and SEQ ID NO. 4 (light).

Bevacizumab variable light chain CDRs are CDR_{L}1: SASQDISNYLN (SEQ ID NO: 12), CDR_{L}2: FTSSLHS (SEQ ID NO: 13) and CDR_{L}3: QQYSTVPWT (SEQ ID NO: 14). Bevacizumab variable heavy chain CDRs are CDR_{H}1: GYTFTNYGMN, CDR_{H}2: WINTYTGEPTYAADFKR (SEQ ID NO: 10), and CDR_{H}3: YPHYYGSSHWYFDV. CDRs are defined by Kabat except CDR_{H}1 uses the composite Kabat/Chothia definition. In some embodiments, a cysteine can be added to the Bevacizumab sequence and the antibody (and/or a variant that includes the 6 CDRs of Bevacizumab) can be conjugated to any one or more of the polymers provided herein.

Another anti-VEGF molecule, derived from the same mouse monoclonal antibody as bevacizumab has been approved as a treatment for wet AMD: ranibizumab (LUCENTIS®(ranibizumab), Genentech/Roche). Ranibizumab is an antibody fragment or Fab. Ranibizumab was produced by affinity maturation of the variable heavy and light chains of bevacizumab. The sequence of the heavy and light chains of ranibizumab (as published by Novartis) is set forth in SEQ ID NO. 5 and 6 respectively. In some embodiments, a cysteine can be added to the ranibizumab sequence and the antibody (and/or a variant that includes the 6 CDRs of ranibizumab) can be conjugated to any one or more of the polymers provided herein.

The Ranibizumab CDRS are the same as Bevacizumab except where an improvement was added after affinity maturation: Ranibizumab variable light chain CDRs are CDR_{L}1: SASQDISNYLN (SEQ ID NO: 12), CDR_{L}2: FTSSLHS (SEQ ID NO: 13) and CDR_{L}3: QQYSTVPWT (SEQ ID NO: 14). Ranibizumab variable heavy chain CDRs are CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11).

In some embodiments, an antibody conjugate of the composition is presented having an anti-VEGF-A antibody bonded at a cysteine outside a variable region of the antibody to a phosphorylcholine containing polymer, wherein the cysteine has been added via recombinant DNA technology. In some embodiments, the polymer is bonded to a single cysteine. In some embodiments, “added by recombinant DNA technology” means that the cysteine residue replaces a non-cysteine amino acid that occurs in the same position in a known or existing antibody or in a consensus antibody sequence. Thus, for example where the antibody is an IgG1 and the heavy chain possess a leucine at EU position 443, the leucine is replaced via recombinant DNA technology with a cysteine (L443C, EU numbering, or 449C in SEQ ID NO: 1). Correspondingly, the native IgG1 sequence at EU position 347 is Q (glutamine) and the Q is replaced with cysteine via recombinant DNA technology to yield Q347C.

In some embodiments, the anti-VEGF-A antibody of the composition comprises a light chain and a heavy chain where the heavy chain has an Fc region. In some embodiments, the cysteine is in the Fc region and the anti-VEGF-A antibody of the composition is an immunoglobulin G (IgG). In some embodiments, the anti-VEGF-A heavy chain has CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11), and position 221 (via sequential counting as in SEQ ID NO. 3) is T, and the anti-VEGF-A light chain has CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14), and Kabat position 4 is L.

In some embodiments, the anti-VEGF-A heavy chain isotype is IgG1. In some embodiments, the IgG1 constant domain has one or more mutations relative to an IgG1 constant domain (e.g. constant region of SEQ ID NO. 3) to modulate effector function. In some embodiments, the effector function mutations are one or more of the following: (EU numbering) E233X, L234X, L235X, G236X, G237X, A327X, A330X, and P331X wherein X is any natural or unnatural amino acid. In some embodiments, the mutations are selected from the group consisting of (EU numbering): E233P, L234V, L234A, L235A, G237A, A327G, A330S, and P331S. In some embodiments, antibody conjugate of the composition has the following mutations (EU numbering): L234A, L235A, and G237A.

In some embodiments, the cysteine residue is in the anti-VEGF-A heavy chain and is Q347C (EU numbering) or L443C (EU numbering). In some embodiments, the cysteine residue is L443C (EU numbering, or 449C in SEQ ID NO: 1). In some embodiments, the sequence of the anti-VEGF-A heavy chain is SEQ ID NO: 1 (with or without the C-terminal lysine), and the sequence of the anti-VEGF-A light chain is SEQ ID NO: 2.

In some embodiments, the phosphorylcholine containing polymer comprises 2-(methacryloyloxyethyl)-2′-(trimethylammonium)ethyl phosphate (MPC) monomers as set forth below:

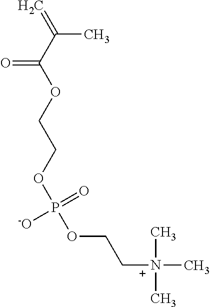

such that the polymer comprises the following repeating units:

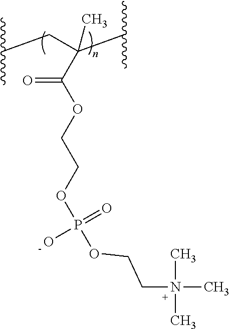

where n is an integer from 1 to 3000 and the wavy lines indicate the points of attachment between monomer units in the polymer.

In some embodiments, the polymer has three or more arms, or is synthesized with an initiator comprising 3 or more polymer initiation sites. In some embodiments, the polymer has 2, 3, 4, 5, 6, 7, 8, 9, 10, 11 or 12 arms, or is synthesized with an initiator comprising 2, 3, 4, 5, 6, 7, 8, 9, 10, 11, or 12 polymer initiation sites. More preferably, the polymer has 3, 6, or 9 arms, or is synthesized with an initiator comprising 3, 6, or 9 polymer initiation sites. In some embodiments, the polymer has 9 arms, or is synthesized with an initiator comprising 9 polymer initiation sites.

In some embodiments, the polymer that is added has a molecular weight between about 300,000 and about 1,750,000 Da (SEC-MALs). In some embodiments, the polymer has a molecular weight between about 500,000 and about 1,000,000 Da. In some embodiments, the polymer has a molecular weight of between about 600,000 to about 900,000 Da. In some embodiments, the polymer has a molecular weight of between about 700,000 to about 800,000 Da. In some embodiments, the polymer has a molecular weight of between about 750,000 to about 850,000 Da. In some embodiments, the polymer has a molecular weight of between about 800,000 to about 850,000 Da. In some embodiments, the polymer has a molecular weight of between about 750,000 to about 800,000 Da.

In some embodiments, any of the antibodies described herein of the composition can be further conjugated to a polymer to form a bioconjugate. The molecular weight of the bioconjugate (in total, SEC-MALs) can be between about 350,000 and 2,000,000 Daltons, for example, between about 450,000 and 1,900,000 Daltons, between about 550,000 and 1,800,000 Daltons, between about 650,000 and 1,700,000 Daltons, between about 750,000 and 1,600,000 Daltons, between about 850,000 and 1,500,000 Daltons, between about 900,000 and 1,400,000 Daltons, between about 950,000 and 1,300,000 Daltons, between about 900,000 and 1,000,000 Daltons, between about 1,000,000 and 1,300,000 Daltons, between about 850,000 and 1,300,000 Daltons, between about 850,000 and 1,000,000 Daltons, and between about 1,000,000 and 1,200,000 Daltons.

In some embodiments, the antibody conjugate is purified. In some embodiments, the polymer aspect of the antibody conjugate is polydisperse, i.e. the polymer PDI is not 1.0. In some embodiments, the PDI is less than 1.5. In some embodiments, the PDI is less than 1.4. In some embodiments, the PDI is less than 1.3. In some embodiments the PDI is less than 1.2. In some embodiments the PDI is less than 1.1.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NO. 1, and the sequence of the anti-VEGF-A light chain is SEQ ID NO. 2, and wherein the antibody is bonded only at C449 in SEQ ID NO. 1 to the polymer. In some embodiments, the polymer has 9 arms and has a molecular weight of between about 600,000 to about 1,000,000 Da.

In some embodiments, the antibody conjugate has an anti-VEGF-A immunoglobulin G (IgG) bonded to a polymer, which polymer comprises MPC monomers, wherein the sequence of the anti-VEGF-A heavy chain is SEQ ID NO. 1, and the sequence of the anti-VEGF-A light chain is SEQ ID NO. 2, and wherein the antibody is bonded only at C443 (EU numbering, or 449C in SEQ ID NO: 1) to the polymer. In some embodiments, the polymer has 9 arms and has a molecular weight of between about 600,000 to about 1,000,000 Da.

In some embodiments, the antibody conjugate has the following structure:

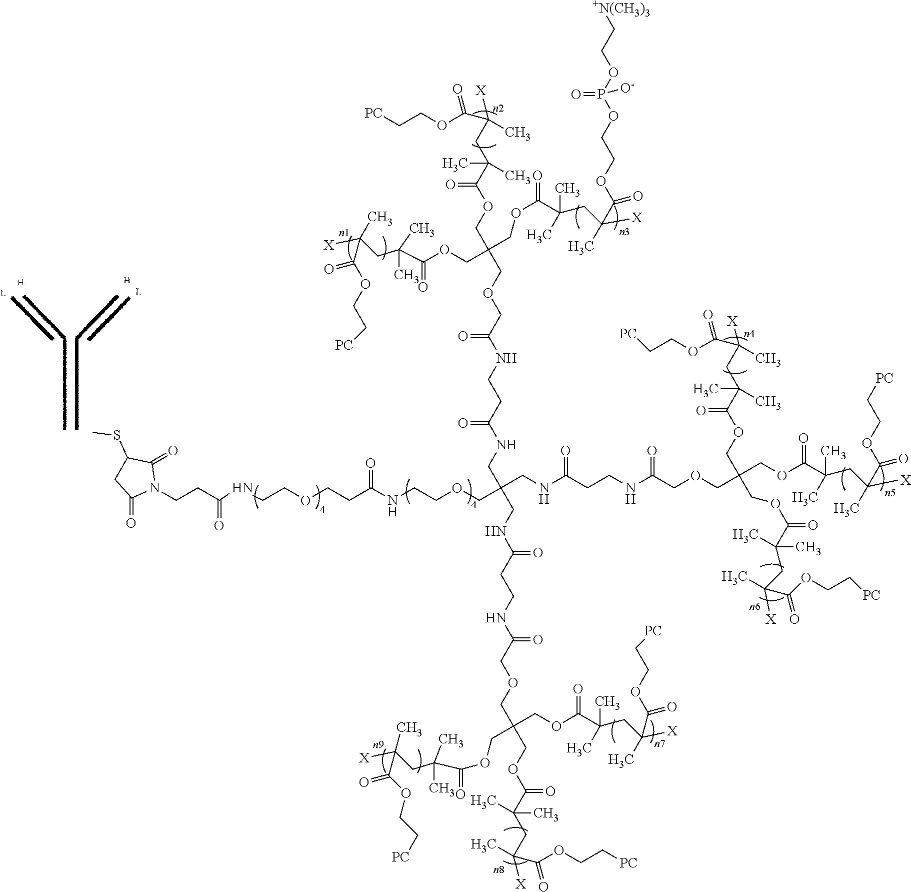

wherein: each heavy chain of the anti-VEGF-A antibody is denoted by the letter H, and each light chain of the anti-VEGF-A antibody is denoted by the letter L; the polymer is bonded to the anti-VEGF-A antibody through the sulfhydryl of C449 of SEQ ID NO: 1, which bond is depicted on one of the heavy chains; PC is,

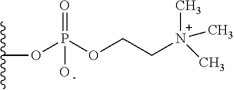 [SMILES: *OP(=O)(O)OCC[N+](C)(C)C]

where the curvy line indicates the point of attachment to the rest of the polymer; wherein X is a) —OR where R is H, methyl, ethyl, propyl, or isopropyl, b) —H, c) any halogen, including —Br, —Cl, or —I, d) —SCN, or e) —NCS; and n1, n2, n3, n4, n5, n6, n7, n8 and n9 are the same or different such that the sum of n1, n2, n3, n4, n5, n6, n7, n8 and n9 is 2500 plus or minus 15%. In some embodiments, the sum of n1, n2, n3, n4, n5, n6, n7, n8 and n9 is about 1500 to about 3500 plus or minus about 10% to about 20%. In some embodiments, X is —OR, where R is a sugar, an aminoalkyl, mono-substituted, poly-substituted or unsubstituted variants of the following residues: saturated C₁-C₂₄ alkyl, unsaturated C₂-C24 alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, —CO—O—R₇, carbonyl —CCO—R₇, —CO—NR₈R9, —(CH₂)ₙ—COOR₇, —CO—(CH)ₙ—COOR₇, —(CH₂)ₙ—NR₈R₉, ester, alkoxycarbonyl, aryloxycarbonyl, wherein n is an integer from 1 to 6, wherein each R₇, R₈ and R₉ is separately selected from the group consisting of a hydrogen atom, halogen atom, mono-substituted, poly-substituted or unsubstituted variants of the following residues: saturated C₁-C₂₄alkyl, unsaturated C₂-C₂₄ alkenyl or C₂-C₂₄ alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, amino, aminocarbonyl, aminocarboyloxy, nitro, azido, phenyl, hydroxy, alkylthio, arylthio, oxysulfonyl, carboxy, cyano, and halogenated alkyl including polyhalogenated alkyl, a 5-membered ring, and a 6-membered ring.

In some embodiments, the antibody conjugate is present in a liquid formulation. In some embodiments, the antibody conjugate is combined with a pharmaceutically acceptable carrier.

In some embodiments, an anti-VEGF-A antibody is presented. The anti-VEGF-A antibody heavy chain has at least the following CDR sequences: CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11). In some embodiments, the anti-VEGF-A heavy chain has those CDRs and in addition has threonine (T) at position 221 (via sequential counting as in SEQ ID NO. 3). In some embodiments, the anti-VEGF-A light chain has at least the following CDRs: CDR_{L}1: SASQDISNYLN (SEQ ID NO: 12), CDR_{L}2: FTSSLHS (SEQ ID NO: 13) and CDR_{L}3: QQYSTVPWT (SEQ ID NO: 14). In some embodiments, the anti-VEGF-A antibody has those CDRs and in addition has leucine (L) at Kabat position 4. In some embodiments, the isotype of the anti-VEGF-A antibody heavy chain is IgG1 and has a CH₁, hinge, CH₂ and CH₃ domains. In some embodiments the light chain isotype is kappa.

In some embodiments, the IgG1 domain of the anti-VEGF-A antibody has one or more mutations to modulate effector function, such as ADCC, ADCP, and CDC. In some embodiments, the IgG1 mutations reduce effector function. In some embodiments the amino acids to use for effector function mutations include (EU numbering) E233X, L234X, L235X, G236X, G237X, G236X, D270X, K322X, A327X, P329X, A330X, A330X, P331X, and P331X, in which X is any natural or non-natural amino acid. In some embodiments, the mutations include one or more of the following: E233P, L234V, L234A, L235A, G237A, A327G, A330S and P331S (EU numbering). In some embodiments, the anti-VEGF-A heavy chain has the following mutations (EU numbering): L234A, L235A and G237A. In some embodiments, the number of effector function mutations relative to a natural human IgG1 sequence is no more than 10. In some embodiments the number of effector function mutations relative to a natural human IgG1 sequence is no more than 5, 4, 3, 2 or 1. In some embodiments, the antibody has decreased Fc gamma binding and/or complement C1q binding, such that the antibody's ability to result in an effector function is decreased. This can be especially advantageous for ophthalmic indications/disorders.

In some embodiments, the anti-VEGF-A antibody comprises one or more of the following amino acid mutations: L234A, L235A, G237A (EU numbering), and L443C (EU numbering, or 449C in SEQ ID NO: 1).

In some embodiments, the anti-VEGF-A antibody is or is part of a human immunoglobulin G (IgG1).

In some embodiments, the VEGF-A antibody comprises a heavy chain constant domain that comprises one or more mutations that reduce an immune-mediated effector function.

In some embodiments an anti-VEGF-A antibody is provided. The anti-VEGF-antibody comprises a heavy chain that comprises a CDR_{H}1 comprising the sequence GYDFTHYGMN (SEQ ID NO: 9), a CDR_{H}2 comprising the sequence WINTYTGEPTYAADFKR (SEQ ID NO: 10), a CDR_{H}3 comprising the sequence YPYYYGTSHWYFDV (SEQ ID NO: 11), a CDR_{L}1 comprising the sequence SASQDISNYLN (SEQ ID NO: 12), a CDR_{L}2 comprising the sequence FTSSLHS (SEQ ID NO: 13), and a CDR_{L}3 comprising the sequence QQYSTVPWT (SEQ ID NO: 14).

Alternatively, the IgG domain can be IgG2, IgG3 or IgG4 or a composite in which constant regions are formed from more than one of these isotypes (e.g., CH1 region from IgG2 or IgG4, hinge, CH2 and CH3 regions from IgG1). Such domains can contain mutations to reduce and/or modulate effector function at one or more of the EU position mentioned for IgG1. Human IgG2 and IgG4 have reduced effector functions relative to human IgG1 and IgG3.

The anti-VEGF-A heavy chain has a cysteine residue added as a mutation by recombinant DNA technology which can be used to conjugate a half-life extending moiety. In some embodiments, the mutation is (EU numbering) Q347C (EU numbering) and/or L443C (EU numbering, or 449C in SEQ ID NO: 1). In some embodiments, the mutation is L443C (EU numbering, or 449C in SEQ ID NO: 1). In some embodiments, the stoichiometry of antibody to polymer is 1:1; in other words, a conjugate has one molecule of antibody conjugated to one molecule of polymer.

The half-life of the anti-VEGF-A antibodies can be extended by attachment of a “half-life (“half life”) extending moieties” or “half-life (“half life”) extending groups”. Half-life extending moieties include peptides and proteins which can be expressed in frame with the biological drug of issue (or conjugated chemically depending on the situation) and various polymers which can be attached or conjugated to one or more amino acid side chain or end functionalities such as —SH, —OH, —COOH, —CONH2, —NH2, or one or more N- and/or O-glycan structures. Half-life extending moieties generally act to increase the in vivo circulatory half-life of biologic drugs.

Examples of peptide/protein half-life extending moieties include Fc fusion (Capon D J, Chamow S M, Mordenti J, et al. Designing CD4 immunoadhesions for AIDS therapy. Nature. 1989. 337:525-31), human serum albumin (HAS) fusion (Yeh P, Landais D, Lemaitre M, et al. Design of yeast-secreted albumin derivatives for human therapy: biological and antiviral properties of a serum albumin-CD4 genetic conjugate. Proc Natl Acad Sci USA. 1992. 89:1904-08), carboxy terminal peptide (CTP) fusion (Fares F A, Suganuma N. Nishimori K, et al. Design of a long-acting follitropin agonist by fusing the C-terminal sequence of the chorionic gonadotropin beta subunit to the follitropin beta subunit. Proc Natl Acad Sci USA. 1992. 89:4304-08), genetic fusion of non-exact repeat peptide sequence (XTEN) fusion (Schellenberger V, Wang C W, Geething N C, et al. A recombinant polypeptide extends the in vivo half-life of peptides and proteins in a tunable manner. Nat Biotechnol. 2009. 27:1186-90), elastin like peptide (ELPylation) (MCpherson DT, Morrow C, Minehan D S, et al. Production and purification of a recombinant elastomeric polypeptide, G(VPGVG19-VPGV, from Escheriachia coli. Biotechnol Prog. 1992. 8:347-52), human transferrin fusion (Prior C P, Lai C—H, Sadehghi H et al. Modified transferrin fusion proteins. Patent WO2004/020405. 2004), proline-alanine-serine (PASylation) (Skerra A, Theobald I, Schlapsky M. Biological active proteins having increased in vivo and/or vitro stability. Patent WO2008/155134 A1. 2008), homo-amino acid polymer (HAPylation) (Schlapschy M, Theobald I, Mack H, et al. Fusion of a recombinant antibody fragment with a homo-amino acid polymer: effects on biophysical properties and prolonged plasma half-life. Protein Eng Des Sel. 2007. 20:273-84) and gelatin like protein (GLK) fusion (Huang Y—S, Wen X—F, Zaro J L, et al. Engineering a pharmacologically superior form of granulocyte-colony-stimulating-factor by fusion with gelatin-like protein polymer. Eur J. Pharm Biopharm. 2010. 72:435-41).

Examples of polymer half-life extending moieties include polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(1-hydroxymethyethylene hydroxymethylformal) (PHF), a zwitterionic polymer, a phosphorylcholine containing polymer and a polymer comprising MPC, Poly (Glyₓ-Sery), Hyaluronic acid (HA), Heparosan polymers (HEP), Fleximers, Dextran, and Poly-sialic acids (PSA).

In one embodiment a half-life extending moiety can be conjugated to an antibody via free amino groups of the protein using N-hydroxysuccinimide (NHS) esters. Reagents targeting conjugation to amine groups can randomly react to ϵ-amine group of lysines, α-amine group of N-terminal amino acids, and 6-amine group of histidines.

However, the anti-VEGF-A antibodies disclosed herein have many amine groups available for polymer conjugation. Conjugation of polymers to free amino groups, thus, might negatively impact the ability of the antibody proteins to bind to VEGF.

In some embodiments, a half-life extending moiety is coupled to one or more free SH groups using any appropriate thiol-reactive chemistry including, without limitation, maleimide chemistry, or the coupling of polymer hydrazides or polymer amines to carbohydrate moieties of the antibody after prior oxidation. In some embodiments maleimide coupling is used. In some embodiments, coupling occurs at cysteines naturally present or introduced via genetic engineering.

In some embodiments, polymers are covalently attached to cysteine residues introduced into anti-VEGF-A antibodies by site directed mutagenesis. In some embodiments, the cysteine residues are employed in the Fc portion of the antibody. In some embodiments, the sites to introduce cysteine residues into an Fc region are provided in WO 2013/093809, U.S. Pat. No. 7,521,541, WO 2008/020827, U.S. Pat. Nos. 8,008,453, 8,455,622 and US2012/0213705, incorporated herein by reference for all purposes. In some embodiments, the cysteine mutations are Q347C (EU numbering) and L443C referring to the human IgG heavy chain by EU numbering.

In some embodiments, conjugates of antibody and high MW polymers serving as half-life extenders are provided. In some embodiments, a conjugate comprises an antibody that is coupled to a zwitterionic polymer wherein the polymer is formed from one or more monomer units and wherein at least one monomer unit has a zwitterionic group is provided. In some embodiments, the zwitterionic group is phosphorylcholine.

In some embodiments, one of the monomer units is HEMA-PC. In some embodiments, a polymer is synthesized from a single monomer which is HEMA-PC.

In some embodiments, some antibody conjugates have 2, 3, or more polymer arms wherein the monomer is HEMA-PC. In some embodiments, the conjugates have 2, 3, 4, 5, 6, 7, 8, 9, 10, 11, or 12 polymer arms wherein the monomer is HEMA-PC. In some embodiments, the conjugates have 3, 6 or 9 arms. In some embodiments, the conjugate has 9 arms.

In some embodiments, polymer-antibody conjugates have a polymer portion with a molecular weight of between 100,000 and 1,500,000 Da. In some embodiments, the conjugate has a polymer portion with a molecular weight between 500,000 and 1,000,000 Da. In some embodiments, the conjugate has a polymer portion with a molecular weight between 600,000 to 800,000 Da. In some embodiments, the conjugate has a polymer portion with a molecular weight between 600,000 and 850,000 Da and has 9 arms. When a molecular weight is given for an antibody conjugated to a polymer, the molecular weight will be the addition of the molecular weight of the protein, including any carbohydrate moieties associated therewith, and the molecular weight of the polymer.

In some embodiments, an anti-VEGF-A antibody has a HEMA-PC polymer which has a molecular weight measured by Mw of between about 100 kDa and 1650 kDa is provided. In some embodiments, the molecular weight of the polymer as measured by Mw is between about 500 kDa and 1000 kDa. In some embodiments, the molecular weight of the polymer as measured by Mw is between about 600 kDa to about 900 kDa. In some embodiments, the polymer molecular weight as measured by Mw is 750 kDa plus or minus 15%.

In some embodiments, the polymer is made from an initiator suitable for ATRP having one or more polymer initiation sites. In some embodiments, the polymer initiation site has a 2-bromoisobutyrate site. In some embodiments, the initiator has 3 or more polymer initiation sites. In some embodiments, the initiator has 3, 4, 5, 6, 7, 8, 9, 10, 11 or 12 polymer initiation sites. In some embodiments, the initiator has 3, 6 or 9 polymer initiation sites. In some embodiments, the initiator has 9 polymer initiation sites. In some embodiments, the initiator is OG1786.

The anti-VEGF-A antibodies can be produced by recombinant expression including (i) the production of recombinant DNA by genetic engineering, (ii) introducing recombinant DNA into prokaryotic or eukaryotic cells by, for example and without limitation, transfection, electroporation or microinjection, (iii) cultivating the transformed cells, (iv) expressing antibody, e.g. constitutively or on induction, and (v) isolating the antibody, e.g. from the culture medium or by harvesting the transformed cells, in order to (vi) obtain purified antibody.

The anti-VEGF-A antibodies can be produced by expression in a suitable prokaryotic or eukaryotic host system characterized by producing a pharmacologically acceptable antibody molecule. Examples of eukaryotic cells are mammalian cells, such as CHO, COS, HEK 293, BHK, SK-Hip, and HepG2. Other suitable expression systems are prokaryotic (e.g., E. coli with pET/BL21 expression system), yeast (Saccharomyces cerevisiae and/or Pichia pastoris systems), and insect cells.

A wide variety of vectors can be used for the preparation of the antibodies disclosed herein and are selected from eukaryotic and prokaryotic expression vectors. Examples of vectors for prokaryotic expression include plasmids such as, and without limitation, preset, pet, and pad, wherein the promoters used in prokaryotic expression vectors include one or more of, and without limitation, lac, trc, trp, recA, or araBAD. Examples of vectors for eukaryotic expression include: (i) for expression in yeast, vectors such as, and without limitation, pAO, pPIC, pYES, or pMET, using promoters such as, and without limitation, AOX1, GAP, GALL, GTH1 or AUG1; (ii) for expression in insect cells, vectors such as and without limitation, pMT, pAc5, pIB, pMIB, or pBAC, using promoters such as and without limitation PH, p10, MT, Ac5, OpIE2, gp64, or polh, and (iii) for expression in mammalian cells, vectors such as, and without limitation, pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and beta-actin.

A formulation or composition of the present disclosure can be prepared using any suitable option. In some embodiments, the formulation is prepared by combining the protein that is not conjugated to a phosphorylcholine-containing polymer with a protein conjugated to a phosphorylcholine-containing polymer. In some embodiments, the formulation is prepared by combining the protein that is not conjugated to a phosphorylcholine-containing polymer with a protein conjugated to a phosphorylcholine-containing polymer in respective amounts sufficient to achieve the desired proportion of conjugated and unconjugated protein in the formulation. For example and without limitation, for a total mass weight concentration of 50 mg/mL, a formulation or therapeutically acceptable composition can be prepared by combining an amount of the unconjugated protein that corresponds to 10 mg/mL in the final composition (percent composition of 20%), with an amount of a protein conjugate that corresponds to 40 mg/mL of the protein portion of the protein conjugate (excluding any contribution of the polymer to the mass weight concentration calculation) in the final composition.

Provided herein is a method of preparing a formulation, comprising combining in a formulation: a first molar amount of a conjugate comprising a first protein conjugated to a phosphorylcholine-containing polymer; and a second molar amount of a second protein that is not conjugated to a phosphorylcholine-containing polymer, wherein the formulation comprises the second protein at about 1% or more (e.g., about 5-90%, 15-25%, 25-35%, 25-40%, etc.) of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein. The formulation can include any suitable amount of the first and second protein, as provided herein.

In some embodiments, preparing the formulation includes adding an amount of a modified protein that does not have a reactive group (e.g., reactive cysteine) for conjugation with the protein before conjugation, and carrying out the conjugation reaction whereby only the reactive proteins are conjugated with the polymer, while the modified protein remains in unconjugated form. In some embodiments, preparing the formulation includes providing a free protein without engineered cysteine for maleimide polymer conjugation, and mixing this in during or after the conjugation and purification process to result in mix formulation.

In some embodiments, the pH of the formulation can be any suitable pH units away from the pI of the second protein, as provided herein. In some embodiments, the pH of the formulation, if desired, can be adjusted to achieve the target pH using any suitable method. In some embodiments, the method includes adjusting the pH, before or after combining. In some embodiments, a method of preparing the compositions described herein comprising the step of adjusting the composition pH away from the isoelectric (pI) point of at least one of the proteins present in the composition to obtain a clear solution is provided.

Provided herein is a method of preparing a formulation, comprising adjusting the pH of a formulation to be about 0.5 pH units away or more from the isoelectric point (pI) of an unconjugated protein comprised in the formulation, wherein the formulation comprises: a first molar amount of a conjugate comprising a first protein conjugated to a phosphorylcholine-containing polymer; and a second molar amount of the unconjugated protein, wherein the unconjugated protein is not conjugated to a phosphorylcholine-containing polymer, wherein the formulation comprises the unconjugated protein at about 1% or more (e.g., about 5-90%, 15-25%, 25-35%, 25-40%, etc.) of a total molar amount of the conjugate and the unconjugated protein, wherein the total molar amount comprises a sum of the first molar amount and the second molar amount.

In some embodiments, a method of preparing a formulation of a conjugated protein and an unconjugated protein includes: providing an initial formulation comprising a conjugate comprising a first protein conjugated to a phosphorylcholine-containing polymer; combining in a second formulation a second protein that is not conjugated to a phosphorylcholine-containing polymer with the conjugate; and adjusting the pH of the initial and/or second formulation to be about 0.5 pH units away or more from the isoelectric point (pI) of the second protein, to thereby prepare a mix formulation comprising the conjugate at a first molar amount and the second protein at a second molar amount, wherein the mix formulation comprises the second protein at about 1% or more (e.g., about 5-90%, 15-25%, 25-35%, 25-40%, etc.) of a total molar amount of the conjugate and the second protein, wherein the total molar amount comprises a sum of the first molar amount and the second molar amount. In some embodiments, the pH of the formulation is adjusted such that the prepared formulation has a suitable pH, as provided herein.

In some embodiments, the formulation before adjusting the pH, before or after the unconjugated protein and the conjugated protein are combined, is at or around the pI of the unconjugated protein. In some embodiments, the unconjugated protein, before combining with the conjugated protein and adjusting the pH, is in a solution that is not or is substantially not turbid. In some embodiments, when the unconjugated protein is combined with the conjugated protein before adjusting the pH, the solution turns turbid. In some embodiments, after adjusting the pH, the formulation of the unconjugated protein and the conjugated protein is or becomes clear and non-turbid (e.g., by visual inspection, or as measured in NTU units, as provided herein).

Adjusting the pH of the formulation can be done using any suitable option. In some embodiments, adjusting the pH comprises substituting a buffering system of the formulation, wherein the buffering system is substituted to a buffering system that buffers at a pH that is about 0.5 pH units or more away from the pI of the protein. In some embodiments, the buffering agent of the buffering system is selected to achieve the desired pH for the formulation. In some embodiments the buffering system includes a buffer selected from but not limited to acetate, phosphate, citrate, glycine, histidine, HEPES, MES, MOPS, and Tris buffers. In some embodiments, adjusting the pH involves substituting an initial buffer (e.g., phosphate buffer) with an acetate buffer. Any suitable option can be used to substitute one buffering system with another, such as without limitation, dialysis, diafiltration, dilution followed by ultrafiltration, chromatography, etc. In some embodiments, adjusting the pH comprises adding a base or acid to raise or lower the pH.

In some embodiments, methods of the present disclosure provide for a low-viscosity formulation of a protein conjugated to a phosphorylcholine-containing polymer. The method can include combining in a formulation: a first molar amount of a conjugate comprising a first protein conjugated to a phosphorylcholine-containing polymer; and a second molar amount of the protein that is not conjugated to a phosphorylcholine-containing polymer, wherein the formulation comprises the protein that is not conjugated to the phosphorylcholine-containing polymer at about 1% or more (e.g., about 5-90%, 15-25%, 25-35%, 25-40%, etc.) of a total molar amount of the conjugate unconjugated proteins, wherein the total molar amount comprises a sum of the first molar amount and the second molar amount, wherein the formulation has a pH that is about 0.5 pH units away or more from the isoelectric point (pI) of the second protein, wherein the formulation has reduced viscosity and/or an enhanced injectability compared to a reference formulation comprising the conjugate at the total molar amount. In some embodiments, the prepared formulation has any suitable viscosity as provided herein. In some embodiments, the reference formulation has a high viscosity, as provided herein.

In any method of preparing a formulation or composition of the present disclosure, in some embodiments, the prepared formulation or composition can be defined as a percent composition (e.g., in mass weight concentration) of one component relative to the total mass weight concentration of the proteins in the composition, as described herein. In some embodiments, a method of preparing a formulation or composition defined in % total molar amount of the second protein (e.g., the unconjugated protein) can be defined in percent composition (e.g., in mass weight concentration) of the second protein relative to the total mass weight concentration of the first and second proteins, given the relevant molecular weight of each protein.

Combining the conjugated protein with the unconjugated protein can be carried out using any suitable option. In some embodiments, an amount of the conjugated protein is combined with an amount of the unconjugated protein at the desired ratio. In some embodiments, the conjugated and unconjugated proteins are combined as part of the process for conjugating the polymer to the protein to be conjugated. For example, the extent of conjugation can be adjusted such that not all of the initially provided unconjugated protein is conjugated as a result of the conjugation reaction. In some embodiments, the unconjugated protein is combined with the conjugate protein at the end of the conjugation process. In some embodiments, the conjugate is prepared by conjugating the protein to the polymer in a conjugation reaction, removing unconjugated protein remaining in the conjugation reaction to generate a purified conjugate, then combining the purified conjugate with an amount of the second protein that is not conjugated to a polymer. In some embodiments, the conjugated and unconjugated proteins are combined through a combination of (i) adding an amount of unconjugated protein and (ii) adjusting the extent of the conjugation reaction. In some embodiments, the conjugation process generates the unconjugated and conjugated protein of the composition or formulation from a common source of the unconjugated protein.

In some embodiments, preparing the formulation includes conjugating the polymer to the first protein. Suitable options for conjugating the polymer to the first protein include those provided herein, and in US patent publication no. 2017/0190766 and 2019/0270806, the entireties of which are incorporated herein by reference.

In some embodiments, preparing the formulation includes carrying out a conjugation reaction of the protein with the polymer using a lower excess of polymer to protein than required to conjugate all the protein with the polymer, such that some fraction of the protein remains unconjugated. In some embodiments, preparing the formulation includes lowering the polymer to protein excess ratio to reduce the conjugation efficiency during the conjugation process, of which there will be more unreacted free protein in the final purified solution, which can result in higher free protein content at the end.

In some embodiments, any of the formulations or compositions herein can be produced by using a low molar excess ratio of polymer to protein by using an optimal process parameter for the antibody conjugate. In some embodiments, lowering the conjugation efficiency of the polymer to the prepared (decapped and reoxidized) protein (e.g., antibody) results in a higher amount of residual, unconjugated protein (e.g., unconjugated antibody or unconjugated fusion construct) relative to conjugated protein (e.g., conjugated antibody or conjugated fusion construct). In some embodiments, a desired ratio/amount of unconjugated protein (e.g., unconjugated antibody or unconjugated fusion construct) to the conjugated protein is obtained by lowering the molar excess of biopolymer to protein, and lowering the conjugation efficiency. In some embodiments, the resultant mixture of the conjugation reaction is reformulated to the desired pH and the desired buffer constituents, without purifying away the unconjugated protein. In some embodiments, the prepared formulation has a mixture of unconjugated protein to conjugated protein, where the ratio is determined by the extent of molar excess of polymer used. In some embodiments, the molar excess of polymer is based on the amounts used to achieve a desired percent residual unconjugated protein (e.g., 20%) after the conjugation reaction of OG1802 to OG1950 to form OG1953 bioconjugate and retaining residual OG1950. In some embodiments, any of the formulations and compositions herein can be produced by using an unconjugated protein (e.g., unconjugated antibody or unconjugated fusion construct) without an engineered cysteine that is mixed with proteins (e.g., antibodies or fusion construct) comprising a free cysteine (e.g., in their Fc region) during the conjugation reaction. In some embodiments, the decapped and refolded protein or antibody undergoes further treatment with alkylation agents such as iodoacetamide (IAM) or N-ethylmaleimide (NEM).

Also provided are any composition or formulation prepared by any one of the methods of the present disclosure.

Also provided is a method of storing a protein, comprising maintaining a protein in a formulation (or therapeutic composition or therapeutically acceptable composition) for at least 2 months and up to 2 years (or more), the formulation (or therapeutic composition or therapeutically acceptable composition) comprising: a first molar amount of a conjugate comprising a first protein conjugated to a phosphorylcholine-containing polymer; a second molar amount of a second protein that is not conjugated to a phosphorylcholine-containing polymer; and a pharmaceutically acceptable carrier, wherein the formulation comprises the second protein at about 1% or more (e.g., about 5-90%, 15-25%, 25-35%, 25-40%, etc.) of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein, wherein the protein comprises an antibody or a fusion construct. The first and second proteins can be any suitable antibody or fusion construct, as described herein. In some embodiments, the first and second proteins are an anti-VEGF antibody (e.g., OG1950, where the conjugated form is OG1953). In some embodiments, the first and second proteins are a fusion construct comprising a VEGF Trap fused to the heavy chain of an anti-IL-6 antibody (e.g., OG2072, where the conjugated form is OG2074).

The formulation (or therapeutic composition or therapeutically acceptable composition) can be any suitable formulation as provided herein. In some embodiments, the formulation (or therapeutic composition or therapeutically acceptable composition) comprises the second protein (the unconjugated protein) at, or at about 1% or more, e.g., about 2% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, or about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less of a total molar amount of the conjugate and the second protein, or optionally, the formulation includes the second protein at a percentage in a range defined by any two of the preceding values (e.g., about 1-95%, 5-90%, 10-80%, 5-50%, 10-40%, 15-35%, 15-25%, 25-35%, 25-40%, 40-95%, 50-80%, etc.) of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at between about 5% and about 50%, or between about 15% and about 30% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at between about 15% and about 25% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at between about 25% and about 35% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at between about 25% and about 40% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at about 20% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) comprises the second protein (the unconjugated protein) at about 30% of a total molar amount of the conjugate and the second protein, wherein the total molar amount comprises a sum of the first molar amount of the conjugate and the second molar amount of the second protein. In some embodiments, the formulation (or therapeutic composition or therapeutically acceptable composition) includes a surfactant (e.g., polysorbate 20), as described herein. In some embodiments, the formulation (or therapeutic composition or therapeutically acceptable composition) includes a surfactant (e.g., polysorbate 20 or polysorbate 80), as described herein. In some embodiments, the formulation (or therapeutic composition or therapeutically acceptable composition) includes a tonicity agent (e.g., sucrose or trehalose), as described herein.

In some embodiments, maintaining the protein includes storing the formulation (or therapeutic composition or therapeutically acceptable composition) at, at about, or at most at 0, 1, 2, 3, 4, 5, 6, 7, 8, 9, 10, 11, 12, 13, 14, 15, 16, 17, 18, 19, 20, 21, 22, 23, 24, 25, 30, 35, 40° C., or optionally it is stored at a temperature in a range defined by any two of the preceding values (e.g., 0-40° C., 0-10° C., 10-20° C., 20-30° C., 30-40° C., etc.). In some embodiments, maintaining the protein includes storing the formulation (or therapeutic composition or therapeutically acceptable composition) at a temperature in the range of 0-10° C. In some embodiments, maintaining the protein includes storing the formulation (or therapeutic composition or therapeutically acceptable composition) at a temperature in the range of 10-20° C. In some embodiments, maintaining the protein includes storing the formulation (or therapeutic composition or therapeutically acceptable composition) at or at about 5° C. In some embodiments, maintaining the protein includes storing the formulation (or therapeutic composition or therapeutically acceptable composition) at or at about 25° C. In some embodiments, maintaining the protein includes storing the formulation (or therapeutic composition or therapeutically acceptable composition) at room temperature. In some embodiments, maintaining the protein includes storing the formulation (or therapeutic composition or therapeutically acceptable composition) under ambient atmospheric pressure.

In some embodiments, the method includes preparing the formulation (or therapeutic composition or therapeutically acceptable composition) with any one of the methods of preparing the formulation (or therapeutic composition or therapeutically acceptable composition) of the present disclosure.

### Method of Conjugating Proteins to Polymers

In some embodiments, a method is presented of preparing a therapeutic protein-half life extending moiety conjugate having the step of conjugating a therapeutic protein which has a cysteine residue added via recombinant DNA technology to a half-life extending moiety having a sulfhydryl specific reacting group selected from the group consisting of maleimide, vinylsulfones, orthopyridyl-disulfides, and iodoacetamides to provide the therapeutic protein-half life extending moiety conjugate.

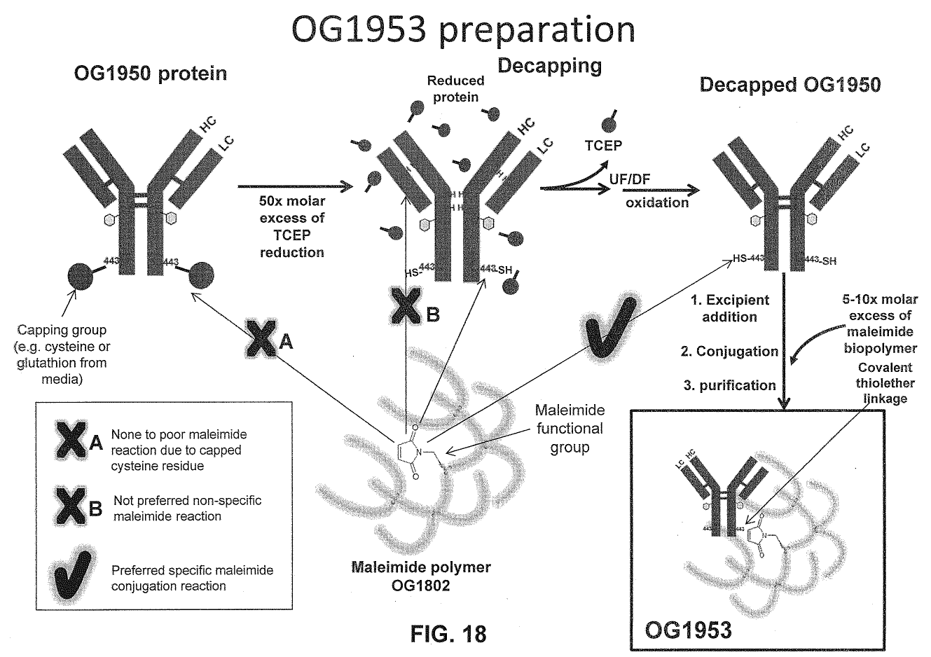

In some embodiments, a method of preparing the OG1953 antibody conjugate from OG1950 is provided. As shown in FIG. 18, the method comprises reducing the OG1950 protein with a 30× molar excess of the TCEP reducing agent (FIG. 18). After reduction, the antibody is oxidized to produce a decapped OG1950 antibody where the inter- and intra- light and heavy chain disulfide bonds naturally occurring in the antibody are formed, but the engineered Cysteine on the heavy chain position L443C (EU numbering, or 449C in SEQ ID NO: 1) remains decapped (FIG. 18). The OG1950 is then conjugated by adding 3.5× molar excess of a maleimide biopolymer. (FIG. 18). The biopolymer links to the OG1950 antibody through a covalent thiolether linkage (FIG. 18). After conjugation, the OG1953 antibody conjugate is purified with both unconjugated antibody and unreacted polymer being removed by CEX chromatography (FIG. 18).

The protein and process described above can be varied as well. Thus, in some embodiments, a process for preparing a conjugated protein (which need not be an antibody or an anti-VEGF antibody) is provided. The process includes reducing one or more cysteines in a protein to form a reduced decapped protein in a solution. After reduction, one or more cysteines of the decapped protein is reoxidized to restore at least one disulfide linkage while ensuring that an engineered cysteine residue in the protein remains in a free thiol form to form a reoxidized decapped protein in the solution. At least one excipient is then added to the solution. The excipient reduces a polymer induced protein precipitation. After the excipient is added, a polymer is added to the solution, which is conjugated to the reoxidized decapped protein at the engineered cysteine residue to form a conjugated protein.

In some embodiments, the molar excess of the reducing agent can be altered to any amount that functions. In some embodiments 5, 10, 20, 30, 40, 50, 60, 70, 80, 90× molar excess of the reducing agent (which need not be TCEP in all embodiments) can be employed. In some embodiments, any antibody (therapeutic or otherwise) can be employed. In some embodiments, these can be the antibody, the antibody conjugate, or both. In some embodiments, 1, 2, 3, 4, 5, 6, 7, 8, 9, 10, 11, 12, 13, 14, 15× molar excess of a maleimide biopolymer can be employed. In some embodiments, there is an excess of decapped protein to polymer. In some embodiments, the amount of the re-oxidized decapped protein is less than the amount of the polymer. In some embodiments, the amount of the re-oxidized decapped protein is 90%, 80%, 70%, 60%, 50%, 40%, 30%, 20%, 10%, 5%, 4%, 3%, 2%, 1% of the amount of the polymer. In some embodiments, 10-15 times as much polymer is used as protein. In some embodiments the amount of the re-oxidized decapped antibody is greater than the amount of the polymer. In some embodiments the amount of the polymer is greater than the amount of the re-oxidized decapped antibody.

In some embodiments, the purification step is optional.

In some embodiments, the method of making an antibody conjugate comprises conjugating an anti-VEGF-A antibody to a phosphorylcholine containing polymer. In some embodiments the method comprises the steps of conjugating an anti-VEGF-A antibody to a phosphorylcholine containing polymer. The anti-VEGF-A antibody comprises an amino residue added via recombinant DNA technology. In some embodiments, the added amino acid residue is a cysteine residue. In some embodiments, the cysteine residue is added outside a variable region of the antibody. The cysteine residue can be added to either the heavy chain or light chain of the antibody.

In some embodiments, the polymer comprises or consists of a phosphorylcholine containing polymer. In some embodiments, the phosphorylcholine containing polymer comprises a sulfhydryl specific reacting group selected from the group consisting of a maleimide, a vinylsulfone, an orthopyridyl-disulfide, and an iodoacetamide. In some embodiments, the sulfhydryl specific reacting group on the phosphorylcholine containing polymer reacts with the cysteine residue on the anti-VEGF-A antibody to make the antibody conjugate.

In some embodiments, the protein to be conjugated can be an antibody, an antibody protein fusion, or a binding fragment thereof. In some embodiments, the protein is not an antibody but is an enzyme, a ligand, a receptor, or other protein or mutants or variants thereof. In some embodiments, the native protein contains at least one disulfide bond and at least one non-native cysteine.

In some embodiments, the excipient can be an acid or a base. In some embodiments, the excipient is a detergent, a sugar, or a charged amino acid. In some embodiments, the excipient assists in keeping the protein in solution during the conjugation to the polymer. In some embodiments, the excipient is added to the solution containing the protein, prior to the addition of the polymer to the solution that contains the protein.

In some embodiments, the conjugation reaction occurs under aqueous conditions between about pH 5.0 to about pH 9.0. In some embodiments, the reaction occurs between 6.0 and 8.5, between 6.5 and 8.0 or between 7.0 and 7.5 or between 8.0 and 9.0 or between 8.3 and 8.7.

In some embodiments, the polymer is conjugated to the protein at 2-37 degrees Celsius. In some embodiments, the conjugation occurs at 0-40 degrees Celsius, 5-35 degrees Celsius, 10-30 degrees Celsius, and 15-25 degrees Celsius.

In some embodiments, the conjugated proteins described herein can be contacted to an ion exchange medium or hydrophobic interaction chromatography or affinity chromatography medium for purification (to separate the conjugated protein from the unconjugated protein and unreacted biopolymer). In some embodiments, the ion exchange medium, hydrophobic interaction chromatography, and/or affinity chromatography medium separates the conjugated protein from the unreacted polymer and from the unconjugated protein.

In some embodiments, the processes described herein and outlined in FIG. 18 involves an excipient that is capable of facilitating and/or maintaining a solubility system. In some embodiments, the process allows the solution to maintain the solubility of the two components meant to interact. This can include the solubility of the protein and the polymer and then the end conjugate as well. In some embodiments, without the excipient approach, the issue can be that while the protein is soluble, when the biopolymer is added, the solubility of the solution (e.g., protein) drops and precipitates form. Of course, when the protein forms precipitates, it is not available for conjugation. Thus, an excipient can be employed to maintain the solubility of the protein in the presence of the biopolymer so the two can couple to form the protein conjugate (or as depicted in FIG. 18, an antibody conjugate). This also allows for the solubility of the conjugate to be maintained.

In some embodiments, the polymers disclosed herein can comprise one or more of the following: a zwitterion, a phosphorylcholine, or a PEG linker bridging a center of a polymer branching point to the maleimide functional group. In some embodiments, any of the polymers provided herein can be added to a protein via the methods provided herein.

In some embodiments, any of the proteins provided herein can be conjugated to any of the polymers provided herein via one or more of the methods provided herein.

In some embodiments, the process(es) provided herein allow(s) for larger scale processing to make and purify protein and/or antibody conjugates. In some embodiments, the volume employed is at least 1 liter, for example 1, 10, 100, 1,000, 5,000, 10,000, liters or more. In some embodiments, the amount of the antibody conjugate produced and/or purified can be 0.1, 1, 10, 100, 1000, or more grams.

In some embodiments, the therapeutic protein may be any of the anti-VEGF-A antibodies described herein having a cysteine residue added via recombinant DNA technology. In some embodiments, the anti-VEGF antibody heavy chain has the following CDRs: CDR_{H}1: GYDFTHYGMN (SEQ ID NO: 9), CDR_{H}2: WINTYTGEPTYAADFKR (SEQ ID NO: 10), and CDR_{H}3: YPYYYGTSHWYFDV (SEQ ID NO: 11). The heavy chain can also have threonine (T) at position 221 (via sequential counting as in SEQ ID NO. 3). In some embodiments, the anti-VEGF light chain has the following CDRs: CDR_{L}1: SASQDISNYLN (SEQ ID NO: 12), CDR_{L}2: FTSSLHS (SEQ ID NO: 13), and CDR_{L}3: QQYSTVPWT (SEQ ID NO: 14). The anti-VEGF-A light chain can also have leucine (L) at Kabat position 4.

In some embodiments, the anti-VEGF-A antibody (and/or conjugate thereof) is IgG1. In some embodiments, the heavy chain has one or more mutations to modulate effector function. In some embodiments, the mutations are to one or more of the following amino acid positions (EU numbering): E233, L234, L235, G236, G237, A327, A330, and P331. In some embodiments, the mutations are selected from the group consisting of: E233P, L234V, L234A, L235A, G237A, A327G, A330S and P331S (EU numbering). In some embodiments, the mutations are (EU numbering) L234A, L235A and G237A.

In some embodiments, the cysteine residue added to the therapeutic protein via recombinant DNA technology should not be involved in Cys-Cys disulfide bond pairing. In this regard, therapeutic proteins may be dimeric. So for example, an intact anti-VEGF-A antibody has two light chains and two heavy chains. If a Cys residue is introduced into the heavy chain for instance, the intact antibody will have two such introduced cysteines at identical positions and the possibility exists that these cysteine residues will form intra-chain disulfide bonds. If the introduced cysteine residues form Cys-Cys disulfide bonds or have a propensity to do so, that introduced Cys residue will not be useful for conjugation. It is known in the art how to avoid positions in the heavy and light chains that will give rise to intra-chain disulfide pairing. See, e.g., U.S. Patent Application No. 2015/0158952.

In some embodiments, the cysteine residue introduced via recombinant DNA technology is selected from the group consisting of (EU numbering) Q347C and L443C. In some embodiments, the cysteine residue is L443C (EU numbering, or 449C in SEQ ID NO: 1). In some embodiments, the heavy chain of the antibody has the amino acid sequence set forth in SEQ ID NO. 1 and the light chain has the amino acid sequence of SEQ ID NO. 2.

In some embodiments, the sulfhydryl specific reacting group is maleimide.

In some embodiments, the half-life extending moiety is selected from the group consisting of polyethylene glycol (PEG), branched PEG, PolyPEG® (Warwick Effect Polymers; Coventry, UK), polysialic acid (PSA), starch, hydroxylethyl starch (HES), hydroxyalkyl starch (HAS), carbohydrate, polysaccharides, pullulane, chitosan, hyaluronic acid, chondroitin sulfate, dermatan sulfate, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyoxazoline, polyethylene-co-maleic acid anyhydride, polystyrene-co-maleic acid anhydride, poly(1-hydroxymethyethylene hydroxymethylformal) (PHF), a zwitterionic polymer, a phosphorylcholine containing polymer and a polymer comprising 2-methacryloyloxy-2′-ethyltrimethylammoniumphosphate (MPC).

In some embodiments, the half-life extending moiety is a zwitterionic polymer. In some embodiments, the zwitterion is phosphorylcholine, i.e. a phosphorylcholine containing polymer. In some embodiments, the polymer is composed of MPC units.

In some embodiments, the MPC polymer has three or more arms. In some embodiments, the MPC polymer has 2, 3, 4, 5, 6, 7, 8, 9, 10, 11 or 12 arms. In some embodiments, the MPC polymer has 3, 6, or 9 arms. In some embodiments, the MPC polymer has 9 arms. In some embodiments, the polymer is synthesized with an initiator comprising 2, 3, 4, 5, 6, 7, 8, 9, 10, 11, 12, or more polymer initiation sites.

In some embodiments, the MPC polymer has a molecular weight between about 300,000 and 1,750,000 Da. In some embodiments, the MPC polymer has a molecular weight between about 500,000 and 1,000,000 Da or between about 600,000 to 900,000 Da.

In some embodiments, the method of preparing a therapeutic protein-half life extending moiety conjugate has an additional step of contacting the therapeutic protein with a thiol reductant under conditions that produce a reduced cysteine sulfhydryl group. As discussed above, it is preferable that the cysteine residue added via recombinant DNA technology are unpaired, i.e. are not involved in Cys-Cys intra chain disulfide bonds or are not substantially involved in such bonding. However, Cys residues which are not involved in such Cys-Cys disulfide bonding and are free for conjugation are known to react with free cysteine in the culture media to form disulfide adducts. See, e.g., WO 2009/052249. A cysteine so derivatized will not be available for conjugation. To free the newly added cysteine from the disulfide adduct, the protein after purification is treated with a reducing agent, e.g., dithiothreitol, or TCEP. However, such treatment with a reducing agent will reduce all of the cysteine residues in the therapeutic protein, including native cysteines many of which are involved in inter and intra chain Cys-Cys disulfides bonds. The native Cys-Cys disulfides are generally crucial to protein stability and activity, and they should be reformed. In some embodiments, all native (e.g., inter and intra) Cys-Cys disulfides are reformed.

To reform native inter and intra-chain disulfide residues, after reduction to remove the cysteine disulfide adducts, the therapeutic protein is exposed to oxidizing conditions and/or oxidizing agents for a prescribed period of time, e.g., 60 minutes to overnight. In some embodiments, ambient air exposure overnight can be used to achieve reformation of the native disulfide bonds. In some embodiments, an oxidizing agent is employed to restore the native disulfides. In some embodiments, the oxiding agent is selected from the group consisting of aqueous CuSO4 and dehydroascorbic acid (DHAA). In some embodiments, the oxidizing agent is DHAA. In some embodiments, the range of DHAA used is in the range of 5-30 equivalents. In some embodiments, the range is 10-20 equivalents. In some embodiments, the range is 15 equivalents.

In some embodiments, the thiol reductant is selected from the group consisting of: Tris[2-carboxyehtyl]phosphine hydrochloride (TCEP), dithiothreitol (DTT), dithioerythritol (DTE), sodium borohydride (NaBH₄), sodium cyanoborohydride (NaCNBH3), β-mercaptoethanol (BME), cysteine hydrochloride and cysteine. In some embodiments, the thiol reductant is TCEP.

In some embodiments, the thiol reductant concentration is between 1 and 100 fold molar excess relative to the therapeutic protein concentration. In some embodiments, the thiol reductant concentration is between 20 to 50 fold molar excess relative to the therapeutic protein concentration. In some embodiments, the thiol reductant is removed following incubation with the therapeutic protein prior to oxidation of the therapeutic protein.

In some embodiments, the method for conjugating a therapeutic protein to a half-life extending moiety has a further step of purifying the therapeutic protein conjugate after conjugation. In some embodiments, the therapeutic protein conjugate is purified using a technique selected from the group consisting of ion exchange chromatography, hydrophobic interaction chromatography, size exclusion chromatography, mixed mode chromatography, and affinity chromatography or combinations thereof.

In some embodiments, the therapeutic protein conjugate retains at least 20% biological activity relative to unconjugated therapeutic protein. In some embodiments, the therapeutic protein conjugate retains at least 50% biological activity relative to unconjugated therapeutic protein. In some embodiments, the therapeutic protein conjugate retains at least 90% biological activity relative to native therapeutic protein.

In some embodiments, the therapeutic protein conjugate has an increased half-life relative to unconjugated therapeutic protein. In some embodiments, the therapeutic protein conjugate has at least a 1.5-fold increase in half-life relative to unconjugated therapeutic protein. In some embodiments, the therapeutic protein conjugate has at least a 5-fold increase in half-life relative to unconjugated therapeutic protein.

In some embodiments, the zwitterionic polymer of the method of conjugating a therapeutic protein to a half-life extending moiety is a radically polymerizable monomer having a zwitterionic group and the method has a further step of polymerizing the free radically polymerizable zwitterionic monomer in a polymerization medium to provide a polymer, the medium comprising: the radically polymerizable zwitterionic monomer; a transition metal catalyst Mₜ^{(q-1)+} wherein Mₜ is a transition metal, q is a higher oxidation state of the metal and q−1 is a lower oxidation state of the metal, wherein the metal catalyst is supplied as a salt of the form Mₜ^{(q-1)+}X′_{(q_1)} wherein X′ is a counterion or group or the transition metal catalyst is supplied in situ by providing the inactive metal salt at its higher oxidation state Mₜ^{q+}X′_{q} together with a reducing agent that is capable of reducing the transition metal from the oxidized inactive state to the reduced active state; a ligand; and an initiator.

To function as an ATRP transition metal catalyst, the transition metal should have at least two readily accessible oxidation states separated by one electron, a higher oxidation state and a lower oxidation state. In ATRP, a reversible redox reaction results in the transition metal catalyst cycling between the higher oxidation state and the lower oxidation state while the polymer chains cycle between having propagating chain ends and dormant chain ends. See, e.g., U.S. Pat. No. 7,893,173.

In some embodiments, the radically polymerizable zwitterionic monomer is selected from the group consisting of

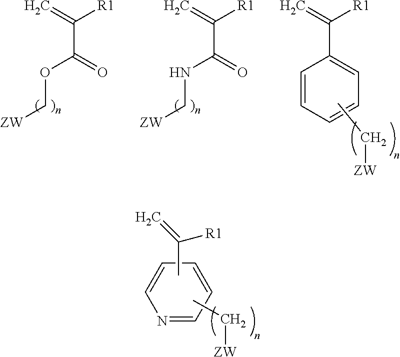

wherein R1 is H or C₁₋₆ alkyl, ZW is a zwitterion and n is an integer from 1-6.

In some embodiments, the radically polymerizable monomer is

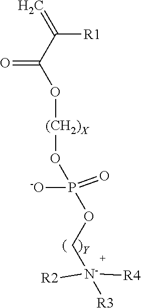

wherein R1 is H or C₁₋₆ alkyl, R2, R3, R4 are the same or different and are H or C₁₋₄alkyl and X and Y are the same or different and are integers from 1-6. In some embodiments, R1, R2, R3 and R4 are each methyl and X and Y are each 2.

In some embodiments, the radically polymerizable monomer is

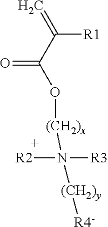

wherein R1 is H or C₁₋₆alkyl, R2 and R3 are the same or different and are H or C₁₋₄alkyl, R4 is PO₄—, SO₃— or CO₂— and X and Y are the same or different and are integers from 1-6. In some embodiments, R1, R2 and R3 are methyl, R4 is PO₄— and X and Y are each 2.

In some embodiments, the monomer is

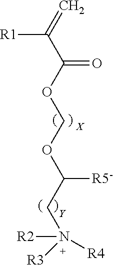

wherein R1 is H or C₁₋₆alkyl, R2, R3 and R4 are the same or different and are H or C₁₋₄alkyl, R5 is PO₄—, SO₃— or CO₂— and X and Y are the same or different and are integers from 1-6. In some embodiments, R1, R2, R3 and R4 are methyl, R5 is PO₄— and X and Y are 2.

In some embodiments, the transition metal Mt is selected from the group consisting of Cu, Fe, Ru, Cr, Mo, W, Mn, Rh, Re, Co, V, Zn, Au, and Ag. In some embodiments, the metal catalyst is supplied as a salt of the form Mₜ^{(q-1)+}X′_{(q_1)}. Mₜ^{(q-1)+} is selected from the group consisting of Cu¹⁺, Fe²⁺, Ru²⁺, Cr²⁺, Mo²⁺, W²⁺, Mn³⁺, Rh³⁺, Re²⁺, Co⁺, V²⁺, Zn⁺, Au⁺, and Ag⁺ and X′ is selected from the group consisting of halogen, C₁₋₆ alkoxy, (SO₄)_{1/2}, (PO₄)_{1/3}, (R7₂PO₄)_{1/2}, (R7₂PO₄), triflate, hexaluorophosphate, methanesulfonate, arylsulfonate, CN and R7CO₂, where R7 is H or a straight or branched C₁₋₆ alkyl group which may be substituted from 1 to 5 times with a halogen. In some embodiments, Mₜ^{(q-1)+} is Cu¹⁺ and X′ is Br.

In some embodiments, Mₜ^{(q-1)+} is supplied in situ. In some embodiments, Mₜ^{q+}X_{q} is CuBr₂. In some embodiments, the reducing agent is an inorganic compound. In some embodiments, the reducing agent is selected from the group consisting of a sulfur compound of a low oxidation level, sodium hydrogen sulfite, an inorganic salt comprising a metal ion, a metal, hydrazine hydrate and derivatives of such compounds. In some embodiments, the reducing agent is a metal. In some embodiments, the reducing agent is Cu⁰.

In some embodiments, the reducing agent is an organic compound. In some embodiments, the organic compound is selected from the group consisting of alkylthiols, mercaptoethanol, or carbonyl compounds that can be easily enolized, ascorbic acid, acetyl acetonate, camphosulfonic acid, hydroxy acetone, reducing sugars, monosaccharides, glucose, aldehydes, and derivatives of such organic compounds.

In some embodiments, the ligand is selected from the group consisting of 2,2′-bipyridine, 4,4′-Di-5-nonyl-2,2′-bipyridine, 4,4-dinonyl-2,2′-dipyridyl, 4,4′,4″-tris(5-nonyl)-2,2′:6′,2″-terpyridine, N,N,N′,N′,N″-Pentamethyldiethylenetriamine, 1,1,4,7,10,10-Hexamethyltriethylenetetramine, Tris(2-dimethylaminoethyl)amine, N,N-bis(2-pyridylmethyl)octadecylamine, N,N,N′,N′-tetra[(2-pyridal)methyl]ethylenediamine, tris[(2-pyridyl)methyl]amine, tris(2-aminoethyl)amine, tris(2-bis(3-butoxy-3-oxopropyl)aminoethyl)amine, tris(2-bis(3-(2-ethylhexoxy)-3-oxopropyl)aminoethyl)amine and Tris(2-bis(3-dodecoxy-3-oxopropyl)aminoethyl)amine. In some embodiments, the ligand is 2,2′-bipyridine.

In some embodiments the initiator has the structure:

R1-R2 [symbol] R3)ₛ

wherein R1 is a nucleophilic reactive group, R2 comprises a linker, and R3 comprises a polymer synthesis initiator moiety having the structure

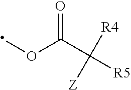

wherein R4 and R5 and are the same or different and are selected from the group consisting of alkyl, substituted alkyl, alkylene, alkoxy, carboxyalkyl, haloalkyl, cycloalkyl, cyclic alkyl ether, alkenyl, alkenylene, alkynyl, alkynylene, cycloalkylene, heterocycloalkyl, heterocycloalkylene, aryl, arylene, arylene-oxy, heteroaryl, amino, amido or any combination thereof; Z is a halogen or CN; and s is an integer between 1 and 20.

In some embodiments, Z is Br and R4 and R5 are each methyl. In some embodiments, R1 is selected from the group consisting of NH₂—, OH—, and SH—.

In some embodiments R2 is alkyl, substituted alkyl, alkylene, alkoxy, carboxyalkyl, haloalkyl, cycloalkyl, cyclic alkyl ether, alkenyl, alkenylene, alkynyl, alkynylene, cycloalkylene, heterocycloalkyl, heterocycloalkylene, aryl, arylene, arylene-oxy, heteroaryl, amino, amido or any combination thereof. In some embodiments, R2 is

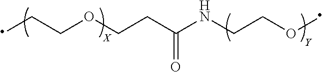

wherein X and Y are the same or different and are integers from 1-20. In some embodiments, X and Y are each 4.

In some embodiments, R3 is

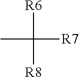

wherein R6, R7 and R8 are the same or different and are selected from the group consisting of

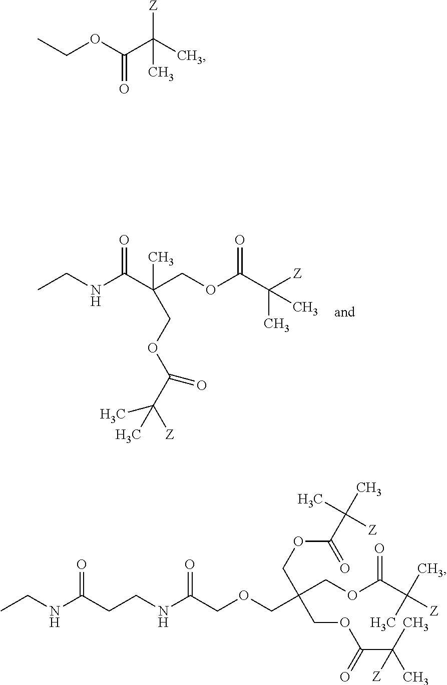

wherein Z is NCS, F, Cl, Br or I. In some embodiments, Z is Br and R6, R7 and R8 are each

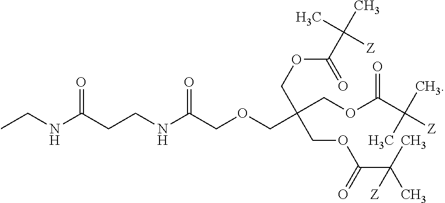

In some embodiments, the initiator has the structure:

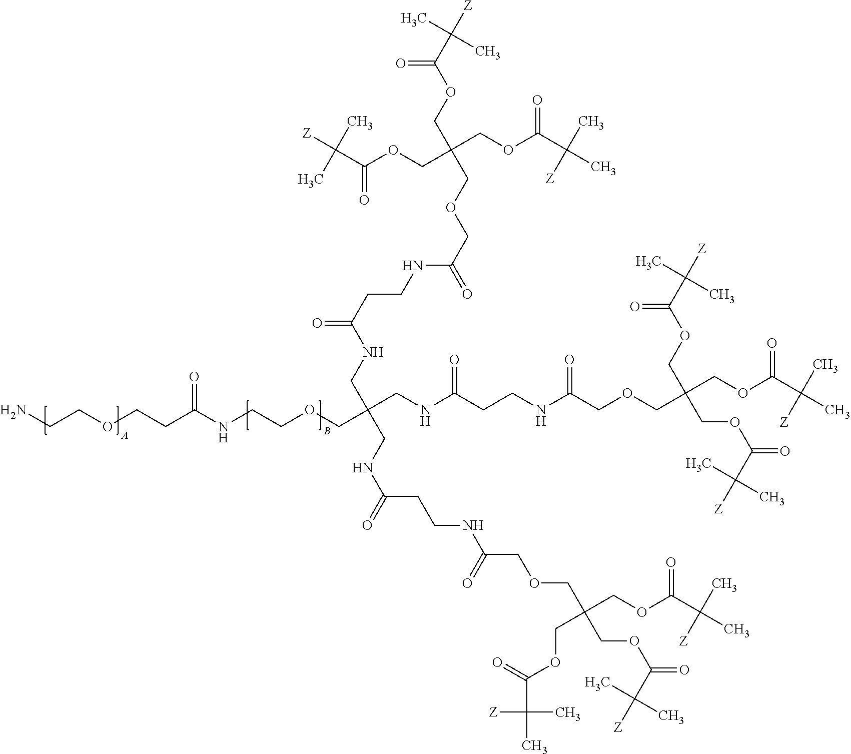

wherein A and B are the same or different and are integers from 2 to 12 and Z is any halide, for example Br. In some embodiments, A and B are each 4.

In some embodiments, the method further has the step of reacting the polymer with a maleimide reagent to provide a polymer having a terminal maleimide. In some embodiments, the maleimide compound is

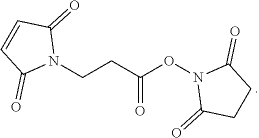 [SMILES: O=C(CCN1C(=O)C=CC1=O)ON1C(=O)CCC1=O]

### Method of Treatment

In some embodiments, a method for treatment or prophylaxis of an ocular disease comprising administering any of the compositions described herein is provided.

Provided herein is a method of treating a subject, comprising: intraocularly administering a therapeutically effective amount of the formulation or composition provided herein to a subject in need thereof (e.g., a subject having an ocular disease). In some embodiments, intraocularly administering the formulation includes using a syringe loaded with the formulation and intraocularly injecting the formulation. In some embodiments, a formulation (e.g., low-viscosity formulation) provided herein has enhanced injectability, e.g., when loaded into a syringe and injected at a site of treatment (e.g., intraocular administration). In some embodiments, a formulation of the present disclosure improves the accuracy of measuring out the formulation compared to a reference formulation without the unconjugated protein. In some embodiments, a formulation of the present disclosure facilitates loading the formulation into a syringe and removing bubbles, compared to a reference formulation without the unconjugated protein. In some embodiments, a formulation of the present disclosure provides a shorter injection time (e.g., about 10 seconds or less, about 8 seconds or less, about 6 seconds or less, about 5 seconds or less, about 4 seconds or less, or a time period in a range defined by any two of the preceding values (e.g., 4-10 seconds, 5-9 seconds, 5-7 seconds, 4-8 seconds, etc.).

In some embodiments, a formulation of the present disclosure reduces the injection force when administering the formulation with a syringe compared to a reference formulation without the unconjugated protein (e.g., a reference composition where all the protein component is conjugated to the polymer, at the same total molar amount). In some embodiments, the injection force to expel the formulation from a syringe fitted with a 27 or 29 gauge needle is about 12 N or less, e.g., about 10 N or less, about 9 N or less, about 8 N or less, about 7 N or less, about 6 N or less, about 5 N or less, or a force value in a range defined by any two of the preceding values (e.g., 5-12 N, 5-10 N, 5-8 N, 6-9 N, etc.). In some embodiments, the method includes administering the formulation using a syringe fitted with a 27 or 29 gauge, or higher gauge, needle.

In some embodiments, the protein is an anti-VEGF-A antibody. In some embodiments, a method is presented for the treatment or prophylaxis of an ocular disease having the step of administering a therapeutic protein selected from the group consisting of an anti-VEGF-A antibody (and conjugates thereof). In some embodiments, any one or more of the antibodies or antibody conjugates, fusion constructs or conjugates thereof provided herein can be used as treatment and/or prophylaxis for an ocular disease. The method includes administering to the subject any one or more of the antibodies and/or antibody conjugates provided herein. In some embodiments, this functionality can be the antibody, the antibody conjugate, or both. In some embodiments, the method includes administering to the subject any one or more of the fusion constructs and/or conjugates thereof provided herein.

In some embodiments a method for treatment or prophylaxis of an ocular disease is provided. The method comprises administering an effective dose of any of the antibody and/or antibody conjugates described herein to a subject in need thereof. In some embodiments, the method includes administering an effective dose of any of the fusion constructs and/or conjugates thereof described herein to a subject in need thereof. In some embodiments a method for treatment or prophylaxis of an ocular disease includes administering an effective dose of any one of the formulations or therapeutically effective compositions described herein that include the antibody and/or antibody conjugates to a subject in need thereof. In some embodiments a method for treatment or prophylaxis of an ocular disease includes administering an effective dose of any one of the formulations or therapeutically effective compositions described herein that include the fusion constructs and/or conjugates thereof to a subject in need thereof. In some embodiments, the disease can be age-related macular degeneration (AMD) or diabetic macular edema (DME). In some embodiments, the disease can be wet AMD. In any method of treatment or prophylaxis of an ocular disease herein, in some embodiments, administering an effective dose of the antibody and antibody conjugates described herein to a subject in need thereof includes administering any one of the formulations or therapeutically effective compositions described herein that include the antibody and/or antibody conjugates to the subject. In any method of treatment or prophylaxis of an ocular disease herein, in some embodiments, administering an effective dose of the fusion constructs and/or conjugates thereof described herein to a subject in need thereof includes administering any one of the formulations or therapeutically effective compositions described herein that include the fusion constructs and/or conjugates thereof to the subject.

In some embodiments, the ocular disease is selected from one or more of the group consisting of diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy. In some embodiments, the ocular disease is diabetic retinopathy.

In some embodiments, the antibody and/or antibody conjugate is administered no more frequently than once a month. In some embodiments, the antibody or conjugate thereof is administered two times per month or weekly. In some embodiments, the antibody or conjugate thereof is administered once every two months, once every three months, once every four months, once every five months, once every six months, once every seven months, once every eight months, once every nine months, once every ten months, once every eleven months, or once every twelve months.

In some embodiments, one or more of the compositions provided herein can allow for a reduction in the consequences of high treatment burdens from the use of intravitreal injection of anti-VEGF agents for the treatment of the wet (proliferative) form of age-related macular degeneration (AMD). Real world outcomes for patients with wet AMD lag behind the clinical outcomes demonstrated in the phase 3 clinical studies such as the MARINA and ANCHOR studies with Lucentis®(ranibizumab) and the VIEW 1 and VIEW 2 studies with Eylea®(aflibercept). An anti-VEGF therapeutic with a longer ocular residence time such that it can be administered less frequently and therefore with a more patient-tolerable profile can bring real world outcomes closer to phase 3 clinical outcomes for more patients.

In some embodiments, compounds, including antibody conjugates and anti-VEGF-A antibodies described herein are used to treat patients who have diabetic macular edema.

In some embodiments, compounds, including antibody conjugates and anti-VEGF-A antibodies described herein are used to treat patients who have background or nonproliferative diabetic retinopathy but have little or no vision impairment. In some embodiments, such patients are dosed less than once a month via intravitreal injection. In some embodiments, such patients are dosed six times a year. In some embodiments, such patients are dosed no more than four times a year. In some embodiments, the patients are dosed no more than three times a year. In some embodiments, the patients are dosed no more than twice a year. In some embodiments, the patients are dosed no more than once a year. In some embodiments, the subject receives the antibody or antibody conjugate via intravitreal injection.

The therapeutic proteins (e.g., both antibodies and antibody conjugates) described herein can be employed by expression of such polypeptides in vivo in a patient, i.e., gene therapy.

There are two major approaches to getting the nucleic acid (optionally contained in a vector) into the patient's cells: in vivo and ex vivo. For in vivo delivery the nucleic acid is injected directly into the patient, usually at the sites where the therapeutic protein is required, i.e., where biological activity of the therapeutic protein is needed. For ex vivo treatment, the patient's cells are removed, the nucleic acid is introduced into these isolated cells, and the modified cells are administered to the patient either directly or, for example, encapsulated within porous membranes that are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, transduction, cell fusion, DEAE-dextran, the calcium phosphate precipitation method, etc. Transduction involves the association of a replication-defective, recombinant viral (including retroviral) particle with a cellular receptor, followed by introduction of the nucleic acids contained by the particle into the cell. A commonly used vector for ex vivo delivery of the gene is a retrovirus.

In some embodiments, the in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinison et al., Cancer Investigation, 14(1): 54-65 (1996)). In some embodiments the vectors for use in gene therapy are viruses, which include adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding the therapeutic protein, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the PRO polypeptide from a host cell in which it is placed. In some embodiments, the signal sequence for this purpose is a mammalian signal sequence. In some embodiments, the signal is the native signal sequence for the therapeutic protein. Optionally, the vector construct may also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis may be used for targeting and/or to facilitate uptake, e.g., capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414(1990). For a review of the currently known gene marking and gene therapy protocols, see, Anderson et al., Science, 256: 808-813 (1992). See also WO 93/25673 and the references cited therein.

Suitable gene therapy and methods for making retroviral particles and structural proteins can be found in, e.g., U.S. Pat. No. 5,681,746.

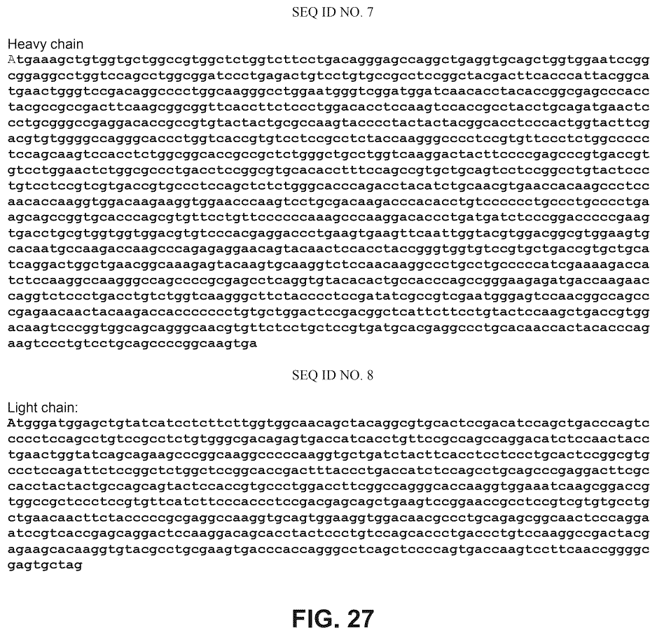

In some embodiments, a method for treatment or prophylaxis of an ocular disease in a mammal is presented in which a nucleic acid molecule that encodes a therapeutic protein selected from the group consisting of an anti-VEGF-A antibody is administered. In some embodiments, the nucleic acid is set forth in FIG. 27.

In some embodiments, the heavy chain is that set forth in SEQ ID NO. 1 (with or without the C-terminal lysine), and the light chain is that set forth in SEQ ID NO. 2. In some embodiments, the nucleic acid molecule is administered via ex vivo gene therapy.

Therapeutic proteins can be incorporated into a pharmaceutical composition with a pharmaceutically acceptable excipient. Pharmaceutical compositions adapted for oral administration may be presented as discrete units such as capsules, as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions can be adapted for nasal administration wherein the excipient is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the excipient is a liquid, for administration as a nasal spray or as nasal drops, include aqueous or oil solutions of the active ingredient. Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine, and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. Pharmaceutical compositions can be substantially isotonic, implying an osmolality of about 250-400 mOsm/kg water.

The pharmaceutical compositions may contain preserving agents, solubilizing agents, stabilizing agents, wetting agents, emulsifiers, sweeteners, colorants, odorants, salts (substances may themselves be provided in the form of a pharmaceutically acceptable salt), buffers, coating agents or antioxidants. They may also contain therapeutically active agents in addition to the substance. The pharmaceutical compositions may be employed in combination with one or more pharmaceutically acceptable excipients. Such excipients may include, but are not limited to, saline, buffered saline (such as phosphate buffered saline), dextrose, liposomes, water, glycerol, ethanol, and combinations thereof.

The antibodies and pharmaceutical compositions containing them may be administered in an effective regime for treating or prophylaxis of a patient's disease including, for instance, administration by oral, intravitreal, intravenous, subcutaneous, intramuscular, intraosseous, intranasal, topical, intraperitoneal, and intralesional administration. Parenteral infusions include intramuscular, intravenous, intraarterial, intraperitoneal, or subcutaneous administration or routes among others. In therapy or as a prophylactic, the active agent may be administered to an individual as an injectable composition, for example as a sterile aqueous dispersion. In some embodiments the agent is isotonic or substantially isotonic. In some embodiments, these can be the antibody, the antibody conjugate, or both.

For administration to mammals, and particularly humans, it is expected that the dosage of the active agent is from 0.01 mg/kg body weight, typically around 1 mg/kg. The physician can determine the actual dosage most suitable for an individual which depends on factors including the age, weight, sex and response of the individual, the disease or disorder being treated, and the age and condition of the individual being treated. The above dosages are exemplary of the average case. There can, of course, be instances where higher or lower dosages are merited. In some embodiments, the dosage can be 0.5 to 20 mg/eye, e.g., 1, 2, 3, 4, 5, 6, 7, 8, 9, 10, 11, 12, 13, 14, 15, 16, 17, 18, or 19 mg.

This dosage may be repeated as often as appropriate (e.g., weekly, fortnightly, monthly, once every two months, quarterly, twice a year, yearly). If side effects develop the amount and/or frequency of the dosage can be reduced, in accordance with normal clinical practice. In one embodiment, the pharmaceutical composition may be administered once every one to thirty days. In one embodiment, the pharmaceutical composition may be administered twice every thirty days. In one embodiment, the pharmaceutical composition may be administered once a week. In some embodiments, the composition comprises an antibody and an antibody conjugate.

The antibodies and pharmaceutical compositions can be employed alone or in conjunction with other compounds, such as therapeutic compounds or molecules, e.g. anti-inflammatory drugs, analgesics, or antibiotics. Such administration with other compounds may be simultaneous, separate, or sequential. The components may be prepared in the form of a kit which may comprise instructions as appropriate. In some embodiments, these can be the antibody, the antibody conjugate, or both.

The antibodies and pharmaceutical compositions disclosed herein can be used for treatment or prophylaxis of disease, particularly the ocular diseases or conditions described herein. In some embodiments, this functionality can be the antibody, the antibody conjugate, or both.

So used, the conjugates are typically formulated for and administered by ocular, intraocular, and/or intravitreal injection, and/or juxtascleral injection, and/or subretinal injection and/or subtenon injection, and/or superchoroidal injection, and/or subconjunctival, and/or topical administration in the form of eye drops and/or ointment. Such antibodies and compositions can be delivered by a variety of methods, e.g. intravitreally as a device and/or a depot that allows for slow release of the compound into the vitreous, including those described in references such as Intraocular Drug Delivery, Jaffe, Ashton, and Pearson, editors, Taylor & Francis (March 2006). In one example, a device may be in the form of a minipump and/or a matrix and/or a passive diffusion system and/or encapsulated cells that release the compound for a prolonged period of time (Intraocular Drug Delivery, Jaffe, Ashton, and Pearson, editors, Taylor & Francis (March 2006).

Formulations or pharmaceutical compositions for ocular, intraocular or intravitreal administration can be prepared by methods and using ingredients known in the art. A main requirement for efficient treatment is proper penetration through the eye. Unlike diseases of the front of the eye, where drugs can be delivered topically, retinal diseases require a more site-specific approach. Eye drops and ointments rarely penetrate the back of the eye, and the blood-ocular barrier hinders penetration of systemically administered drugs into ocular tissue. Accordingly, usually the method of choice for drug delivery to treat retinal disease, such as AMD and CNV, is direct intravitreal injection. Intravitreal injections are usually repeated at intervals which depend on the patient's condition, and the properties and half-life of the drug delivered.

Therapeutic antibodies and related conjugates generally are placed into a container having a sterile access port, for example, an intravenous solution bag or vial having a stopper pierceable by a hypodermic injection needle. Such compositions may also be supplied in the form of pre-filled syringes.

A “stable” formulation or composition is one in which the protein or protein conjugated to a polymer of other half-life extending moiety therein essentially retains its physical stability and/or chemical stability and/or biological activity upon storage. By “stable” is also meant a formulation which exhibits little or no signs of instability, including aggregation and/or deamidation. For example, the formulations provided may remain stable for at least two years when stored as indicated at a temperature of 5-8° C.

Various analytical techniques for measuring protein stability are available in the art and are reviewed in Peptide and Protein Drug Delivery, 247-301 (Vincent Lee ed., New York, N.Y., 1991) and Jones, 1993 Adv. Drug Delivery Rev. 10: 29-90, for examples. Stability can be measured at a selected temperature for a selected time period. In some embodiments, the storage of the formulations is stable for at least 6 months, 12 months, 12-18 months, or for 2 or more years.

A protein, such as an antibody or fragment thereof, “retains its physical stability” in a pharmaceutical composition or formulation if it shows no signs of aggregation, precipitation, deamidation and/or denaturation upon visual examination of color and/or clarity, or as measured by UV light scattering or by size exclusion chromatography.

A protein “retains its chemical stability” in a pharmaceutical composition or formulation, if the chemical stability at a given time is such that the protein is considered to still retain its biological activity. Chemical stability can be assessed by detecting and quantifying chemically altered forms of the protein. Chemical alteration may involve size modification (e.g., clipping), which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation), which can be evaluated by ion-exchange chromatography, for example. An antibody “retains its biological activity” in a pharmaceutical composition or formulation, if the biological activity of the antibody at a given time is within about 10% (within the errors of the assay) of the biological activity exhibited at the time the pharmaceutical composition or formulation was prepared as determined in an antigen binding assay, for example.

A protein-polymer conjugate “retains its chemical stability” the chemical bond between the protein and the polymer is maintained intact, e.g., it is not hydrolyzed or otherwise disrupted. The protein part of the conjugate retains its chemical stability as described above.

By “isotonic” is meant that the composition or formulation of interest has essentially the same osmotic pressure as human blood or the vitreous for intravitreal injections. Isotonic compositions or formulations will generally have an osmotic pressure from about 250 to 400 mOsm. Isotonicity can be measured using a vapor pressure or ice-freezing type osmometer, for example.

As used herein, “buffer” refers to a buffered solution that resists changes in pH by the action of its acid-base conjugate components. In some embodiments, the buffer has a pH from about 3.0 to about 8.0; for example from about 4.5 to 8.0; or about pH 6.0 to about 7.5; or about 6.0 to about 7.0, or about 6.5-7.0, or about pH 7.0 to about 7.5; or about 7.1 to about 7.4. A pH of any point in between the above ranges is also contemplated. In some embodiments, the buffer has a pH from about 4.0 to about 5.0.

In some embodiments, “PBS” phosphate buffered saline, Tris based buffers and histidine based buffers are used. In some embodiments, an acetate buffer (e.g., sodium acetate buffer at pH 4.0-5.0, e.g., at about pH 4.5, or about 5.0) is used. In some embodiments, a composition of formulation of the present disclosure includes a surfactant. In some embodiments, a suitable surfactant includes, without limitation, a polysorbate. In some embodiments, the polysorbate is polysorbate 20, polysorbate 40, or polysorbate 80. In some embodiments, the polysorbate is polysorbate 20. In some embodiments, the polysorbate is present in the composition or formulation at about 0.001%-0.1% (w/w), or about 0.01%-0.05%, or about 0.02%-0.03%. In some embodiments, the polysorbate is present in the composition or formulation at a concentration of, of about, or of at least 0.001, 0.002, 0.005, 0.01, 0.02, or 0.025% (w/w) or at, about, or at a concentration of at most 0.1, 0.09, 0.08, 0.07, 0.06, 0.05, 0.04, 0.03, or 0.025% (w/w), or a percentage (w/w) in a range defined by any two of the preceding values.

In some embodiments, the PBS buffer is made up of at least Na₂HPO₄, KH₂PO₄ and NaCl adjusted so as to provide the appropriate pH. In some embodiments, the buffer may contain other pharmaceutical excipients such as KCl and other salts, detergents and/or preservatives so as to provide a stable storage solution.

A “preservative” is a compound which can be included in the composition or formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

In some embodiments, pharmaceutical compositions or formulations, to be safe for human use or for animal testing, should have sufficiently low levels of endotoxin. “Endotoxin” is lipopolysaccharide (LPS) derived from the cell membrane of Gram-negative bacteria. Endotoxin is composed of a hydrophilic polysaccharide moiety covalently linked to a hydrophobic lipid moiety (lipid A). Raetz C R, Ulevitch R J, Wright S D, Sibley C H, Ding A, Nathan C F. 1991. Gram-negative endotoxin: an extraordinary lipid with profound effects on eukaryotic signal transduction. FASEB J. 5(12):2652-2660. Lipid A is responsible for most of the biological activities of endotoxin, i.e., its toxicity. Endotoxins are shed in large amount upon bacterial cell death as well as during growth and division. They are highly heat-stable and are not destroyed under regular sterilizing conditions. Extreme treatments with heat or pH, e.g., 180-250° C. and over 0.1 M of acid or base must be used (Petsch D, Anspach F. 2000. Endotoxin removal from protein solutions. J Biotechnol. 76: 97-119). Such conditions of course would be highly detrimental to biological drugs.

In the biotech and pharmaceutical industries, it is possible to find endotoxin during both production processes and in final products. As bacteria can grow in nutrient poor media, including water, saline and buffers, endotoxins are prevalent unless precautions are taken. Endotoxin injection into an animal or human causes a wide variety of pathophysiological effects, including endotoxin shock, tissue injury and even death. Ogikubo Y, Ogikubo Y, Norimatsu M, Noda K, Takahashi J, Inotsume M, Tsuchiya M, Tamura Y. 2004. Evaluation of the bacterial endotoxin test for quantifications of endotoxin contamination of porcine vaccines. Biologics 32:88-93.

Pyrogenic reactions and shock are induced in mammals upon intravenous injection of endotoxin at low concentrations (1 ng/mL) (Fiske J M, Ross A, VanDerMeid RK, McMichael J C, Arumugham. 2001. Method for reducing endotoxin in Moraxella catarrhalis UspA2 protein preparations. J Chrom B. 753:269-278). The maximum level of endotoxin for intravenous applications of pharmaceutical and biologic product is set to 5 endotoxin units (EU) per kg of body weight per hour by all pharmacopoeias (Daneshiam M, Guenther A, Wendel A, Hartung T, Von Aulock S. 2006. In vitro pyrogen test for toxic or immunomodulatory drugs. J Immunol Method 313:169-175). EU is a measurement of the biological activity of an endotoxin. For example, 100 μg of the standard endotoxin EC-5 and 120 μg of endotoxin from Escherichia coli O111:B4 have activity of 1 EU (Hirayama C, Sakata M. 2002. Chromatographic removal of endotoxin from protein solutions by polymer particles. J Chrom B 781:419-432). Meeting this threshold level has always been a challenge in biological research and pharmaceutical industry (Berthold W, Walter J. 1994. Protein Purification: Aspects of Processes for Pharmaceutical Products. Biologicals 22:135-150; Petsch D, Anspach F B. 2000. Endotoxin removal from protein solutions. J Biotech 76:97-119).

The presence of endotoxin in drugs to be delivered via intravitreal injection is of particular concern. Intravitreal injection of drug (penicillin) was first performed in 1945 by Rycroft. Rycroft B W. 1945. Penicillin and the control of deep intra-ocular infection. British J Ophthalmol 29 (2): 57-87. The vitreous is a chamber where high levels of drug can be introduced and maintained for relatively long periods of time. The concentration of drug that can be achieved via intravitreal injection far exceeds what can be generated by topical administration or by systemic administration (e.g. intravenous).

One of the most dangerous complications potentially arising from intravitreal injections is endophthalmitis. Endophthalmitis falls into two classes: infectious and sterile. Infectious endophthalmitis is generally caused by bacteria, fungi or parasites. The symptoms of infectious endophthalmitis include severe pain, loss of vision, and redness of the conjunctiva and the underlying episclera. Infectious endophthalmitis requires urgent diagnosis and treatment. Possible treatments include intravitreal injection of antibiotics and pars plana vitrectomy in some cases. Enucleation may be called for to remove a blind and painful eye. See, e.g., Christy N E, Sommer A. 1979. Antibiotic prophylaxis of postoperative endophthalmitis. Ann Ophthalmol 11 (8): 1261-1265.

Sterile endophthalmitis in contrast does not involve an infectious agent and can be defined as the acute intraocular inflammation of the vitreous cavity that resolves without the need of intravitreal antibiotics and/or vitreoretinal surgery. If a vitreous microbiological study has been done, it needs to be negative culture proven to sustain a diagnosis of sterile endophthalmitis. Marticorena J, Romano V, Gomez-Ulla F. 2012 “Sterile Endophthalmitis after Intravitreal Injections” Med Inflam. 928123.

It has been observed that intravitreal injection of biological drugs contaminated with endotoxin can result in sterile endophthalmitis. Marticorena, et al. Bevacizumab (Avastin) is approved by the Food and Drug Administration for the treatment of glioblastoma and of metastatic colorectal cancer, advanced nonsquamous non-small-cell lung cancer and metastatic kidney cancer. Bevacizumab is also widely used off label as a treatment for wet AMD. Bevacizumab comes from the manufacturer as a 100 mg/4 ml dose. This solution cannot be directly used for intravitreal injection and should be compounded by a pharmacist. Clusters of sterile endophthalmitis have been observed and are theorized to be caused by inadvertent contamination of bevacizumab by endotoxin by the compounding pharmacist.

Given the dire clinical results of intravitreal injection of endotoxin, the total amount of endotoxin that can be given to a patient via intravitreal dosing is highly limited. In some embodiments, a solution having an antibody or antibody-conjugate is provided having an endotoxin level that does not exceed 5.0 EU/ml. In some embodiments, the endotoxin level does not exceed 1.0 EU/ml. In some embodiments, the endotoxin level does not exceed 0.5 EU/ml. In some embodiments, the endotoxin level does not exceed 0.2 EU/ml. In some embodiments, the endotoxin level does not exceed 2, 1, 0.5, 0.2, 0.1, 0.09, 0.08, 0.07, 0.06, 0.05, 0.04, 0.03, 0.02 or 0.01 EU/ml.

Two commonly used FDA-approved tests for the presence of endotoxin are the rabbit pyrogen test and Limulus Amoebocyte Lysate (LAL) assay (Hoffman S, et al. 2005. International validation of novel pyrogen tests based on human monocytoid cells J. Immunol. Methods 298:161-173; Ding J L, Ho B A. 2001. New era in pyrogen testing. Biotech. 19:277-281). The rabbit pyrogen test was developed in the 1920s and involves monitoring the temperature rise in a rabbit injected with a test solution. However, use of the rabbit pyrogen test has greatly diminished over the years due to expense and long turnaround time. Much more common is the LAL test. LAL is derived from the blood of a horseshoe crab and clots upon exposure to endotoxin.

One of the simplest LAL assays is the LAL gel-clot assay. Essentially, the LAL clotting assay is combined with a serial dilution of the sample in question. Formation of the gel is proportional to the amount of endotoxin in the sample. Serial dilutions are prepared from the sample and each dilution assayed for its ability to form LAL gel. At some point a negative reaction is contained. The amount of endotoxin in the original sample can be estimated from the dilution assay.

Other LAL tests have also been developed, including the turbidimetric LAL assay (Ong K G, Lelan J M, Zeng K F, Barrett G, Aourob M, Grimes C A. 2006. A rapid highly-sensitive endotoxin detection system. Biosensors and Bioelectronics 21:2270-2274) and the chromogenic LAL assay (Haishima Y, Hasegawa C, Yagami T, Tsuchiya T, Matsuda R, Hayashi Y. 2003. Estimation of uncertainty in kinetic-colorimetric assay of bacterial endotoxins. J Pharm Biomed Analysis. 32:495-503). The turbidimetric and chromogenic assays are much more sensitive and quantitative than the simple gel-clot dilution assay.

In some embodiments a method of reducing the amount of endotoxin in a composition having an antibody disclosed herein is provided. The method having the steps of contacting the composition with an affinity chromatography resin that binds to the antibody; eluting the antibody from the affinity chromatography resin to form an affinity chromatography eluent having the antagonist; contacting the affinity chromatography eluent with an ion-exchange resin that binds the antibody; and eluting the antibody from the ion-exchange resin, wherein the antibody eluted from the ion-exchange resin is substantially free from endotoxin.

The above method for reducing the amount of endotoxin, or other method or process recited herein, can be performed in the order described in the steps above or it can optionally be performed by varying the order of the steps or even repeating one or more of the steps. In one embodiment, the method of reducing the amount of endotoxin in a composition is performed in the order of the described steps. In some embodiments, the affinity chromatography resin contacting, washing and eluting steps are repeated in the same order more than one time before contacting the affinity chromatography eluent with the ion exchange resin. The method can also include a filtering step using, for example, a 0.1 micron, 0.22 micron, or 0.44 micron filter, that can be performed on either one or more of the eluents removed after each resin binding step.

In certain instances, the steps of contacting the composition with affinity chromatography resin, washing and eluting the antibody from the affinity chromatography resin can be repeated more than one time before contacting the first eluent with an ion-exchange resin. In one embodiment, the affinity chromatography resin comprises a recombinant Protein A (“rProteinA”) resin. One example of a suitable recombinant Protein A resin is MabSelect Sure (Cytiva). In another embodiment, a suitable affinity chromatography resin would comprise a protein G chromatography resin. In other embodiments, a suitable affinity chromatography resin comprises a mixed Protein A/Protein G resin.

In some embodiments, the ion exchange resin comprises an anion-exchange resin. As will be known by the person skilled in the art, ion exchangers may be based on various materials with respect to the matrix as well as to the attached charged groups. For example, the following matrices may be used, in which the materials mentioned may be more or less cross-linked: POROS XS (Thermo Fisher Scientific, Waltham, MA), agarose based (such as SP35 Praesto Jetted SP35 IEX (Purolite, King of Prussia, PA, SmartSep (YMC, Shimogyo, Japan), cellulose based (such as DEAE Sephacel®), dextran based (such as Sephadex®), silica based and synthetic polymer based. For the anion exchange resin, the charged groups, which are covalently attached to the matrix, may, for example, be diethylaminoethyl, quaternary aminoethyl, and/or quaternary ammonium. In some embodiments the anion-exchange resin comprises a quaternary amine group. An exemplary anion-exchange resin that has a quaternary amine group is a POROS XQ resin (Thermo Fisher Scientific, Waltham, MA).

In other aspects, if the endotoxin levels are higher than desired after subjecting the composition to the aforementioned anion-exchange chromatography step, the composition may in the alternative be subjected to a cation exchange resin. In some embodiments, any endotoxin in the composition should have a differential binding to the ion-exchange resin than the protein in question to allow purification of the protein from the endotoxin. In this regard, endotoxin is negatively charged and will generally bind to an anion exchange resin. If both the protein and the endotoxin bind to the anion exchange resin, purification of one from the other may be effectuated by using a salt gradient to elute the two into different fractions. The relative binding of the protein to a particular resin may also be affected by changing the pH of the buffer relative to the pI of the protein. In some embodiments, cation-exchange chromatography is the sole ion-exchange chromatography employed.

In some embodiments, if the endotoxin levels are too high after the anion exchange resin, the composition may be further subjected to a second ion-exchange step, for example, by contacting the compositions with a cation exchange resin and followed by a wash step, then elution from the ion-exchange resin. In some embodiments, the cation exchange resin comprises a sulfonic group for binding. Exemplary cation exchange resins include Poros XS (CEX) (Thermo Fisher Scientific, Waltham, MA).

In some embodiments, after the solution of antibody protein is produced, there are a number of steps prior to final formulation of the protein. In some embodiments, a phosphorylcholine-containing polymer (which in some embodiments can be a half-life extending moiety) is conjugated to the protein. The conjugate is then formulated into a final drug formulation which is injected into the patients. In some embodiments, the conjugate is again purified on an ion-exchange resin which can be a cation-exchange resin. In other embodiments, the protein is formulated. In all cases, normal laboratory procedures should be employed to prevent the introduction of endotoxin contaminants into the protein sample or into the protein-polymer conjugate.

Also provided is a kit configured to deliver the formulations and compositions herein. A kit can include: a pre-filled syringe comprising a low-viscosity formulation comprising: a conjugate comprising a first protein conjugated to a phosphorylcholine-containing polymer; and a second protein that is not conjugated to a phosphorylcholine-containing polymer; and a syringe needle configured to fit on the syringe, wherein the gauge of the needle is 27 or more. The syringe can include any of the formulations provided herein. In some embodiments, the needle gauge is 27 or more, 29 or more, or 30 or more. In some embodiments, the syringe and needle are configured for administering the formulation intraocularly. In some embodiments, the kit includes instructions for using the syringe and administering the formulation to subject.

### Additional Embodiments

In some embodiments, a formulation (or therapeutically acceptable composition) comprises a first molar amount of a conjugate having a first protein conjugated to a phosphorylcholine-containing polymer and a second molar amount of a second protein that is not conjugated to a phosphorylcholine-containing polymer, wherein the formulation or composition comprises the second protein at about 0.1% or more (e.g., at about 0.2%, at about 0.3%, at about 0.4%, at about 0.5% or more) of a total molar amount of the conjugate and the second protein, and a pharmaceutically acceptable carrier, wherein the total molar amount comprises a sum of the first molar amount and the second molar amount. In some embodiments, a formulation (or therapeutically acceptable composition) comprises a conjugate having a first protein conjugated to a phosphorylcholine-containing polymer and a second protein the second protein that is not conjugated to a phosphorylcholine-containing polymer, and a pharmaceutically acceptable carrier, wherein the percent composition of the second protein relative to the total protein mass weight concentration of the first protein and the second protein in the composition is about 0.1% or more (e.g., about 0.2%, about 0.3%, about 0.4%, about 0.5% or more).

Additional non-limiting embodiments of the present disclosure are provided below.

### Viscosity

Without being limited by theory, increasing the amount of unconjugated mAb while keeping other factors the same e.g., such as dose of the bioactive (the mAb, whether unconjugated or conjugated), the concentration of the composition or formulation (in mg/mL of bioactive antibody), the volume of administration limit, can lead to an increase in mass of the components in a small volume and therefore a high viscosity. So keeping the total amount of protein the same at say a 50 mg/mL concentration of antibody (inclusive of both conjugated and unconjugated antibody) and decreasing the amount of the conjugated version can result in more unconjugated protein and less conjugated protein and thus less biopolymer, and therefore a lower total mass. By keeping viscosity low according to the formulations and methods provided herein, the manufacturability and dose handling and injectability and potential safety concerns can be improved. In some embodiments, decreasing viscosity while keeping the overall dose administered the same manufacturability can be improved, such as transfer of material and filtering (e.g., through two 0.2 micron filters using a single pump). In some embodiments, decreasing viscosity at high overall dose improves manufacturability by reducing the volume required to fill vials and syringes (to achieve a desired extractable volume). Without being bound by theory, the higher the viscosity then the extractable volume will be lower for a vial. For example, loading a syringe can involve drawing up the solution from the vial up into a dosing syringe through an 18-gauge filter needle to remove any particulates (for example glass shards exfoliated from the glass vial), and then once in the dosing syringe, removing any bubbles formed during the loading by manipulating the plunger rod up and down, and then aligning the plunger to the dose line on the syringe to set the dose (for example, 100 microliter dose). All of these steps can require a certain amount of volume in the vial and the dosing syringe, and if the extractable volume is too low because the viscosity is high and the solution is not free flowing and is sticking more to the wall of the vial, then there is a risk of not having sufficient volume extracted from the vial to be able to set and achieve a precise and accurate and sufficient dose volume of for example 100 microliters. In some embodiments (for example and without limitation, for preparing a pre-filled syringe), filling a formulation into vials or syringes can include the use of a vacuum filling process, in which a vacuum is created inside the vial or syringe, and then the filling needle enters the vacuum, preferably the needle is programmed to descend close to the bottom of the vial or syringe, and then as the solution flows through the needle dispensing the product, the needle is pulled up automatically at a predefined rate. In some embodiments, the needle may be a closing needle. The higher the viscosity, the more likely the need to use a vacuum filling process or a closing needle which add cost and complexity to the manufacturing process.

In some embodiments, for ophthalmology and intravitreal injected biologics, a vial filled drug product is pulled up into a dosing syringe, bubbles (if any) are removed, and then expressed through a needle into the patient's eyes in an ophthalmology clinic setting. Without being limited by theory, the more viscous the formulation, the more skill, time and manipulation may be required to draw up the material into the dosing syringe and the harder to remove the bubble. Once transferred into the dosing syringe and the needle is inserted through the sclera into the patient's vitreous, the more viscous, then the more force and the more time it takes to push the plunger rod/plunger and expel the dose of medicine into the eye. The more force (injectability force, as measured in Newtons) and the more time, in context of the patient's eyeball generally in motion, the more risk of contamination, also the more risk of hitting the back of the eye (the retina), or of hitting the lens or other anatomical structures of the eye during the dose expression/administration.

In some embodiments, filling the formulation directly into a prefilled syringe (rather than a vial) removes the issues of dose transfer from the vial into the dosing syringe. In some embodiments, filling directly into a PFS includes: providing dose accuracy, e.g., for 100 microliter dose volume, for example, fill 200 microliter total dose with 10% precision/accuracy, which may be a 180-220 microliter range. To achieve this, high accuracy of the filling system is needed. Various steps make this difficult with high viscosity: often the need to use a rotary piston pump for precise control of the volume moving through the tubing and the volume/mass filled (rather than a peristaltic pump); needle needs to go down and then rise as it is filling; use of a vacuum to evacuate the air at the base of the syringe (otherwise a large unpredictably sized bubble is present below the filled viscous liquid); use of a closing needle to avoid drops of filling liquid which can be dripping slowly off the end of the needle and risk touching the side of the syringe which causes the syringe to be glued to the rising needle, increasing the time and risk of contamination; placement of the plunger above the filled medicine in the pre-filled syringe; use of placement tube to position the plunger and an insertion rod to push the plunger down towards the top of the liquid; removal of the bubble on top of the liquid, which bubble does not readily move when the filled syringe is inverted so that it can be evacuated to avoid injecting the bubble into the eye. In some embodiments, with 15%, 20%, etc, of unconjugated antibody, the viscosity is reduced to the point that when the syringe is turned upside down (with needle side pointing toward the sky), the bubble pops from the plunger side up to the needle/Luer Lock side and the air (with needle attached) can be evacuated in the process of aligning/setting the final dose by aligning the plunger with the dose line on the syringe, evacuating the air along with the excess volume of dose (200 microliters filled into the syringe, then setting dose at 100 microliter, with excess being expressed out of the needle end while also creating the fully-liquid-path from pre-filled syringe (PFS) inner chamber through needle hub and through needle. In contrast, with ~100% conjugated mAb, and a viscosity of >1,000 cps or >1,250 cps, the bubble stays fixed, and may be removed only with difficulty by manipulation of the plunger via the plunger rod.

In some embodiments, the pre-filled syringe, after filling and plunger placement: then the filled and closed syringes (on a high-speed line, for example a 10-headed filler filling for example 1,000 units per head per hour, so batch size in a single shift of 50,000-150,000 syringes) can be inspected with an automated visual inspection system. In some embodiments, the formulations can be pumped using peristaltic pumps, in order to achieve high accuracy (for example, filling 200 microliters into a pre-filled syringe.

### Clinical Immediacy

In some embodiments, a formulation of the present disclosure can provide a pre-determined balance of basal and bolus (basal-bolus regimen) effects, basal being long-acting in its duration of effect and bolus being more immediate and short-lived in its effect. In some embodiments, the formulations of the present disclosure provide these effects together into a single formulation for ophthalmology intravitreal injection. In some embodiments, one injection can be administered to the patient to provide these effects. In some embodiments, the formulation provides (i) immediacy to control the disease in an immediate manner directly after injection, and (ii) a long duration of effect, i.e. a long durability.

In some embodiments, a patient can benefit from (1) a higher immediacy (bolus effect) after injection to control the disease in the immediate post-injection period and this gives time for the basal effects to kick in over time and deliver a multi-month or longer durability of effect, and (2) immediacy (bolus) and durability (basal) with flexibility to be treated as often as monthly to treat very high disease severity. In some embodiments, a basal-bolus integrated medicine for intravitreal ophthalmology medicines provides broad flexibility of dosing, from once monthly to once every six or nine months or less frequent and to have a pre-defined balance of immediacy (bolus) and durability (basal) in each injected dose.

In some embodiments, the long-acting basal is provided by the conjugate (protein conjugated to the phosphorylcholine biopolymer) in which the residence time is long, i.e. the half-life in the eye is long and in which the exit rate from the vitreous to additional ocular tissues of action (retina, choroid) is slow providing for the long duration of effect; and the immediacy bolus is provided by the unconjugated protein in which the residence time is relatively short, i.e. the half-life in the eye is short and in which the exit rate from the vitreous to the additional ocular tissues of action (retina, choroid) is fast providing for a strong immediacy but not for a long durability.

Comparative half-life of some proteins and conjugates are known. For example, in the standard rabbit intravitreal injection model system in which a drug intended to impact the retina is injected into the vitreous humor and then at different time points the amount of the drug is measured in each ocular tissue of interest. Without being bound by theory, following intravitreal injection the drug then diffuses from the vitreous to reach: the layers of the retina composed of the retina support cells and then the retina photoreceptor cells; and then reaching the retinal pigment epithelium (RPE) layer; then traversing Bruch's membrane; then reaching the capillaries and large and small blood vessels of the choroid; and from there clearance from the eye into the systemic circulation. Smaller proteins have the shortest half-life. And conjugated proteins have the longest half-life. With a longer half-life, it can take longer to load the retina, RPE and choroid with drug and can lead to more variability in clinical efficacy, i.e. bioactivity, across a broad range of disease severity and disease localization across different patients and disease status. In some embodiments, a defined amount of free protein and a defined amount of conjugate allows a desired immediacy (similar amount of free protein VEGF binding sites as marketed proteins) and a desired amount of durability (a large amount of protein VEGF binding sites with a long durability), and a formulation having a desired basal-bolus regimen or profile can be designed and developed as a single formulation and a single infrequent intravitreal injection therapy.

Additional non-limiting embodiments are set forth according to the following numbered arrangements.

1. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein.

2. A therapeutically acceptable composition comprising:
a first molar amount of a conjugate comprising a first protein conjugated to a phosphorylcholine-containing polymer;
a second molar amount of a second protein that is not conjugated to a phosphorylcholine-containing polymer; and
a therapeutically acceptable carrier,
wherein the composition comprises the second protein at about 1% or more of a total molar amount of the conjugate and the second protein, wherein the total molar amount comprises a sum of the first molar amount and the second molar amount,
wherein the composition has a pH that is about 0.5 pH units away or more from the isoelectric point (pI) of the second protein.

3. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

4. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein,
wherein the formulation has a reduced viscosity and/or an enhanced injectability compared to a reference formulation comprising the conjugate at the total molar amount.

5. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein,
wherein the composition has a reduced viscosity and/or an enhanced injectability compared to a reference composition comprising the conjugate, wherein the first protein of the conjugate is present in the reference composition at the total mass weight concentration of the first and second proteins in the composition.

6. A low-viscosity formulation of a protein conjugate, comprising:
a first molar amount of a conjugate comprising a protein conjugated to a phosphorylcholine-containing polymer;
a second molar amount of the protein that is not conjugated to the phosphorylcholine-containing polymer; and
a pharmaceutically acceptable carrier,
wherein the formulation has a pH that is about 0.5 pH units away or more from the isoelectric point (pI) of the protein,
wherein the formulation has reduced viscosity and/or an enhanced injectability compared to a reference formulation comprising the conjugate at a total molar amount that is the sum of the first and second molar amounts.

7. A low-viscosity therapeutically acceptable composition of a protein conjugate, comprising
a first molar amount of a conjugate comprising a protein conjugated to a phosphorylcholine-containing polymer;
a second molar amount of the protein that is not conjugated to the phosphorylcholine-containing polymer; and
a pharmaceutically acceptable carrier,
wherein the composition has a pH that is about 0.5 pH units away or more from the isoelectric point (pI) of the protein,
wherein the composition has reduced viscosity and/or an enhanced injectability compared to a reference composition comprising the conjugate at a total molar amount that is the sum of the first and second molar amounts.

8. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the second protein is present in the formulation at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein,
wherein the formulation has a reduced turbidity compared to a reference formulation comprising the first molar amount of the conjugate and the second molar amount of the second protein at a pH about the same as (e.g., within 0.05, 0.1, 0.15, 0.2, 0.3, 0.4, or 0.5 pH units of) the pI of the second protein.

9. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein,
wherein the composition has a reduced turbidity compared to a reference composition comprising the second protein at the percent composition relative to the total protein mass weight concentration of the first protein and the second protein in the reference composition, at a pH about the same as, or within 0.05, 0.1, 0.15, 0.2, 0.3, 0.4, or 0.5 pH units of the pI of the second protein.

10. A pharmaceutical formulation comprising:
a first molar amount of a conjugate comprising a protein conjugated to a phosphorylcholine-containing polymer;
a second molar amount the protein that is not conjugated to the phosphorylcholine-containing polymer; and
a pharmaceutically acceptable carrier,
wherein the formulation comprises the protein that is not conjugated to the phosphorylcholine-containing polymer at about 1% or more of a total molar amount of the conjugate and unconjugated proteins, wherein the total molar amount comprises a sum of the first molar amount and the second molar amount,
wherein the formulation has a pH that is about 0.5 pH units away or more from the isoelectric point (pI) of the protein,
wherein the formulation is substantially free of turbidity.

11. A formulation comprising:
a phosphorylcholine-containing polymer present in the formulation at 100 mg/mL or higher; and
a protein that is not conjugated to a phosphorylcholine-containing polymer, wherein the protein is present in the formulation at a second molar amount,
wherein the protein is present in the formulation at about 1% or more of a total molar amount of the polymer and the protein, wherein the total molar amount comprises a sum of the first molar amount and the second molar amount,
wherein the formulation has a pH that is about the same as (e.g., within 0.05, 0.1, 0.15, 0.2, 0.3, 0.4, or 0.5 pH units away or more from) the isoelectric point (pI) of the protein.

12. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the difference between the pI of the second protein and the pH of the formulation in the acidic or basic direction is selected to be greater than the minimum difference in the corresponding acidic or basic direction between the pI of the second protein and the pH for a reference formulation comprising:
a third molar amount of the conjugate comprising the first protein conjugated to the phosphorylcholine-containing polymer;
a fourth molar amount of the second protein that is not conjugated to the phosphorylcholine-containing polymer; and
the pharmaceutically acceptable carrier,
wherein a first total molar amount comprising a sum of the first molar amount and the second molar amount, and a second total molar amount comprising a sum of the third molar amount and the fourth molar amount are substantially the same, wherein the second molar amount is greater than the fourth molar amount,
wherein the reference formulation is substantially free of turbidity.

13. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the difference between the pI of the second protein and the pH of the formulation is selected to be greater than the minimum difference between the pI of the second protein and the pH for a reference formulation comprising:
the conjugate comprising the first protein conjugated to the phosphorylcholine-containing polymer;
the second protein that is not conjugated to the phosphorylcholine-containing polymer; and
the pharmaceutically acceptable carrier,
wherein the percent composition of the second protein relative to the total protein mass weight concentration of the first protein and the second protein in the composition is higher than the percent composition of the second protein relative to the total protein mass weight concentration of the first protein and the second protein in the reference composition,
wherein the reference composition is substantially free of turbidity.

14. The formulation or composition of arrangement 12 or 13, wherein the pH of the formulation is selected to be lower than the maximum pH for the reference formulation, wherein the pH of the formulation and a maximum pH for the reference formulation are lower than the pI of the second protein.

15. The formulation or composition of arrangement 12 or 13, wherein the pH of the formulation is selected to be higher than a minimum pH for the reference formulation, wherein the pH of the formulation and the minimum pH for the reference formulation are higher than the pI of the second protein.

16. The formulation or composition of any one of the preceding arrangements, wherein the formulation has a pH that is about 0.5 pH units or more lower than the pI of the second protein.

17. The formulation or composition of any one of the preceding arrangements, wherein the formulation is storage stable.

18. The formulation or composition of any one of the preceding arrangements, wherein the formulation is substantially free of turbidity.

19. The formulation or composition of any one of the preceding arrangements, wherein the formulation has a viscosity of about 1,000 mPa·s or less.

20. The formulation or composition of any one of the preceding arrangements, wherein the formulation has a polydispersity index (PDI) of about 1.2 or less.

21. The formulation or composition of any one of the preceding arrangements or arrangements 54-72, wherein the formulation comprises the second protein at any one of the following percentages of the total molar amount of the conjugate and the second protein: about 2%, about 5%, about 10%, about 15%, about 20%, about 25%, about 30%, about 35%, about 40%, about 50%, about 60%, about 70%, about 80%, or about 90%.

22. The formulation or composition of any one of the preceding arrangements, wherein the formulation comprises the second protein at between about 5% to about 50% of the total molar amount of the conjugate and the second protein.

23. The formulation or composition of any one of the preceding arrangements or arrangements 54-72, wherein a ratio of the molecular weight of the second protein to the polymer in the formulation is at least 1:2.

24. The formulation or composition of any one of the preceding arrangements or arrangements 54-72, wherein a ratio of the molecular weight of the second protein to the polymer in the formulation is between about 1:2 and about 1:10.

25. The formulation or composition of any one of the preceding arrangements or arrangements 54-72, wherein the first protein and the second protein have the same function.

26. The formulation or composition of any one of the preceding arrangements or arrangements 54-72, wherein the first protein and the second protein are the same protein.

27. The formulation or composition of any one of the preceding arrangements or arrangements 54-72, wherein the first protein and the second protein are at least 85% identical to each other in amino acid sequence.

28. The formulation or composition of any one of the preceding arrangements or arrangements 54-72, wherein the first protein has a molecular weight of about 50 kDa or more.

29. The formulation or composition of any one of the preceding arrangements or arrangements 54-72, wherein the second protein has a molecular weight of about 50 kDa or more.

30. The formulation or composition of any one of the preceding arrangements or arrangements 54-72, wherein the phosphorylcholine-containing polymer has a molecular weight of about 100 kDa or more.

31. The formulation or composition of any one of the preceding arrangements or arrangements 54-72, wherein the phosphorylcholine-containing polymer is present in the formulation at a concentration of about 100 mg/mL or more.

32. The formulation or composition of any one of the preceding arrangements or arrangements 54-72, wherein the phosphorylcholine-containing polymer comprises 2-(methacryloyloxyethyl)-2′-(trimethylammonium)ethyl phosphate (MPC) monomers as set forth below:

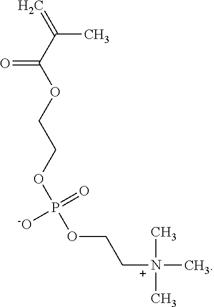

33. The formulation or composition of any one of the preceding arrangements or arrangements 54-72, wherein the phosphorylcholine-containing polymer comprises three or more arms or is synthesized with an initiator comprising 3 or more polymer initiation sites.

34. The formulation or composition of any one of the preceding arrangements or arrangements 54-72, wherein the phosphorylcholine-containing polymer has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.

35. The formulation or composition of any one of the preceding arrangements or arrangements 54-72, wherein the protein, first protein and/or second protein is an antibody or a fusion construct.

36. The formulation or composition of any one of the preceding arrangements or arrangements 54-72, wherein the protein, first protein and/or second protein is an anti-VEGF antibody, a fusion construct comprising a VEGF Trap fused to the heavy chain of an anti-IL-6 antibody, an IL-6 antibody, a fusion construct comprising a PDGFR extracellular trap fused to the heavy chain of an anti-VEGF-A antibody, a VEGF trap-Fc fusion protein, an anti-HTRA1 antibody, or an anti-complement factor D (CFD) antibody.

37. The formulation or composition of arrangement 36, wherein the first protein and/or second protein is the anti-VEGF antibody.

38. The formulation or composition of arrangement 37, wherein the first protein is a first anti-VEGF antibody and the second protein is a second anti-VEGF antibody, wherein the first and second anti-VEGF antibodies comprise different complementarity determining region (CDR) sequences.

39. The formulation or composition of arrangement 37, wherein the first protein is a first anti-VEGF antibody and the second protein is a second anti-VEGF antibody, wherein the first and second anti-VEGF antibodies comprise the same complementarity determining region (CDR) sequences.

40. The formulation or composition of arrangement 37, wherein the anti-VEGF antibody comprises:
a CDR_{H}1 that is the CDR_{H}1 in SEQ ID NO: 1;
a CDR_{H}2 that is the CDR_{H}2 in SEQ ID NO: 1;
a CDR_{H}3 that is the CDR_{H}3 in SEQ ID NO: 1;
a CDR_{L}1 that is the CDR_{L}1 in SEQ ID NO: 2;
a CDR_{L}2 that is the CDR_{L}2 in SEQ ID NO: 2; and
a CDR_{L}3 that is the CDR_{L}3 in SEQ ID NO: 2.

41. The formulation of arrangement 37, wherein the anti-VEGF antibody comprises:

a heavy chain comprising a complementarity
determining region 1
(CDR_{H}1):
(SEQ ID NO: 9)
GYDFTHYGMN,
CDR_{H}2:
(SEQ ID NO: 10)
WINTYTGEPTYAADFKR
CDR_{H}3:
(SEQ ID NO: 11)
YPYYYGTSHWYFDV;
and
a light chain comprising
CDR_{L}1:
(SEQ ID NO: 12)
SASQDISNYLN,
CDR_{L}2:
(SEQ ID NO: 13)
FTSSLHS,
and
CDR_{L}3:
(SEQ ID NO: 14)
QQYSTVPWT.

42. The formulation or composition of arrangement 40 or 41, wherein the heavy chain comprises an amino acid sequence at least 80% identical to SEQ ID NO:1, and the light chain comprises an amino acid sequence at least 80% identical to SEQ ID NO:2.

43. The formulation or composition of any one of arrangements 36-42, wherein the second protein is the anti-VEGF Fab.

44. The formulation or composition of any one of arrangements 37-43, wherein the second protein is an anti-VEGF antibody selected from bevacizumab, ranibizumab, brolucizumab, faricimab, or is aflibercept.

45. The formulation or composition of arrangement 36, wherein the first protein and/or second protein is the fusion construct comprising a VEGF Trap fused to the heavy chain of an anti-IL-6 antibody.

46. The formulation or composition of arrangement 45, wherein the fusion construct comprises:
a CDR_{H}1 that is the CDR_{H}1 in SEQ ID NO: 105;
a CDR_{H}2 that is the CDR_{H}2 in SEQ ID NO: 105;
a CDR_{H}3 that is the CDR_{H}3 in SEQ ID NO: 105;
a CDR_{L}1 that is the CDR_{L}1 in SEQ ID NO: 106;
a CDR_{L}2 that is the CDR_{L}2 in SEQ ID NO: 106; and
a CDR_{L}3 that is the CDR_{L}3 in SEQ ID NO: 106.

47. The formulation or composition of arrangement 45, wherein the fusion construct comprises:

a heavy chain comprising a complementarity
determining region 1
(CDR_{H}1):
(SEQ ID NO: 134
PFAMH,
CDR_{H}2:
(SEQ ID NO: 135)
KISPGGSWTYYSDTVTD,
and
CDR_{H}3:
(SEQ ID NO: 136)
QAWGYYALDI;
and
a light chain comprising
CDR_{L}1:
(SEQ ID NO: 137)
SASISVSYLY,
CDR_{L}2:
(SEQ ID NO: 138)
DDSSLAS,
and
CDR_{L}3:
(SEQ ID NO: 139)
QQWSGYPYT.

48. The formulation or composition of arrangement 46 or 47, wherein the heavy chain comprises an amino acid sequence at least 80% identical to SEQ ID NO:105, and the light chain comprises an amino acid sequence at least 80% identical to SEQ ID NO:106.

49. The formulation or composition of any one of the preceding arrangements or arrangements 54-72, wherein the conjugate comprises a heavy chain and a light chain.

50. The formulation or composition of arrangement 49, wherein the polymer is conjugated to a non-native cysteine outside a variable region of the heavy chain.

51. The formulation or composition of arrangement 50, wherein the cysteine is Q347C (EU numbering) or L443C (EU numbering).

52. The formulation or composition of any one of arrangements 49-51, wherein the conjugate comprises the following structure:

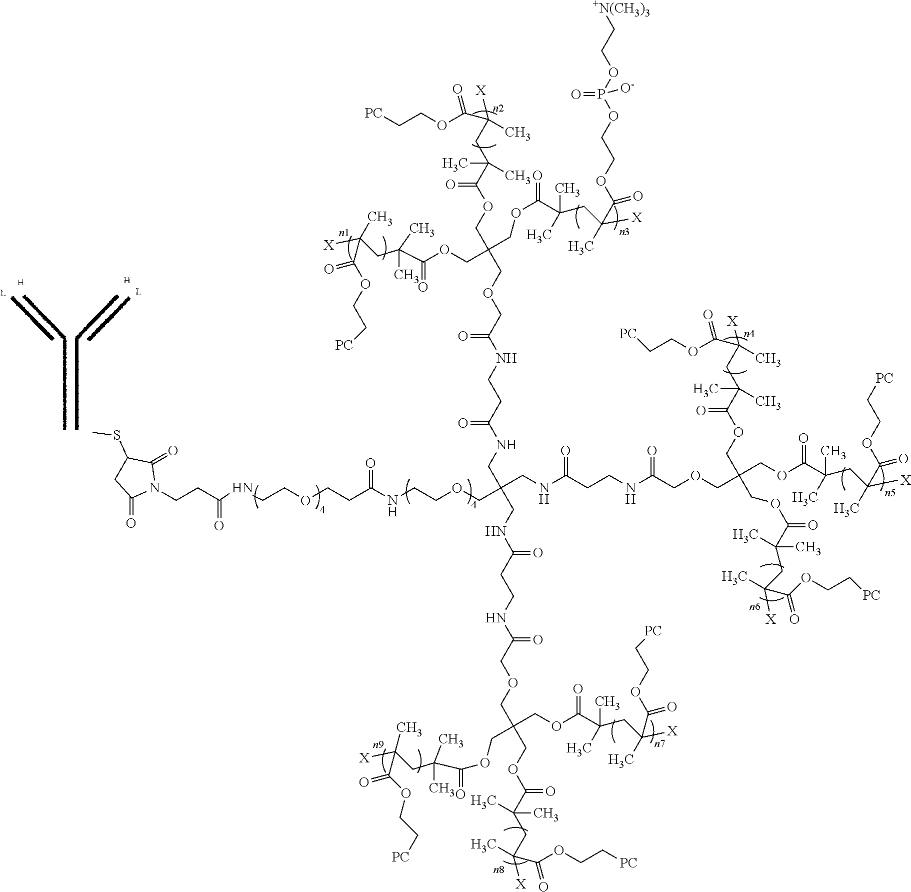

wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is

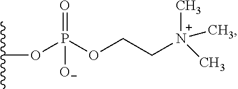 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%.

53. The formulation or composition of any one of the preceding arrangements or arrangements 54-72, wherein the pharmaceutically acceptable carrier comprises a buffer comprising a buffering agent having a pKa that is within about 1 pH unit of the pH of the formulation.

54. The formulation or composition of any one of the preceding arrangements or arrangements 54-72, wherein the pharmaceutically acceptable carrier comprises a buffer selected from: acetate, phosphate, glycine, histidine, HEPES, and Tris.

55. A formulation comprising:
a first molar amount of a conjugate comprising a first protein conjugated to a polymer; and
a second molar amount of a second protein that is not conjugated to a polymer,
wherein the formulation comprises the second protein at about 1% or more of a total molar amount of the first protein and the second protein, wherein the total molar amount comprises a sum of the first molar amount and the second molar amount.

56. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more.

57. A therapeutically acceptable composition comprising:
a first molar amount of a conjugate comprising a first protein conjugated to a polymer; and
a second molar amount of a second protein that is not conjugated to a polymer,
wherein the composition comprises the second protein at about 1% or more of a total molar amount of the first protein and the second protein, wherein the total molar amount comprises a sum of the first molar amount and the second molar amount.

58. A formulation comprising:
a first molar amount of a first protein that is conjugated to a polymer; and
a second molar amount of a second protein that is not conjugated to a polymer,
the further improvement comprising: the formulation comprises the second protein at about 1% or more of a total molar amount of the conjugate and the second protein, wherein the total molar amount comprises a sum of the first molar amount and the second molar amount.

59. A therapeutically acceptable composition comprising:
a first protein that is conjugated to a polymer; and
a second protein that is not conjugated to a polymer,
the further improvement comprising: the percent composition of the second protein relative to the total protein mass weight concentration of the first protein and the second protein in the composition is about 1% or more.

60. The formulation or composition of any one of arrangements 55-58, wherein the formulation or composition comprises the second protein at about 5-50% of the total molar amount of the conjugate and the second protein.

61. A formulation comprising:
a conjugate comprising a first protein conjugated to a polymer; and
a second protein that is not conjugated to a polymer,
wherein a first molar amount of the conjugate and a second molar amount of the second protein has been combined in the formulation such that the second molar amount is about 1% or more of a total molar amount of the conjugate and the second protein, wherein the total molar amount comprises a sum of the first molar amount and the second molar amount.

62. A therapeutically acceptable composition comprising:
a conjugate comprising a first protein conjugated to a polymer; and
a second protein that is not conjugated to a polymer,
wherein a first molar amount of the conjugate and a second molar amount of the second protein has been combined in the composition such that the second molar amount is about 1% or more of a total molar amount of the conjugate and the second protein, wherein the total molar amount comprises a sum of the first molar amount and the second molar amount.

63. A therapeutically acceptable composition comprising:
a conjugate comprising a first protein conjugated to a polymer; and
a second protein that is not conjugated to a polymer,
wherein the second protein at a percent composition relative to the total protein mass weight concentration of the first protein and the second protein in the composition of about 1% or more has been combined with the conjugate, wherein the remainder of the total protein mass weight concentration comprises the first protein.

64. The formulation of arrangement 61 or the composition of arrangement 62, wherein the second molar amount is about 5-50% of the total molar amount of the conjugate and the second protein.

65. The formulation or composition of any one of arrangements 57-64, wherein the polymer is a phosphorylcholine-containing polymer.

66. A formulation comprising:
a conjugate comprising a first protein conjugated to a phosphorylcholine-containing polymer, wherein the polymer has 9 arms and a molecular weight of between 600,000 and 1,000,000 Da, wherein the polymer is present in the formulation at about 100 mg/mL or more; and
a second protein that is not conjugated to a polymer, wherein the second protein is present in the formulation at 5-15 mg/mL.

67. The formulation or composition of any one of arrangements 55-66, wherein the first and second proteins are a therapeutic protein.

68. The formulation or composition of any one of arrangements 55-67, wherein the first protein and second protein are the same.

69. The formulation or composition of any one of arrangements 55-67, wherein the first protein and second protein are different proteins.

70. An intraocular therapeutic composition comprising an anti-VEGF-A antibody at about 50 mg/mL of protein, the anti-VEGF-A antibody comprising:

a heavy chain comprising a complementarity
determining region 1
(CDR_{H}1):
(SEQ ID NO: 9)
GYDFTHYGMN,
CDR_{H}2:
(SEQ ID NO: 10)
WINTYTGEPTYAADFKR,
and
CDR_{H}3:
(SEQ ID NO: 11)
YPYYYGTSHWYFDV;
and
a light chain comprising
CDR_{L}1:
(SEQ ID NO: 12)
SASQDISNYLN,
CDR_{L}2:
(SEQ ID NO: 13)
FTSSLHS,
and
CDR_{L}3:
(SEQ ID NO: 14)
QQYSTVPWT,

wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody,
wherein the antibody conjugate comprises the anti-VEGF-A antibody conjugated to a phosphorylcholine-containing polymer at a non-native cysteine outside a variable region of the antibody, wherein the phosphorylcholine-containing polymer is present in the composition at about 100 mg/mL or more, wherein the phosphorylcholine-containing polymer has 9 arms and a molecular weight of between 600,000 and 1,000,000 Da,
wherein the pH of the composition is about 5.5 or lower.

71. An intraocular therapeutic composition comprising an anti-VEGF-A antibody at about 50 mg/mL of protein, the anti-VEGF-A antibody comprising:
a heavy chain comprising an amino acid sequence of SEQ ID NO: 1 (with or without the C-terminal lysine); and
a light chain comprising an amino acid sequence of SEQ ID NO: 2,
wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody,
wherein the antibody conjugate comprises the following structure:

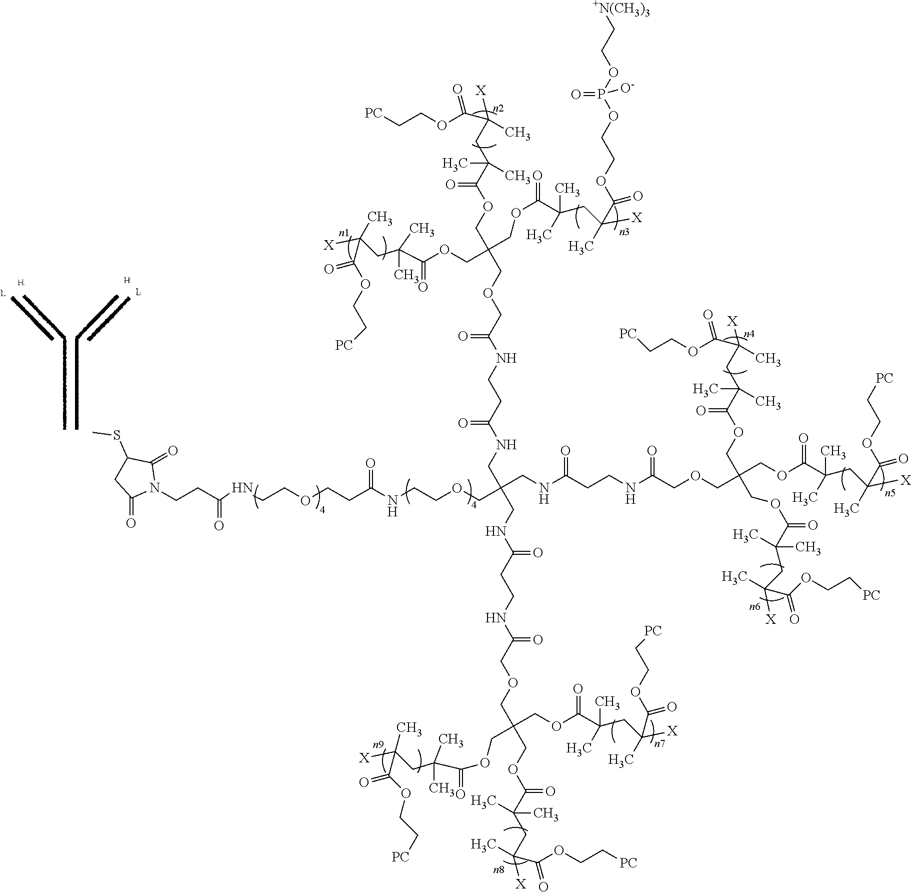

wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is

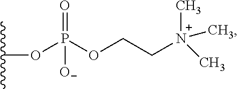 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%,
wherein the phosphorylcholine-containing polymer is present in the composition at about 100 mg/mL or more,
wherein the pH of the composition is about 5.5 or lower.

72. An intraocular therapeutic composition comprising a fusion construct at about 53 mg/mL of protein, the fusion construct comprising a VEGF trap fused to an anti-IL-6 antibody, wherein the fusion construct comprises:
a heavy chain comprising an amino acid sequence of SEQ ID NO:105 (with or without the C-terminal lysine); and
a light chain comprising an amino acid sequence of SEQ ID NO:106,
wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct,
wherein the conjugate comprises the fusion construct conjugated to a phosphorylcholine-containing polymer, wherein the phosphorylcholine-containing polymer is present in the composition at about 100 mg/mL or more, wherein the pH of the composition is about 5 or lower.

73. An intraocular therapeutic composition comprising a fusion construct at about 50 mg/mL of protein, the fusion construct comprising a VEGF trap fused to an anti-IL-6 antibody, wherein the fusion construct comprises:
a heavy chain comprising a complementarity determining region 1 (CDR_{H}1): PFAMH (SEQ ID NO: 134), CDR_{H}2: KISPGGSWTYYSDTVTD (SEQ ID NO: 135), and CDR_{H}3: QAWGYYALDI (SEQ ID NO: 136); and
a light chain comprising CDR_{L}1: SASISVSYLY (SEQ ID NO: 137), CDR_{L}2: DDSSLAS (SEQ ID NO: 138), and CDR_{L}3: QQWSGYPYT (SEQ ID NO: 139),
wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct,
wherein the conjugate comprises the following structure:

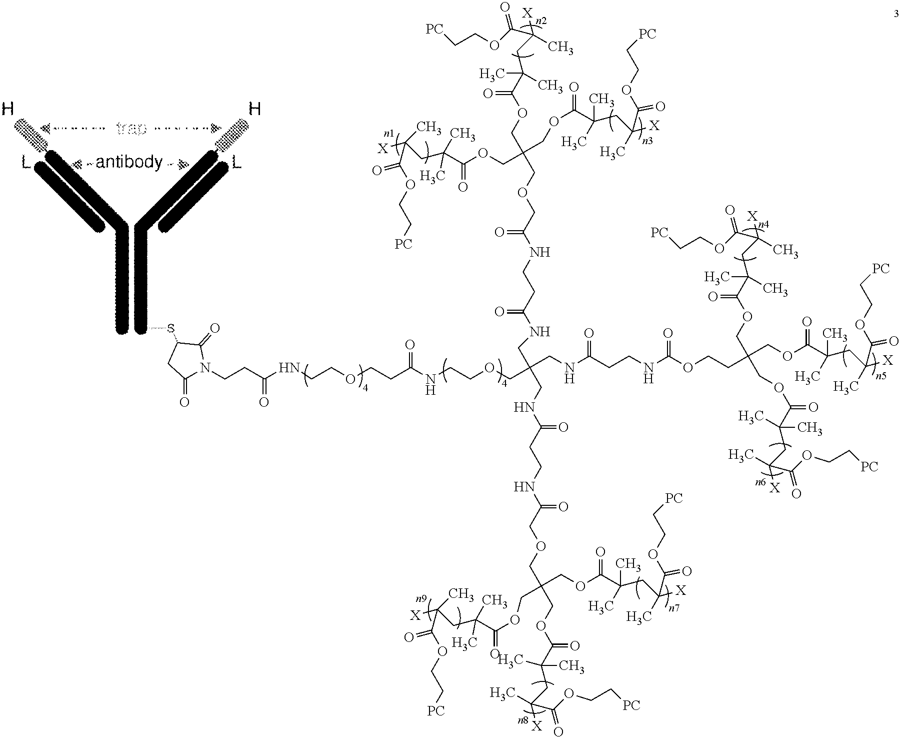

wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is

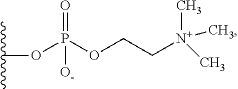

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%,
wherein the phosphorylcholine-containing polymer is present in the composition at about 100 mg/mL or more, wherein the pH of the composition is about 5 or lower.

74. The formulation or composition of any one of the preceding arrangements, wherein the formulation or composition comprises the second protein at more than 5% of a total molar amount of the conjugate and the second protein, wherein the total molar amount comprises a sum of the first molar amount and the second molar amount.

75. A method of preparing a formulation, comprising combining in a formulation:
a first molar amount of a conjugate comprising a first protein conjugated to a phosphorylcholine-containing polymer; and
a second molar amount of a second protein that is not conjugated to a phosphorylcholine-containing polymer,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein.

76. A method of preparing a therapeutically acceptable composition, comprising combining in a therapeutically acceptable composition:
a first molar amount of a conjugate comprising a first protein conjugated to a phosphorylcholine-containing polymer; and
a second molar amount of a second protein that is not conjugated to a phosphorylcholine-containing polymer,
wherein the composition comprises the second protein at about 1% or more of a total molar amount of the conjugate and the second protein, wherein the total molar amount comprises a sum of the first molar amount and the second molar amount,
wherein the composition has a pH that is about 0.5 pH units away or more from the isoelectric point (pI) of the second protein.

77. A method of preparing a therapeutically acceptable composition, comprising combining in a therapeutically acceptable composition:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

78. A method of preparing a formulation, comprising adjusting the pH of a formulation to be about 0.5 pH units away or more from the isoelectric point (pI) of an unconjugated protein comprised in the formulation, wherein the formulation comprises:
a first molar amount of a conjugate comprising a first protein conjugated to a phosphorylcholine-containing polymer; and
a second molar amount of the unconjugated protein, wherein the unconjugated protein is not conjugated to a phosphorylcholine-containing polymer,
wherein the formulation comprises the unconjugated protein at about 1% or more of a total molar amount of the conjugate and the unconjugated protein, wherein the total molar amount comprises a sum of the first molar amount and the second molar amount.

79. The method of arrangement 78, further comprising:
providing an initial formulation comprising the conjugate;
combining in a second formulation the second protein that is not conjugated to a phosphorylcholine-containing polymer with the conjugate; and
adjusting the pH of the initial and/or second formulation to be about 0.5 pH units away or more from the isoelectric point (pI) of the second protein, to thereby prepare the formulation comprising the conjugate at the first molar amount and the second protein at the second molar amount.

80. A method of preparing a therapeutically acceptable composition, comprising adjusting the pH of a composition to be about 0.5 pH units away or more from the isoelectric point (pI) of an unconjugated protein comprised in the composition, wherein the composition comprises:
a first molar amount of a conjugate comprising a first protein conjugated to a phosphorylcholine-containing polymer; and
a second molar amount of the unconjugated protein, wherein the unconjugated protein is not conjugated to a phosphorylcholine-containing polymer,
wherein the composition comprises the unconjugated protein at about 0.1% or more of a total molar amount of the conjugate and the unconjugated protein, wherein the total molar amount comprises a sum of the first molar amount and the second molar amount.

81. A method of preparing a therapeutically acceptable composition, comprising adjusting the pH of a therapeutically acceptable composition to be about 0.5 pH units away or more from the isoelectric point (pI) of an unconjugated protein comprised in the composition, wherein the composition comprises:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

82. The method of arrangement 81, further comprising
providing an initial composition comprising the conjugate;
combining in a second composition the second protein that is not conjugated to a phosphorylcholine-containing polymer with the conjugate; and
adjusting the pH of the initial and/or second composition to be about 0.5 pH units away or more from the isoelectric point (pI) of the second protein, to thereby prepare the composition comprising the conjugate at the first molar amount and the second protein at the second molar amount.

83. The method of any one of arrangements 75-82, wherein adjusting the pH comprises substituting a buffering system of the formulation, wherein the buffering system is substituted to a buffering system that buffers at a pH that is about 0.5 pH units or more away from the pI of the protein.

84. The method of any one of arrangements 75-83, wherein adjusting the pH of the formulation reduces turbidity of the formulation.

85. The method of arrangement 84, wherein the turbidity is reduced by about 10% or more.

86. The method of any one of arrangements 75-85, wherein the turbidity of the formulation before adjusting the pH is about 700 NTU or more, as measured at OD 600 nm and calibrated against a 4000 NTU Formazin calibration standard.

87. The method of any one of arrangements 75-86 or arrangements 128-133, comprising preparing the first protein conjugated to the phosphorylcholine-containing polymer by conjugating the first protein to the phosphorylcholine-containing polymer.

88. The method of arrangement 87, wherein conjugating the first protein to the phosphorylcholine-containing polymer comprises allowing a sulfhydryl-specific reacting group comprised in the phosphorylcholine-containing polymer to react with a cysteine residue in the first protein.

89. A method of preparing a low-viscosity formulation of a protein conjugated to a phosphorylcholine-containing polymer, comprising combining in a formulation:
a first molar amount of a conjugate comprising a first protein conjugated to a phosphorylcholine-containing polymer; and
a second molar amount of the protein that is not conjugated to a phosphorylcholine-containing polymer,
wherein the formulation comprises the protein that is not conjugated to the phosphorylcholine-containing polymer at about 1% or more of a total molar amount of the conjugate unconjugated proteins, wherein the total molar amount comprises a sum of the first molar amount and the second molar amount,
wherein the formulation has a pH that is about 0.5 pH units away or more from the isoelectric point (pI) of the second protein,
wherein the formulation has reduced viscosity and/or an enhanced injectability compared to a reference formulation comprising the conjugate at the total molar amount.

90. A method of preparing a low-viscosity therapeutically acceptable composition of a protein conjugated to a phosphorylcholine-containing polymer, comprising combining in a therapeutically acceptable composition:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein,
wherein the composition has reduced viscosity and/or an enhanced injectability compared to a reference composition comprising the conjugate, wherein the first protein of the conjugate is present in the reference composition at the total mass weight concentration of the first and second proteins in the composition.

91. The method of any one of arrangements 75-90, wherein the prepared formulation has a pH that is about 0.5 pH units or more lower than the pI of the unconjugated protein.

92. The method of any one of arrangements 75-91, wherein the prepared formulation is storage stable.

93. The method of any one of arrangements 75-92, wherein the prepared formulation is substantially free of turbidity.

94. The method of any one of arrangements 75-93, wherein the prepared formulation has a viscosity of about 1000 mPas·s or less.

95. The method of any one of arrangements 75-94, wherein the prepared formulation has a polydispersity index (PDI) of about 1.2 or less.

96. The method of any one of arrangements 75-95 or arrangements 128-133, wherein the formulation comprises the unconjugated protein at any one of the following percentages of the total molar amount of the conjugate and the unconjugated protein: about 5%, about 10%, about 15%, about 20%, about 25%, about 30%, about 35%, about 40%, about 50%, about 60%, about 70%, about 80%, or about 90%.

97. The method of any one of arrangements 75-96 or arrangements 128-133, wherein the formulation comprises the unconjugated protein at between about 5% to about 50% of the total molar amount of the conjugate and the second protein.

98. The method of any one of arrangements 75-97 or arrangements 128-133, wherein a ratio of the molecular weight of the second protein to the polymer in the formulation is at least 1:2.

99. The method of any one of arrangements 75-98 or arrangements 128-133, wherein a ratio of the molecular weight of the second protein to the polymer in the formulation is between about 1:2 and about 1:10.

100. The method of any one of arrangements 75-99 or arrangements 128-133, wherein the first protein and the second protein have the same function.

101. The method of any one of arrangements 75-100 or arrangements 128-133, wherein the first protein and the second protein are the same protein.

102. The method of any one of arrangements 75-101 or arrangements 128-133, wherein the first protein and the second protein are at least 85% identical to each other in amino acid sequence.

103. The method of any one of arrangements 75-102 or arrangements 128-133, wherein the first protein has a molecular weight of about 50 kDa or more.

104. The method of any one of arrangements 75-103 or arrangements 128-133, wherein the unconjugated protein has a molecular weight of about 50 kDa or more.

105. The method of any one of arrangements 75-104 or arrangements 128-133, wherein the phosphorylcholine-containing polymer has a molecular weight of about 100 kDa or more.

106. The method of any one of arrangements 75-105 or arrangements 128-133, wherein the phosphorylcholine-containing polymer is present in the formulation at a concentration of about 100 mg/mL or more.

107. The method of any one of arrangements 75-106 or arrangements 128-133, wherein the phosphorylcholine-containing polymer comprises 2-(methacryloyloxyethyl)-2′-(trimethylammonium)ethyl phosphate (MPC) monomers as set forth below:

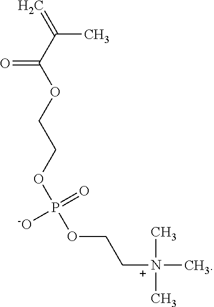

108. The method of any one of arrangements 75-107 or arrangements 128-133, wherein the phosphorylcholine-containing polymer comprises three or more arms or is synthesized with an initiator comprising 3 or more polymer initiation sites.

109. The method of any one of arrangements 75-108 or arrangements 128-133, wherein the phosphorylcholine-containing polymer has 2, 3, 4, 5, 6, 7, 8, 9, 10, 11 or 12 arms or is synthesized with an initiator comprising 2, 3, 4, 5, 6, 7, 8, 9, 10, 11 or 12 polymer initiation sites.

110. The method of any one of arrangements 75-109 or arrangements 128-133, wherein the first protein and/or unconjugated protein is an antibody or a fusion construct.

111. The method of any one of arrangements 75-110 or arrangements 128-133, wherein the first protein and/or unconjugated protein is an anti-VEGF antibody, a fusion construct comprising a VEGF Trap fused to the heavy chain of an anti-IL-6 antibody, or a VEGF trap-Fc fusion protein.

112. The method of arrangement 111, wherein the first protein and/or unconjugated protein is the anti-VEGF antibody.

113. The method of arrangement 112, wherein the first protein is a first anti-VEGF antibody and the unconjugated protein is a second anti-VEGF antibody, wherein the first and second anti-VEGF antibodies comprise different complementarity determining region (CDR) sequences.

114. The method of arrangement 112, wherein the first protein is a first anti-VEGF antibody and the unconjugated protein is a second anti-VEGF antibody, wherein the first and second anti-VEGF antibodies comprise the same complementarity determining region (CDR) sequences.

115. The method of arrangement 112, wherein the anti-VEGF antibody comprises:
a CDR_{H}1 that is the CDR_{H}1 in SEQ ID NO: 1;
a CDR_{H}2 that is the CDR_{H}2 in SEQ ID NO: 1;
a CDR_{H}3 that is the CDR_{H}3 in SEQ ID NO: 1;
a CDR_{L}1 that is the CDR_{L}1 in SEQ ID NO: 2;
a CDR_{L}2 that is the CDR_{L}2 in SEQ ID NO: 2; and
a CDR_{L}3 that is the CDR_{L}3 in SEQ ID NO: 2.

116. The method of arrangement 112, wherein the anti-VEGF antibody comprises:

a heavy chain comprising a complementarity
determining region 1
(CDR_{H}1):
(SEQ ID NO: 9)
GYDFTHYGMN,
CDR_{H}2:
(SEQ ID NO: 10)
WINTYTGEPTYAADFKR,
and
CDR_{H3}:
(SEQ ID NO: 11)
YPYYYGTSHWYFDV;
and
a light chain comprising
CDR_{L}1:
(SEQ ID NO: 12)
SASQDISNYLN,
CDR_{L}2:
(SEQ ID NO: 13)
FTSSLHS,
and
CDR_{L}3:
(SEQ ID NO: 14)
QQYSTVPWT.

117. The method of arrangement 115 or 116, wherein the heavy chain comprises an amino acid sequence at least 80% identical to SEQ ID NO:1, and the light chain comprises an amino acid sequence at least 80% identical to SEQ ID NO:2.

118. The method of any one of arrangements 111-117 or arrangements 128-133, wherein the unconjugated protein is the anti-VEGF Fab.

119. The method of any one of arrangements 111-118 or arrangements 128-133, wherein the unconjugated protein is an anti-VEGF antibody selected from bevacizumab, ranibizumab, brolucizumab, faricimab, or is aflibercept.

120. The method of arrangement 111, wherein the first protein and/or second protein is the fusion construct comprising a VEGF Trap fused to the heavy chain of an anti-IL-6 antibody.

121. The method of arrangement 120, wherein the fusion construct comprises:
a CDR_{H}1 that is the CDR_{H}1 in SEQ ID NO: 105;
a CDR_{H}2 that is the CDR_{H}2 in SEQ ID NO: 105;
a CDR_{H}3 that is the CDR_{H}3 in SEQ ID NO: 105;
a CDR_{L}1 that is the CDR_{L}1 in SEQ ID NO: 106;
a CDR_{L}2 that is the CDR_{L}2 in SEQ ID NO: 106; and
a CDR_{L}3 that is the CDR_{L}3 in SEQ ID NO: 106.

122. The method of arrangement 120, wherein the fusion construct comprises:

a heavy chain comprising a complementarity
determining region 1
(CDR_{H}1):
(SEQ ID NO: 134)
PFAMH,
CDR_{H}2:
(SEQ ID NO: 135)
KISPGGSWTYYSDTVTD,
and
CDR_{H}3:
(SEQ ID NO: 136)
QAWGYYALDI;
and
a light chain comprising
CDR_{L}1:
(SEQ ID NO: 137)
SASISVSYLY,
CDR_{L}2:
(SEQ ID NO: 138)
DDSSLAS,
and
CDR_{L}3:
(SEQ ID NO: 139)
QQWSGYPYT.

123. The method of arrangement 121 or 122, wherein the heavy chain comprises an amino acid sequence at least 80% identical to SEQ ID NO:105, and the light chain comprises an amino acid sequence at least 80% identical to SEQ ID NO:106.

124. The method of any one of arrangements 75-123, wherein the conjugate comprises a heavy chain and a light chain.

125. The method of arrangement 124, wherein the polymer is conjugated to a non-native cysteine outside a variable region of the heavy chain.

126. The method of arrangement 125, wherein the cysteine is Q347C (EU numbering) or L443C (EU numbering).

127. The method of any one of arrangements 124-126, wherein the conjugate comprises the following structure:

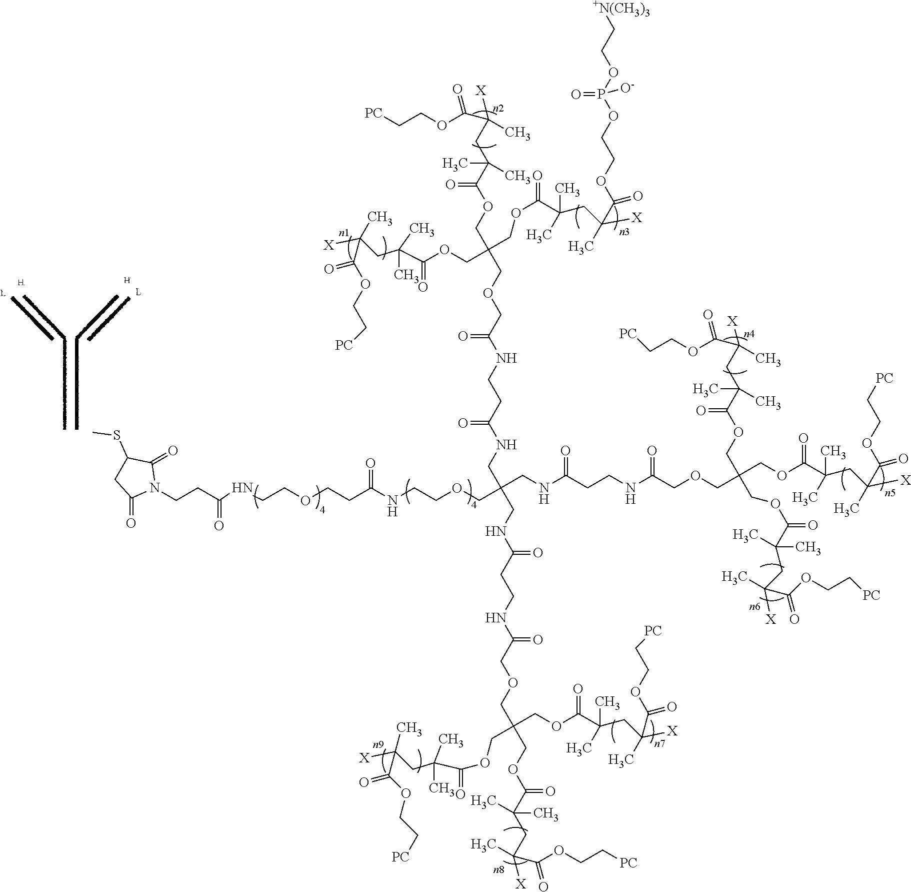

wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is

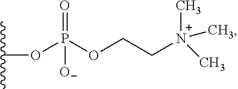 [SMILES: *OP(=O)([O-])OCC[N+](C)(C)C]

where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%.

128. A method of preparing a formulation, comprising combining in a formulation:
a first molar amount of a conjugate comprising a first protein conjugated to a phosphorylcholine-containing polymer; and
a second molar amount of a second protein that is not conjugated to a phosphorylcholine-containing polymer,
wherein the formulation comprises the second protein at about 1% or more of a total molar amount of the conjugate and the second protein, wherein the total molar amount comprises a sum of the first molar amount and the second molar amount.

129. A method of preparing a therapeutically acceptable composition, comprising combining in a therapeutically acceptable composition:
a first molar amount of a conjugate comprising a first protein conjugated to a phosphorylcholine-containing polymer; and
a second molar amount of a second protein that is not conjugated to a phosphorylcholine-containing polymer,
wherein the composition comprises the second protein at about 1% or more of a total molar amount of the conjugate and the second protein, wherein the total molar amount comprises a sum of the first molar amount and the second molar amount.

130. A method of preparing a therapeutically acceptable composition, comprising combining in a therapeutically acceptable composition:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more.

131. The method of any one of arrangements 128-130, wherein the formulation or composition comprises the second protein at about 5-50% of the total molar amount of the conjugate and the second protein.

132. The method of any one of arrangements 128-131, wherein the polymer is a phosphorylcholine-containing polymer.

133. The method of any one of arrangements 128-132, wherein the first and second proteins are a therapeutic protein.

134. A formulation or composition made by the method of any one of arrangements 75-133.

135. A method of treating a subject, comprising:
intraocularly administering a therapeutically effective amount of the formulation or composition of any one of arrangements 1-74 to a subject in need thereof.

136. A method of treating a subject, comprising:
intraocularly administering a therapeutically effective amount of a low-viscosity formulation to a subject in need thereof, wherein the formulation comprises:
   a first concentration of a conjugate comprising a first anti-VEGF antibody conjugated to a phosphorylcholine-containing polymer;
   a second concentration of an anti-VEGF agent that is not conjugated to a phosphorylcholine-containing polymer; and
   a pharmaceutically acceptable carrier,
   wherein the formulation has a pH that is about 0.5 pH units away or more from the isoelectric point (pI) of the anti-VEGF agent.

137. The method of arrangement 136, wherein the anti-VEGF agent is the first anti-VEGF antibody, a second anti-VEGF antibody or a VEGF trap-Fc fusion protein.

138. The method of any one of arrangements 135-137, wherein the subject has an ocular disease selected from: diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy. 139. A kit comprising:
a pre-filled syringe comprising a low-viscosity formulation comprising:
   a conjugate comprising a first protein conjugated to a phosphorylcholine-containing polymer; and
   a second protein that is not conjugated to a phosphorylcholine-containing polymer; and
   a syringe needle for injection of the low-viscosity formulation, wherein the gauge of the needle is 27 or higher.

140. The kit of arrangement 139, wherein the low-viscosity formulation is the formulation or composition of any one arrangements 1-74, or is made by the method of any one of arrangements 75-133.

141. Use of the formulation or composition or kit of any one arrangements 1-74 for the treatment of a subject in need thereof.

142. Use of the formulation or composition of any one arrangements 1-74 for the preparation of a medicament for treating a subject in need thereof.

143. The use of arrangement 141 or 142, wherein the subject has an ocular disease selected from: diabetic retinopathy, choroidal neovascularization (CNV), age-related macular degeneration (AMD), diabetic macular edema (DME), pathological myopia, von Hippel-Lindau disease, histoplasmosis of the eye, central retinal vein occlusion (CRVO), branched central retinal vein occlusion (BRVO), corneal neovascularization, retinal neovascularization, retinopathy of prematurity (ROP), subconjunctival hemorrhage, and hypertensive retinopathy.

144. The composition or formulation or method of any one of the preceding arrangements, wherein the pI is a theoretically determined pI.

145. The composition or formulation or method of any one of the preceding arrangements, wherein the pI is an empirically determined pI.

146. The composition or formulation or method of any one of the preceding arrangements, wherein the composition or formulation comprises polysorbate 20.

147. The composition or formulation or method of any one of the preceding arrangements, wherein the composition or formulation comprises about 0.01-0.05% (w/w) polysorbate 20.

148. The composition or formulation or method of any one of the preceding arrangements, wherein the pharmaceutically acceptable carrier is acceptable for administering directly into an eye of a patient.

149. The composition or formulation or method of any one of the preceding arrangements, wherein the first protein comprises any one of the amino sequences or a combination thereof set forth in SEQ ID NOs: 1-6, 15-133, 156-161 or Tables 0.1, 0.2, 0.3, 0.4, 0.5, 0.6 and 0.7, and FIGS. 12-17, 54, 55A, and 55B.

150. The composition or formulation or method of any one of the preceding arrangements, wherein the second protein comprises any one of the amino sequences or a combination thereof set forth in SEQ ID NOs: 1-6, 15-133, 156-161 or Tables 0.1, 0.2, 0.3, 0.4, 0.5, 0.6 and 0.7, and FIGS. 12-17, 54, 55A, and 55B.

151. A formulation comprising about 40 to about 60 mM sodium acetate, about 0.01% to about 0.04% polysorbate 20, about 40 to about 60 mg/mL (total protein concentration) of a mixture of OG1950 and OG1953, the mixture containing about 15% to about 25% OG1950 and about 75% to about 85% OG1953 by molar amount, at pH about 4.5 to about 5.5.

152. A formulation comprising, consisting of, or consisting essentially of, about 50 mM sodium acetate, about 0.025% polysorbate 20, about 50 mg/mL (total protein concentration) of a mixture of OG1950 and OG1953, the mixture containing about 20% OG1950 and about 80% OG1953 by molar amount, at about pH 5.

153. A method of storing a protein, comprising maintaining a protein in a formulation for at least 2 months and up to 2 years, the formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein,
wherein the protein comprises an antibody or a fusion construct.

### EXAMPLES

### Example 1. Route 1 Synthesis of OG1802

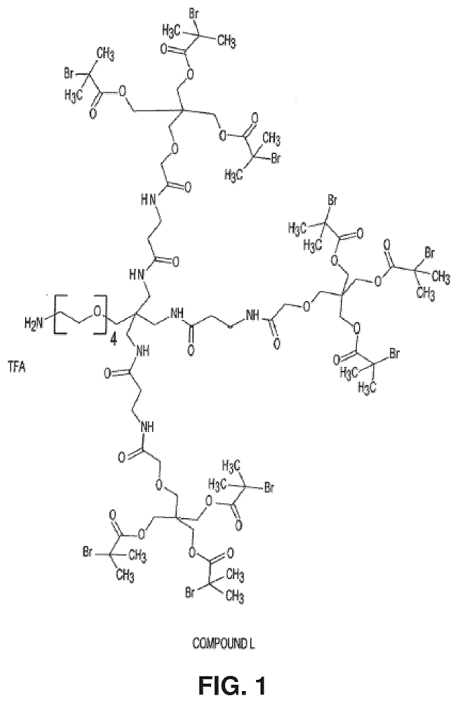

A first route for the synthesis of OG1802 is as follows. First, TFA/amine salt initiator (Compound L) having the structure shown in FIG. 1 was synthesized as follows.

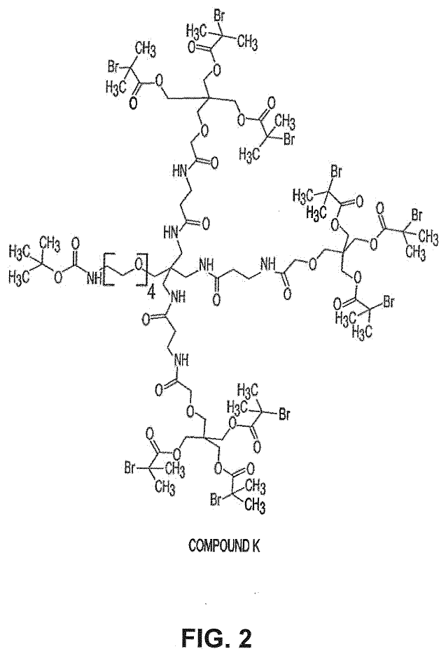

First, Compound K, having the structure shown in FIG. 2 was synthesized as follows. Into a 200 mL round bottom flask under nitrogen was placed Compound J (OG1563) (1.9 g, 2.67 mmol, 3.3 equiv)

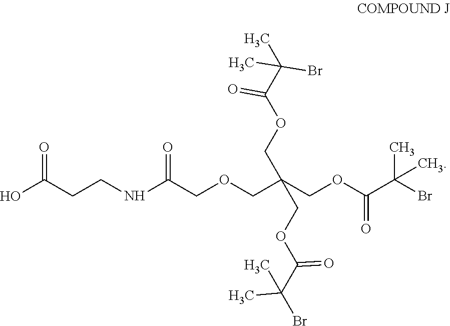 [SMILES: CC(C)(Br)C(=O)OCC(COCC(=O)NCCC(=O)O)(COC(=O)C(C)(C)Br)COC(=O)C(C)(C)Br]

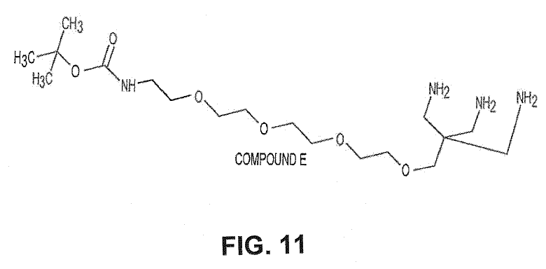

and Compound E (0.525 g, 0.81 mmol, 1.0 equiv) (see FIG. 11) followed by dimethylformamide (10 mL) then diisopropylethylamine (2.5 mL, 14.6 mmol, 18 equiv). The flask was cooled to 0° C. using an ice bath. To this was added propylphosphonic anhydride solution (50 wt. % in ethyl acetate, 2.5 mL, 4.04 mmol, 5 equiv) over −6 minutes.

The reaction was warmed to room temperature and stirred for 15 minutes. The reaction was quenched by adding water (20 mL), saturated aqueous sodium bicarbonate (20 mL) and ethyl acetate (100 mL). The organic layer was separated and the aqueous layer extracted with ethyl acetate (75 mL). The combined organic layers were washed with saturated aqueous sodium bicarbonate (30 mL), 0.5 M aqueous citric acid (40 mL), water (25 mL), and saturated aqueous sodium chloride (40 mL), then dried (sodium sulfate), filtered and concentrated under vacuum. The residue which was used without further purification resulted in 2.0 g (0.80 mmol, 99%) of Compound K.

1H NMR (400 MHz DMSO-d6): δ=1.36 (s, 9H, OCCH3), 1.90 (s, 54H, CC(CH3)2Br), 2.31 (t, J=7.2 Hz, 6H, CCH2CH2NH), 2.98 (d, J=5.6 Hz, 6H, CCH2NH), 3.04 (q, J=6.0 Hz, 2H, OCH2CH2NH), 3.18 (s, 2H, OCH2C), 3.3-3.37 (m, 8H, CH2), 3.47-3.55 (m, 12H, CH2), 3.58 (s, 6H, OCH2C), 3.87 (s, 6H, O═CCH2O), 4.27 (s, 18H, CCH2OC═O), 6.74 (br t, 1H, CH2NHC═O), 7.69 (t, J=6.8 Hz, 3H, CH2NHC═O), 7.84 (t, J=6.0 Hz, 3H, CH2NHC═O). LC-MS (ES, m/z): [(M+2H-boc)/2]+ Calcd for (C84H136Br9N7O33+2H-Boc)/2=1196.6; Found 1196.6.

Next Compound L (FIG. 1) was synthesized as follows: into a 100 mL round bottom under nitrogen was added Compound K (2.0 g, 0.8 mmol), dichloromethane (10 mL) followed by trifluoroacetic acid (5 mL). The reaction was stirred at room temperature for 30 minutes. The reaction was concentrated under a vacuum. The reaction was diluted using dichloromethane (10 mL) and concentrated under a vacuum. The residue was dissolved using acetonitrile (10 mL), filtered through a syringe filter (Acrodisc CR25, PN 4225T) and loaded onto a preparatory HPLC column and eluted with 60% acetonitrile in water (with 0.1% trifluoroacetic acid) up to 98% acetonitrile (with 0.1% trifluoroacetic acid). The tubes containing product were pooled, concentrated under vacuum, frozen and placed on a lyophilizer. This resulted in 990 mgs (0.4 mmol, 50% over 2 steps) Compound L as a white powder.

1H NMR (400 MHz DMSO-d6): δ=1.90 (s, 54H, CC(CH3)2Br), 2.31 (t, J=7.2 Hz, 6H, CCH2CH2NH), 2.97-3.0 (m, 8H, CCH2NH and OCH2CH2NH), 3.17 (s, 2H, OCH2C), 3.3 (q, 6H, CH2CH2NHC═O), 3.4-3.59 (m, 20H, CH2), 3.87 (s, 6H, O═CCH2O), 4.27 (s, 18H, CCH2OC═O), 7.69-7.84 (m, 9H, both CH2NHC═O and NH3+). LC-MS (ES, m/z): [(M+2H)/2]+ Calcd for (C84H136Br9N7O33+2H)/2=1196.6; Found 1197.4.

Next, compound L was used as an initiator to synthesize MPC polymer. Initiator is typically prepared as a stock solution in DMF of about 100 mg/mL. The initiator and the ligand (2,2′-bipyridyl) were introduced into a Schlenk tube. The resultant solution was cooled to −78° C. using a dry ice/acetone mixture, and was degassed under vacuum for 10 min. The tube was refilled under Argon and the catalyst (CuBr unless otherwise indicated), kept under Argon, was introduced into the Schlenck tube (the Molar ratio of atom bromine on the initiator/catalyst (CuBr)/ligand was kept at 1/1/2). The solution became dark brown immediately. The Schlenk tube was sealed and immediately purged by applying a short cycle vacuum/Argon. A solution of HEMA-PC was prepared by mixing a defined quantity of monomer, prepared in a glovebox kept under nitrogen, with 200 proof degassed ethanol. The monomer solution was added drop wise into the Schlenk tube (via cannula) (and homogenized by light stirring). The temperature was maintained at −78° C. A thorough vacuum was applied to the reaction mixture for at least 10 to 15 min. until bubbling from the solution ceased. The tube was then refilled with Argon and warmed to room temperature. The solution was stirred, and as the polymerization proceeded, the solution became viscous. After 3 to 8 hours or just left overnight, the reaction was quenched by direct exposure to air in order to oxidize Cu (I) to Cu (II), the mixture became blue-green in color, and was passed through a silica column in order to remove the copper catalyst. The collected solution was concentrated by rotary evaporation and the resulting mixture was either precipitated with tetrahydrofuran or dialyzed against water followed by freeze drying to yield a free-flowing white powder. Table 1.1 below sets forth polymer data for polymer employing compound L as an initiator.

TABLE 1.1

| Theor. MW | | | | | |
|---|---|---|---|---|---|
| (kDa) | Polymer ID No. | Initiator | Mn(kDa) | Mp(kDa) | PDI |
| 500 | 130 | L | 490 | 530 | 1.1 |
| 750 | 150 | L | 645 | 750 | 1.1 |

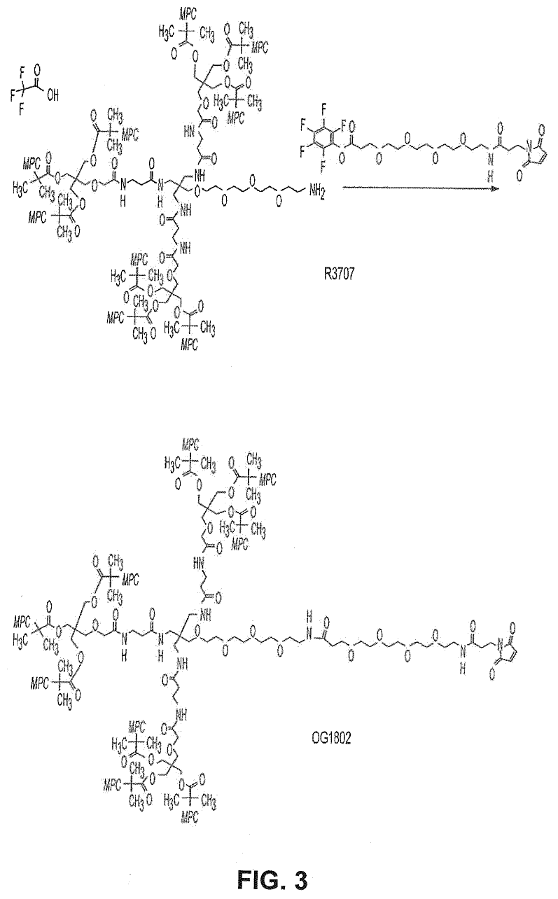

Next, the maleimide Mal-PEG4-PFP ester was snapped on (as set forth in FIG. 3) to the 750 kDa polymer referred to above to provide OG1802. Into a 20 mL vial was placed Polymer R3707 (750 kDa polymer made using L as initiator, 515 mg) and dissolved using ethanol (4.0 mL) after stirring for 40 minutes. To this was added a 1% solution of 4-methylmorpholine in acetonitrile (22 uL). In a separate vial was dissolved Mal-PEG4-PFP (1.97 mg) in acetonitrile (1.0 mL) and this solution was added to the polymer solution over ~2 minute at room temperature and the resulting solution was stirred for overnight. The reaction was diluted with 0.1% aqueous trifluoroacetic acid (2 mL) (pH ~5) followed by water (~12 mL), filtered through a syringe filter (Acrodisc Supor, PN 4612) and placed evenly into 3 Amicon centrifuge membrane dialysis tubes (30,000 mwco). The tubes were diluted and mixed with water (~5 mL each), placed into centrifuge (rpm 3200) for 25 minutes. The filtrate is removed for analysis while the retentate is diluted and mixed with water (~10 mL/tube). The centrifuge procedure is repeated 5 more times, after which the retentate is removed and placed into a vial. The Amicon membrane tubes were rinsed with water (2×~2 mL each tube) and this combined with the retentate. The retentate solution was filtered through a syringe filter (Acrodisc Supor, PN 4612), frozen and placed on a lyophilizer. This resulted in 485 mgs as a white powder.

### Example 2. Synthesis of Initiator OG1786

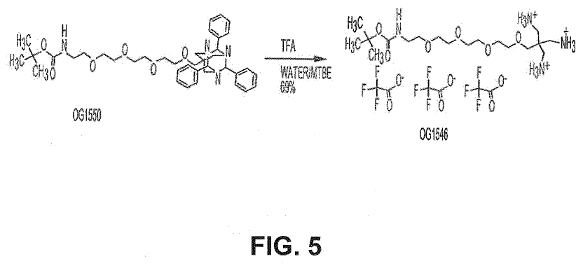

OG1786 is the nine-arm initiator for polymer synthesis used as a precursor in the synthesis of OG1802. Each arm is terminated with a 2-bromoisobutyrate which is capable of initiating polymerization under ATRP. OG1786 is a salt of trifluoro acetic acid (TFA) as shown in FIG. 4. OG1786 is prepared as follows. First, OG1550 is reacted with TFA (trifluoro acetic acid) to produce OG1546 as depicted in FIG. 5.

In a 1 L round bottom flask equipped with a magnetic stir bar and an addition funnel was added OG1550 (14.8 g), methyl tert-butyl ether (MTBE) (350 ml) and water (30 ml). The mixture was stirred to dissolve the OG1550, then cooled in an ice bath. To this mixture was added a solution of trifluoroacetic acid (4.9 ml) in water (90 ml) dropwise over 90 minutes. After addition is complete the mixture was stirred an additional 15 minutes then removed from the ice bath and allowed to warm to room temperature. The mixture was stirred (after removal from the ice bath) for a further 4-5 hours, until tlc showed ~5% starting material remaining, and the pH of the aqueous was between 3 and 4 (pH paper).

The mixture was partitioned. The MTBE layer was washed with water (30 ml). Combine aqueous layers then the aqueous extracted with MTBE (150 ml). This second MTBE phase was washed with water (30 ml). The combined aqueous layers were washed with a third portion of MTBE (100 ml). The third MBTE phase was washed with water (25 ml). The aqueous layers were again combined (~250 ml, pH ~4, by pH paper).

The product was collected by lyophilization. 11.5 g white solid was obtained. This material is extremely hygroscopic, so best handled under nitrogen. The product was confirmed by LCMS.

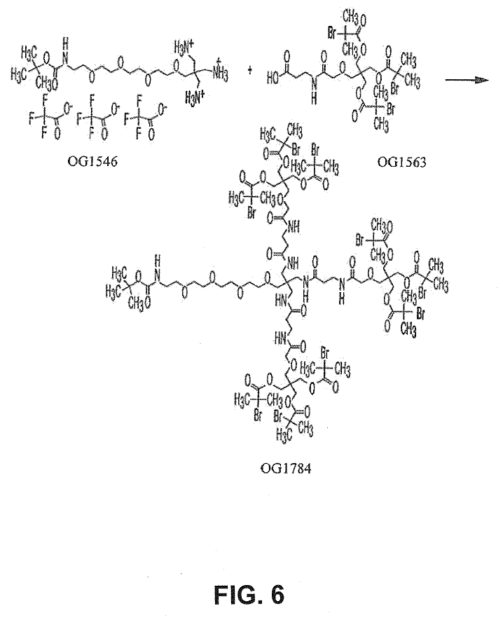

The prepared OG1546 was then reacted with OG1563 to yield OG1784 (as depicted in FIG. 6).

In a 250 ml flask under nitrogen equipped with a stir bar was added OG1546 (hygroscopic, 9.0 g), followed by N,N-dimethylformamide (110 ml). The mixture was stirred at room temperature until all OG1546 dissolved (about 15 minutes), then OG1563 (29.9 g) was added, and the mixture stirred a further 3 minutes until the OG1563 had also been dissolved. The resulting solution was cooled in an ice bath, and N,N-diisopropylethylamine (37.6 ml) was added over 3 minutes, followed by propylphosphonic anhydride (T3P), 50% in ethyl acetate (34.5 ml) dropwise over 5 minutes (T3P addition is exothermic). After T3P addition was complete, the flask was removed from the cooling bath and allowed to reach room temperature. Samples were then taken at 5 minute intervals for LCMS analysis. The reaction showed very light yellow/tan color.

After 20 minutes the reaction was cooled again in an ice bath and 5 ml water added. The mixture was then removed from the cooling bath and a further 50 ml water portion added, followed by 50 ml 0.5 M citric acid then isopropylacetate (300 ml). The mixture was partitioned. The aqueous phase (~300 ml) was extracted with additional isopropyl acetate (150 ml). The aqueous phase was AQ1 for HPLC test. The combined organics were washed with aqueous citric acid (115 ml, 65 mM, which was the mixture of 15 ml of 0.5 M citric acid plus 100 ml water), and the aqueous phase was AQ2 (pH-3). The organic phase was washed with water/saturated sodium chloride (100 ml/25 ml), and the aqueous phase was AQ3 (pH-3). The organic phase was finally washed with saturated sodium chloride (100 ml), and the aqueous phase was AQ4. None of the AQ fractions contained any significant product (data not provided). The organic phase confirmed the product via LCMS. The product was dried over sodium sulfate (80 g), filtered and rinsed with isopropyl acetate (75 ml), and concentrated on a rotary evaporator to a tan oil (33.2 g). The crude was stored overnight under nitrogen.

The next day the crude was allowed to come to room temperature, then dissolved in acetonitrile/water (46 ml/12 ml) and filtered using an HPLC filter disk (Cole-Parmer PTFE 0.2 μm, product number 02915-20). The filtrate was split into three equal portions and purified in three runs.

The filtrate was loaded onto a RediSep Rf Gold C18 column (275 g, SN 69-2203-339, Lot #24126-611Y) equilibrated with 50% acetonitrile/water. The material was eluted at 100 ml/min using the following gradient (solvent A: water, solvent B: acetonitrile). All the relevant fractions were checked by HPLC. The fractions adjudged to be pure enough were pooled (from all three runs) and concentrated (bath temperature kept at about 20° C.) on rotovap, then partitioned between dichloromethane (100 ml) and water (5 ml)/saturated sodium chloride (25 ml). The aqueous was extracted twice more with dichloromethane (2×30 ml). The combined organics were dried over sodium sulfate (35 g), filtered, rinsed with DCM (30 ml), and concentrated. The product and purity were confirmed by LCMS methods. The isolated yield and the purity of the R5172 and R5228 lots are shown in Table 2.1.

TABLE 2.1

| OG1784 lot | R5172 | R5228 |
|---|---|---|
| OG1546 used | 5.3 g | 9.0 g |
| OG1563 used | 17.6 g | 29.9 g |
| Isolated yield | 53% | 58% |
| Purity (a/a 210 nm) | 99.3% | 100.0% |

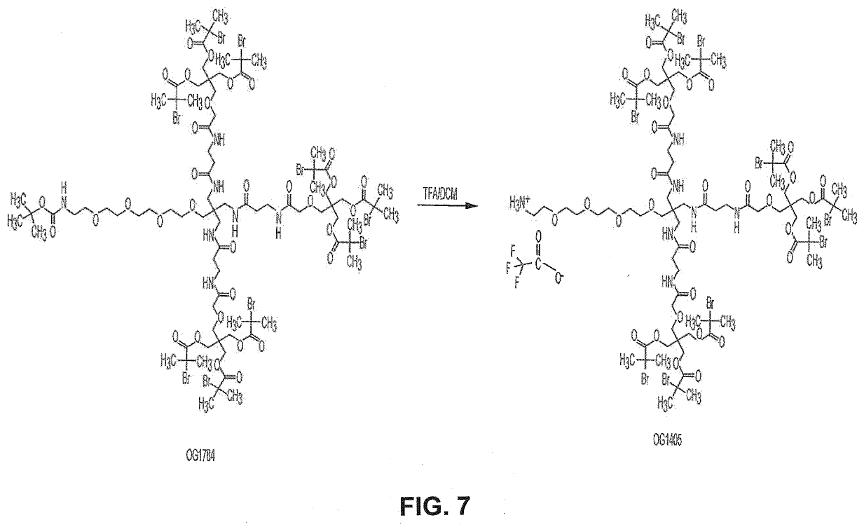

Next OG1405 was prepared from OG1784 as depicted in FIG. 7. In a 500 ml round bottom flask equipped with a magnetic stir bar was added OG1784 (20.9 g), followed by dichloromethane (50 ml) then trifluoroacetic acid (20 ml). The mixture was stirred at room temperature and HPLC analysis showed complete deprotection in 23 minutes. The mixture was concentrated on a rotary evaporator, redissolved in dichloromethane (25 ml) and re-concentrated, then redissolved in acetonitrile (25 ml) and re-concentrated. The product was confirmed by LCMS. The material from above (OG1405, 34.5 g, assume 21.0 g as quantitative yield) was used as a crude oil in the next step. No purification is needed.

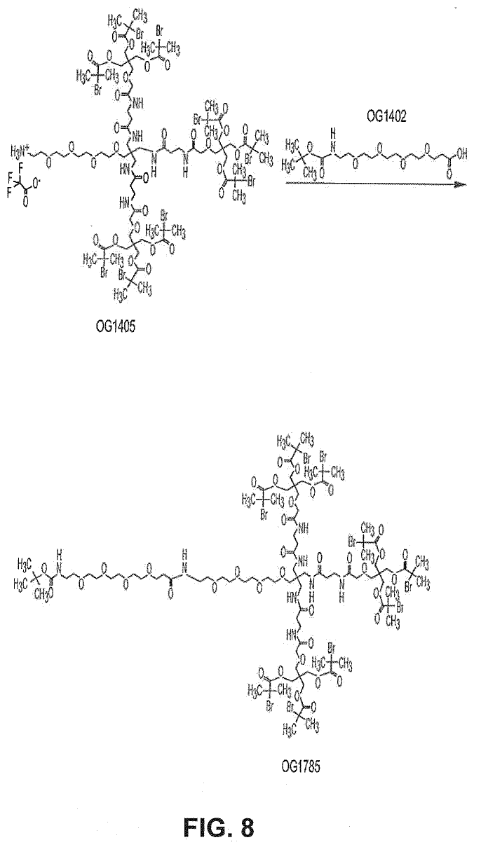

Next, OG1405 was reacted with OG1402 to prepare OG1785 as set forth in FIG. 8. In a 500 ml flask under nitrogen equipped with a stir bar was placed OG1402 (5.5 g), followed by acetonitrile (70 ml), then N,N-diisopropylethylamine (26.3 ml) and T3P solution (see above) (7.9 ml). The solution was stirred at room temperature for 30 minutes, then cooled in an ice water bath and a solution of OG1405 (crude oil from above, 34.5 g) in acetonitrile (70 ml) added. The mixture was warmed to room temperature. After 20 minutes the reaction was cooled in an ice water bath and quenched with water (5 ml). The mixture was then concentrated under vacuum using a rotary evaporator to half volume. Samples were taken for LCMS.

More water (50 ml), followed by 0.5 M citric acid (75 ml) and isopropyl acetate (175 ml) was added. The mixture was partitioned in 5 minutes. The aqueous was extracted with additional isopropyl acetate (50 mL). The combined organics were washed with aqueous citric acid (0.13 M, 30 ml, consist of 10 ml of 0.5 M citric acid and 20 ml water). The organics were then washed with the mixture of saturated sodium chloride (25 ml) and water (25 ml), then finally washed with the saturated sodium chloride (25 ml). They were then dried over sodium sulfate (124 g), filtered and rinsed with isopropyl acetate (30 ml), and concentrated under rotary evaporator to a tan oil (27.3 g). Samples were taken for LCMS analysis.

The oil was dissolved in acetonitrile/water (3:1, 15 ml/5 ml), filtered through an HPLC filter disk (Cole-Parmer PTFE membrane 0.2 μm, product number 02915-20) and split into three equal portions, each of which were individually purified as follows.

Portions were loaded onto Redi-Sep Gold C18 column (275 g, SN—69-2203-339, Lot 241234-611W) equilibrated at 50% solvent B (acetonitrile)/50% solvent A (water). The material was then purified by reverse phase HPLC with a solvent A: water/solvent B: acetonitrile gradient. Appropriate fractions were pooled and partitioned between dichloromethane (150 ml) and water (5 ml)/saturated sodium chloride (25 ml). The aqueous was extracted twice with dichloromethane (2×50 ml). Combined organics were dried over sodium sulfate (60 g), filtered and rinsed with dichloromethane (40 ml) and concentrated. Structure and purity were confirmed by various analytics including LCMS: OG1785 was isolated as a foamy solid (R5329, 19.0 g, 83% yield, 95.1% purity (a/a 210 nm), stored under nitrogen at 4° C.

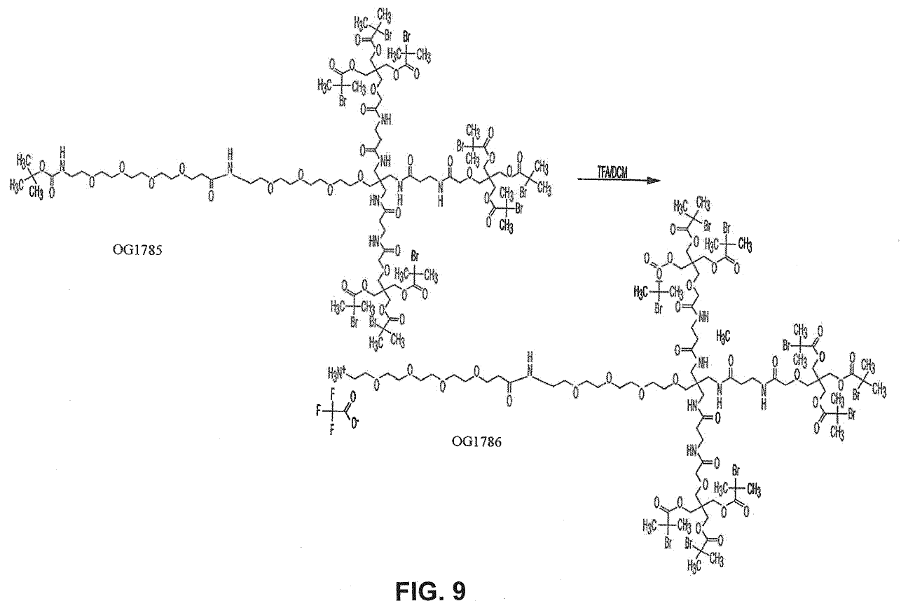

Next, the tert-butyloxycarbonyl protecting group on OG1785 was removed using trifluoroacetic acid (TFA) to produce OG1786 as depicted in FIG. 9.

### Example 3. Synthesis of Polymer OG1801

Polymer OG1801 is made first from the initiator OG1786. OG1801 has an amine functionality, which is more stable (than maleimide) during polymer synthesis. To synthesize polymer OG1801, a modified version of ATRP is used wherein the copper species (Cu(I)) is generated in situ by adding metallic copper to Cu (II). Starting materials and reagents needed in the reaction are calculated based on batch input of the monomer (HEMA-PC) OG47, as well as the targeted molecular weight (MW).

Weighed 50 g monomer OG47 in glove box and added 200 mL of degassed EtOH to dissolve the monomer at room temperature; sampled for monomer concentration test. Weighed Cu (II), Bpy, Cu(0) in a 500 mL flask; purged with Argon, while adding monomer solution to the flask; sealed the flask with stopper and vacuumed for 25 min until no bubbles. The reaction changed color gradually from light green to dark green, then to light brown; weighed ~200 mg of initiator OG1786 in glove box, and dissolved in ~2000 μL of DMF under room temperature to make 100 mg/mL stock solution; sampled for initiator concentration and purity test; added the initiator solution to the flask under Argon. The reaction solution became dark brown and started thickening over time; sealed the system and let the reaction occur over 2 days.

OG1801 was then prepared for addition of the maleimide and catalyst (copper) was removed as follows: A prepacked RediSep® Rf normal phase silica column is used to remove the catalyst. The size of the column is chosen based on the copper amount in the reaction mixture. For instance, a 330 g column (Cat. #69-2203-330, Column size 330 g, CV=443 mL) was used for a 50 g batch of OG1801. Teflon tubing is used for all the connection as EtOH is the elute solvent.

After copper removal, all the fractions were transferred to a round bottom flask in batches, and evaporated the EtOH by rotary evaporator at 45-50° C. at reduced pressure to dryness. In this step, EtOH volume collected from condensation was monitored to make sure EtOH removal was >90%. The polymer was dissolved in 250 mL of WFI and filtered using a 0.2 um filter. It resulted in a clear to light yellow polymer solution at ~150 mg/mL. The solution could be stored at 2-8° C. up to 3 months before use.

### Example 4. Synthesis of Polymer OG1802

Starting materials and reagents needed in the reaction are calculated based on batch input of OG1801. The linker is 3-maleimidopropionic acid, NHS ester. Added 30 ml of 0.5 M sodium phosphate (in WFI, pH 8) to 50 g polymer solution (~150 mg/mL). Let stir for 1 min; pH was 8.0 by pH paper. Weighed 204.8 mg of linker and dissolved in DMF 4.1 mL to make 50 mg/mL stock sln. Added linker solution dropwise 815 uL per minute to the polymer sln with strong stirring. Took 5 min to added 4095 uL of linker solution. Reacted at room temperature for 30 min. Quenched reaction with 20 mL of 5% acetic acid to achieve a final pH of 5. Filtered the solution using 1 L vacuum filter (0.2 um).

OG1802 (see FIG. 10) is then purified as follows: Millipore TFF cassettes are used for polymer purification in aqueous system. Started with concentrating the polymer solution to 250 mL (~200 mg/mL). Added the fresh WFI from reservoir and adjusted the flow rate of the fresh WFI feed to the same as the permeate (~~2 mL/min). The UF/DF was set up at 2-8° C. overnight. Typically 2.5 L of WFI was used (10× volume ratio to the polymer solution). A sample of retentate was collected for purity test. The targeted purity was >98%. Filtered the polymer solution by 0.2 μM 1 L filter bottle. The polymer solution could be stored at 2-8° C. for up to 3 months before conjugation.

### Example 5. Alternative Phosphorylcholine Polymers

A HEA-PC polymer was synthesized as described below. HEA-PC (2-(acryloyloxy)ethyl-2-(trimethylammonium)ethyl phosphate), which is an acrylate as opposed to the methacrylate HEMA-PC described above, has the following structure:

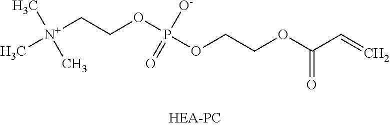 [SMILES: C=CC(=O)OCCOP(=O)([O-])OCC[N+](C)(C)C]

HEA-PC was polymerized to the initiator shown in Example 1 as compound L.

TABLE 5.1

| Reactant | Name | Amount | | MW |
|---|---|---|---|---|
| Initiator | Compound L (see above) | 1.65 | mg | 2505.5 |
| Monomer | HEA-PC | 0.461 | g | 281.24 |
| Catalyst | Cu (I) Bromide | 1.2 | mg | 143.45 |
| Ligand | Tris [2-(dimethylamino)ethyl]amine | 2.73 | mg | 230.39 |
| | (Me6TREN) | | | |
| Solvent A | N,N-Dimethylformamide (DMF) | 21.85 | μl | 73.09 |
| Solvent B | Water | 0.7 | ml | 18.02 |
| Solvent C | Methanol | 0.7 | ml | 32.04 |

Prepared a stock solution of initiator at 200 mg/mL by dissolving 2.2 mg of initiator in 11 μl of dry DMF and a 200 mg/ml solution of ligand by dissolving 4.6 mg of Me6TREN in 23 μL of dry DMF. Dispense 8.25 μl of the stock solution of initiator and 13.6 μl of the ligand into a tube. Degas at −78° C. for 5 min then refill with Argon and add 1.2 mg of CuBr. Degas and refill with Argon. Add a stock solution of HEA-PC in methanol (weigh out 0.461 g of HEA-PC and dissolve it in 0.5 mL of methanol) to the solution inside the reactor at −78° C. Rinse the vial with 200 μl of methanol and add it inside the reactor at −78° C. and then 0.5 mL of distilled water then another 200 μl of water. Degas thoroughly until no bubbling is seen and all heterogeneity disappears (solid particulates dissolve or disappear). Refill with 4 psi of Argon and let the reaction proceed at RT for an hour. The reaction was already viscous. The reaction was allowed to proceed for about one hour. A solution of bipyridine in methanol (5 mg in 0.5 uL) was added. Another 2-3 ml of methanol was added and the catalyst was allowed to oxidize overnight at 4° C. Conversion determined by 1H NMR was estimated to be 94%.

The next day the polymer was dialyzed and subjected to SEC/MALS analysis using Shodex SB806M_HQ column (7.8×300 mm) in 1×PBS pH 7.4 at 1 ml/min, giving a PDI of 1.157, Mn of 723.5 kDa, Mp of 820.4 kDa and Mw of 837.2 kDa (before dialysis PDI is 1.12, Mn=695 kDa, Mp=778 kDa). Next a maleimide functionality was added to the polymer so that it could be conjugate to a protein.

Next, the maleimide Mal-PEG4-PFP (see Example 1 above) ester was snapped on to the HEA-PC polymer as shown in Example 1. The resulting maleimide functionalized HEA-PC polymer can then be conjugated to sulfhydryl groups as discussed herein for HEMA-PC polymers.

An acrylamide PC polymer was also made using the monomer 2-(acrylamyl)ethyl-2-(trimethylammonium)ethyl phosphate (Am-PC), having the following structure:

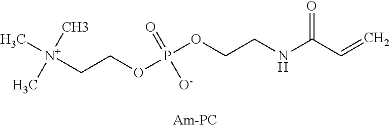 [SMILES: "Am-PC" ; C=CC(=O)NCCOP(=O)([O-])OCC[N+](C)(C)C]

The Am-PC was used for polymerization employing a 3 arm initiator (a TFA salt) having the structure:

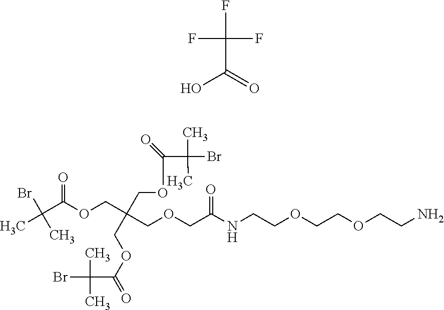 [SMILES: O=C(O)C(F)(F)F ; CC(C)(Br)C(=O)OCC(COCC(=O)NCCOCCOCCN)(COC(=O)C(C)(C)Br)COC(=O)C(C)(C)Br]

The synthesis of the Am-PC polymer was conducted as follows:

TABLE 5.2

| Reactant | Name/Identity | Amount | | MW |
|---|---|---|---|---|
| Initiator | 3-arm initiator (see above) | 2.2 | mg | 885.35 |
| Monomer | Am-PC | 0.5 | g | 280.26 |
| Catalyst (I) | Copper (I) Bromide | 1 | mg | 143.45 |
| Catalyst (II) | Copper (II) Bromide | 0.2 | mg | 223.35 |
| Ligand | Tris[2-(dimethylamino)ethyl]amine | 3.94 | mg | 230.39 |
| | (Me6TREN) | | | |
| Solvent A | N,N-Dimethylformamide (DMF) | 31.7 | μl | 73.09 |
| Solvent B | Water | 1 | ml | 18.02 |
| Solvent C | Methanol | 1 | ml | 32.04 |

A stock solution of ligand at 200 mg/mL was prepared by dissolving 9 mg of Me6TREN in 45 uL of dry DMF. Add 19.7 uL of the stock solution to a reaction vessel. Prepare a stock solution of initiator at 200 mg/mL by dissolving 6.5 mg of material in 32.5 uL of DMF. Add 11 uL of the initiator stock solution to the ligand from above. Degas for 5 mn. Add 1 mg of CuBr. Prepared a stock solution of CuBr2 at 200 mg/mL by dissolving 4 mg CuBr2 in 20 μL of DMF. Add 0.5 g of monomer (AmPC) to 1 mL of methanol (slow dissolution/viscous solution), followed by luL of the stock solution of CuBr2. Add the monomer solution dropwise to the reaction mixture above. Rinse with 1 mL of water. Degas the reaction mixture thoroughly (freeze-thaw). Let the reaction proceed for 24 hours.

Afterwards the Am-PC polymer may be dialyzed. The molecular weight of the above polymer was determined by SEC/MALS: Mn is 215 kDa, Mp: 250 kDa, PDI is 1.17. Conversion was estimated by 1H NMR to be 94%. A maleimide functionality can be added to the Am-PC polymer as discussed above for HEMA-PC and HEA-PC. Maleimide functionalized Am-PC polymer can be conjugated to a protein as described above.

### Example 6. Reverse Ellman's Assay for Calculating Free Maleimide in a Compound

After addition of the maleimide functionality to polymer OG1801 to form OG1802 (see above), an Ellman's assay was used to determine the amount of functional maleimide (i.e. conjugatable) in a sample. Thiol converted Ellman's reagent (DTNB) to TNB-then to TNB2- in water at neutral and alkaline pH, which gave off a yellow color (measured at 412 nm). A standard curve was established with cysteine. Since the maleimide reacts with thiol, this assay measured the thiol (cysteine) left. The inhibition was calculated as the (original thiol-thiol left after maleimide polymer addition)/(original thiol) and is expressed as a percentage.

Reagents Employed in Assay: A standard curve was prepared using the cysteine from 62.5 μM to 2 μM. Polymer stock solutions were prepared by dissolving the powder in 1×PBS pH7.4 (reaction buffer) and mixing thoroughly. An equal molar of polymer and cysteine solutions were mixed and allowed to react at 27° C. for 30 minutes. The 150 μM of DTNB solution was added into the cysteine standards and polymer/cysteine reactions and the color was developed at 27° C. for 5 minutes. OD at 412 nm was read on the Spectramax plate reader and percent inhibition was calculated with the Softmax Pro software and the cysteine standard curve.

### Example 7. Protein Sequence of Antibody (OG1950) Comprising an Anti-VEGF-A Antibody Heavy Chain with an L443C (EU Numbering, or 449C in SEQ ID NO: 1) Mutation and an Anti-VEGFA-Antibody Light Chain

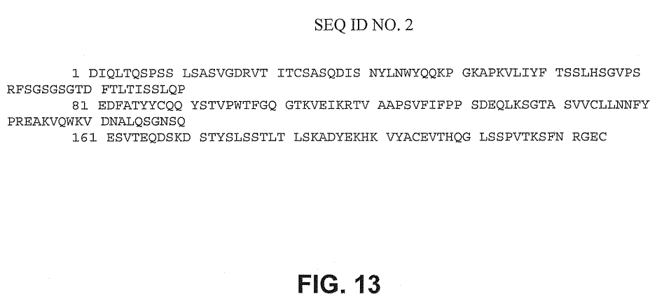

An anti-VEGF-A antibody with an L443C (EU numbering) mutation having the sequence set forth below in SEQ ID NO: 1 (FIG. 12) (heavy chain) was cloned. An anti-VEGF-A antibody light chain having the sequence set forth in SEQ ID NO: 2 (FIG. 13) below was cloned.

### Example 8a. Purification and Decapping of OG1950

The OG1950 heavy and light chains may be cloned into expression plasmids and transfected into CHO cells. Cells can be grown up in appropriate media and harvested. OG1950 may be purified using techniques described above. The OG1950 cysteine at position 443 (L443C (EU numbering)) residue is typically “capped” or oxidized by chemicals in the cell culture media and is not available for conjugation. In this regard, purified OG1950 may be subjected to a decapping (i.e. reducing) procedure to remove the cap and enable the free (i.e. those not involved in Cys-Cys disulfide bonds) cysteine residue to be conjugated to the maleimide functionality of a polymer. Decapping may be done by mixing purified OG1950 protein with a 30× molar excess for 1 hour at 25° C. of the reducing agent TCEP (3,3′,3″-Phosphanetriyltripropanoic acid). The reduction reaction with TCEP may be monitored by SDS-PAGE. Following denaturation, the OG1950 protein may be washed by TFF using a Pellion XL Ultrafiltration Cassette with 20 mM Tris pH7.5, 150 mM NaCl, 0.5 mM TCEP buffer to remove the cap. The TCEP reagent may then be removed in the same TFF setup with 20 mM Tris pH7.5, 150 mM NaCl. Reduced OG1950 may then be allowed to refold using air (ambient oxygen) which again is followed by SDS-PAGE as an assay.

### Example 8b. Purification and Decapping of OG1950

The OG1950 heavy and light chains may be cloned into expression plasmids and transfected into CHO cells. Cells can be grown up in appropriate media and harvested. OG1950 may be purified using techniques described above. The OG1950 cysteine at position 443 (L443C (EU numbering)) residue is typically “capped” or oxidized by chemicals in the cell culture media and is not available for conjugation. In this regard, purified OG1950 may be subjected to a decapping (i.e. reducing) procedure to remove the cap and enable the free (i.e. those not involved in Cys-Cys disulfide bonds) cysteine residue to be conjugated to the maleimide functionality of a polymer. Decapping may be done by mixing purified OG1950 protein with a 30× molar excess for 1 hour at 25° C. of the reducing agent TCEP (3,3′,3″-Phosphanetriyltripropanoic acid). The reduction reaction with TCEP may be monitored by SDS-PAGE. Following reduction, the OG1950 protein can be washed by TFF using a Pellicon XL Ultrafiltration Cassette with 30 kDa MWCO membrane from Millipore with 20 mM Tris pH 7.5, 150 mM NaCl, 0.5 mM TCEP buffer to remove the cap and the excess TCEP by using the same buffer without TCEP. Reduced OG1950 can then be allowed to reoxidize using DHAA at ambient temperature for 1 hour followed by TFF for removal of DHAA to form reoxidized decapped OG1950. The decapping status is monitored by SDS-PAGE assay.

### Example 9. Conjugation of OG1950 to MPC Polymer

Reoxidized decapped OG1950 may then be conjugated to polymer OG1802. An excess of OG1802 is used (at least 1× molar excess). Conjugation can be monitored by SDS-PAGE or Cation-exchange chromatography (CEX-HPLC) and driven to near completion. OG1953 conjugate may be purified via cation exchanger chromatography and buffer exchanged into the formulation buffer by TFF. OG1953 conjugate may be purified chromatographically as described above.

Conjugation efficiency is monitored with CEX-HPLC as follows. OG1950 and OG1953 conjugate at pH 5 are protonated and bound to a CEX column. The polymer shields the protein charge and results in weaker binding to CEX as compared to the unconjugated OG1950 protein and elutes at a lower conductivity. On the other hand, the OG1802 polymer is net charge neutral and is not bound to the column. Reduction of the OG1950 protein peak and OG1802 polymer peak with the appearance and increase in the conjugate peak indicates conjugation. Near complete absence of the OG1950 peak indicates a quantitative conjugation event.

### Example 10. OG1950 SPR Binding Kinetics

This Example illustrates binding of OG1950 to VEGF-165 in single cycle kinetics BIAcore™ experiment.

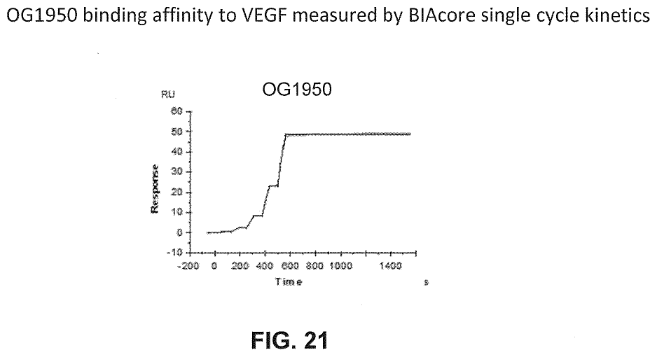

SPR interaction analysis of OG1950 to human VEGF-165 was performed on a BIAcore™ T200 system (GE Healthcare) equipped with a protein A chip (GE Healthcare). A single-cycle kinetics method was implemented. Antibody was captured at 25 μg/mL in HBS-EP⁺ buffer (0.01 M 4-(2-hydroxyethyl)-1-piperazineethanesulfonic acid, 0.15 M sodium chloride, and 0.05% polysorbate 20) with a pulse of 25 s at a flow rate of 10 pI/min. Subsequently, recombinant human VEGF-165 (R&D Systems) was applied at various concentrations with a pulse of 60 s at a flow rate of 30 pI/min and a final dissociation time of 1000 s. The experiment was carried out at 25° C. The surface was regenerated with 10 mM glycine pH 1.7 for 1 min at a flow rate of 50 μL/min. Binding kinetics analysis was performed using the Biacore T200 evaluation software with responses globally fit to a 1:1 interaction. Results are summarized in Table 10.1 and FIG. 21.

TABLE 10.1
BINDING KINETICS OF OG1950 TO VEGF-165

| | ka (1/Ms) | kd (1/s) | KD(M) |
|---|---|---|---|
| OG1950 | 3.35E+05 | 6.77E−08 | 2.02E−13 |

OG1950 exhibits <1 pM KD in a 1:1 binding fit model (beyond instrument sensitivity).

### Example 11—Conjugation and Purification of OG1953 Using Cation Exchanger Chromatography

OG1950 protein expression and protein preparation: OG1950 protein was expressed in mammalian GSCHOK1 expression system followed by purification using a Protein A affinity column. The purity of the Protein A column purified OG1950 was over 90% based on size exclusion chromatography and SDS-PAGE. The engineered specific cysteine residue of OG1950 was available for thiol conjugation to the OG1802 biopolymer. The thiol reacting chemical group of OG1802 was to react to form stable covalent linkage, which forms the OG1953 bioconjugate. To accomplish this, 1 mg of OG1950 was fully reduced with Tris-(2-Carboxyethyl)phosphine, Hydrochloride (TCEP) reducing agent followed by removal of TCEP via buffer exchange using a 30 KDa spin concentrator. The fully reduced OG1950 protein was then allowed to reoxidize to ensure all expected protein disulfide linkage was formed except the engineered cysteine residue (decap OG1950), which remained in a reduced form for conjugation to the biopolymer via thiol specific conjugation chemistry.

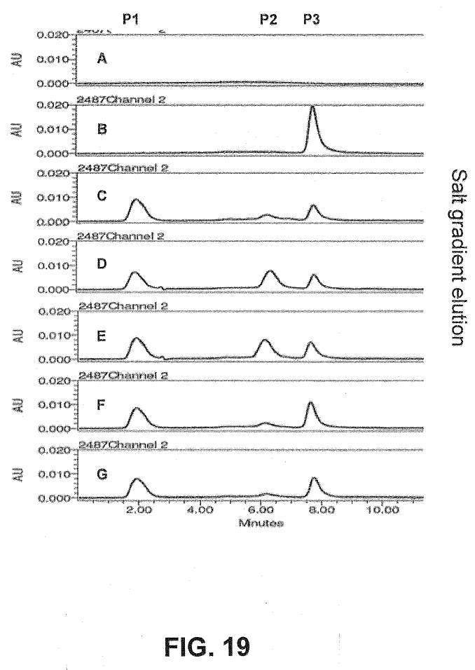

Conjugation reaction: The conjugation reactions were performed by mixing 100 ug of decap OG1950 with 15× molar excess of OG1802 biopolymer with final protein concentration at 2 mg/mL in Tris buffer (20 mM Tris, 100 mM NaCl, pH 7.5). Various additives were evaluated in different conjugation reactions as shown in Table 11.1. in order to compare their impact on the conjugation efficiency. All reactions were set up in a fixed volume and incubated at 4° C. for 20 hours. Table 11.1 and FIG. 19 shows ion Exchanger analysis (A280 absorbance) and fractionation of the conjugation reactions A through G. Reaction A contained buffer only; reaction B contained OG1950 antibody only; reaction C contained OG1950 antibody+OG1802 polymer; reaction D contained OG1950 antibody+OG1802 polymer+sucrose; reaction E contained OG1950 antibody+OG1802 polymer+trehalose; reaction F contained OG1950 antibody+OG1802 polymer+glutamic acid; reaction G contained OG1950 antibody+OG1802 polymer+aspartic acid. Reactions B-G were started with 100 ug OG1950 protein input with a fixed total reaction volume. Upon reaction completion, equal volumes of B-G reactions were injected for IEX analysis. The conjugation efficiency comparison is shown in FIG. 19. Peak 1(P1) represents the excess biopolymer (OG1802) that was not conjugated to the protein and unable to bind to the ion exchanger column and therefore eluted as unbound fraction in each reaction; Peak 2 (P2) represents the antibody polymer bioconjugate in each reaction; Peak 3 (P3) represents the free antibody that was not conjugated to the polymer in each reaction.

TABLE 11.1

| | | (P2) | (P3) | Conjugation |
|---|---|---|---|---|
| | | OG1953 Conjugate | OG1950 Protein | efficiency |
| | Samples | (Au × Second) | (AU × Second) | P2/[P3 of B] |
| A | Buffer blank injection | NA | NA | NA |
| B | OG1950 protein control | 0 | 410129 | NA |
| C | OG1950 + OG1802 biopolymer | 37334 | 125884 | 9% |
| D | C + Sucrose | 198064 | 110356 | 48% |
| E | C + Trehalose | 202682 | 139313 | 49% |
| F | C + Glutamic acid | 30401 | 220577 | 7% |
| G | C + Aspartic acid | 29054 | 175266 | 7% |

As can be seen from the results above, various excipients allowed for significantly higher conjugation efficiency. Without intending to be limited by theory, such excipients which assist in maintaining the solubility of the ingredients help the conjugation efficiency.

Cation exchanger analysis of the OG1953 conjugation reaction: Upon conjugation reaction completion, 5 ul of each reaction mixture was diluted 3-fold with a column equilibration buffer (20 mM sodium acetate pH 5.5) for cation exchanger chromatography (IEX) analysis and separation using a Shodex SP-825 HPLC column. IEX was performed under bind and elution mode where the reaction mixture was first diluted to lower the salt concentration so both conjugate and unreacted protein would bind to the column, followed by a salt gradient elution where increasing NaCl concentration resulted in elution of the conjugate (OG1953) and unconjugated free protein (OG1950) at different retention times due to the differential charge variation. IEX analysis results, as shown in FIG. 19 show that the OG1953 conjugate (peak P2) separated very well from the unreacted free polymer (OG1802) as shown in peak P1 and unreacted free protein (OG1950) as shown in P3.

Scale up purification of the OG1953 conjugate: Conjugation reactions D and E were pooled and further separated with the IEX where the conjugate Peak (P2) eluted fractions were collected and concentrated for activity analysis.

### Example 12—Effect of Anti-VEGF Molecules on Biotin-VEGF Binding to VEGFR Using ELISA

The abilities of OG1950, OG1953 and other anti-VEGF molecules to inhibit binding of Biotin-VEGF-165 to VEGFR were tested on an ELISA assay. 96 well ELISA plates were coated with 1 ug/mL recombinant VEGF R1-Fc protein (R&D systems part #321-FL-050). Plates were Incubated o/n at RT. Plates were then washed and blocked with blocking buffer (1% BSA (Sigma Aldrich, Product #A7906, heat shock fraction, pH 7, ≥98%) in 1×PBS, pH7.4) for >=90 min with gentle shaking. 3-fold dilution series of test samples were made. Starting from 400 nM samples were mixed with biotin-VEGF (R&D systems, part # custom06). The final concentration of biotin-VEGF was 4 ng/mL (100 pM). The top concentration of sample dilution series was 85 ug/mL. After dilutions were made, samples were incubated at RT for >=30 min and then washed 3×. After washing, 100 μL of the sample/biotin-VEGF mixture was transferred to each well. Plates were incubated for >=90 min at RT with gentle shaking. After incubation 100 ul of the 1:1500 diluted SA-HRP (R&D systems, part #A7906) was added to each well. Incubated plates were protected from light for approximately 20 min. Plates were then washed 3×. After washing 100 ul TMB substrate (R&D systems, part #DY998) was added. Plates were incubated and protected from light for approximately 30 min. Color development was monitored. Color development was stopped by adding 50 ul of stop solution. Plates were read at 450 nm. Results are shown in FIG. 20

These results show that the OG1953 antibody/conjugate reached a higher maximal inhibition of Biotin-VEGF binding to VEGFR when compared to OG1950 (the unconjugated antibody) as well as the commercially available VEGF inhibitors Lucentis®(ranibizumab) and Avastin®(bevacizumab). In a typical VEGF ligand VEGF receptor binding assay, addition of OG1953 results in inhibition 97-98% of VEGF ligand binding to the VEGF receptor. This is compared to adding OG1950, which results in inhibition of 75-87% of VEGF ligand/receptor binding and adding Lucentis®(ranibizumab) or Avastin®(bevacizumab), each of which only results in inhibition of 68-78% of VEGF ligand/receptor binding. Furthermore, previous studies have shown that adding either OG1950, Lucentis®(ranibizumab), or Avastin®(bevacizumab) at concentrations of 400 nM does not result in 100% inhibition of binding of the VEGF ligand to the VEGF receptor.

FIG. 20 also shows that the other antibody conjugate anti-VEGF Bio Conjugate (anti-VEGF BC) reached a higher maximal inhibition of binding of VEGF to the VEGF receptor when compared to the antibody alone (anti-VEGF). Together these results show that (i) the OG1953 antibody conjugate is more effective at inhibiting VEGF ligand binding to the VEGF receptor when compared to currently available VEGF inhibitors and (ii) conjugating VEGF antibodies at a site outside of the region of the active site can result increased inhibition of VEGF ligand binding to the VEGF receptor.

In some embodiments, provided herein are anti-VEGF antibody conjugates that display a superior (or at least equal) level of blocking ability, as compared to the anti-VEGF antibody alone.

### Example 13—Method of Determining Binding of OG1950 to Fc Gamma Receptor I and IIIa

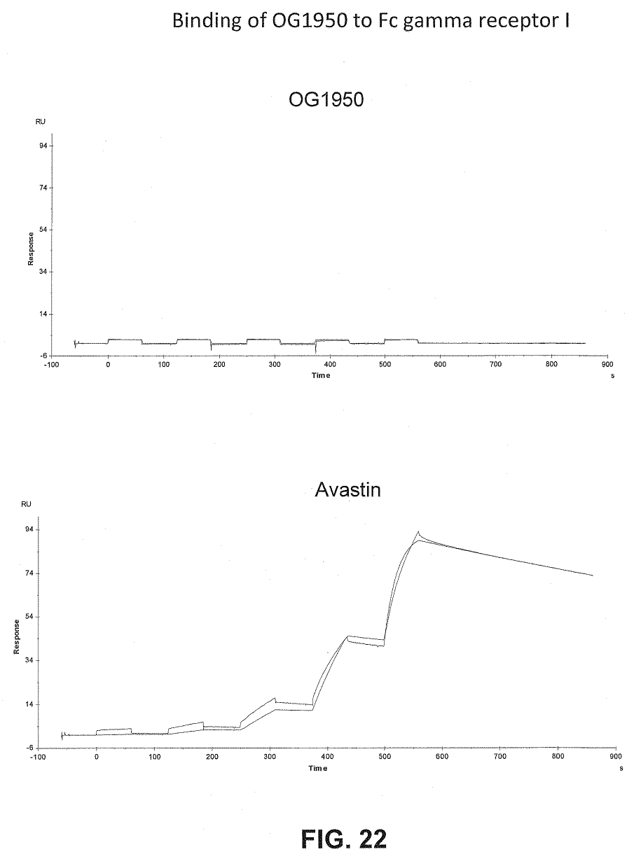

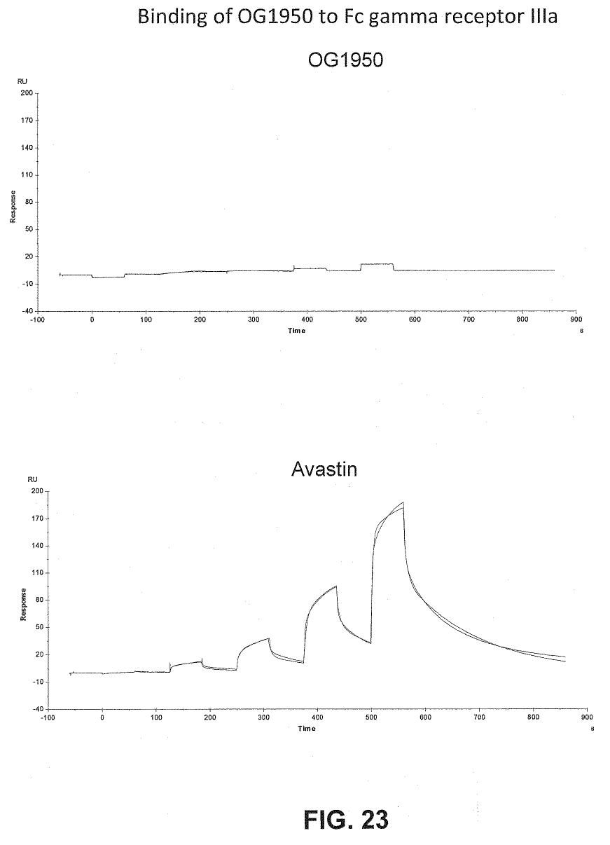

Binding kinetics experiments were performed at 25° C. using a BIAcore T200. An anti-his antibody was immobilized on a CM5 chip. Histidine-tagged FcγRI and FcγRIIIa at a concentration of 0.5 μg/mL prepared in HBS-EP buffer (0.01M HEPES pH7.4, 0.15M NaCl, 3 mM EDTA, 0.005% Tween-20) were injected independently for 60-s using a flow rate of 5 μL/min in the active flow cell only. Antibody candidate and Avastin®(bevacizumab), used as a positive control, were then injected over the reference and active flow cell using 60-s injections at 30 μL/mL, applying single-cycle kinetics method. Antibody concentrations in the range of 0.48 to 300 nM were used for FcγRI and 7.8 nM to 2000 nM for FcγRIIIa. Following each run, flow cells were regenerated with a 60 s injection of 10 mM glycine pH 1.7 using a flow-rate of 50p/mL. Data was double referenced, using subtraction of both reference flow cell and blank cycles. Analysis was performed using BIA evaluation software. Results are shown in FIGS. 22 and 23. Results show that OG1950 showed no significant binding to either Fc gamma receptor I and IIIa in this assay.

### Example 14—Method of Determining Binding of OG1950 to Human Complement Protein C1q

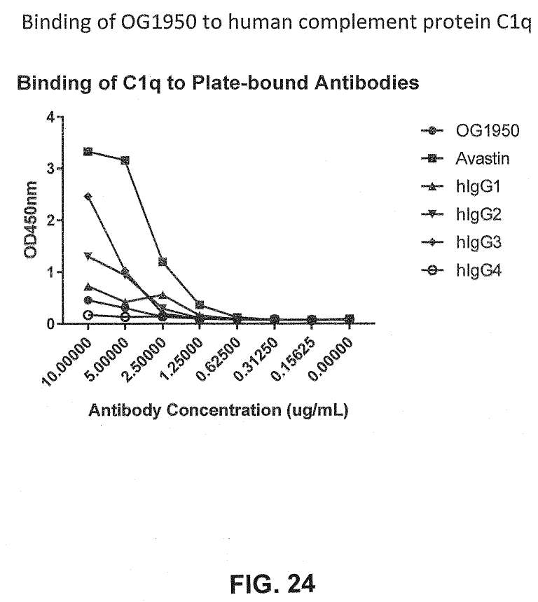

Complement engagement liabilities were assessed by C1q ELISA binding. The antibody panel was titrated 1:2 from a top concentration of 10 ug/mL in 1×PBS for overnight coating at 4C. Plates were then blocked After a 2 hour blocking step in 1% BSA. Purified human C1q was then applied at 5 ug/mL in 1% BSA for 2 hours at room temperature followed by detection with HRP-conjugated anti-human C1q antibody and TMB development. Results are shown in FIG. 24. Results show that C1q has more binding affinity for Avastin®(bevacizumab) relative to OG1950 at antibody concentrations between 10 μg/mL and 0.625 ug/mL.

In some embodiments, OG1950 has less than 10% of the binding of that of Avastin. In some embodiments, OG1950 has less binding to C1q than Avastin®(bevacizumab).

### Example 15—Effect of Anti-VEGF Agents to VEGF Stimulated HRMVEC Proliferation

Human retinal microvascular endothelial cell (HuRMVEC) proliferation assays were performed as follows: cells were maintained in CSC complete medium (cell systems, #4Zo-500) supplemented with 2% of CultureBoost (cell systems, #4CB-500) and 0.2% of Bac-off (cell systems, #4ZO-643), seeded in 96-well plates in assay medium (serum free medium (cell systems, #4Z3-500-S) with 5% FBS) at density of 10,000 cells per well. VEGF inhibitors were first added at the indicated concentrations to each well. Thirty minutes later, VEGF 165 (R&D systems, #293-VE-500/CF) was added to a final concentration of 1.3 nM. After 3 days, cells were incubated with WST-1 cell proliferation assay reagent and read at OD450 nM.

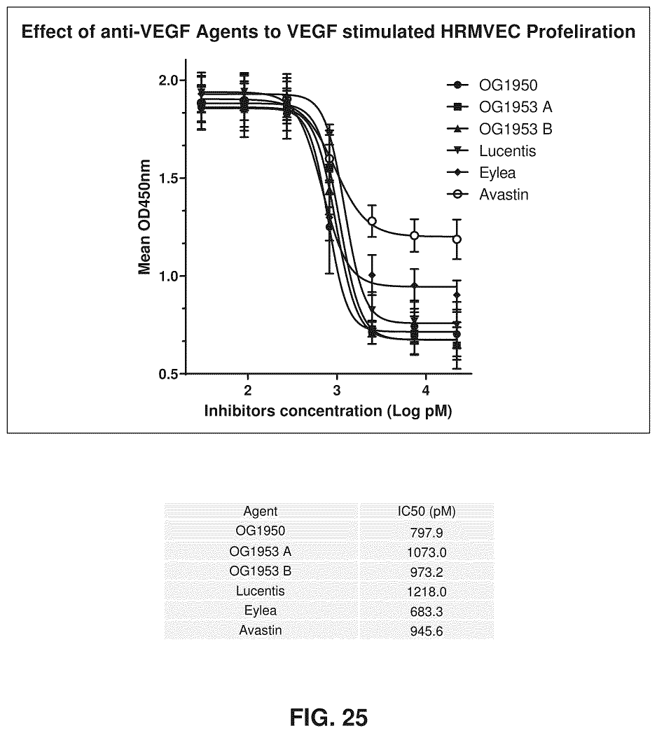

Results demonstrated that two independent preps of OG1953 (OG1953A and OG1953B) both show potent inhibition to the HuRNVEC proliferation. Both maximal inhibition and IC50 of OG1953 is comparable to that of antibody alone OG1950 in this assay. Maximal inhibition of OG1953 is also significantly better than that of Avastin®(bevacizumab) and Eylea®(aflibercept). The results (including IC50 values and their comparison with Lucentis®(ranibizumab), Eylea®(aflibercept), and Avastin®(bevacizumab)) are shown in FIG. 25.

### Example 16—Single Cycle Kinetics (SCK) of VEGF Binding to Anti-VEGF Agents Captured on a Protein A Chip at 25 Degrees

Binding kinetics was performed at 25° C. using a BIAcore T200 on Avastin®(bevacizumab), OG1950 and OG1953. Briefly, anti-VEGF agents were captured on a Protein A chip (GE). 1 ug/ml OG1950 and Avastin®(bevacizumab) were flowed at 25 ul/min for 2 mins. 10 ug/ml OG1953 was flowed at 10 uls/min for 10 mins. VEGF (recombinant; R&D systems) was flowed over captured antibodies for 240 seconds contact time each at 0.56 nM, 1.67 nM, 5 nM, 15 nM, and 45 nM for single cycle kinetics, and dissociated for 30 minutes. Analysis was performed using BiaEvaluation software (GE). All sensorgrams were double reference subtracted and fit using a 1:1 Langmuir binding model. Off-rate of these anti-VEGF agents might be under-estimated in this experiment, due to the disassociation between anti-VEGF agents and Protein A capture.

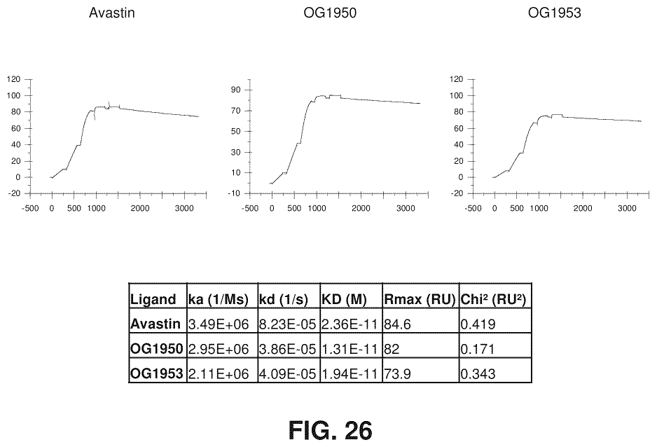

The results are presented in FIG. 26, including the calculated KD, ka, and kd values.

### Example 17—Preformulation Study of Mixing Antibody OG1950 with Either OG1801 Polymer Solution or OG1953 Conjugate Solution

Using the standard conjugation reaction process setup, the OG1950 conjugation reaction mixture was found to be cloudy with precipitate immediately present upon mixing. Further investigation revealed the precipitate was the protein itself, which in turn resulted in poor conjugation efficiency observed via either SDS-PAGE or ion exchanger analysis as the protein was lost by precipitation instead of participating in the conjugation reaction.

Initial troubleshooting experiments performed revealed conditions that did not result in a clear reaction solution included (1) reduction of polymer molar excess ratio from over 10 to less than 5; (2) preadjusting the reaction solution pH to more acidic (e.g. pH 5.0) or basic (e.g. pH 8.5) from the standard neutral pH range (e.g. pH 6.5-7.5); (3) testing of other IgG1 protein samples with similar or different isoelectric point (pI) as compared to OG1950; (4) buffer exchanged the sample storage buffer into 1×PBS pH 7.4 or 20 mM Tris buffer pH 7.4, 100 mM NaCl; and (5) preadjusting the OG1802 solution with 20 mM Tris buffer pH 7.4. Additional efforts have been done to try resolve such issue, which includes: (1) swap addition sequence of protein and conjugate solution; (2) slow addition of polymer to protein or vice versa: (3) dilute the polymer solution first to 20 mg/ml and combine with the protein solution followed by concentration and buffer exchanged to the original OG1953 formulation with a combined final total concentration of 50 mg/ml; (4) let the cloudy solution to be incubated at either refrigeration (2-8° C.) or ambient temperature for over a week; (5) lowering free protein to as low as 5% in the mixture. However, all of these measures did not result in a clear solution. The cloudy solutions in different experiments were centrifuged, and a white pellet has been observed. Determination of the protein concentration in the mixture showed a significantly lower protein concentration, and SDS-PAGE analysis of the solution showed only trace amount of free antibody that was unproportional to the expected free protein added, a good indication that the precipitation is due to free protein precipitation.

Protein is known to carry net surface charge that helps protein solubility in aqueous solution. The amino acids are referred to as hydrophilic amino acids which include arginine, lysine, aspartic acid, and glutamic acid. At neutral pH 7 the side chains of these amino acids carry charges—positive for arginine and lysine, negative for aspartic acid, and glutamic acid. Altering the solution pH could modulate the intrinsic protein solubility which Is therefore in some of the troubleshoot experiments mentioned above such as (2) this approach was applied. In theory, proteins solubility in aqueous solution differs depending on the level of hydrophobic or hydrophilic properties of the surface. Proteins with surfaces that have greater hydrophobic properties will readily precipitate. The addition of ions (e.g., NaCl or other salt) creates an electron shielding effect that nullifies some activity between water particles and the protein, reducing solubility as the proteins bind with each other and begin to aggregate. In the current situation, it was hypothesized that the biopolymer directly or indirectly modulates the protein surface charge and/or exposed surfaces and/or adjacent water microenvironment in a manner that allows and/or promotes intermolecular hydrophobic interactions which can result in protein precipitation.

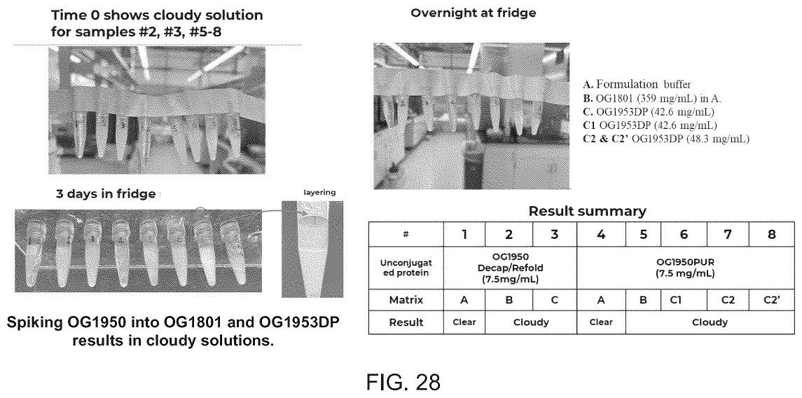

Next, to determine (1) the stability of OG1950DECAP, (2) the stability of OG1950PUR with biopolymer in the formulation, and (3) the stability of OG1950PUR mixed in OG1953, the following experiment was conducted, and the results are shown in FIG. 28.

The experiment was performed as shown below:
1. Prepare 50 ml OG1953 formulation buffer stock solution (12.5 mM sodium phosphate pH 6.5, 0.025% PS20) and sterile filtered with a 0.2 um filter.
2. 3 mL of OG1950DECAP_R12208B was concentrated using Vivaspin 2, 30 kDa MWCO, from 15.64 mg/mL to ~50 mg/mL (=~930 uL).
   2a. The concentrated OG1950DECAP_R12208D solution was sterile filtered with 0.2 μm filter.
   2b. The protein concentration of the concentrated and sterile filtered OG1950DECAP_R12208D solution was measured.
3. 0.5 mL of OG1950PUR_R12208 was concentrated using Vivaspin 500, 30 kDa MWCO, from 50 mg/mL to ~150 mg/mL (=~160 uL).
   3a. The concentrated OG1950PUR_R12208C solution was sterile filtered with a 0.2 μm filter.
   3b. The protein concentration of the concentrated and sterile filtered OG1950PUR_R12208C solution was measured.
4. Table 17.1 shows the reagents used in this example. Reactions were set up according to Tables 17.2.
5. In a BioSafety laminar flow hood, retrieve 8 clean 1.5 ml screwcap eppendorf tubes and set on a tube rack. In reference to Table 2 for the required volume of each reagent for each reaction, use a positive displacement pipettor fitted with pipet tip, aliquot either the OG1801 polymer stock solution or OG1953DP as shown in Reagents table below (Table 17.1) into the corresponding Rxn # vial, then add the formulation buffer stock and the volume of WFI needed, and lastly add slowly the OG1950 stock solution into the reaction mixture. Due to the viscous nature of each reaction, each reaction was prepared carefully and individually.

For an injectable eye medicine, the injectable volume can be limited to 50-100 ul per dose per eye. Delivering a 5 mg dose per eye per dosing (at 100 μl) based on the anti-VEGFR antibody (OG1950) will require a 50 mg/ml combined protein formulation. For a 15% free protein formulation, the OG1953 will be lowered from a 50 mg/ml conjugate-only formulation to 42.5 mg/ml in order to make room for 7.5 mg/ml free protein (15%).

OG1953-conjugate stock solution or OG1953DP at 50 mg/ml based on the protein moiety contained about 300 mg/ml OG1802 polymer in the formulation, this was a clear, colorless and highly viscous solution. This solution was formulated in pH 6.5 sodium phosphate containing 0.025% polysorbate 20 (PS20). The OG1950 antibody stock solution at 50 mg/ml was also clear and colorless or off white when formulated at phosphate buffer at pH 7.2. The pI of OG1950 is about 7.4, and this suggests that the OG1950 antibody remains soluble even at a buffer pH close to its pI at a concentration as high as 50 mg/ml. Mixing 1 part OG1950 stock solution with 5.7 parts OG1953 stock solution as described above resulted in a mixed solution of 7.5 mg/ml OG1950 as free protein to 42.5 mg/ml OG1953 conjugate. This combination formed a 15% mixture as defined elsewhere in the current context. However, this mixture turned turbid immediately as shown in FIG. 28.

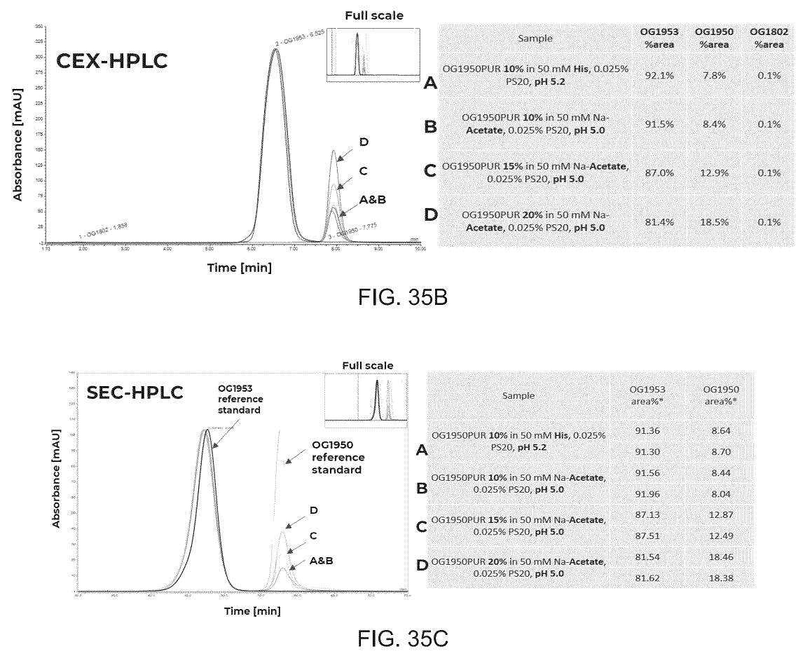

Additional efforts were made to resolve the increased turbidity, which includes: (1) swap addition sequence of protein and conjugate solution; (2) slow addition of polymer or OG1953 conjugate stock solution to protein or vice versa; (3) dilute the polymer solution first to 20 mg/ml and combine with the protein solution followed by concentration and buffer exchanged to the original OG1953 formulation with a combined final total concentration of 50 mg/ml as shown in FIG. 35A, panel A; (4) let the cloudy solution to be incubated at either refrigeration (2-8° C.) or ambient temperature for over a week; (5) lowering free protein to as low as 5% in the mixture; and (6) preadjust the pH of the OG1950 protein stock from 7.2 to 6.5 prior to mixing with the OG1953 conjugate stock solution. However, these measures did not result in a clear solution. The cloudy solutions in different experiments were centrifuged, and white pellet or a fluffy white layer of solution on the bottom of tube has been observed, determination of the protein concentration in the supernatant showed significantly lower protein concentration and SDS-PAGE analysis of the solution show only trace amount of free antibody that is unproportional to the expected free protein added, a good indication that the precipitation is due to free protein precipitation.

The initial attempts to lower the pH to 6.5 did not result in a clear solution. The original OG1953 injectable formulation at neutral pH of 7.2 is closer to the physiological condition of the vitreous in the eyes. Hence the initial efforts for the mixed formulation as described above sought to preserve the existing formulation at neutral pH of 7.2. Additional excipient and formulation consideration was performed as described in Examples 18-209 to identify conditions that are favorable to form a clear solution for the mix formulation.

TABLE 17.1
Reagents chart
Reagents

| | | | Conc. | MW | |
|---|---|---|---|---|---|
| Sample name | Description | BatchR# | (mg/mL) | (kDa) | Formulation |
| OG1801 | Biopolymer without | R15479 | 359 | 800 | Water for injection |
| | maleimide functional group | | | | (WFI) |
| OG1950PUR | Purified OG1950 Antibody | R12208 | 50.4 | 150 | PBS pH 7.2 |
| | Intermediate | | | | |
| OG1950PUR | Concentrated purified | R12208C | 143.4 | 150 | PBS pH 7.2 |
| | OG1950PUR | | | | |
| OG1950DECAP | Decapped OG1950 | R12208B | 15.64 | 150 | 20 mM Tris pH 8.5, |
| | antibody | | | | 50 mM NaCl, 5 mM |
| | | | | | EDTA |
| OG1950DECAP | Concentrated | R12208D | 40.7 | 150 | 20 mM Tris pH 8.5, |
| | OG1950DECAP | | | | 50 mM NaCl, 5 mM |
| | | | | | EDTA |
| OG1953DP | OG1953 Drug Product | R16346 | 50.8 | 950 | 12.5 mM Na-Phosphate |
| | | | | | pH 6.5, 0.025% Tw20 |

| OG1953DP formulation buffer = | | 12.5 mM Na-Phosphate |
|---|---|---|
| | | pH 6.5, 0.025% Tw20 |
| OG1953DP formulation buffer stock = | | 500 mM Na-Phosphate |
| | | pH 6.5, 1% Tw20 |
| Concentration of OG1802 in OG1953DP = | 266.7 | |

TABLE 17.2
REACTION OVERVIEW

| | | OG1950 | | OG1801 | | OG1953DP | |
|---|---|---|---|---|---|---|---|
| Sample | | | Conc. | | Conc. | | Conc. |
| ID | Description | Batch # | (mg/mL) | Batch # | (mg/mL) | Batch # | (mg/mL) |
| 1 | 15% | R12208D | 40.686 | NA | NA | NA | NA |
| | OG1950DECAP in | | | | | | |
| | DP formulation | | | | | | |
| | buffer | | | | | | |
| 2 | 15% | R12208D | 40.686 | R15479 | 359 | NA | NA |
| | OG1950DECAP in | | | | | | |
| | OG1801 | | | | | | |
| 3 | 15% | R12208D | 40.686 | NA | NA | R16346 | 50.8 |
| | OG1950DECAP in | | | | | | |
| | OG1953DP | | | | | | |
| 4 | 15% OG1950PUR in | R12208 | 50.4 | NA | NA | NA | NA |
| | DP formulation | | | | | | |
| | buffer | | | | | | |
| 5 | 15% OG1950PUR in | R12208 | 50.4 | R15479 | 359 | NA | NA |
| | OG1801 | | | | | | |
| 6 | 15% OG1950PUR in | R12208 | 50.4 | NA | NA | R16346 | 50.8 |
| | OG1953DP | | | | | | |
| 7 | 15% OG1950PUR in | R12208C | 143.366 | NA | NA | R16346 | 50.8 |
| | OG1953DP (at ~50 | | | | | | |
| | mg/mL) | | | | | | |
| 8 | 15% OG1950PUR in | R12208C | 143.4 | NA | NA | R16346 | 50.8 |
| | OG1953DP (half | | | | | | |
| | volume of #7, no | | | | | | |
| | spin) | | | | | | |

### Example 18—Design of Experiment Matrix for Spiking OG1950 into OG1953 or OG1801

DOE design matrix: Table 18.1 shows the input of 4 category of factors including (1) Active Pharmaceutical Ingredient (API) such as OG1950 and OG1953, each at 3 concentration levels; (2) Tonic factors such as sugars and salt, each is also at 3 levels; (3) the buffer species is restricted to the existing phosphate buffer; and (4) special excipient such as histidine, methionine, and detergents were also included in the design.

TABLE 18.1

| Category | | | | | Total | Tonic | | | | Total |
|---|---|---|---|---|---|---|---|---|---|---|
| name | API | #Strength (mg/ml) | | | count | factors | #strength (%) | | | count |
| Specific | OG1953DP* | 30 | 40 | 50 | 3 | Trehalose | 0 | 3 | 6 | 3 |
| components | OG1950 | 5 | 10 | 15 | 3 | Sucrose | 0 | 3 | 6 | 3 |
| | | | | | 0 | NaCl | 0 | 10 mM | 20 mM | 3 |

| | | |
|---|---|---|
| Subtotal | 9 | 27 |
| combination | | |

| Category | | | | | Total | Special | | | | Total |
|---|---|---|---|---|---|---|---|---|---|---|
| name | Buffering | #strength | | | count | excipient | #strength | | | count |
| Specific | sodium | 0 | 10 mM | 20 mM | 3 | Histidine | 0 | 10 mM | 20 mM | 3 |
| components | phosphate | | | | | | | | | |
| | | | | | 0 | Methionine | 0 | 7 mM | | 2 |
| | | | | | 0 | PS20 | 0 | 0.023% | 0.05% | 3 |
| | | | | | 0 | PS80 | 0 | 0.02% | 0.05% | 3 |
| Subtotal | | 3 | | | | | 54 | | | |
| combination | | | | | | | | | | |

*For OG1801 as input, its concentrations are 160, 213, and 267 mg/ml as equivalent concentrations to
OG1953 at 30, 40, and 50, respectively

| | |
|---|---|
| Total max possibilities | 39,366 |
| Total minimal combination (Hi, Lo) | 1026 |

The provided matrix allows a potential for 39,366 combinations. The readout is turbidity measure at absorbance 600 nm. Based on the input on Table 18.1, 30 combinations of all different factors were proposed, where sample ID 1-30 involved mixing OG1801 polymer with OG1950 antibody. The combinations of OG1801 with OG1950 and the results of the experiment are described in Example 19 below. The same matrix was performed using OG1953, where the OG1801 polymer solution was replaced with OG1953 as shown in the last two columns with sample ID 31-60. The combinations of OG1953 with OG1950 and the results of the experiment are described in Examples 20 below.

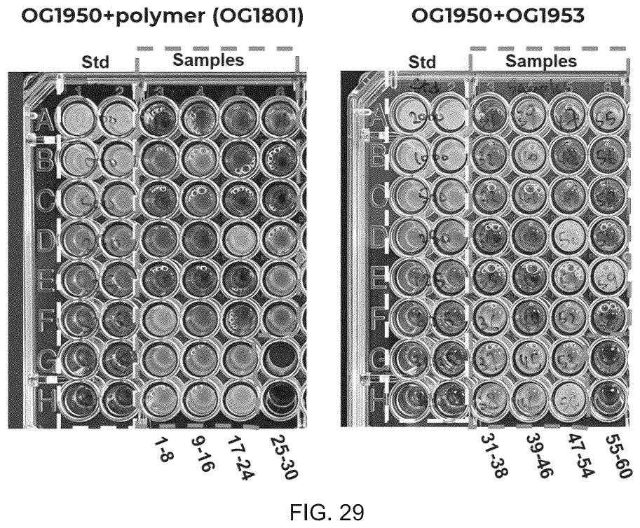

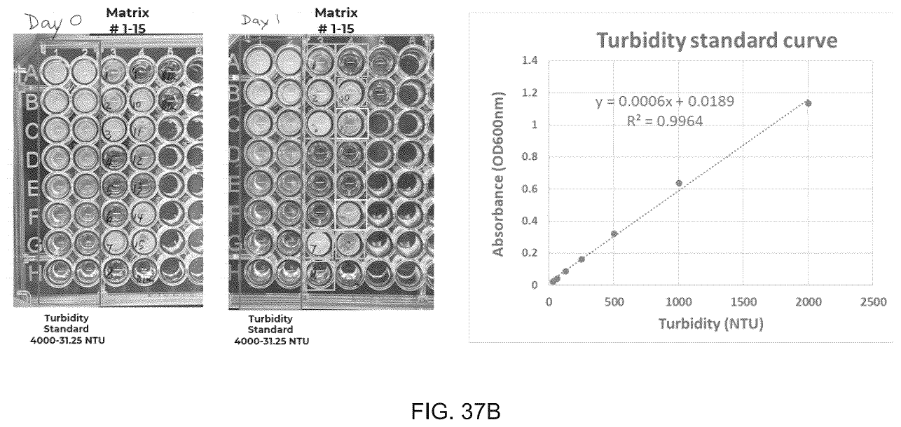

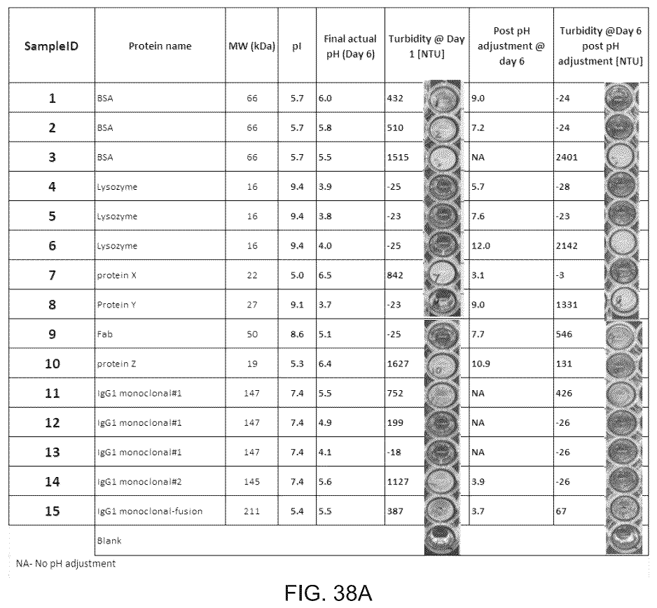

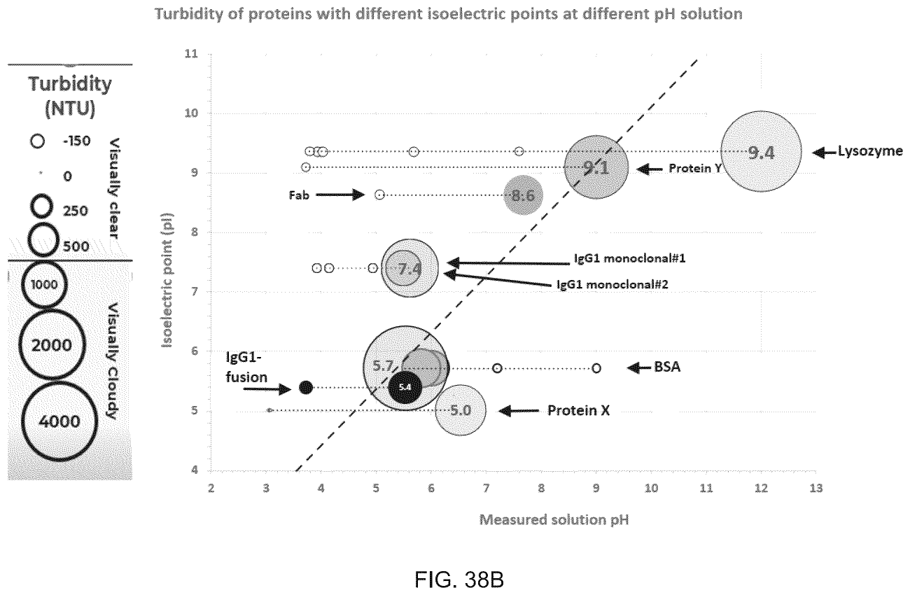

The experiments were conducted using a clear and flat bottom 96-well microtiter plate, as follows. First add and mix the protein and buffer diluent components in the wells, then to each well slowly add and mix in the polymer or conjugate stock solution. Let the plate incubate at ambient temperature for at least 30 min before any measurement. Then the turbidity of each well will be measured at OD600 nm in a plate reader. With reference to a turbidity standard curve using a 4000 NTU Formazin calibration standard that is twofold serially diluted in duplicate (see for example FIG. 38A right panel is a Formazin standard curve). Preliminary assay development showed that the linear range of the Formazin standard curve can be used to detect turbidity below 500 NTU, which is not readily visible by naked eyes. It should be noted that the Formazin standard curve at Y intercept is typically higher than zero (see for example, FIG. 37B on the right panel), and the turbidity value can be negative when the unknown sample absorbance reading is lower than the Y intercept value. The assay allowed quantitative measurements of the turbidity for scoring and as response input for data analysis. The microtiter plates with the formulation matrix set up were sealed with a plate sealer to prevent evaporation during the incubation at ambient temperature for up to 3 days, and each day the turbidity of each well in the plate was read. FIG. 29 is a visual representation of the performance in each condition. Software deconvolution was performed to search for trends related to a factor. None of these tested factors was identified as influencing the precipitation trend.

The pH of each sample #1-#60 was measured using a microprobe pH meter which is able to measure as low as 100 μl solution in the microtiter plate. The pH measurement was performed towards the end of the experiment to prevent microbial growth which could affect the experiment. In addition, these samples were highly viscous solutions, and frequent pH measurement was avoided to better maintain the solution volume.

### Example 19—Formulation DOE for Various Excipients and Matrix Combination Screening

A formulation design of experiment study (DOE) was performed for various excipients and matrix combination screening as described in Example 18, to obtain a formulation with low turbidity. The reagents used are shown in Table 19.1.

A mixture of OG1801 and excipients/matrix components in a 96-well PCR plate was prepared. In a 96-well assay plate, the required amount of OG1950 solution was transferred to the well, and then dispensed slowly while mixing the mixture of OG1801 and excipients/matrix components. The 30 different combinations of factors designed as discussed in Example 18 are shown in Table 19.2.

TABLE 19.1
Stock solution summary

| | | |
|---|---|---|
| Description | Concentration | Formulation |

| L-histidine | 200 | mM | In WFI |
|---|---|---|---|
| Sucrose | 50% | | In WFI |
| Trehalose | 50% | | In WFI |
| L-methionine | 200 | mM | In WFI |
| Polysorbate 20 | 1% | | In WFI |
| Polysorbate 80 | 1% | | In WFI |
| Sodium chloride | 5M | | In WFI |
| Purified OG1950 Antibody | 50.4 | mg/ml | PBS pH 7.2 |
| Intermediate (OG1950PUR) | | | |
| Polymer without maleimide | 359 | mg/ml | WFI |

functional group (OG1801)

TABLE 19.2
REACTION OVERVIEW

| | OG1801 or | | | | | | | | |
|---|---|---|---|---|---|---|---|---|---|
| | OG1953 | OG1950 | Trehalose | Sucrose | NaCl | Histidine | Methionine | PS20 | PS80 |
| | [g/L] | [%] | [%] | [%] | [mM] | [mM] | [mM] | [%] | [%] |
| 1 | 194.9 | 5 | 6 | 6 | 0 | 20 | 3.5 | 0.05 | 0 |
| 2 | 273.3 | 5 | 0 | 0 | 20 | 20 | 7 | 0.05 | 0 |
| 3 | 276.9 | 5 | 0 | 0 | 20 | 20 | 0 | 0.05 | 0.025 |
| 4 | 195.9 | 10 | 6 | 0 | 0 | 20 | 7 | 0.05 | 0.05 |
| 5 | 235.6 | 5 | 6 | 6 | 20 | 0 | 0 | 0.05 | 0 |
| 6 | 197.9 | 15 | 6 | 6 | 0 | 0 | 7 | 0 | 0.025 |
| 7 | 278.9 | 10 | 0 | 6 | 20 | 0 | 0 | 0 | 0 |
| 8 | 212.6 | 15 | 0 | 6 | 20 | 20 | 7 | 0 | 0 |
| 9 | 225.2 | 15 | 6 | 0 | 20 | 20 | 0 | 0 | 0 |
| 10 | 209.0 | 15 | 6 | 3 | 20 | 0 | 7 | 0.05 | 0 |
| 11 | 237.4 | 10 | 3 | 3 | 10 | 10 | 3.5 | 0.025 | 0.025 |
| 12 | 257.2 | 5 | 0 | 6 | 20 | 0 | 7 | 0.025 | 0.05 |
| 13 | 248.2 | 5 | 6 | 0 | 20 | 10 | 7 | 0 | 0.05 |
| 14 | 265.8 | 5 | 0 | 3 | 0 | 20 | 0 | 0 | 0.05 |
| 15 | 226.6 | 15 | 0 | 6 | 0 | 10 | 0 | 0.05 | 0 |
| 16 | 201.5 | 15 | 6 | 6 | 0 | 0 | 0 | 0 | 0.05 |
| 17 | 237.4 | 10 | 3 | 3 | 10 | 10 | 3.5 | 0.025 | 0.025 |
| 18 | 199.7 | 5 | 6 | 6 | 20 | 20 | 0 | 0 | 0.05 |
| 19 | 249.6 | 5 | 0 | 6 | 0 | 0 | 7 | 0.05 | 0.05 |
| 20 | 279.9 | 15 | 0 | 0 | 20 | 0 | 3.5 | 0 | 0.05 |
| 21 | 341.2 | 5 | 0 | 0 | 0 | 0 | 0 | 0 | 0 |
| 22 | 284.8 | 5 | 6 | 0 | 10 | 0 | 7 | 0 | 0 |
| 23 | 217.6 | 15 | 6 | 0 | 0 | 20 | 0 | 0.025 | 0 |
| 24 | 50 | 15 | 3 | 0 | 20 | 0 | 0 | 0.05 | 0.05 |
| 25 | 30 | 15 | 0 | 0 | 0 | 20 | 7 | 0 | 0.05 |
| 26 | 30 | 5 | 6 | 0 | 0 | 0 | 0 | 0.05 | 0.05 |
| 27 | 30 | 5 | 3 | 6 | 0 | 20 | 7 | 0 | 0 |
| 28 | 30 | 15 | 0 | 6 | 10 | 20 | 0 | 0.05 | 0.05 |
| 29 | 30 | 15 | 0 | 0 | 0 | 0 | 7 | 0.05 | 0 |
| 30 | 40 | 15 | 3 | 6 | 20 | 20 | 7 | 0.05 | 0.05 |

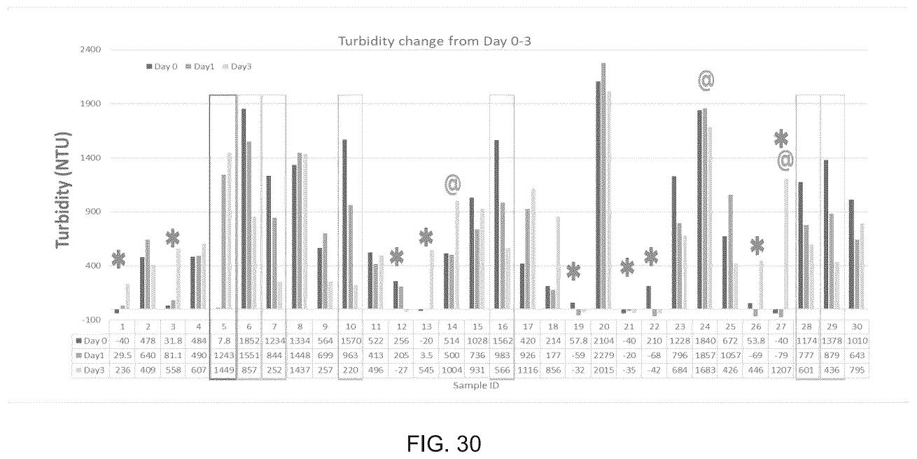

The left panel in FIG. 29 shows the result after incubation for 3 days at ambient temperature. The results were essentially the same for day 0 and day 2. FIG. 30 shows the turbidity change of all 30 conditions from day 0-3. Conditions #1, 3, 12, 13, 19, 21, 22, 26, and 27 (* in FIG. 30) showed very low turbidity. Some conditions including #6, 7, 10, 16, 28, and 29 had reduced turbidity over time (green boxes in FIG. 30). Condition #5 had significant cloudy issues (red box in FIG. 30). @ in FIG. 30 indicates evaporation observed on day 3. The clear or low turbidity conditions had either lower polymer or protein concentration relative to a formulation with at least 15% free protein in a 200-300 mg/ml polymer solution background.

### Example 20—Formulation DOE for Various Excipients and Matrix Combination Screening

A formulation DOE was performed for various excipients and matrix combination screening as described in Example 18, to obtain a formulation with low turbidity. The reagents used are shown in Table 20.1.

The pH of OG1950 was adjusted with Phosphoric acid to target pH (~pH5 and -pH6) first. Next, a mixture of OG1953 and excipients/matrix components were combined in a clear and flat-bottom 96-well ELISA plate. Then, the required amount of pH adjusted OG1950 solution was transferred to the well and mixed slowly in the well. The 30 different combinations of factors designed as discussed in Example 18 are shown in Table 20.2.

TABLE 20.1
Stock reagent Summary

| Description | Concentration | | Formulation |
|---|---|---|---|
| L-histidine | 200 | mM | In water |
| Sucrose | 50% | | In water |
| Trehalose | 50% | | In water |
| L-methionine | 200 | m | In water |
| Polysorbate 20 | 1% | | In water |
| Polysorbate 80 | 1% | | In water |
| Sodium chloride | 5M | | In water |
| Purified OG1950 | 50.4 | mg/ml | PBS pH 7.2 |
| Antibody Intermediate | | | |
| OG1953 conjugate | 50.8 | mg/ml | 12.5 mM Na-Phosphate pH 6.5, |

0.025% Tw20

TABLE 20.2
REACTION OVERVIEW

| Sample | OG1953 | OG1950 | Trehalose | Sucrose | NaCl | Histidine | Methionine | PS20 | PS80 |
|---|---|---|---|---|---|---|---|---|---|
| ID | [g/L] | [%] | [%] | [%] | [mM] | [mM] | [mM] | [%] | [%] |
| 31 | 30 | 5 | 6 | 6 | 0 | 20 | 3.5 | 0.05 | 0 |
| 32 | 50 | 5 | 0 | 0 | 20 | 20 | 7 | 0.05 | 0 |
| 33 | 30 | 5 | 0 | 0 | 20 | 20 | 0 | 0.05 | 0.025 |
| 34 | 50 | 10 | 6 | 0 | 0 | 20 | 7 | 0.05 | 0.05 |
| 35 | 50 | 5 | 6 | 6 | 20 | 0 | 0 | 0.05 | 0 |
| 36 | 50 | 15 | 6 | 6 | 0 | 0 | 7 | 0 | 0.025 |
| 37 | 30 | 10 | 0 | 6 | 20 | 0 | 0 | 0 | 0 |
| 38 | 50 | 15 | 0 | 6 | 20 | 20 | 7 | 0 | 0 |
| 39 | 30 | 15 | 6 | 0 | 20 | 20 | 0 | 0 | 0 |
| 40 | 30 | 15 | 6 | 3 | 20 | 0 | 7 | 0.05 | 0 |
| 41 | 40 | 10 | 3 | 3 | 10 | 10 | 3.5 | 0.025 | 0.025 |
| 42 | 30 | 5 | 0 | 6 | 20 | 0 | 7 | 0.025 | 0.05 |
| 43 | 30 | 5 | 6 | 0 | 20 | 10 | 7 | 0 | 0.05 |
| 44 | 50 | 5 | 0 | 3 | 0 | 20 | 0 | 0 | 0.05 |
| 45 | 50 | 15 | 0 | 6 | 0 | 10 | 0 | 0.05 | 0 |
| 46 | 30 | 15 | 6 | 6 | 0 | 0 | 0 | 0 | 0.05 |
| 47 | 40 | 10 | 3 | 3 | 10 | 10 | 3.5 | 0.025 | 0.025 |
| 48 | 50 | 5 | 6 | 6 | 20 | 20 | 0 | 0 | 0.05 |
| 49 | 50 | 5 | 0 | 6 | 0 | 0 | 7 | 0.05 | 0.05 |
| 50 | 50 | 15 | 0 | 0 | 20 | 0 | 3.5 | 0 | 0.05 |
| 51 | 40 | 5 | 0 | 0 | 0 | 0 | 0 | 0 | 0 |
| 52 | 50 | 5 | 6 | 0 | 10 | 0 | 7 | 0 | 0 |
| 53 | 50 | 15 | 6 | 0 | 0 | 20 | 0 | 0.025 | 0 |
| 54 | 50 | 15 | 3 | 0 | 20 | 0 | 0 | 0.05 | 0.05 |
| 55 | 30 | 15 | 0 | 0 | 0 | 20 | 7 | 0 | 0.05 |
| 56 | 30 | 5 | 6 | 0 | 0 | 0 | 0 | 0.05 | 0.05 |
| 57 | 30 | 5 | 3 | 6 | 0 | 20 | 7 | 0 | 0 |
| 58 | 30 | 15 | 0 | 6 | 10 | 20 | 0 | 0.05 | 0.05 |
| 59 | 30 | 15 | 0 | 0 | 0 | 0 | 7 | 0.05 | 0 |
| 60 | 40 | 15 | 3 | 6 | 20 | 20 | 7 | 0.05 | 0.05 |

The right panel in FIG. 29 shows the result after incubation for 3 days at ambient temperature. The results were essentially the same for day 0 and day 2. The turbidity pattern was similar to the result when OG1801 polymer solution was used for sample ID 1-30 as shown in Example 19.

The pH for samples #31-60 were measured and the result is summarized in Table 20.3 below together with the turbidity measurements:

TABLE 20.3

| Sam- | | | Turbidity | Turbidity | Turbidity | pH |
|---|---|---|---|---|---|---|
| ple | OG1950 | OG1953 | Day 0 | Day 1 | Day 3 | day |
| ID | (g/l) | (g/l) | (NTU) | (NTU) | (NTU) | 3 |
| 31 | 2.5 | 27.6 | −41.4 | −32.4 | −34.5 | 5.73 |
| 32 | 2.5 | 38.7 | 808.6 | 425.6 | 453.9 | 5.91 |
| 33 | 2.5 | 39.2 | 522.6 | 469.6 | 516.5 | 5.97 |
| 34 | 5 | 27.7 | −33.4 | −34.4 | −33.1 | 5.86 |
| 35 | 2.5 | 33.3 | 230.6 | 39.6 | 73.1 | 6.40 |
| 36 | 7.5 | 28 | 1484.6 | 1073.6 | 896.5 | 6.52 |
| 37 | 5 | 30 | 1348.6 | 1007.6 | 866.7 | 6.45 |
| 38 | 7.5 | 30.1 | 1508.6 | 909.6 | 1009.9 | 5.89 |
| 39 | 7.5 | 30 | 1552.6 | 1045.6 | 891.5 | 5.91 |
| 40 | 7.5 | 29.6 | 1856.6 | 1555.6 | 1377.3 | 6.48 |
| 41 | 5 | 33.6 | 916.6 | 571.6 | 549.3 | 6.06 |
| 42 | 2.5 | 30 | 144.6 | −2.4 | 33.9 | 6.40 |
| 43 | 2.5 | 30 | −23.4 | −28.4 | −26.5 | 5.96 |
| 44 | 2.5 | 37.6 | 4.6 | −32.4 | −31.9 | 5.87 |
| 45 | 7.5 | 32.1 | 1632.6 | 1201.6 | 1066.1 | 6.06 |
| 46 | 7.5 | 28.5 | 1494.6 | 1175.6 | 1289.5 | 6.48 |
| 47 | 5 | 33.6 | 900.6 | 735.6 | 703.5 | 6.05 |
| 48 | 2.5 | 28.3 | −41.4 | −34.4 | −34.7 | 5.81 |
| 49 | 2.5 | 35.3 | 352.6 | 395.6 | 414.1 | 6.48 |
| 50 | 7.5 | 39.6 | 2582.6 | 2523.6 | 2416.9 | 6.57 |
| 51 | 2.5 | 40 | 876.6 | 931.6 | 860.7 | 6.52 |
| 52 | 2.5 | 40.3 | 1082.6 | 999.6 | 968.5 | 6.44 |
| 53 | 7.5 | 30.8 | 1258.6 | 527.6 | 576.7 | 5.90 |
| 54 | 7.5 | 34.9 | 2310.6 | 2221.6 | 2097.1 | 6.53 |
| 55 | 7.5 | 33.8 | 1524.6 | 827.6 | 997.3 | 6.00 |
| 56 | 2.5 | 37.1 | 60.6 | −34.4 | −26.5 | 6.44 |
| 57 | 2.5 | 32.3 | −37.4 | −30.4 | −29.3 | 5.82 |
| 58 | 7.5 | 26.9 | 304.6 | −20.4 | −9.7 | 5.89 |
| 59 | 7.5 | 38.9 | 2426.6 | 1981.6 | 1756.1 | 6.54 |
| 60 | 7.5 | 22 | −37.4 | −32.4 | −32.1 | 5.80 |

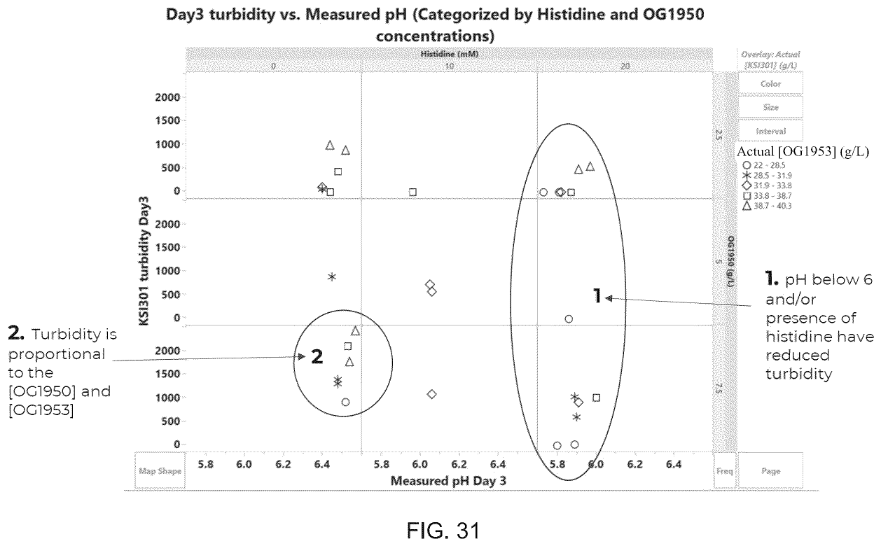

Findings: Data analysis is summarized in FIG. 31. The turbidity is the Y-axis and the lower X-axis is divided into three categories based on the three levels of Histidine concentrations at 0, 10 and 20 mM. The top, middle and bottom panels represent the OG1950 concentrations from 2.5 to 7.5 mg/ml, and the various shape of the symbols represent different concentrations of OG1953, from 28-40 mg/ml (expressed as the concentration of the antibody portion). Based on this result, it was concluded that (1) presence of histidine appears to have beneficial effects on turbidity as the absence resulted in high turbidity; and/or lower pH below 6 have low turbidity sampling as shown in the pH 5.8 with 20 mM histidine; (2) increase in turbidity is also proportional to the concentration of unconjugated OG1950 and OG1953 conjugate. All other excipients tested did not have significant impact on turbidity. Based on this observation, additional rounds of matrix focusing on the potential impact of histidine (see example 21) and pH (see example 22) on the turbidity of the combination formulation were performed.

### Example 21—Impact of Histidine on Formulation

For the experiment below, the formulations were prepared by first adjusting pH of OG1950 with Phosphoric acid to target pH (~pH 5 and ~pH 6) and then mixing the pH adjusted OG1950 and OG1801.

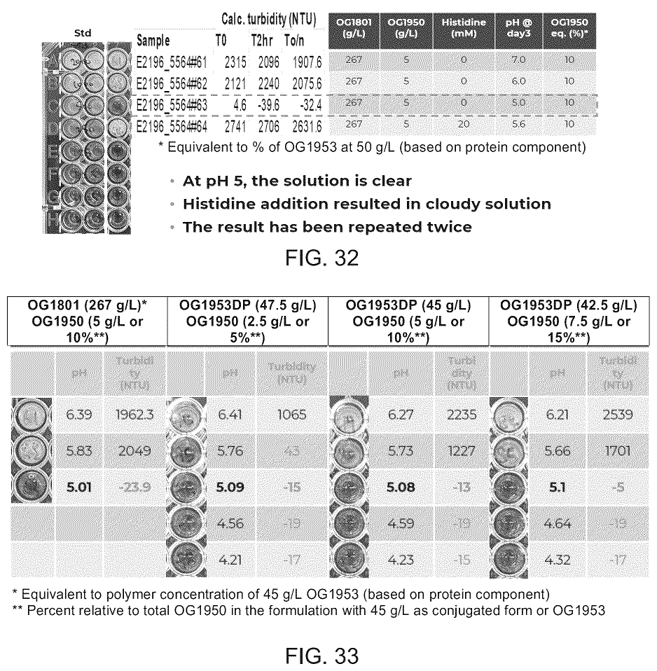

The following procedure was used.
1. Prepare mixture of OG1801 and excipients/matrix components in a 96-well PCR plate, as shown in Table 21.1.
2. In a 96-well assay plate, first transfer the required amount of OG1950 solution to the well, and then dispense slowly while mixing the mixture of OG1801 polymer stock solution and excipients/matrix components.
3. The result is shown in FIG. 32. All mix formulations for samples #61-64 contained 10% unconjugated OG1950 in 266.7 mg/ml OG1801 polymer. All mixtures turned turbid except the sample with the final pH at 5 with no histidine.

TABLE 21.1

| | | | | | | | | For initial OG1950 pH adjustment | | | |
|---|---|---|---|---|---|---|---|---|---|---|---|
| | | | | | | | Vol. DP | | Vol. | Est. | |
| | Target | Target | | Vol. | | | form. | Vol. | Phosphoric | OG1950 | |
| | OG1801 | OG1950 | Total | OG1950 | Vol. | Vol. | buffer | Initial | acid | conc. post | |
| | Conc. | Conc. | Vol. | stock | Histidine | OG1801 | needed | OG1950 | added | pH adj. | Final |
| SampleID | (mg/mL) | (mg/mL) | (uL) | (uL) | (uL) | (uL) | (uL) | (uL) | (uL) | (mg/mL) | pH |
| 61 | 266.7 | 5 | 200 | 19.8 | 0 | 148.6 | 31.6 | 50 | 0 | 50.4 | 6.5 |
| 62 | 266.7 | 5 | 200 | 21.8 | 0 | 148.6 | 29.6 | 50 | 5 | 45.8 | 6 |
| 63 | 266.7 | 5 | 200 | 23.8 | 0 | 148.6 | 27.6 | 50 | 10 | 42.0 | 5 |
| 64 | 266.7 | 5 | 200 | 23.8 | 20 | 148.6 | 7.6 | 50 | 10 | 42.0 | 5 |

### Example 22—Impact of pH on the Turbidity of Various Percentage of Free Protein (5%-15%) to OG1953 Conjugate Solution

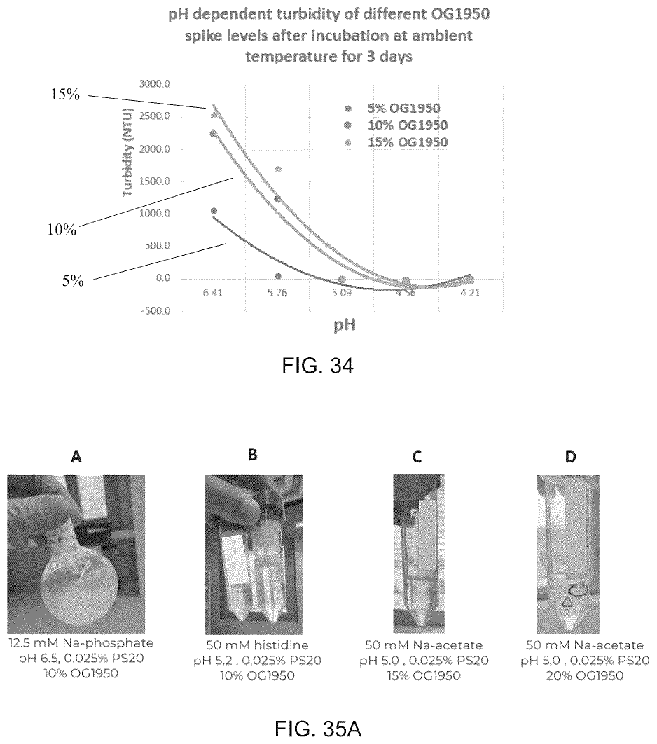

This experiment yielded the following results regarding relationships between pH and turbidity. FIG. 33 depicts the visual view of the plate for the conditions and FIG. 34 graphically depicts correlation of pH and turbidity for 5% OG1950, 10% OG1950, and 15% OG1950 with OG1953 with concentration range from 42.5-47.5 mg/ml (based on protein component). The result clearly shows a trend where the solutions turbidity is the lowest at around pH 5 or lower at each of the different percentages of free protein.

The experiment was conducted using a clear and flat bottom 96-well microtiter plate, with the following procedure. First add and mix the protein and buffer diluent components in the wells, then to each well slowly add and mix in the polymer or conjugate stock solution. Let the plate incubate at ambient temperature for at least 30 min before any measurement. A summary of the reagents used is shown in Table 22.1. The formulations were set up as shown in Table 22.2.

TABLE 22.1
Stock reagent summary

| Description | Concentration | |
|---|---|---|
| L-histidine | 200 | mM |
| L-methionine | 200 | mM |
| Sodium chloride | 5M | |
| Purified OG1950 Antibody Intermediate | 50.4 | mg/ml |
| OG1801 polymer solution | 359 | mg/ml |
| OG1953 bioconjugate | 50.8 | mg/ml |

TABLE 22.2
Experimental matrix overview

| SampleID | OG1953DP [g/L] | OG1950 [g/L] | OG1950 [%] |
|---|---|---|---|
| 74 | 47.5 | 2.5 | 5 |
| 75 | 47.5 | 2.5 | 5 |
| 76 | 47.5 | 2.5 | 5 |
| 77 | 47.5 | 2.5 | 5 |
| 78 | 47.5 | 2.5 | 5 |
| 79 | 45 | 5 | 10 |
| 80 | 45 | 5 | 10 |
| 81 | 45 | 5 | 10 |
| 82 | 45 | 5 | 10 |
| 83 | 45 | 5 | 10 |
| 84 | 42.5 | 7.5 | 15 |
| 85 | 42.5 | 7.5 | 15 |
| 86 | 42.5 | 7.5 | 15 |
| 87 | 42.5 | 7.5 | 15 |
| 88 | 42.5 | 7.5 | 15 |

The result of turbidity and measured pH of the different matrices for day 0 and day 3 incubation at ambient temperature is shown below in Table 22.3. This data shows a correlation between low turbidity and low pH (see FIG. 34).

TABLE 22.3

| | |
|---|---|
| Turbidity (NTU) | Measured pH |

| Sample | | T = 3 | | T = 3 |
|---|---|---|---|---|
| ID | Time 0 | days | Time 0 | days |
| 74 | 1484.2 | 1065.0 | 6.44 | 6.41 |
| 75 | 104.2 | 43.0 | 5.81 | 5.76 |
| 76 | −37.8 | −15.0 | 5.1 | 5.09 |
| 77 | −41.8 | −19.0 | 4.6 | 4.56 |
| 78 | −37.8 | −17.0 | 4.22 | 4.21 |
| 79 | 2368.2 | 2235.0 | 6.34 | 6.27 |
| 80 | 1098.2 | 1227.0 | 5.76 | 5,73 |
| 81 | −37.8 | −13.0 | 5.07 | 5.08 |
| 82 | −39.8 | −19.0 | 4.6 | 4.59 |
| 83 | −37.8 | −15.0 | 4.28 | 4.23 |
| 84 | 2690.2 | 2539.0 | 6.29 | 6.21 |
| 85 | 1792.2 | 1701.0 | 5.68 | 5.66 |
| 86 | −33.8 | −5.0 | 5.09 | 5.1 |
| 87 | −41.8 | −19.0 | 4.58 | 4.64 |
| 88 | −37.8 | −17.0 | 4.26 | 4.32 |

### Example 23—Scale Up Testing of the pH Dependent Turbidity of Mix Formulation

This example shows scaling up the results of Example 22. The mix formulation was also tested at 10-20 mL final process scale. See table 23.1 below for the formulation matrix. “Molar ratio” refers to the ratio of the amount (in moles) of OG1950 (“free”) to OG1953 (“conjugate”) in the formulation (based on the antibody portion of the conjugate).

TABLE 23.1

| | | | | | Percent | Molar ratios |
|---|---|---|---|---|---|---|
| Sample | | [OG1950] | [OG1953] | [Total] | free | (free/ |
| ID | Formulation | mg/ml | mg/ml | mg/ml | OG1950 | conjugate) |
| A | 12.5 mM sodium acetate pH | 5 | 45 | 50 | 10% | 0.11 |
| | 6.5, 0.025% PS20, 10% OG1950 | | | | | |
| B | 50 mM histidine pH 5.2, | 5 | 45 | 50 | 10% | 0.11 |
| | 0.025% PS20, 10% OG1950 | | | | | |
| C | 50 mM sodium acetate pH 5.0, | 7.5 | 42.5 | 50 | 15% | 0.18 |
| | 0.025% PS20, 15% OG1950 | | | | | |
| D | 50 mM sodium acetate pH 5.0, | 10 | 40 | 50 | 20% | 0.25 |
| | 0.025% PS20, 20% OG1950 | | | | | |

The following procedure was used. The process starts at the elution step of the Cation-Exchange (CEX) chromatography step. The pooled fraction of the OG1953 conjugate at about 2 grams per liter (g/L) was first concentrated to 6.0 g/L using Tangential Flow Filtration (TFF) at a membrane charge of approximately 105 grams per square meter (g/m²). Then, the required amount of purified OG1950 was added to OG1953 conjugate to obtain the targeted free protein to conjugate ratio, for example, of 15% free protein to 85% OG1953 conjugate. The addition of the free OG1950 increased the membrane charge to approx. 124 g/m2. The protein solution was subjected to a seven-fold diafiltration buffer exchange step as shown in the table above without the PS20 followed by a concentration to 20 g/L, formulation by polysorbate addition and further concentration to 50 g/L.

The result is shown in FIG. 35A, which confirms at scale that at pH 6.5 formulation, extensive precipitation is formed, as shown in panel A. All other tested formulations result in clear solutions, though at 20% OG1950 formulation as shown in panel D, the solution had a hazy but transparent appearance. Both the pH 5 formulation of histidine and sodium acetate produced a clear solution (panel B & C). The result confirmed that lowering pH to 5 can result in a clear mix formulation of up to 20% free OG1950 in relation to OG1953 conjugate.

Formation of turbidity can avoided by lowering the pH to 5. Next, the following factors were considered to determine the quality of the coformulation: (1) whether the free protein remains in free form in the mixture; (2) the accuracy of the target free protein amount during and after the manufacturing of the final product; (3) the long-term stability of OG1953 conjugate and OG1950 free protein in the mixture and how to monitor these two populations of molecules separately; and (4) how to evaluate the potency of the mix formulation.

To monitor the purity and stability for the conjugate and the free protein in a mixed formulation manner, the following considerations were made:
4. OG1953 is a protein-biopolymer conjugate with a polydisperse nature, therefore it can produce varying degree of charge shielding to the protein and also has a varying size, these properties produce a long elution tail in column chromatography which can be an interference factor, as it can mask baseline separation for neighboring peaks. Such an impact can be pronounced when some contaminant peaks are present in low quantities and when they elute very close to the conjugate peak.
5. For separation based on size, protein aggregates can be masked by the tailing conjugate fraction in a size exclusion chromatography. This can be addressed using an analytical size exclusion chromatography column in the desired size exclusion distribution range that can (i) cover the ultra-high MW conjugate and any of its aggregated forms, and also (ii) cover the smaller MW range which can separate the smaller antibody and any antibody aggregates.

Analysis of the mix formulation can be accomplished by exploring the different properties of the OG1953 conjugate and OG1950 free protein. OG1950 has a theoretical pI of 7.81 and 7.55 with and without the C-terminal lysine, respectively; the experimental pI further depending on glutathionylation status and presence of C-terminal lysine ranged between 7.5-8.2. The molecule is protonated at acidic pH which allows it to bind Cation-exchange resin with sodium acetate buffer at pH 5 at low ionic strength, such as no salt condition. At such pH, the conjugate OG1953 binds more weakly due to the polymer charge shielding effect on the OG1950 and is therefore eluted earlier in the sodium chloride gradient as compared to the OG1950 free protein. An analytical CEX method was developed based on such differential charge variation.

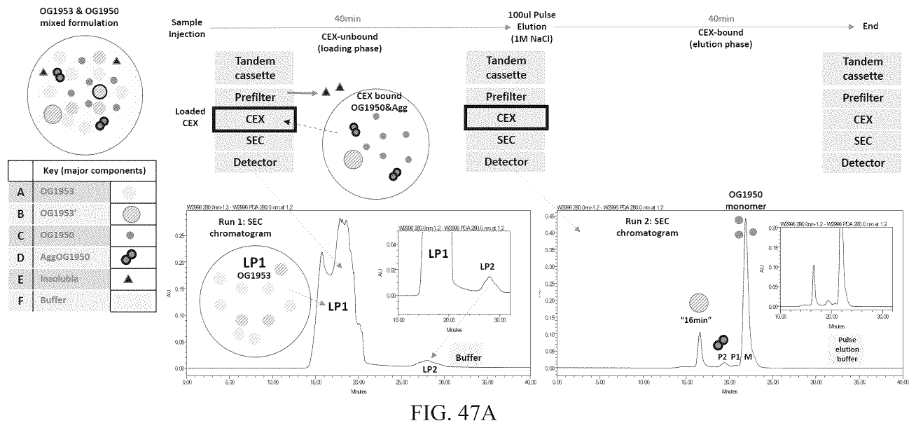

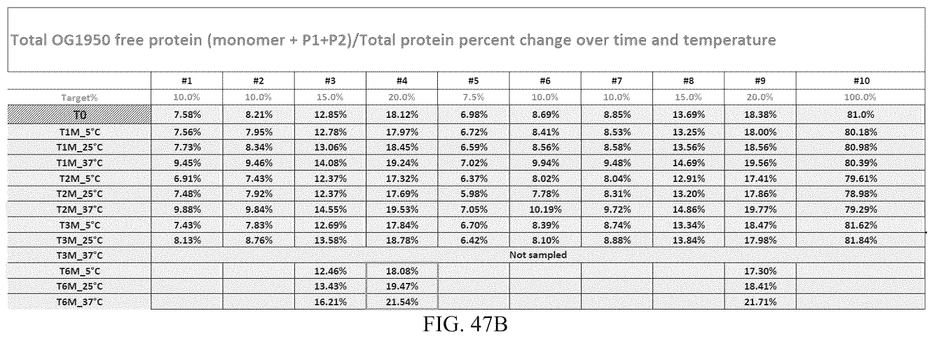

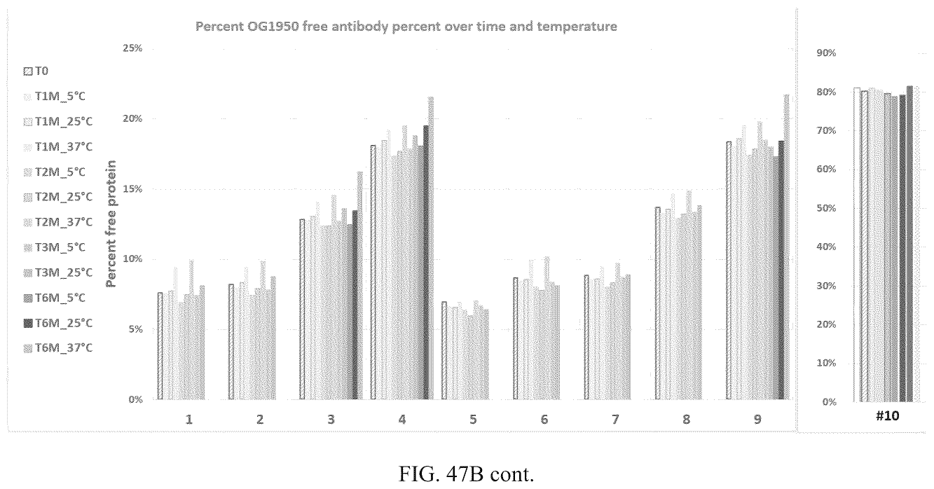

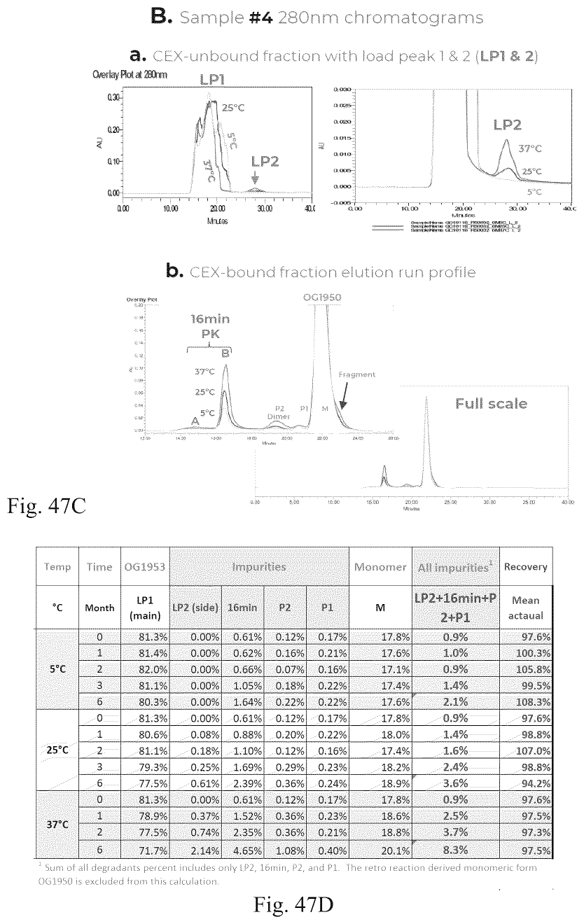

Alternatively, the molecular weights of OG1953 and OG1950 are ~1,000 kDa and 146 kDa, respectively. The dramatic size difference of the two molecules can be separated by analytical size exclusion chromatography (SEC-HPLC). An appropriate column with appropriate exclusion limit at such range should be selected. The analysis of OG1953 and OG1950 can involve using two different SEC columns with different exclusion limits. This is because a column with resolution at ultra-high molecular weight range has limited to no resolution for the low molecular weight range and vice versa. However, these two assays can provide a reasonable readout for the mix population in order to determine if the percent free protein meets the required specification. For long term stability of the OG1950 protein, its aggregated forms such as dimer, trimer and multimers are masked by the OG1953 trailing peak by SEC, whereas the aggregated forms do not resolve well by CEX method, most likely due to the highly similar charge density of the monomeric form as compared to the aggregated forms. To improve the CEX or SEC methods, a combination of the two methods in an integrated and orthogonal manner was developed (see Examples 26, 27, FIG. 47A).

The result of the analytical CEX and SEC-HPLC analysis is shown in FIGS. 35B and 35C, respectively. FIG. 35B left panel shows the overlayed chromatograms of the injected sample runs for the four formulations. The major peak at 6.5 min on the left is the OG1953 conjugate and the smaller peak at 7.9 min is the OG1950 free antibody. The table on the right is the peak area distribution of the two peaks for each sample. The percent peak area was very close to the intended mixing target for each formulation. FIG. 35C, left panel shows the overlay chromatograms of the SEC-HPLC analysis results, the major peak at 47 min is the OG1953 conjugate and the OG1950 free protein elutes at 58 min. However, the detected free protein peaks by both methods were about 2% lower than expected for all samples.

### Example 24—Impact of pH on the Turbidity of 10% OG2072 Fusion Protein to 90% OG2074 Conjugate Solution or OG1801

The mix formulation experiment was tested for a fusion protein OG2072 and its conjugated form with OG1802, OG2074. The theoretical pI of OG2072 is 8.2. In this experiment, OG2072 protein is mixed with OG2074 to 10% composition, and each set is tested at pH 4.79 and 6. The matrix is as shown in Table 24.1 below.

TABLE 24.1
Matrix overview

| | OG2074 | OG1801 | OG2072 | OG2072 |
|---|---|---|---|---|
| SampleID | [g/L] | [g/L] | [g/L] | [%] |
| 89 | | 219.9 | 5.25 | 10 |
| 90 | | 219.9 | 5.25 | 10 |
| 91 | 47.25 | | 5.25 | 10 |
| 92 | 47.25 | | 5.25 | 10 |
| | 10% OG2072/ | 10% OG2072/ | | |
| | 90% OG1801 | 90% OG1953DP | | |
| OG2072 @ pH 6 | SampleID = 89 | SampleID = 91 | | |
| OG2072 @ pH 4.79 | SampleID = 90 | SampleID = 92 | | |

Briefly, the reagents as shown in the Reagent summary in Table 24.2 were used. The experiment was conducted using a clear and flat bottom 96-well microtiter plate, with the following procedure. First, the protein and buffer diluent components were added and mixed in the wells, then to each well the polymer or conjugate stock solution were slowly added and mixed. Let the plate incubate at ambient temperature for at least 30 minutes before any measurement.

TABLE 24.2

| OG# | Description | Batch # | Concentration |
|---|---|---|---|
| OG1801 | Biopolymer without | R15479 | 359 |
| | maleimide functional | | |
| | group | | |
| OG2072 | Purified OG2072 fusion | R15861 | 20 |
| | protein | | |
| | Concentrated OG2072 | R15861A | 53.89 |
| OG2074 | OG2072 + OG1801 | SFO Lot #70231 | 52.5 |

TABLE 24.3

| | Actual OG2074 | Turbidity (NTU) | | Measured pH | |
|---|---|---|---|---|---|
| Sample ID | conc. (mg/mL) | Time 0 | T = 3 days | Time 0 | T = 3 days |
| 89 | | 1774.2 | 223.0 | 6.13 | 6.06 |
| 90 | | 342.2 | −13.0 | 4.43 | 4.41 |
| 91 | 47.4 | 286.2 | 653.0 | 5.35 | 5.25 |
| 92 | 47.2 | 234.2 | 201.0 | 5.2 | 5.18 |

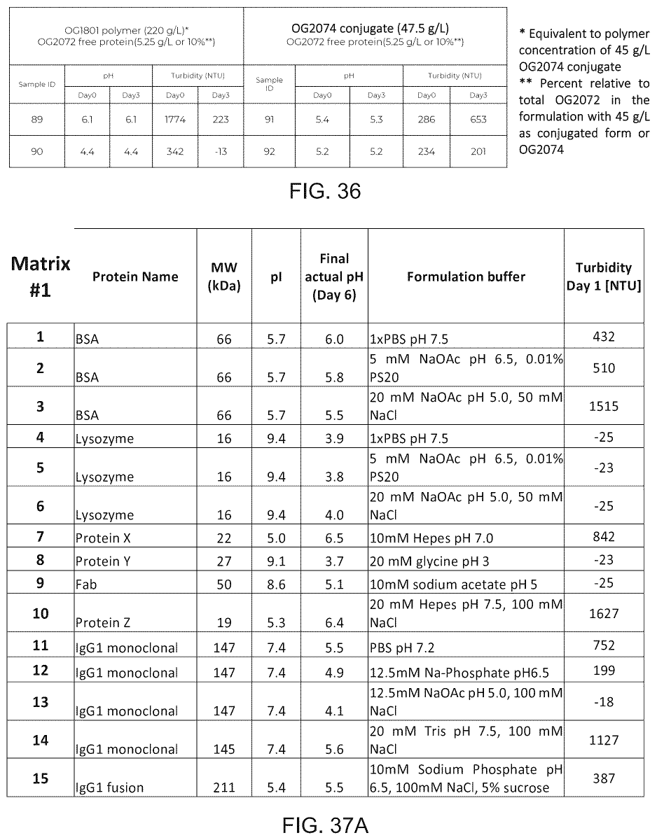

The turbidity result is depicted in FIG. 36C, which shows the OG2072 free protein mixing with either OG1801 polymer or OG2074 conjugate at a final free protein percent composition of 10% at pH 6 is turbid, and incubation for 3 days resulted in reduced turbidity. At pH lower than 5, the solution remains clear. At pH 4.4, the solution is clear without any incubation time.

In a separate embodiment, the OG2072 free protein present in either 20% or 30% of the total protein concentration with OG2074 conjugate were prepared, and these solutions were clear. For the 30% OG2072 formulation, the pH was 5.0. The total protein concentration in this example is at least 50 mg/ml.

To prepare the OG2072 protein, the OG2072 heavy and light chains may be cloned into expression plasmids and transfected into CHO cells. Cells can be grown up in appropriate media and harvested. OG2072 may be purified using techniques described above. The OG2072 cysteine at position L660C (or L443C per EU numbering) residue is typically “capped” or oxidized by chemicals in the cell culture media and is not available for conjugation. In this regard, purified OG2072 may be subjected to a decapping (i.e. reducing) procedure to remove the cap and enable the free (i.e. those not involved in Cys-Cys disulfide bonds) cysteine residue to be conjugated to the maleimide functionality of a polymer. Decapping may be done by mixing purified OG2072 protein with a 30× molar excess of the reducing agent TCEP (3,3′,3″-Phosphanetriyltripropanoic acid) at 20° C. for 45 minutes. The reduction reaction with TCEP may be monitored by SDS-PAGE, SEC-HPLC and/or CE-SDS. Following reduction, the OG2072 protein can be washed by Tangential Flow Filtration (TFF) using a Pellicon 3 Ultrafiltration Cassette with 30 kDa MWCO cellulose based membrane from Millipore with 20 mM Tris pH 8.5, 50 mM NaCl, 2 mM EDTA (Ethylenediaminetetraacetic acid) buffer to remove the cap and the excess TCEP. Reduced OG2072 can then be allowed to reoxidize using DHAA at 20° C. for 1 hour followed by a TFF step for removal of DHAA to form reoxidized decapped OG2072. The decapping status is monitored by SDS-PAGE assay, SEC-HPLC, CE-SDS, and/or icIEF.

### Conjugation of OG2072 to MPC Polymer

Decapped OG2072 may be conjugated to polymer OG1802. An excess of OG1802 is used (2.5-4.5 fold molar excess). Conjugation can be monitored by SDS-PAGE and/or CEX-HPLC and driven to near completion. OG2072 conjugate may be purified via cation-exchange chromatography and buffer exchanged into the formulation buffer by TFF.

### Example 25—Formulation Evaluation of Turbidity for Various Proteins at the 15% Free Protein Equivalent Conditions

The experimentation was undertaken to compose a formulation of polymer OG1801 with various proteins, to evaluate the effect of OG1801 polymer on turbidity of the formulations of each of the various proteins using the following conditions: (1) polymer at 300 mg/ml (equivalent concentration of the polymer concentration in an OG1953/OG1950 formulation containing 15% free protein); (2) free protein concentration at 7.5 mg/mL.

The reaction was carried out in a clear flat bottom well 96 well ELISA plate, with the following procedure. To prepare the reaction matrix, pipet the serially diluted turbidity standard on the plate, then pipet the required volume of protein solution and dilution buffer to the well, finally use a positive displacement pipettor and pipet tips to transfer the polymer solution into each well. Let the plate equilibrate at ambient temperature for at least 30 min before reading the turbidity using a plate reader. Take a picture of the plate. Seal the plate with a PCR plate sealer to prevent evaporation. Let the plate continue to incubate at room temperature for six days before reading the pH and adjusting the pH and read the turbidity (see FIG. 37. Reaction set up and measured turbidity are shown in FIG. 37A.

### Findings—

Turbidity increased when the solution pH was close to the pI of the protein (see FIGS. 37A, 37B, 38A, 38B). FIGS. 37A and 37B depict the screening result of mixing various proteins other than OG1950 antibody with a fixed concentration of OG1801 polymer. The conditions aim to mimic the 15% free protein mixed with 85% OG1953 conjugate to evaluate if the precipitation property is a more general phenomenon.

FIGS. 38A and 38B summarize the results of adjusting the pH of the samples up or down to evaluate the effect on turbidity. The result demonstrated that the turbidity is reversible or reproducible dependent on the pI of the protein. FIG. 38A is the plate view of the experiment with the measured pH of each condition. FIG. 38B is the Bubble plot of isoelectric point versus the solution pH. The size of the bubble represents the degree of turbidity measured for each condition at or after pH adjustment. The dotted line depicts where the solution pH would be close to the pI.

A clear solution was observed when the solution pH was away from the pI. Turbidity increased independent of the protein, molecular weight, and concentration (see FIGS. 37A, 38B). The dissolution of the turbid solution seemed reversible, as shown visually and in the turbidity measurements (FIGS. 37B (left and middle panels), 38A).

### Example 26—Experimental Composition Formulations for High Free OG1950 Up to 90% or with a OG468 Fab from 4.6%-93%

This experiment was performed: (1) to test the potential range of the mix formulations such as up to 90% OG1950; (2) to evaluate the mix formulation behavior of a 50 kDa Fab (OG468) and a broad range of mixing ratio, such as from 4.6-93%; (3) to demonstrate that the mix formulation forms a clear solution, contains expected amount of free protein, and the free protein is a well-behaved, with correct molecular weight and with migration behavior comparable to the pre-mixing form or standard, using the Tandem chromatography method.

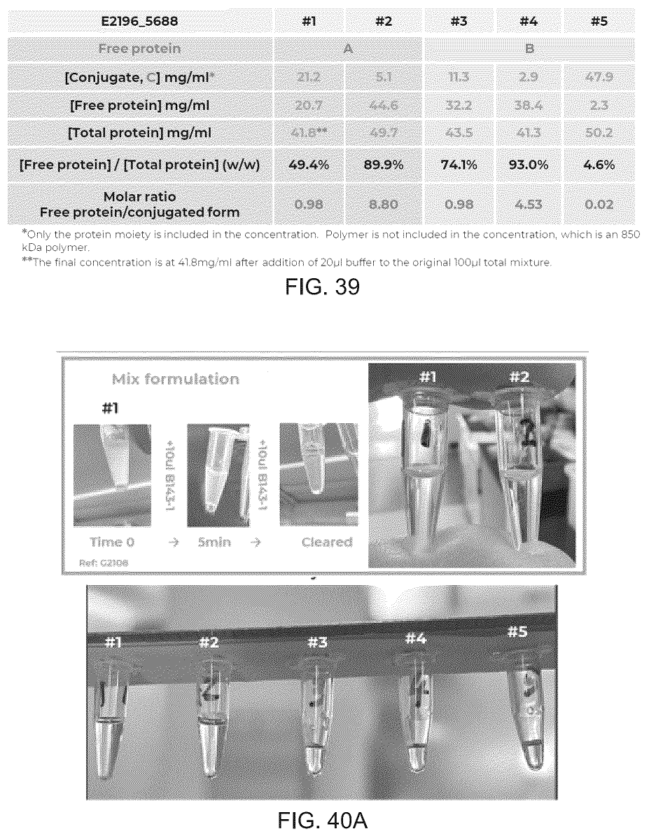

Parameters of the experiment FIG. 39. Table 26.1 is the reagent table and table 26.2 is the experimental setup table.

TABLE 26.1
Reagents

| | | | | Conc. | Total MW | Bioactive | |
|---|---|---|---|---|---|---|---|
| ID | Name | Description | Batch # | (mg/mL) | (kDa) | MW (kDa) | Formulation |
| C | OG1953DP | OG1953 | R16346 | 50.8 | 950 | 146 | 12.5 mM Na- |
| | | Drug | | | | | Phosphate pH |
| | | Product | | | | | 6.5, 0.025% |
| | | | | | | | Tw20 |
| A | OG1950PUR | IgG | R16441 | 49.6 | 146 | 146 | 10 mM PBS pH |
| | | | | | | | 7.2 |
| B | OG468 | Fab | R1136k | 40.8 | 50 | 50 | 10 mM sodium |
| | | | | | | | acetate pH 5.0 |
| D | OG1953_{—} | OG1953 + | R50031 | 49.8 | NA | 146 | 50 mM sodium |
| | R50031 | 15% OG1950 | | | | | acetate buffer |
| | | | | | | | pH 5.0, 0.025% |
| | | | | | | | Tw20 |

TABLE 26.2
Reaction setup

| | | | | Free | | | Final | | Final | Total | | Molar |
|---|---|---|---|---|---|---|---|---|---|---|---|---|
| | | OG1953 | Free | Protein | | Diluent | total | Final | [Free | final | | ratio |
| | | vol. | Protein | vol | Diluent | vol. | vol | [OG1953] | protein] | conc. | % free | (free/ |
| SampleID | OG1953 | (ul) | used | (ul) | used | (ul) | (ul) | mg/ml | mg/ml | (mg/ml) | protein | conj) |
| 1 | C | 50 | A | 50.0 | B143-1* | 20 | 120 | 21.2 | 20.7 | 41.8 | 49.4% | 0.98 |
| 2 | C | 10 | A | 90.1 | NA | 0.0 | 100 | 5.1 | 44.6 | 49.7 | 89.8% | 8.80 |
| 3 | C | 7 | B | 25.0 | NA | 0 | 32 | 11.1 | 31.8 | 43.0 | 74.1% | 0.98 |
| 4 | C | 1.5 | B | 25.0 | NA | 0 | 27 | 2.9 | 38.5 | 41.3 | 93.0% | 4.53 |
| 5 | C | 25 | B | 1.5 | NA | 0.0 | 27 | 47.9 | 2.3 | 50.2 | 4.6% | 0.02 |

*B143-1 contains 50 mM Sodium Acetate pH 5, 0.025% PS20

TABLE 26.3
sample preparation for analysis

| | | Final | | Dilution | | |
|---|---|---|---|---|---|---|
| | vol for | working | Stock | buffer | Injected | ug free |
| Sample | inject | conc. | needed | needed | vol | protein |
| ID | (ul) | (mg/ml) | (ul) | (ul) | (ul) | inject |
| 1 | 100 | 10 | 48.4 | 51.6 | 10 | 100 |
| 2 | 100 | 10 | 22.4 | 77.6 | 10 | 100 |
| 3 | 30 | 10 | 9.4 | 20.6 | 15 | 150 |
| 4 | 30 | 10 | 7.8 | 22.2 | 15 | 150 |
| 5 | 30 | 2 | 26.0 | 4.0 | 65 | 130 |

Inject the specified volume for Tandem method chromatography analysis using a WatersHPLC system equipped with a 2695 Alliance Solvent management system and a 2785 Dual Wavelength detector.

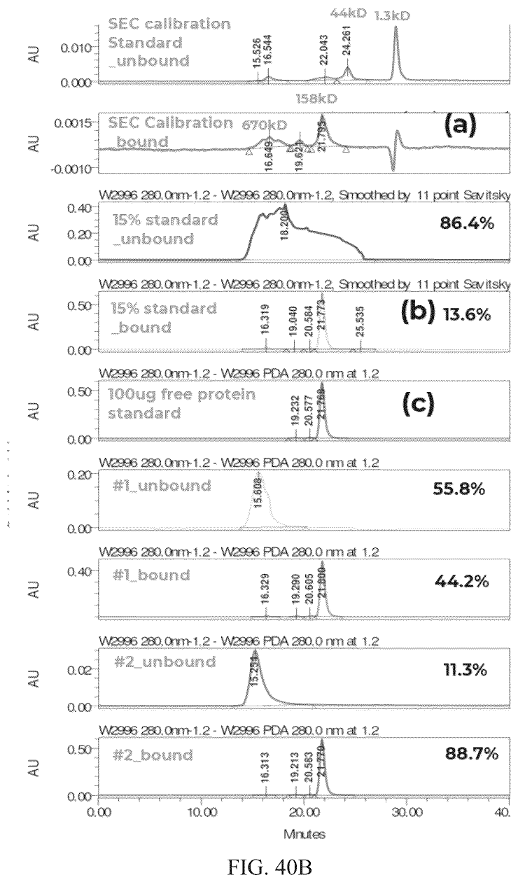

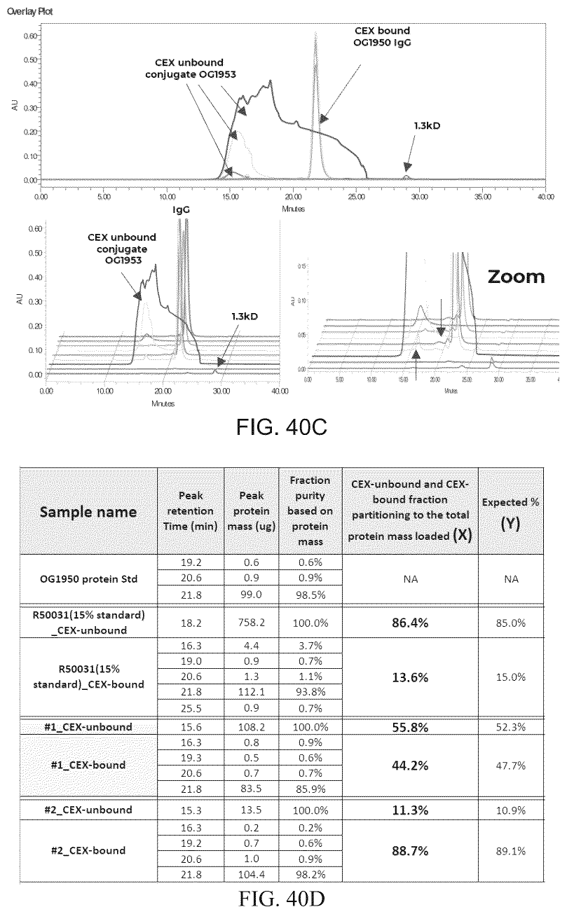

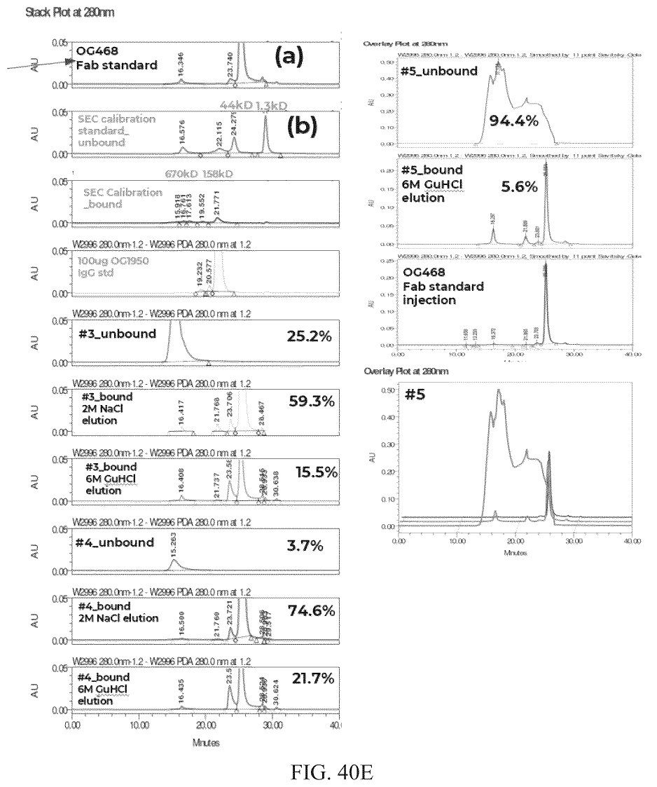

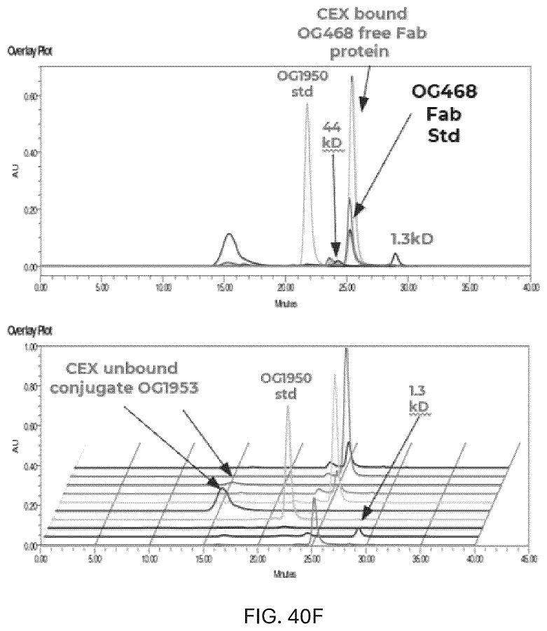

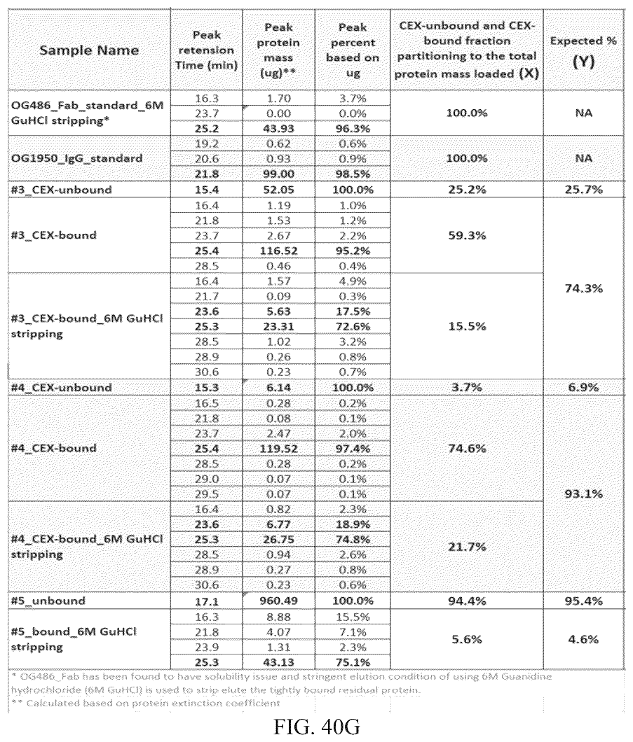

Findings: Mixture #1 (FIGS. 9, 40A) was turbid at first but became cleared upon addition of 20 uL buffer. Mild precipitation was observed upon microfuge centrifugation at 5K rpm for 5 minutes. All other formulations (FIG. 39), #2-#5, showed no cloudiness at all.

FIG. 40A and FIG. 40B are the sample analysis of the #1 & 2 samples using Tandem method analysis. Elution peak(s) of free protein were present at the expected retention time of 21.8 min, whereas the conjugate was eluted as a broad peak at ~15.6 min. This was confirmed by (a) SEC calibration standard; (b) 15% mix formulation standard (R50031); and (c) OG1950 free protein standard injection. The percentage on the right of each chromatogram represents the partition of CEX-unbound (conjugate) to CEX-bound fraction (free IgG) which were in agreement with the expected percent free protein as shown in FIG. 40D. A close-up overlay of all curves in FIG. 40C shows the overlap of eluted IgG peaks which were aligned with both the OG1950free protein standard and IgG standard contained in the SEC calibration standard. (FIG. 40C, Bottom left panel). Offset overlay of various injections for clear illustration of the separation between unbound CEX conjugate fraction and the CEX bound IgG fraction. (FIG. 40C, Bottom right panel) is a close-up view to show the aggregated form of IgG that is detected as shown in the arrows. FIG. 40D is the peak analysis table showing the agreement with and recovery of the mixed in free protein in the expected ratio as compared to the conjugate unbound fraction.

Similar results were observed for samples #3-#4 for the OG468 Fab (see FIGS. 40E-40G). Tandem method analysis shows elution peak(s) of free protein at the expected retention time of about 25.3 min whereas the conjugate eluted at 15-15.2 min as a broad peak (FIG. 40E). This was confirmed by (a) OG468 Fab standard injection; and (b) SEC calibration standard. FIG. 40F, top panel shows an overlay of all curves from FIG. 40E, which shows the eluted OG468 Fab peaks are aligned with the standard OG468 standard and was eluted later than the IgG standard but eluted later than the 44 kDa standard of the SEC calibration standard. FIG. 40F, Bottom panel shows an offset overlay of various injections for clear illustration of the separation between unbound CEX conjugate fraction and the CEX bound Fab fraction. FIG. 40G shows the peak area comparison of CEX bound (free protein) to unbound fraction (conjugate) and the agreement to the expected ratios (Compare column (X) to column (Y)). For sample #5, there was only 6M GuHCl elution as OG468 Fab was found to bind tightly to the CEX column and was unable to be eluted with 1M NaCl and only recovered by 6M GuHCl elution; which was later repeated for #3&4 with 2M NaCl as elution buffer but was only about 60% and 75% eluted for samples #3 and #4, respectively. However, the accumulated total eluted CEX bound fraction was in agreement with the expected number as shown in column (Y).

### Example 27—Stability of OG1953 Conjugate Solution Containing Free OG1950 Protein from 7.5%-20% at a Combined Total Protein Concentration of 50-65 mg/mL Protein Concentration and Incubated at 3 Different Temperatures Including 5° C., Ambient Stress Temperature at 25° C., and Accelerated Stress Condition at 37° C. For a Period of 3-6 Months

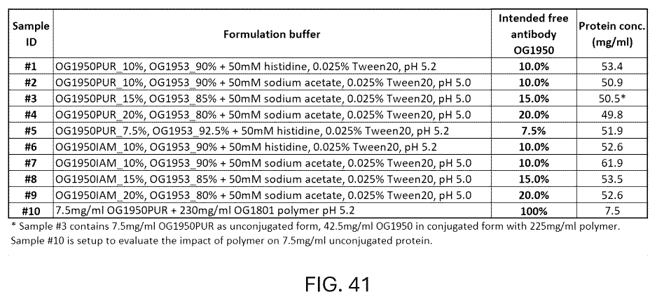

Experiments were carried out to assess the following: (1) Evaluate the long-term stability of OG1953 conjugate solution containing 7.5%, 10%, 15% and 20% free antibody OG1950; (2) Evaluate the various factors that represent the quality of each formulation such as color and clarity by visual inspection; aggregation or degradation at different temperature by using size exclusion chromatography (SEC-HPLC); ion-exchanger chromatography (IEX-HPLC); SEC with Multi-angle light scattering (MALS) detector; and a tandem HPLC method that allows monitoring of the stability of either the conjugate or free protein population separately; and potency; (3) Evaluate the maintenance of the percent free antibody (OG1950) in 50 mg/ml total formulation; (4) Compare and contrast the impact of iodoacetamide (IAM) treatment (alkylation) on the protein stability (#6-9 in FIG. 41); and (5) Compare against a control formulation to evaluate the impact of OG1801 polymer on free antibody (OG1950) at 15% equivalent concentration (i.e. 227 mg/ml OG1801 polymer and 7.5 mg/ml OG1950) (#10 in FIG. 41).

FIG. 41 shows the sample setup of OG1953/OG1950 formulations for testing long-term stability. The formulations of OG1953 conjugate contained free proteins from 7.5-20% with a total combined protein concentrations of 50-65 mg/ml. Most samples remained colorless and clear from 1-3 months at all temperatures tested. Samples that were turbid were formulated in histidine buffer, and the turbidity was proportional to the increasing temperatures such as 25° C. and 37° C. conditions. The turbidity induced by histidine may be a different mechanism or type from the turbidity caused by the OG1801 polymer or OG1953 conjugate. The turbidity in the histidine-containing formulations did not appear to impact the quality of the formulation in terms of potency, strength, purity and free protein ratio over time and temperatures.

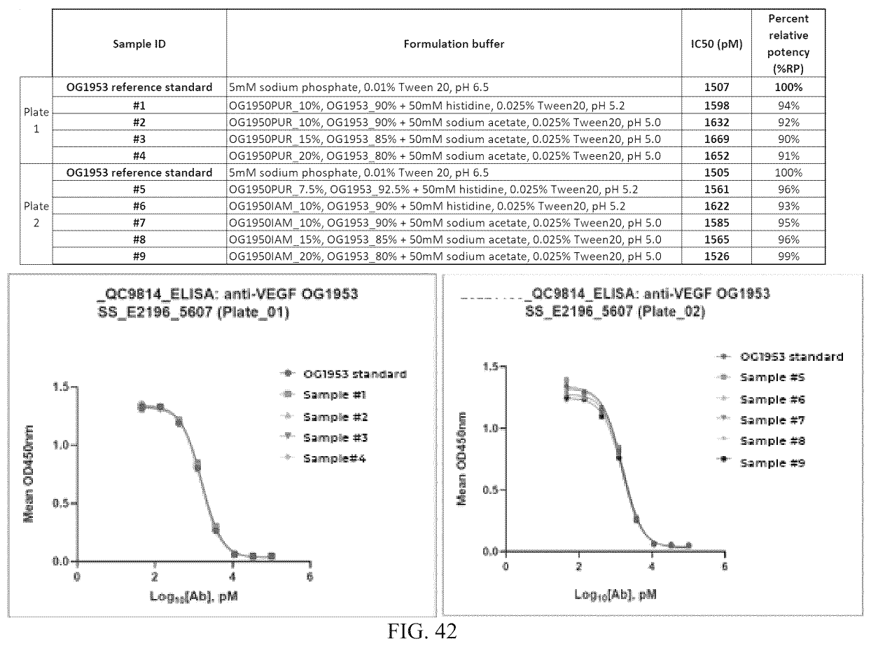

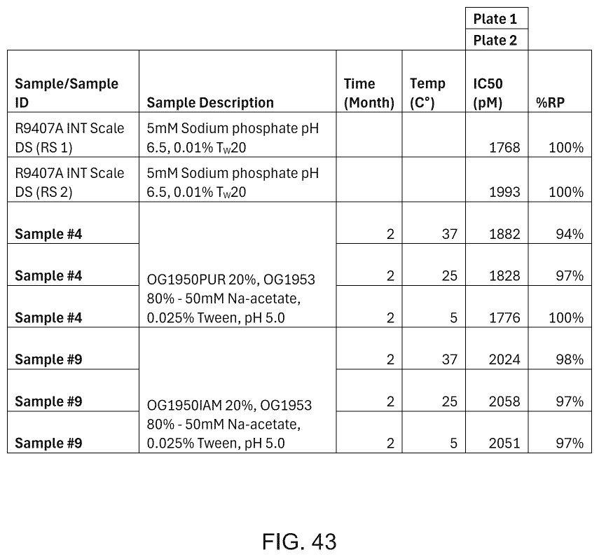

The potency of each formulation was tested using ELISA as described in Example 12 except for 1) the BSA used for blocking and washing was changed to Product #A7030 (Sigma Aldrich; heat shock fraction, protease free, fatty acid free, essentially globulin free, pH 7, ≥98%); 2) the plate coating with VEGF-R1 is performed at 4° C. instead of room temperature; and 3) the HRP conjugated streptavidin was diluted to 1:1000 instead of 1:1500. All formulations showed comparable potency relative to the reference standard (OG1953) at time 0 (FIG. 42). Formulations #4 and #9 retained activity for at least 2 months at 5, 25, or 37° C. (FIG. 43), and formulation #4 retained activity up to 6 month at all temperatures.

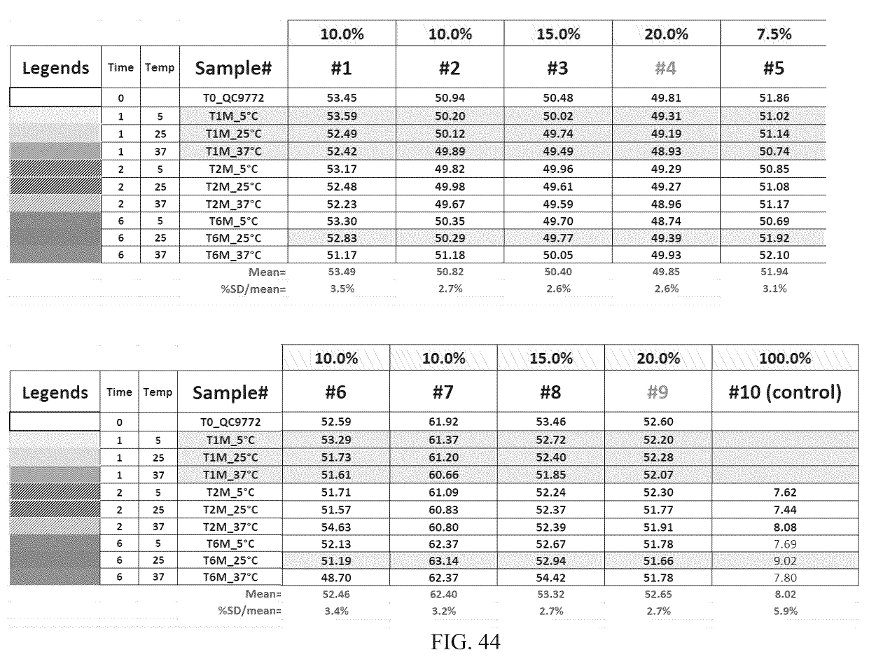

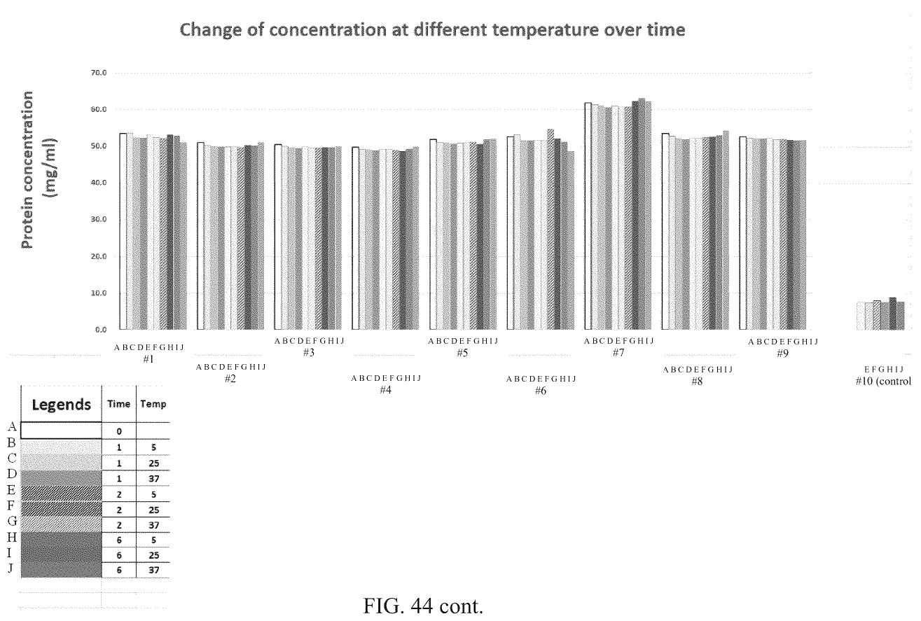

The protein concentration of each formulation was assayed using the SoloVPE OD280 nm method. All formulations showed stable protein concentration for all samplings up to the 6-month sampling period at 5, 25° C. and 37° C. (FIG. 44). See FIG. 41 for sample setup.

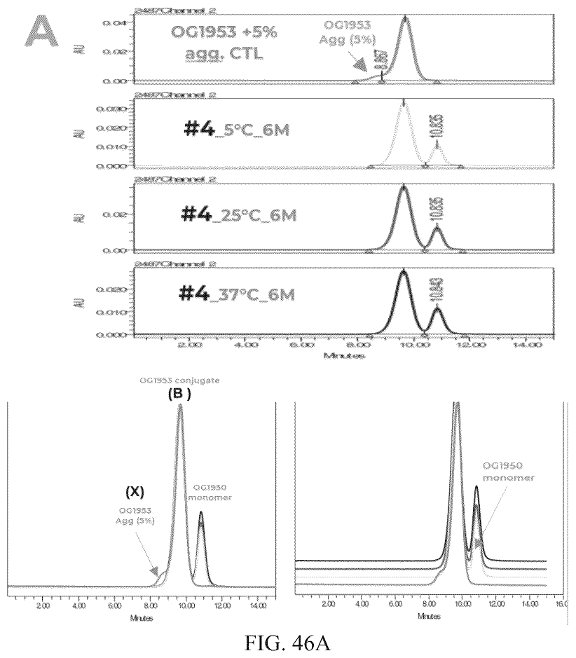

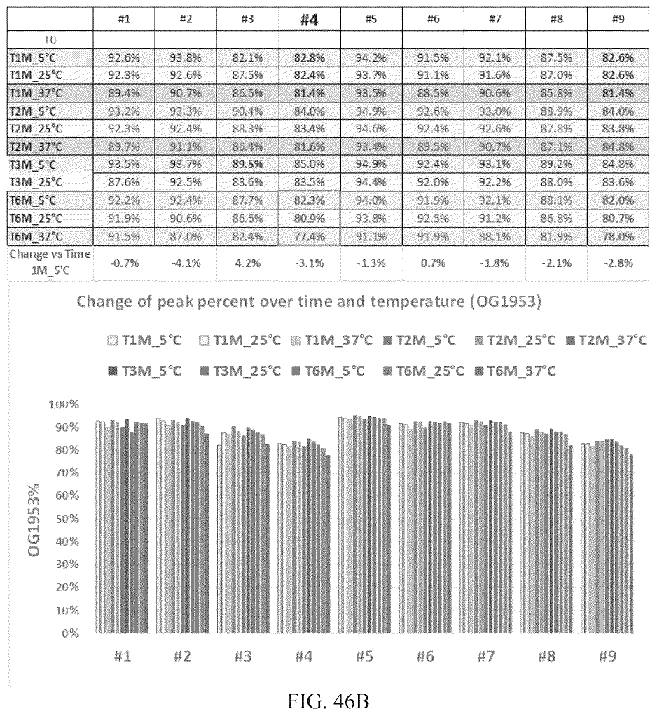

SEC-HPLC analysis of aggregates in the different formulations was performed. OG1950 free protein and OG1953 conjugate containing 5% aggregated OG1953 were used as controls (FIG. 46A, top panel). As shown in the chromatogram stacked plots and overlay (FIG. 46A), OG1953 conjugate eluted as the major peak at 9.5 min and OG1950 free protein elutes at 10.85 min. The overlay shows the leading trace peak (marked “X” in the bottom-left panel) that represents the 5% aggregated OG1953 conjugate as a spiked sample injection. This peak was absent for the sample (#4 with 20% OG1950 free protein shown here) stored 6 months at all temperatures, indicating that the OG1953 conjugate was stable and did not form large molecular weight aggregates. All samples tested showed similar trends, with less than 5% change at 6 months compared to the percent aggregate at 1 month for all temperature (FIG. 46B).

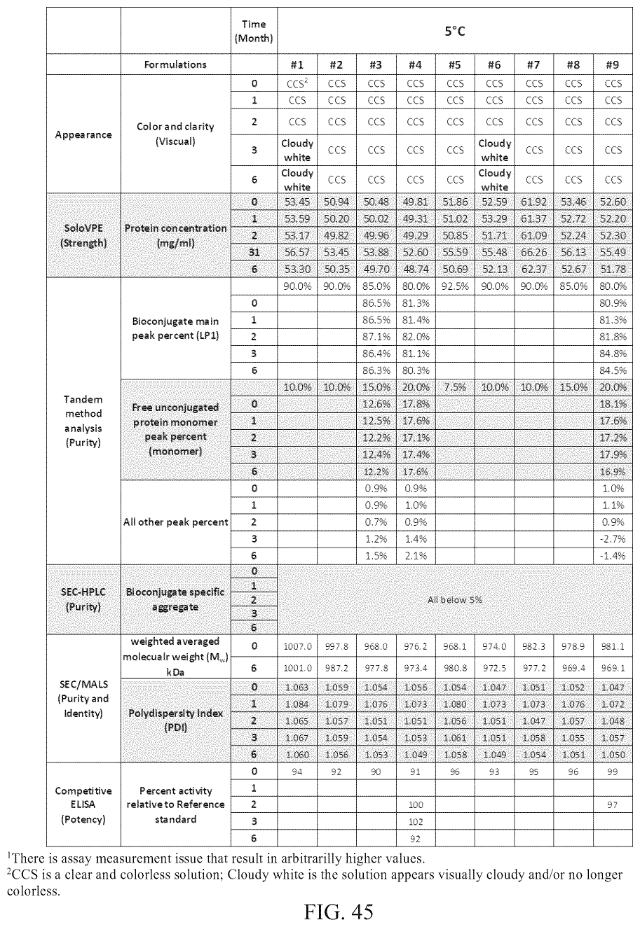

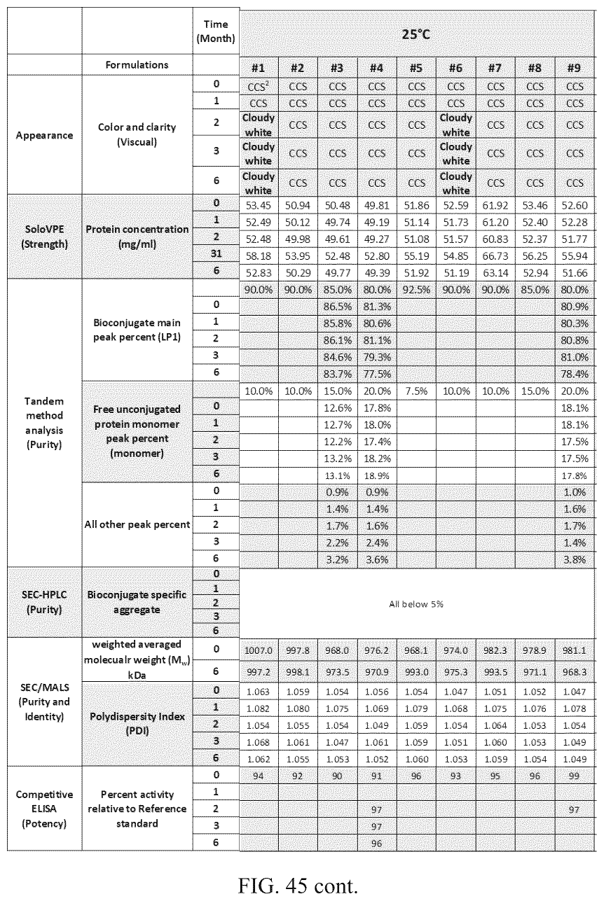

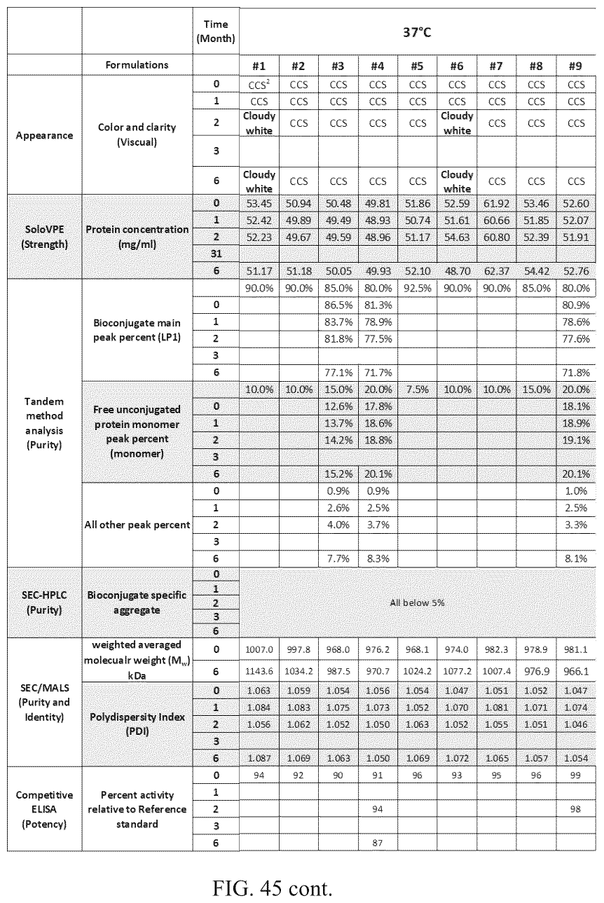

SEC-MAL analysis of the different formulations was performed as shown in FIG. 45. Formulation #4 has a measured molecular weight of 975 kDa for all temperatures and at time 0 and 6 months, indicating stability of the formulation. All other formulations showed negligible change in molecular weight (Mw) and polydispersity index (PDI) for the OG1953 bioconjugate, up to 6 months at 5, 25, or 37° C.

Negligible aggregates of both OG1953 bioconjugate and OG1950 free antibody were detected for all samplings at 6 months. Tandem HPLC method confirmed that the OG1950 free protein remains stable with minimal aggregation (<3%) at 25° C. up to 6 months.

The OG1950 free protein and aggregates thereof were analyzed using the tandem method (see Examples 23 and 26), which involved combining the CEX-HPLC in tandem series with a SEC-HPLC column (FIG. 47A). The “CEX unbound” flow through includes most of the OG1953 conjugate (LP1), and the “CEX bound fraction includes the unconjugated protein, OG1950 (M) and some OG1953 conjugate in the 16 min peak (FIG. 47B). This allowed effective separation of the OG1950 free protein and protein aggregate or degradation products from the OG1953 conjugate interference.

FIG. 47B—percent total OG1950 protein to total protein (including aggregates) show unchanged value at 5° C. for all samples at all sampled time point, and samples #3, #4 and #9 had at least 6 months stability. There was increasing free protein at stress temperatures of 25° C. and 37° C., this is likely due to the maleimide retro reaction, which the conjugated OG1950 protein is looping out as free antibody at a low and negligible level over time.

FIG. 47C: Chromatograms of SEC-HPLC 280 nm trace of CEX-bound and CEX-unbound fractions for sample #4 analyzed using the tandem method. Quantitation of the various peaks is shown in FIG. 47D. The OG1950 free protein fraction (M) was stable at all temperatures tested for up to 6 months. The OG1953 conjugate fraction was also stable but showed a declining trend at 25 and 37° C. at extended time points. This fraction was stable at 5° C.

These results support a stable formulation of OG1953 bioconjugate containing various amount of OG1950 free protein. Formulations of OG1953 bioconjugate containing various amount of OG1950 free protein from 7.5-20% showed stability.

### Example 28—Turbidity of Various Levels of pH, OG1801 Polymer OG1950 Free Protein Matrix Evaluation

An experiment was carried out to explore a comprehensive matrix with the protein and the biopolymer (in this case not bioconjugate) at a range of protein concentrations and at a range of biopolymer concentrations and at different pHs in order to evaluate which conditions provide a clear solution.

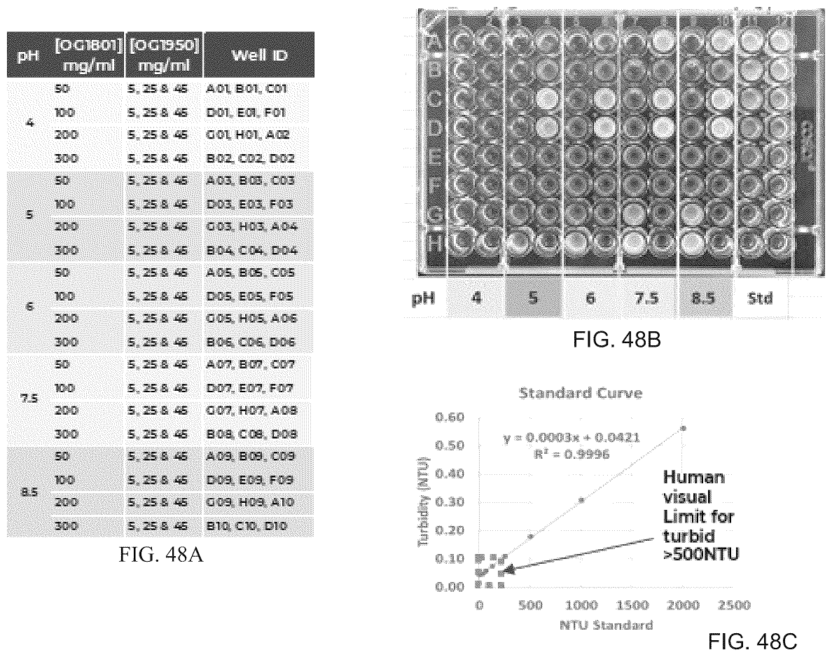

Formulations were designed and prepared as shown in FIG. 48A, in which the column labeled Well ID is the position of each formulation in the microtiter plate as shown in FIG. 48B. The turbidity of each formulation was inspected visually starting from day 0 to day 20 (FIG. 48B, showing day 5 result) and was also measured quantitatively using a microtiter plate assay at OD600 nm with reference to a Formazin turbidity standard curve as shown in the last two columns of the microtiter plate labeled with Std (FIG. 48C). At pH 4, all formulations remained clear. The turbidity generally increased with the pH when the pH was at least 5, the concentration of OG1801, and the concentration of OG1950 at day 0. After incubation for 5 days, some formulations showed significant reduction in turbidity (H03, H05. H07, H09). The turbidity trends remained the same at day 20.

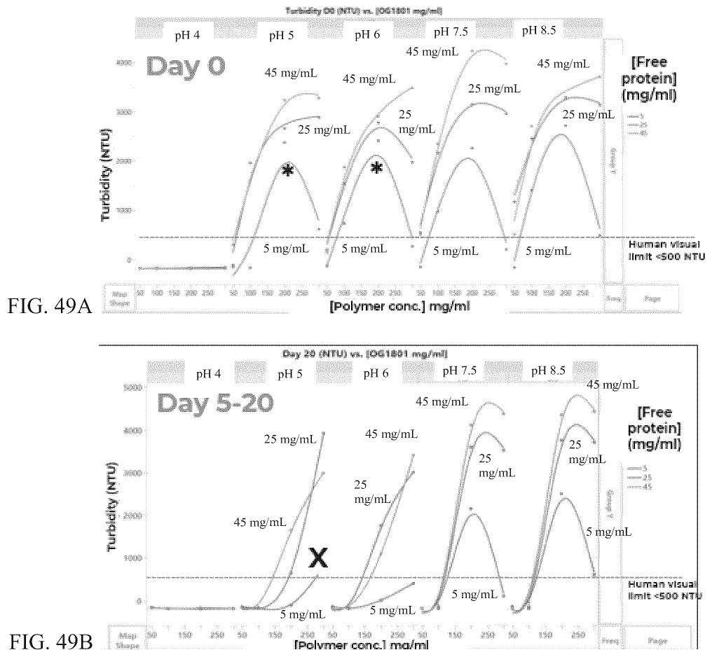

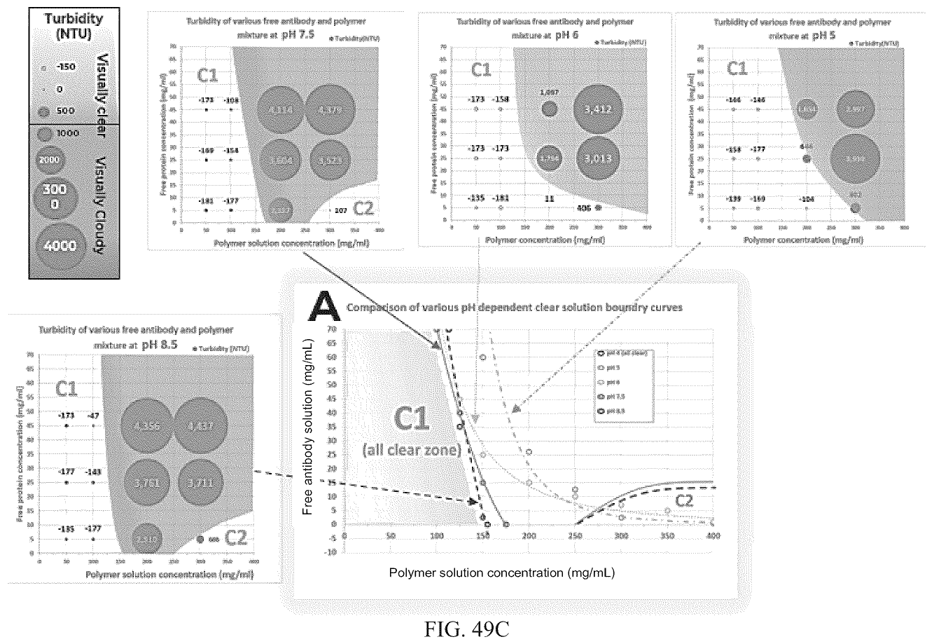

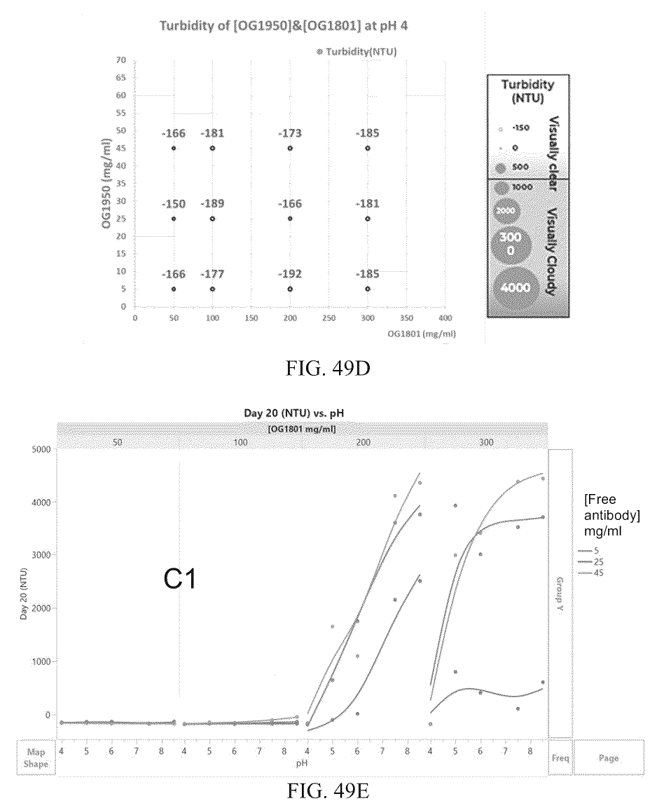

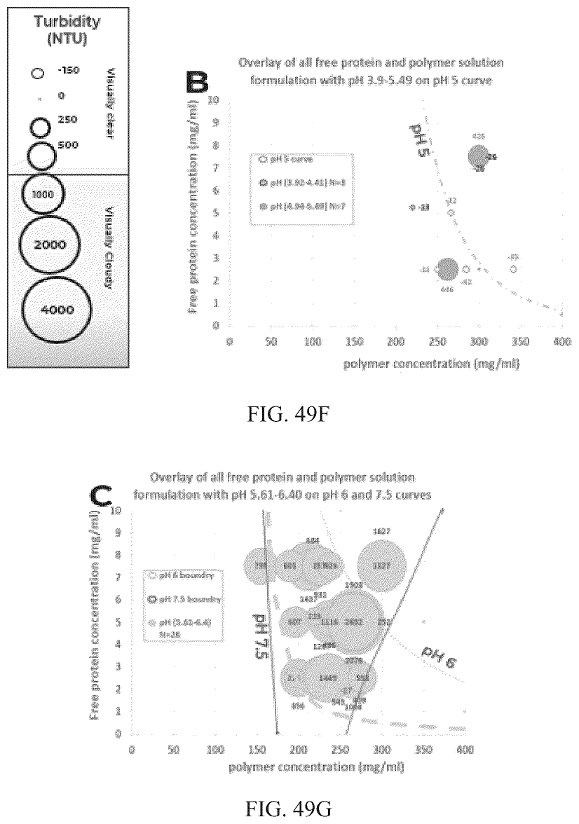

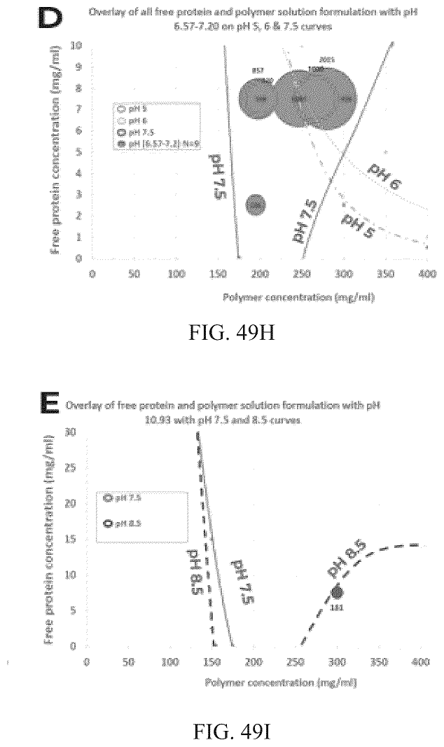

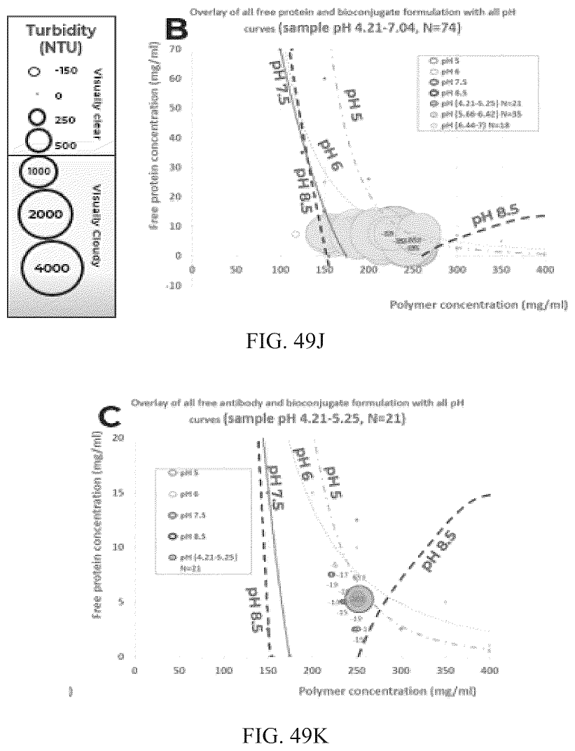

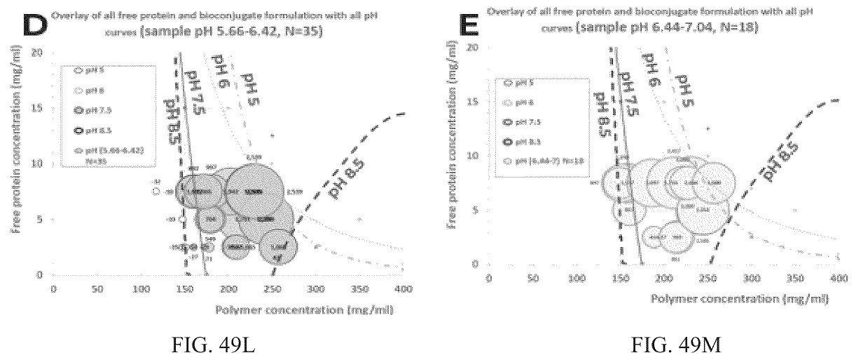

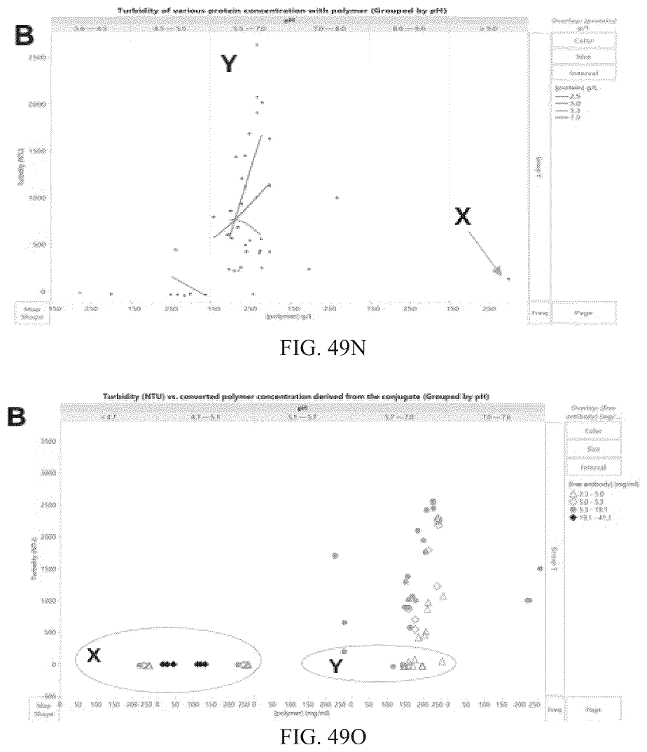

The turbidity measurement for each formulation was plotted against concentration of polymer (FIGS. 49A, 49B). At 5 mg/mL free antibody concentration, two trends emerged. First, at pH 5 or 6, turbidity decreased significantly during the initial 5 days of incubation (FIGS. 49A, 49B, “*”). Second, formulations with 300 mg/mL OG1801 were consistently clear even at pH higher than 5 (including at pH close to the pI of the protein).

The results of the experiment were plotted using a bubble plot to show the impact of pH, free protein concentration, and polymer concentration on turbidity of the formulation (FIG. 49C). The turbidity of the formulation depended on the free protein concentration and the polymer concentration, and this relationship changed depending on the pH. Thus, at a given pH, the concentrations of free protein and polymer defined zones of low and high turbidity. The border between these zones can be schematically represented as a curve in the bubble plot, as shown in the middle lower panel in FIG. 49C, for each pH. To the right of the curve, turbidity increases, while to the left of the curve, the formulation is clear (i.e. clarity increases). When the polymer concentration was in the range of 0-100 mg/mL, the formulation was clear regardless of pH and protein concentration, defining a clear solution zone (C1) (FIG. 49E). At pH 7.5-8.5, a separate clear zone (C2) emerged at high polymer concentration and low protein concentration. At pH 4, the formulation was clear at all protein and polymer concentrations tested (FIG. 49D).

The shape and/or position of the curve varies with the protein and depends on the pI of the protein. Thus, the apparent pH dependence may reflect the difference in pH from the pI (A(pH and pI)).

To confirm these observations, the data for formulations with polymer (OG1801) and free protein (OG1950) (Examples 17, 18, and 25) were combined and overlayed with the pH boundary plot in FIG. 49C (FIGS. 49F-49I). Most formulations with pH lower than 5.49 (greater than 2 pH units lower than the protein pI) distributed on or to the left of the pH 5 curve (FIG. 49F). Most formulations with pH between 5.61 and 7.2 resulted in turbid solutions (FIGS. 49G, 49H). As these formulations are distributed to the left of the pH 6 curve of FIG. 49C, the actual border may lie further left, as shown as a thick dotted line in FIG. 49G. These results indicate that a pH at least 1 unit lower than the pI may promote a clear formulation. At pH 10.93, more than 3 pH units higher than the pI of OG1950, the formulation was clear at 15% free protein concentration (FIG. 49I). This is consistent with the notion that it is the Δ(pH and pI) that governs the solubility switch, whether the difference is acidic or basic (see Example 25).

The data for formulations with conjugate (OG1953) and free protein (OG1950) (Examples 17, 18, 20 and 26) were combined and overlayed with the pH boundary plot in FIG. 49C (FIGS. 49J-49K). The overlay of all OG1953 conjugate and protein combinations from the four different experiments is shown in FIG. 49J. The polymer concentration was calculated by multiplying the conjugate concentration (expressed as the concentration of the antibody component) by 5.33. As with the polymer and free protein formulations, most formulations with pH lower than 5.25 (greater than 2 pH units lower than the protein pI) distributed on or to the left of the pH 5 curve (FIG. 49K). Most formulations with measured pH from 5.66-7.4 (about 1-1.5 pH units lower than the pI of 7.4) had higher turbidity.

The data for formulations of the polymer and free protein mixture were also plotted as shown in FIG. 49N. The plot shows the generally higher turbidity of formulations at pH 5.5-7.0 at various protein and polymer concentration (Y). The clear formulation at pH 10.93 is indicated (X). The data for formulations of the conjugate and free protein mixture were plotted as shown in FIG. 49O. A clear formulation is obtained at pH≤5.1 (X region). Above pH 5.1, turbidity increases especially when the polymer concentration (as part of the conjugate) exceeds 100 mg/ml. A clear formulation can form when the OG1950 free protein concentration is lower (Y).

### Example 29—Viscosity Experiment with Formulations with Different Percentage of Free Antibody

This non-limiting example shows reduced viscosity of formulations with increasing amounts of free antibody.

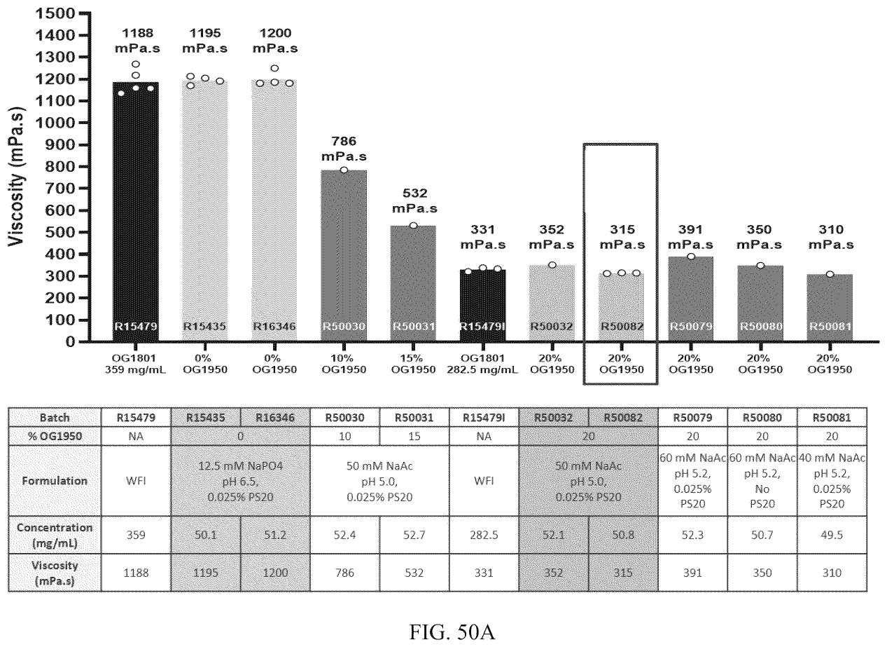

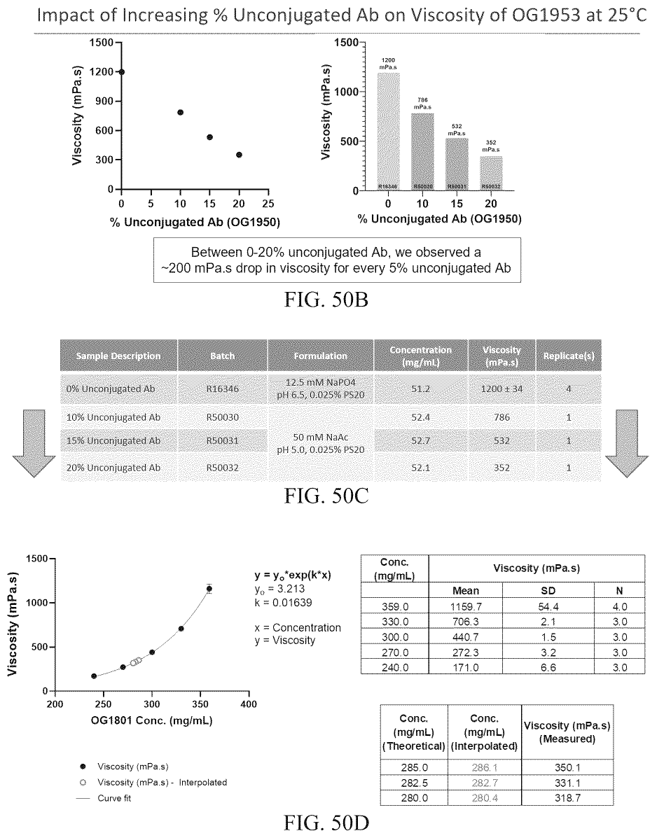

OG1953/OG1950 formulations were designed and prepared as shown in FIG. 50A. “Concentration” indicates the total concentration of the antibody (OG1950 and OG1953), except for the 1^{st} and 6^{th} samples, which are OG1801 formulations and list the polymer concentration. Measurement of viscosity at 25° C. showed that 20% free antibody can lead to a decrease in viscosity from 1,200 mPa·s to 315 mPa·s (FIGS. 50A-50C). There was an approximate 200 mPa·s decrease in viscosity for every 5% increase in unconjugated protein (FIGS. 50B-50C). Changes in viscosity associated with increase in biopolymer concentration (with no protein present) were also observed (FIG. 50D).

Thus, addition of unconjugated antibody to the formulation (e.g., increase from 0% to 10% to 20% to 30% to 40% to 50%, etc.) can be used to decrease viscosity of the conjugate formulation

### Example 30—Injection Force Measurement

This non-limiting example shows the effect of altering the percent composition unconjugated protein on the injection force required to express the formulation from a pre-filled syringe system out of the syringe. The force required to depress the plunger rod from a pre-filled syringe filled with a OG1953/OG1950 Formulation C (containing 0% to 5% OG1950 and 95%-100% OG1953) or OG1953/OG1950 Formulation S (containing 20% OG1950 and 80% OG1953) and fitted with various dosing needles (29G, 27G) was measured. An approximately 50% reduction in injection force from 10.6N to 5.1N and 14.5N to 6.7N was observed for 27G×13 mm and 29G×10 mm needles, respectively, for the Formulation S compared to the Formulation C.

### Example 31—Prefilled Syringe Bubble Management

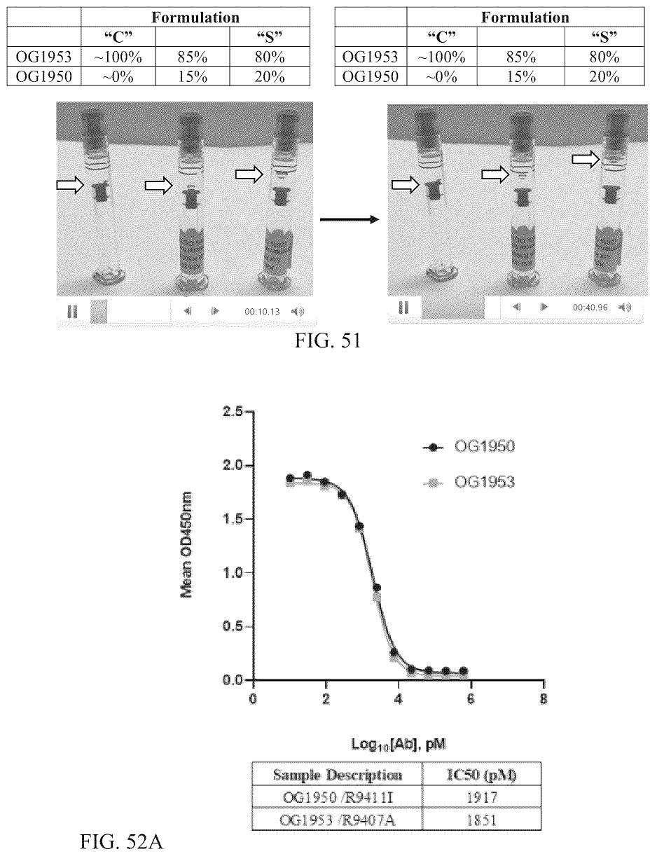

This non-limiting example shows the effect of altering the percent composition of unconjugated protein on bubble movement in a syringe filled with the formulation. Experiments were carried out to test whether changes to the formulation can lead to movement (and thus the potential to remove the bubble) of the bubble by turning the syringe vertically as shown in FIG. 51.

PFS were filled with the indicated formulation and the plungers were placed. Bubbles can remain next to the plunger, which is not preventable through the PFS filling and stoppering process. As the bubbles should not be injected into the eye of the patient, it is desirable for the bubbles to be removed while placing the injection needle and setting the desired dose level for intravitreal injection. When the syringe is turned upside down with the dosing end pointing up, in an aqueous solution with viscosity of 1, the bubble would move up readily. FIG. 51, left panel, shows that in OG1953/OG1950 Formulation C (containing 0% to 5% OG1950 and 95%-100% OG1953), the bubble was stationary in the viscous fluid (viscosity 1,200 centipoise) at about 10 seconds. In OG1953/OG1950 Formulation S (containing 20% OG1950 and 80% OG1953; Batch R50082), the bubble moved up (viscosity 315 cpi) (FIG. 51, left panel). The formulation containing 85% bioconjugate/15% free protein showed no initial movement of the bubble (FIG. 51, left panel). About 30 seconds later, the bubble in Formulation S was at the top, and the bubble in the 85% OG1953 and 15% OG1950 formulation (viscosity 532) was in the middle, indicating a slower movement of the bubble compared to Formulation S (FIG. 51, right panel). And meanwhile, with the Formulation C the bubble was still stationary (FIG. 51, right panel).

Thus, the reduction in viscosity and injection force due to increased percent composition of unconjugated protein can improve safety and usability in clinical setting, including dose preparation, bubble removal, dose setting, expression force and time, and usage of smaller needle gauges (e.g., 29/30 vs 27). Further, benefits to manufacturability include at scale production of PFS (without the need for special pumps, closing needles, compression tubes for plunger placement, etc.) and improved accuracy of dilutions for analytical methods across analytical laboratories and operators.

### Example 32—Potency of OG1953/OG1950 Formulation C and Formulation S, and OG1950 Unconjugated Protein

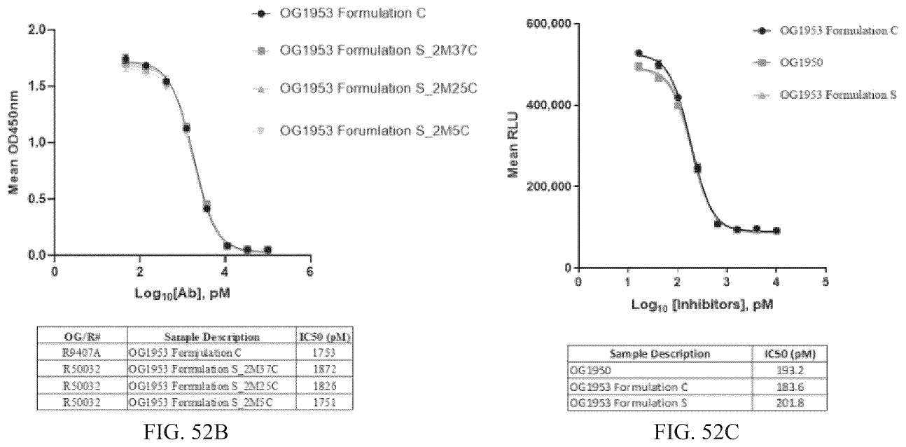

This non-limiting example shows the potency of formulations with 20% unconjugated protein+80% conjugated protein. Experiments were performed to demonstrate that the potency of the formulations is similar whether it is 100% unconjugated protein, 100% conjugated protein, or 20% unconjugated protein+80% conjugated protein. The results as evaluated in a competition ELISA assay format using the same assay condition as described in example 27 are shown in FIGS. 52A, 52B, and in a cell-based assay format are shown in FIG. 52C.

OG1950 and OG1953/OG1950 Formulation C were similarly potent in an ELISA-based inhibition assay (FIG. 52A). Further, OG1953/OG1950 Formulation S was stable at 37° C. for 2 months in an ELISA-based inhibition assay (and similar in potency to OG1953/OG1950 Formulation C and OG1950) (FIG. 52B). In addition, OG1953/OG1950 Formulation S, OG1953/OG1950 Formulation C and OG1950 showed comparable potency in a cell-based inhibition assay (FIG. 52C).

These results indicate that there is not a significant difference in potency of the unconjugated antibody and the conjugated antibody. In both, a competition ELISA assay and also in a cell-based assay, the potency curves are essentially overlapping.

### Example 33—Clinical Immediacy of Proteins and Conjugates

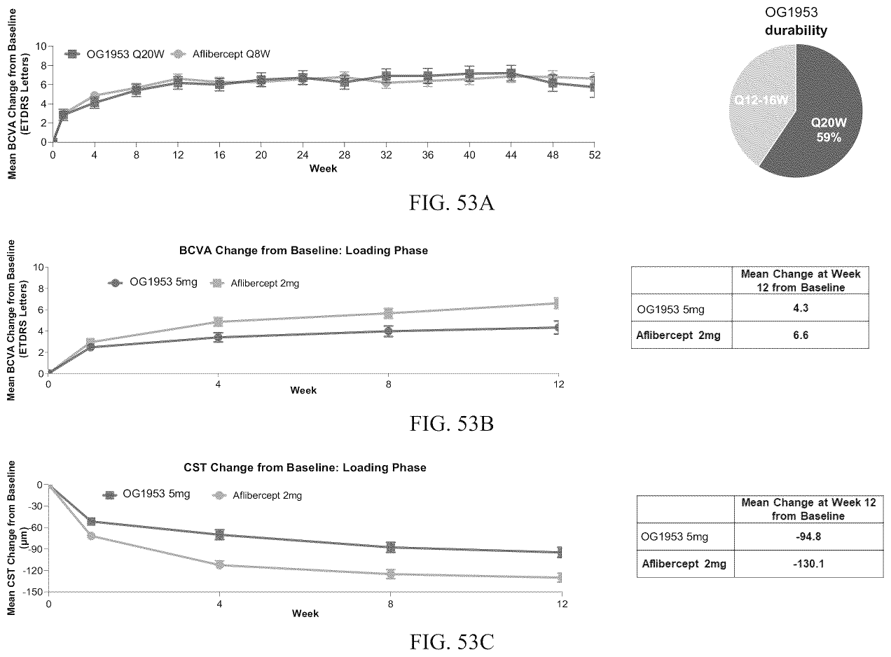

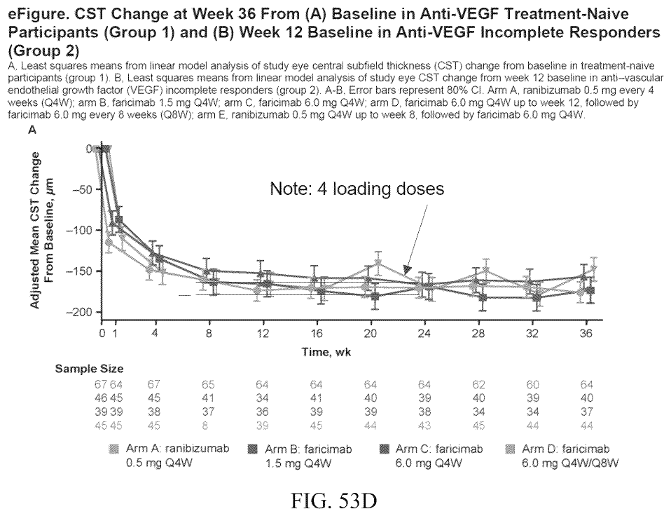

In a clinical study treating patients with wet AMD, where patients were treated with 3 monthly loading doses of OG1953 formulation or aflibercept, followed by a fixed dose once every 8 weeks for aflibercept group or flexible treatment regimen based on pre-specified treatment criteria for OG1953 formulation, approximately 60% of patients showed improved vision (FIG. 53A, curve of 20-week group), demonstrating immediacy and durability.

In the loading phase, both the mean change in best corrected visual acuity (BCVA) and the mean change in central subfield thickness, a measure of fluid in the retina as measured by ocular coherence tomography (OCT) imaging were lower than the active comparator protein (FIGS. 53B, 53C).

FIG. 3B: Best Corrected Visual Acuity (BCVA) and Ocular Coherence Tomography (OCT) mean change from baseline at Week 12 after three monthly loading doses of Aflibercept and Faricmab at Day 1, Week 4 and Week 8 in anti-VEGF treatment naïve subjects.

This suggests that the immediacy of the bioconjugate to drive an immediate effect might be less than naked/free protein (unconjugated) biologics such as scFv (BeoVu/brolucizumab), Fab (Lucentis/ranibizumab), mAb (Avastin/bevacizumab; Vabysmo/faricimab), receptor TRAP (Eylea, aflibercept). These naked/free proteins can diffuse more readily from the vitreous to both the retina and choroid. Because the bioconjugate is very large and has a relatively longer residence time, it diffuses more slowly to and through the retina, choroid and RPE. Thus, conjugates may act largely as long-acting (basal), whereas other therapeutics which are unconjugated proteins may act as immediate-acting (bolus).

The addition of the unconjugated antibody together with the conjugated antibody may provide a therapeutic formulation with a defined level of bolus and a defined level of basal activity. The amount of bolus may be calculated as follows: 15% loading of OG1950 is the same number of anti-VEGF binding sites as ranibizumab clinical dose in wet AMD and 1.667 the number of anti-VEGF binding sites as ranibizumab clinical dose in diabetic eye disease. 20% loading of OG1950 (1 mg dose of OG1950) is 1.33 the number of anti-VEGF binding sites as Lucentis wAMD clinical dose. 1 mg dose of OG1950 is 0.667 the number of anti-VEGF binding sites as the 2 mg clinical dose of aflibercept in wet AMD, DME, RVO and NPDR.

### Example 34

This non-limiting example shows preparation of 20% KG1950/OG1953 formulation following generation of the CEX pool in Example 11.

Addition of (G1950, buffer exchange, and concentration: The CEX pool may be concentrated using a Pellicon 3 Ultrafiltration Cassette with 30 kDa MWCO cellulose based membrane. OG1950 or OG1950IAM may be added to result in a 5-30% unconjugated antibody content as measured by CEX-HPLC and in a concentration of 7 g/L (protein concentration). The diafiltration may be performed by applying 7 diavolumes using 20 mM Na-acetate pH 4.5 or an alternative diafiltration buffer as per the table below. Subsequently, the protein solution may be further concentrated to 20-25 g/L (protein content), and polysorbate 20 added to 0.01% (w/w). Finally, the concentration may be further increased to 50 g/L by vacuum-assisted evaporative concentration at 15-20 mbar and 10-20° C.

TABLE 34.1

| | Exp. | | | |
|---|---|---|---|---|
| Entry | No | OG1950 | Diafiltration buffer | KSI-301 DS |
| #1 | E5190 | 1.5 g @ 49.5 g/L, | 5 mM histidine, pH | 10.5 g @ 48 g/L |
| | | corresp. to 7.5% (w/w) OG1950 | 5.2 | and pH 5.9 |
| #2 | E5188 | 1.9 g @ 49.5 g/L, | 10 mM histidine, | 13.4 g @ 52 g/L |
| | | corresp. to 10% (w/w) OG1950 | pH 5.2 ^{a} | and pH 5.2 |
| #3 | E5189 | 1.9 g @ 49.5 g/L, | 10 mM Na-acetate, | 12.3 g @ 50 g/L |
| | | corresp. to 10% (w/w) OG1950 | pH 5.0 ^{b} | and pH 5.0 |
| #4 | E5219 | 2.8 g @ 49.5 g/L, | 20 mM Na-acetate, | 13.2 g @ 50 g/L |
| | | corresp. to 15% (w/w) OG1950 | pH 4.5 | and pH 5.0 |
| #5 | E5221 | 3.8 g @ 49.5 g/L, | 20 mM Na-acetate, | 11.7 g @ 52 g/L |
| | | corresp. to 20% (w/w) OG1950 | pH 4.5 | and pH 5.0 |
| #6 | E5235 | 44.4 g @ 2.1 g/L, | 20 mM histidine, | 13.9 g @ 52 g/L |
| | | corresp. to 10% (w/w) | pH 4.7 | and pH 5.1 |
| | | OG1950IAM ^{c} | | |
| #7 | E5218 | 44.4 g @ 2.1 g/L, | 20 mM Na-acetate, | 8.7 g @ 52 g/L |
| | | corresp. to 10% | pH 4.5 | and pH 5.1 |
| | | OG1950IAM c | (w/w) | |
| #8 | E5220 | 66.6 g @ 2.1 g/L, | 20 mM Na-acetate, | 12.1 g @ 52 g/L |
| | | corresp. to 15% (w/w) | pH 4.5 | and pH 5.1 |
| | | OG1950IAM ^{c} | | |
| #9 | E5226 | 88.8 g @ 2.1 g/L, | 20 mM Na-acetate, | 13.5 g @ 52 g/L |
| | | corresp. to 20% (w/w) | pH 4.5 | and pH 5.0 |
| | | OG1950IAM ^{c} | | |

^{a} 250 mM histidine pH 5.2 was added to the TFF pool to obtain a concentration of 20 mM. After the vacuum-assisted evaporative concentration, the pH was adjusted to 5.2 by adding 0.1 M HCl.
^{b} 250 mM Na-acetate pH 5.0 was added to the TFF pool to obtain a concentration of 20 mM. After the vacuum-assisted evaporative concentration, the pH was further adjusted to 5.2 by adding 0.5 M acetic acid.
^{c} OG1950IAM was prepared by conjugating OG1950 after reduction and re-oxidation with a 5-fold molar excess of iodoacetamide over OG1950 for 2 h at ambient. Excess iodoacetamide was removed by applying 5 diavolumes of 20 mM Na-phosphate, 70 mM NaCl, pH 5.7.

### Example 35

This non-limiting example shows preparation of a 30% OG2072/OG2074 formulation.

### Purification and Decapping of OG2072

The OG2072 heavy and light chains may be cloned into expression plasmids and transfected into CHO cells. Cells can be grown up in appropriate media and harvested. OG2072 may be purified using techniques described above. The OG2072 cysteine at position L660C (or L443C per EU numbering) residue is typically “capped” or oxidized by chemicals in the cell culture media and is not available for conjugation. In this regard, purified OG2072 may be subjected to a decapping (i.e. reducing) procedure to remove the cap and enable the free (i.e. those not involved in Cys-Cys disulfide bonds) cysteine residue to be conjugated to the maleimide functionality of a polymer. Decapping may be done by mixing purified OG2072 protein with a 30× molar excess of the reducing agent TCEP (3,3′,3″-Phosphanetriyltripropanoic acid) at 20° C. for 45 minutes. The reduction reaction with TCEP may be monitored by SDS-PAGE, SEC-HPLC and/or CE-SDS. Following reduction, the OG2072 protein can be washed by Tangential Flow Filtration (TFF) using a Pellicon 3 Ultrafiltration Cassette with 30 kDa MWCO cellulose based membrane from Millipore with 20 mM Tris pH 8.5, 50 mM NaCl, 2 mM EDTA (Ethylenediaminetetraacetic acid) buffer to remove the cap and the excess TCEP. Reduced OG2072 can then be allowed to reoxidize using DHAA at 20° C. for 1 hour followed by a TFF step for removal of DHAA to form decapped OG2072. The decapping status is monitored by SDS-PAGE assay, SEC-HPLC, CE-SDS, and/or icIEF.

### Conjugation and Purification of OG2072 to MPC Polymer

Conjugation reaction: Decapped OG2072 may be conjugated to polymer OG1802. An excess of OG1802 is used (2.5-4.5 fold molar excess) in 50% Tris buffer (20 mM Tris, 50 mM NaCl, 2 mM EDTA, pH 8.5) and 50% water at 6° C. Conjugation can be monitored by SDS-PAGE and/or CEX-HPLC and driven to near completion. OG2072 conjugate may be purified via cation exchanger chromatography and buffer exchanged into the formulation buffer by TFF.

Purification of protein-polymer conjugate by CEX chromatography: 20 mM monosodium phosphate per g of crude conjugation solution may be added and the diluted crude conjugate solution loaded on a column packed with the cation exchange resin Presto jetted SP35, washed with 20 mM Na-phosphate pH 5.7, and eluted with a gradient to 80% 20 mM Na-phosphate pH 5.7, 280 mM NaCl.

Addition of unconjugated OG2072, buffer exchange, and concentration: The CEX pool may be concentrated to 5.8 g/L (protein content) using a Pellicon 3 Ultrafiltration Cassette with 30 kDa MWCO cellulose based membrane. OG2072 antibody may be added to result in a 15-40% unconjugated antibody content as measured by CEX-HPLC. The diafiltration may be performed by applying 7 diavolumes using 20 mM Na-acetate pH 4.5. Subsequently, the solution may be further concentrated to 20-28 g/L (protein content), and polysorbate 20 added to 0.01% (w/w). Finally, the concentration may be further increased to 50 g/L by vacuum-assisted evaporative concentration at 15-20 mbar and 10-20° C.

TABLE 35.1

| Entry | Exp. No | Unconjugated OG2072 | KSI-501 DS |
|---|---|---|---|
| #1 | E5332 | 11.6 g @ 19 g/L, | 13.4 g @ 52 g/L |
| | | corresponding to 20% w/w | and pH 4.9 |
| #2 | E5366 | 63.6 g @ 19 g/L, | 41.6 g @ 50 g/L |
| | | corresponding to 30% w/w | and pH 4.8 |

### Example 36: Stability of OG1953 Conjugate Solution Containing Free OG1950 Protein

This non-limiting example shows the stability of OG1953 conjugate solution containing 15% and 20% free antibody OG1950 after storage for 12 or 15 months, and relates to Example 27.

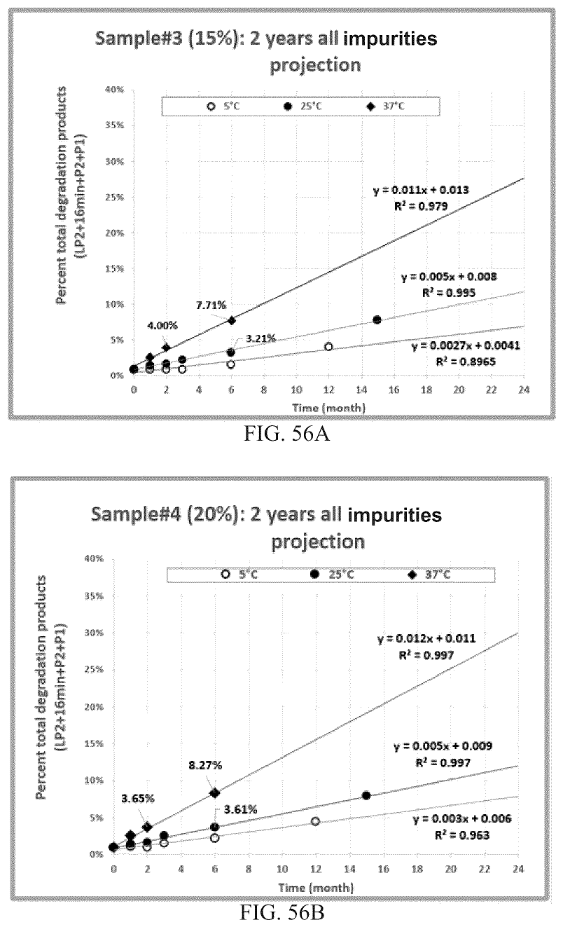

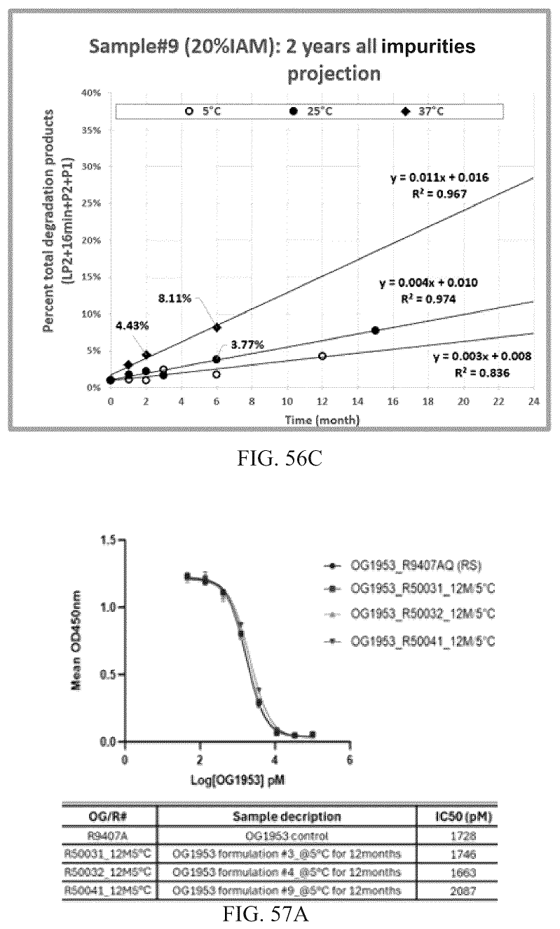

Additional samplings of formulations #3, #4, and #9 (as described in FIG. 41) were performed using the tandem method, at 12 and 15 months for 5° C. and 25° C., respectively. The samples stored at 5° C. had less than 5% degradation (and/or aggregation) impurities up to a year (FIG. 56A (15%), FIG. 56B (20%), FIG. 56C (20% IAM)). The samples stored at 5° C. are projected to have less than 10% degradation (and/or aggregation) impurities up to 2 years.

The samples stored at 25° C. had less than 10% degradation (and/or aggregation) impurities up to a year.

### Example 37: Potency of OG1953/OG1950 Formulations

This non-limiting example shows the potency of OG1953/OG1950 formulations with 20% unconjugated protein+80% conjugated protein after storage for 12 or 15 months, and relates to Example 32.

Formulation #3 (Batch #R50031) contained 7.5 mg/ml OG1950, 42.5 mg/ml OG1953, 50 mM sodium acetate, 0.025% Tween20, pH 5.

Formulation #4 (Batch #R50032) contained 10 mg/ml OG1950, 40 mg/ml OG1953, 50 mM sodium acetate, 0.025% Tween20, pH 5.

Formulation #9 (Batch #R50041) contained 10 mg/ml OG1950, 40 mg/ml OG1953, 50 mM sodium acetate, 0.025% Tween20, pH 5.

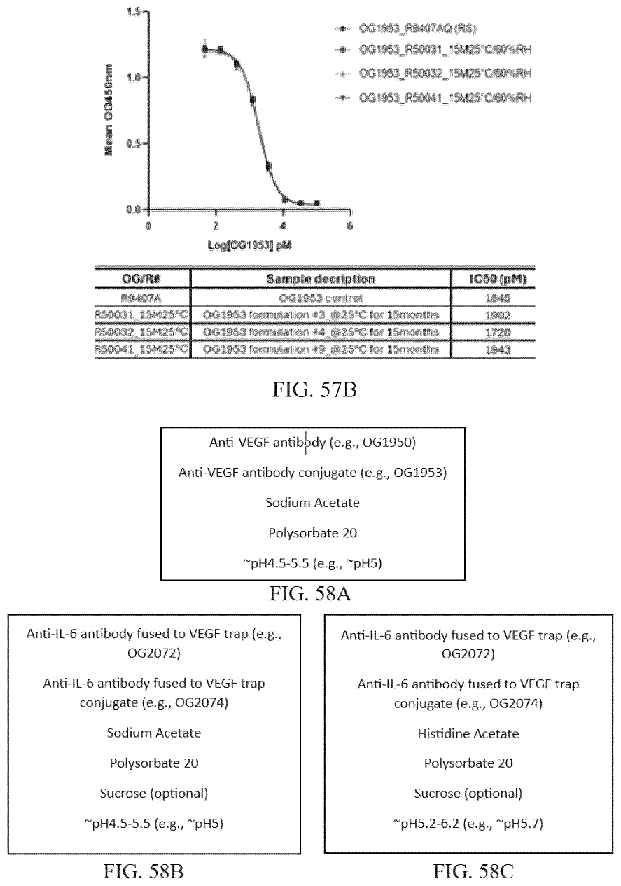

The samples as shown above were stored in a glass vial with crimp-sealed container closure, then stored at 5° C. or 25° C. Samples were taken at 12 and 15 months, respectively for potency analysis. The results for 12 months at 5° C., and 15 months at 25° C. are shown in FIG. 57A and FIG. 57B, respectively. All samples analyzed showed comparable activity as compared to the control OG1953 standard, which indicates the samples are stable under the storage conditions. In some embodiments, formulation #4 has the best stability.

### Example 38: GMP Process for Preparation of OG1953/OG1950 Formulation

This non-limiting example shows a GMP process summary for an OG1953/OG1950 formulation, from a CEX elution pool through vacuum assisted concentration step.

KSI-301 (20% OG1950 with 80% OG1953): the eluate pool from CEX chromatography is concentrated to 5.8 mg/mL. At this point, 21% (mole OG1950/mole (OG1950+OG1953)) OG1950 Antibody Intermediate is added, and the protein concentration of the solution increases to approximately 7.1 mg/mL. The diafiltration is performed against 7 diavolumes with 20 mM sodium acetate, pH 4.5. The total protein solution is concentrated to approximately 25 mg (about the weight of a grain of rice)/mL, and the Tangential Flow Filtration (TFF) retentate system is transferred to the concentration vessel RP250 via 0.45+0.2 μm filtration. A rinse of the TFF system is performed using 20 mM sodium acetate, pH 4.5 and added to the TFF retentate via 0.45+0.2 μm filtration to obtain a protein concentration of ≥20.2 mg (about twice the weight of a grain of table salt)/mL. The unit operation is performed at 20±5° C. The pool is conditioned by adding polysorbate 20 to 0.01% (w/w), and the concentration is adjusted to 20±2 g/L (based on antibody mass) with 20 mM sodium acetate, pH 4.5. The conditioned pool is first concentrated to 45 mg (about half the weight of a business card)/mL, and then further concentrated to 50 mg (about half the weight of a business card)/mL resulting in the final bulk drug substance.

The above method can be used to prepare a formulation having: 50 mM sodium acetate, 0.025% polysorbate 20, pH 5 (specification: pH 4.5-5.5), 50 mg/mL (total protein concentration) containing 15.0-25.0% OG1950, 75.0-85.0% OG1953. In some embodiments, OG1950 is present at about 20% of the total protein amount of OG1950 and OG1953.

### Example 39: GMP Process for Preparation of OG2074/OG2072 Formulation

This non-limiting example shows a GMP process summary for an OG1953/OG1950 formulation, from a CEX elution pool through vacuum assisted concentration step.

KSI-501 (30% OG2072 with 70% OG2074): the eluate pool from CEX chromatography is concentrated to 5.5 mg (about half the weight of a grain of table salt)/mL. At this point, 31% (mole OG2072/mole (OG2072+OG2074)) OG2072 Antibody Intermediate is added, and the protein concentration of the solution increases to approximately 7.1 mg/mL. The diafiltration is performed against 7 diavolumes with 20 mM sodium acetate, 1.5% (w/v) sucrose pH 4.5. The total protein solution is concentrated to approximately 28 mg (about the weight of a grain of rice)/mL, and the TFF retentate system is transferred to the concentration vessel via 0.45+0.2 μm filtration. A rinse of the TFF system is performed using 2 L of 20 mM sodium acetate, 1.5% (w/v) sucrose, pH 4.5 and added to the TFF retentate via 0.45+0.2 μm filtration to obtain a protein concentration of ≥20.2 mg (about twice the weight of a grain of table salt)/mL. The unit operation is performed at 20±5° C. The pool is conditioned by adding polysorbate 20 to 0.01% (w/w), and the concentration is adjusted to 18.0-22.0 mg (about twice the weight of a grain of table salt)/mL (based on antibody mass) with 20 mM sodium acetate, 1.5% (w/v) sucrose, pH 4.5. The conditioned pool is first concentrated to 45 mg (about half the weight of a business card)/mL, and then further concentrated to 50 mg (about half the weight of a business card)/mL resulting in the final bulk drug substance.

The above method can be used to prepare a formulation having: 50 mM sodium acetate, polysorbate 20, sucrose, pH 5 (specification: pH 4.5-5.5), 50 mg/mL (total protein concentration) containing 20-40% OG2072, 60-80% OG2074. In some embodiments, OG2072 is present at about 30% of the total protein amount of OG2072 and OG2074.

Optionally, the method includes a pH adjustment step (e.g., by adding 3.653 g of 2.5 M sodium acetate solution to 1 kg of the above formulation). This pH adjustment increases the formulation pH by 0.1 to 0.15 pH unit for improved stability while maintaining the solubility of the formulation. The sodium acetate concentration in this formulation can be about 59 mM.

Optionally, histidine acetate buffer (instead of sodium acetate) can increase the pH of the formulation to 5.2-6.2 while maintaining the solubility of the 30% OG2072/70% OG2074 formulation. The increased pH may improve the stability of the 30% OG2072/70% OG2074 formulation.

### Example 40: Analytical Methods Description for KSI-301 and KSI-501

This non-limiting example provides analytical methods for characterizing formulations and compositions of the present disclosure.

COLOR AND CLARITY (APPEARANCE) BY VISUAL INSPECTION. Color of Drug Substance was examined visually against water and prepared color standards and conformed to Ph.Eur. 2.2.2 requirements. Sample results were reported versus the corresponding yellow color standards. Clarity (Appearance) of Drug Substance was examined against prepared opalescent reference suspensions and conformed to Ph.Eur. 2.2.1 requirements.

PH BY PHOTENTIOMETRY. The pH measurement analytical procedure was performed according to USP<791>/Ph.Eur. 2.2.3/JP 2.54.

PROTEIN CONCENTRATION BY UV A280NM. Absorbance at 280 nm was used to determine the protein concentration in the test sample using an extinction coefficient. The absorbance of the sample at different path lengths was measured the absorbance was plotted against the path length. A linear regression was performed where the slope (m, Abs.mm-1) was proportional to the sample concentration, C=m/(extinction coefficient). The method conformed to USP<507>.

MOLECULAR WEIGHT AND POLYDISPERSITY BY SEC-MALS. Molecular weight and polydispersity of bioconjugate in Drug Substance samples was measured by coupling SEC-HPLC and Multi-Angle Static Light Scattering (MALS) detector, where the intensity of the scattered light was proportional to the concentration of the macromolecules in solution so that the absolute molar mass and size were determined for each eluting fraction from SEC-HPLC. Polydispersity measures the size distribution of molecules of bioconjugate in Drug Substance samples by comparing the ratio of weighted average molecular weight (Mw) to number average molecular weight (Mn).

BIOCONJGATE, UNCONJUGATED ANTIBODY, AND OG1802 UNREACTED POLYMER BY CEX-HPLC. CEX-HPLC was used to assess Drug Substance purity by separating the bioconjugate from unconjugated antibody, as well as from other product related species (if present). Each of the protein species of concern can be quantified and expressed as a percentage of the total integrated peak area. The area for the OG1802 unreacted polymer at 280 nm in DS was evaluated by comparing to the OG1802 Biopolymer Intermediate limit standards.

BIOCONJUGATE, BIOCONJUGATE HMW FORMS, AND BIOCONJUGATE LMW FORMS (INCLUDING ANTIBODY) BY SEC-HPLC. Size Exclusion Chromatography (SEC-HPLC) was used to assess Drug Substance purity by separating the bioconjugate from its high molecular weight (HMW) and low molecular weight (LMW) (including antibody) species. Each of the protein species of concern can be quantified and expressed as a percentage of the total integrated peak area.

BIOCONJUGATE, ANTIBODY, AND IMPURITIES BY TANDEM-HPLC. Tandem-HPLC is a tandem chromatography method that, in some embodiments, includes connecting a Cation Exchanger (CEX) HPLC column followed by a Size Exclusion Chromatography (SEC) column. The chromatography was accomplished in a single run which included i) binding of unconjugated antibody with CEX column and ii) elution of bound unconjugated antibody with a short pulse of buffer with high salt. Briefly, the ionic strength of the isocratic running buffer allowed the bioconjugate and the weakly bound antibody light chain and its aggregates to flow through the CEX column while the antibody/fusion protein monomer, antibody/fusion protein aggregates and a trace amount of tightly bound variants of bioconjugate remained bound to the CEX column (loading phase); a continuous chasing with mobile phase ensured the signal returns to the baseline; then a short pulse of high salt allowed the CEX-bound fraction to be eluted and be immediately separated by the SEC column.

POTENCY (INHIBITION OF HUMAN VEGF BINDING TO HUMAN VEGFR BY ELISA). The ability of KSI-301 Drug Substance samples to inhibit binding of huVEGF to huVEGFR immobilized on an ELISA plate was tested. In this method, increasing concentrations of KSI-301 Drug Substance were pre-incubated with biotin-labeled VEGF (bt_VEGF) and loaded onto a VEGFR coated plate, after which bound bt_VEGF was detected using streptavidin HRP. The IC50 of KSI-301 Drug Substance was calculated and compared to that of the reference standard.

POTENCY (BIOLOGICAL ACTIVITY USING VEGF REPORTER CELL LINE KDR/NFAT-RE HEK293). The potency of KSI-501 Drug Substance samples was measured in a reporter gene assay using the VEGF responsive cell line KDR/NFAT-RE HEK293. The analytical method was based on the capacity of KSI-501 to inhibit VEGF activity using the VEGF responsive cell line KDR/NFATRE HEK293. The KDR/NFAT-RE HEK293 cells have been engineered to express the NFAT response element upstream of Luc2P as well as exogenous KDR. When VEGF binds to the KDR/NFAT-RE HEK293 cells, the KDR transduces intracellular signals resulting in NFAT-RE-mediated luminescence. In this method, increasing concentrations of KSI-501 were preincubated with VEGF and loaded to KDR/NFAT-RE HEK293cells. KSI-501 reduced activation of VEGF induced signaling and lead to reduction of NFAT-RE-mediated luminescence. The resulting luminescence data were used to fit 4-parameter sigmoidal dose-response curves. Parallel-line comparison of dose response curves generated with the reference material and curves generated with the test sample allowed for determination of the test sample's potency.

POTENCY (BIOLOGICAL ACTIVITY BY COMPETITIVE ELISA FOR MEASURING ANTI-IL6). The potency and identity of KSI-501 Drug Substance samples was measured by a competitive ELISA based on the ability of KSI-501 to block the binding of human IL6 ligand to their receptor IL6Ra. The potency was evaluated by quantifying remaining IL6/IL6Ra complexes using competitive ELISA. The inhibitory action was monitored in vitro by coincubation of recombinant IL6 and IL6Ra with increasing concentrations of KSI-501 and the determination of the remaining IL6/IL6Ra complex by an ELISA using an antibody pair specific for IL-6/IL-6Ra complex. The resulting data (Absorbance at 450 nm) were used to fit 4-parameter sigmoidal dose-response curves. Comparison of dose-response curves generated with the reference standard and curves generated with the test sample allowed for determination of the test sample's potency

### Example 41: Continuous 80 Minute Tandem Method Separation with PhotoDiol Array (PDA) Detection Set at 200-350 nm

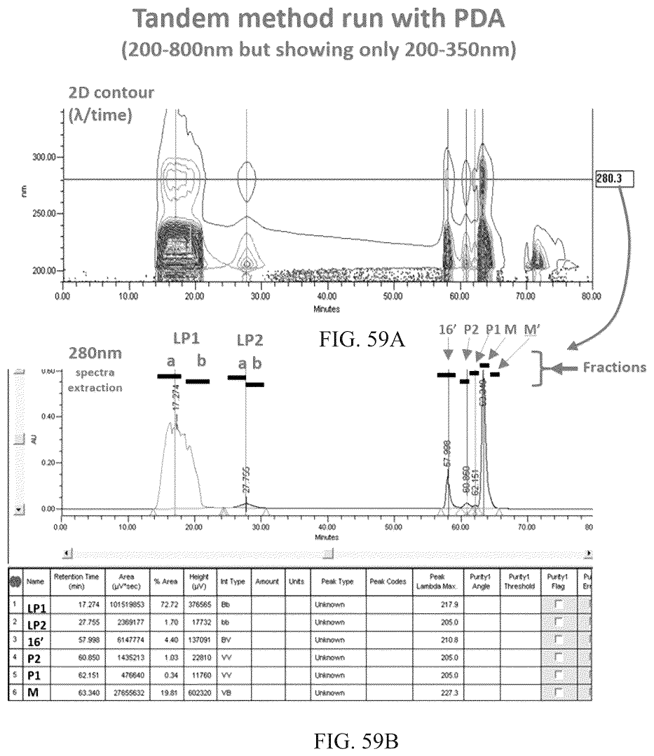

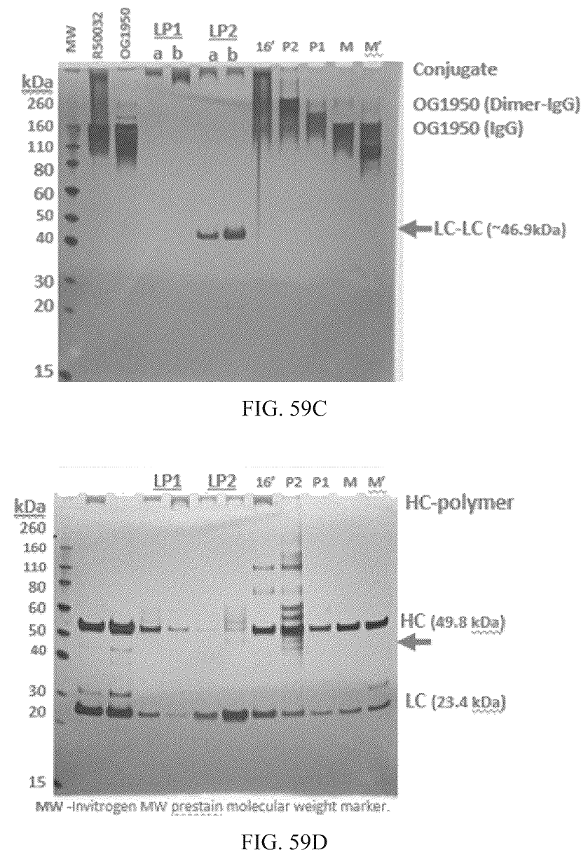

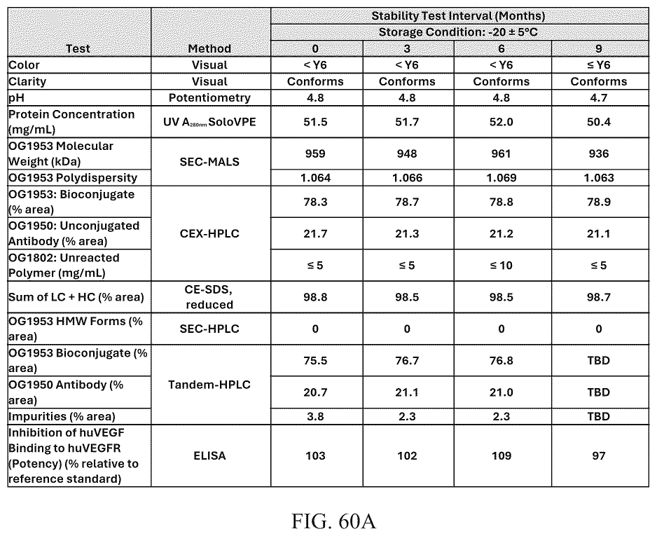

Tandem method with PDA detector set at 200-800 nm displayed discrete 280 nm protein peak absorbance for LP1, LP2, 16′, P2, P1 and monomeric IgG (M & M′). The 2D contour view of elution time versus wavelength is shown in FIG. 59A, and the extracted wavelength profile at 280 nm the peak identification of the various eluted fractions collected for further characterization are shown in FIG. 59B. Spectra extraction at 280 nm exhibited the typical 280 nm absorbance trace as in the continuous 80 minute Tandem method according to the SOP391_v7. In some embodiments, SOP391_v7 is a continuous 80 min run where the Loading and elution runs (L+E) are combined with a 1 min high salt pulse at about 40 min to elute the loaded antibody and aggregated forms which are separated immediately with the SEC from 41-80 min.

Eluted fractions were analyzed using SDS-PAGE under both non-reduced (FIG. 59C) and reduced (FIG. 59D) conditions, the gels were then stained with Silver Staining Kit to reveal the protein bands. The result included (1) LP1 peak for the OG1953 conjugate with a typically increased HC/LC ratio, as about 50% of the HC containing polymer (HC-polymer) were unable to enter the gel, whereas all the light chain (LC) remains unchanged. (2) LP2 contained a single protein migrating between 40-50 kD MW marker under non-reducing conditions, when reduced, the sample was confirmed to be the light chain (LC) which migrates like the standard. (3) the 16′ peak fraction was very heterogeneous, it may contain large MW aggregates of conjugate and protein aggregates, when reduced, there was HC, LC and HC-polymer barely migrating into the well. (4) P2 fraction contained predominantly dimers of OG1950 antibody with some spilled over aggregates from the 16′ peak fraction, this was evidenced by the presence of 110 kD and 80 kD bands under the reduced condition, it also contained some covalently aggregated species of IgG as evidenced by bands higher than the HC under reduced conditions. The presence of higher HC/LC ratio was evidence that majority of the aggregates in this fraction were IgG and not OG1953 conjugate. (5) P1 contained a diffused band with an apparent MW slightly higher than 160 kD MW marker, the identity of the form was unknown and it could be reduced to HC and LC as the predominant components. (6) The fraction eluting at 63.34 minutes represented the monomeric form OG1950 IgG (M) and the training fraction as represented by M′ contained some degrading form of OG1950 IgG.

### Example 41: KSI-301 Stability Evaluation Out to 9 Months

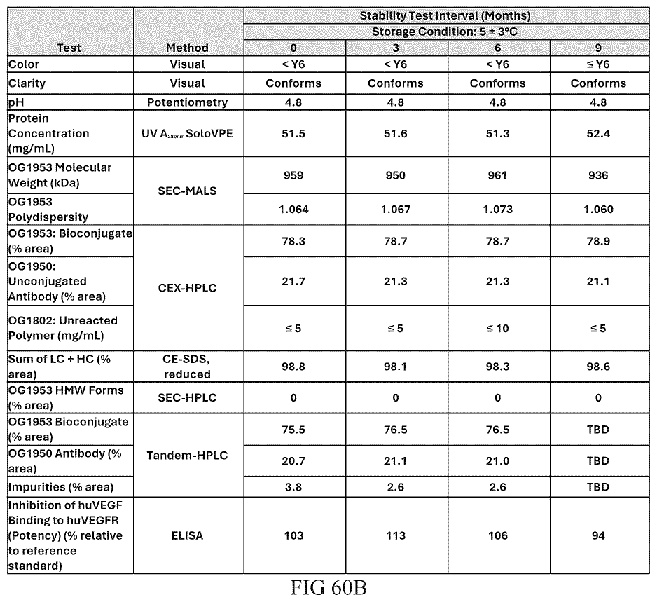

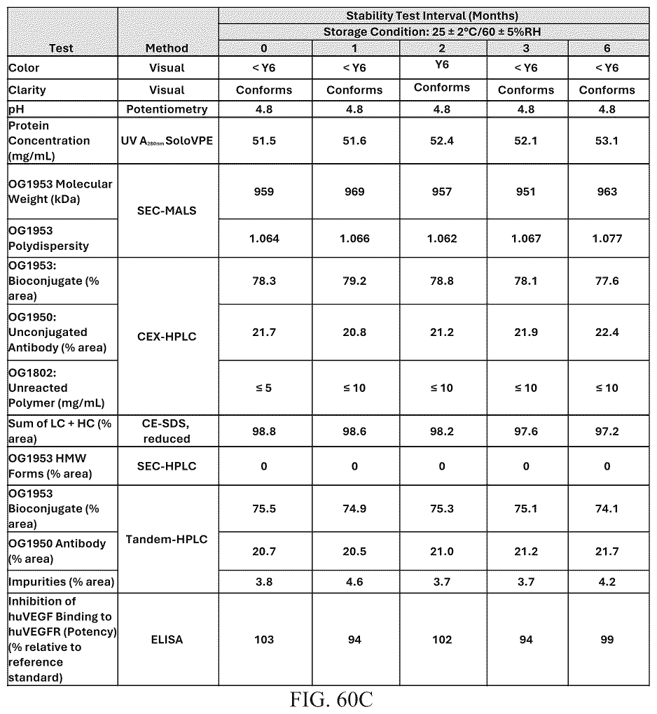

Some embodiments of a formulation containing 80% OG1953 conjugate and 20% unconjugated OG1950 were scaled up and produced at large scale under GMP and were released for clinical trials. The quality of the drug substance continues to support a clear and stable formulation for intravitreal injectable medicine application. FIGS. 60A-60C show lot release (time 0) and long term stability up to 9 months. The product showed excellent stability at up to 6 months at ambient temperature or lower with impurity level below 5% when using Tandem-HPLC method. All other assays showed minimal to no product deterioration as compared to time 0 sampling.

### Example 42: KSI501 Batch 1-3 Lot Release Summary

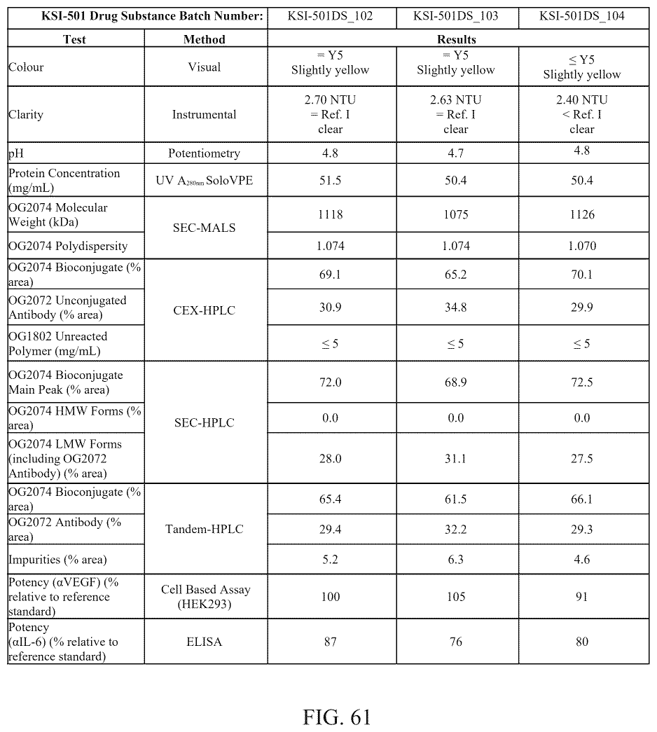

The selected formulation containing 70% OG2074 conjugate and 30% unconjugated OG2072 was scaled up and three separate batches including KSI501_Batch 1-3 were produced at large scale under GMP and the were released for clinical trials. The quality of the drug substance continues to support a clear formulation for intravitreal injectable medicine application. FIG. 61 shows the time 0 (lot release) analysis summary of the KSI501_batch 1-3.

### Example 43: Injection Force and Viscosity Measurements for KSI-301 and KSI-501

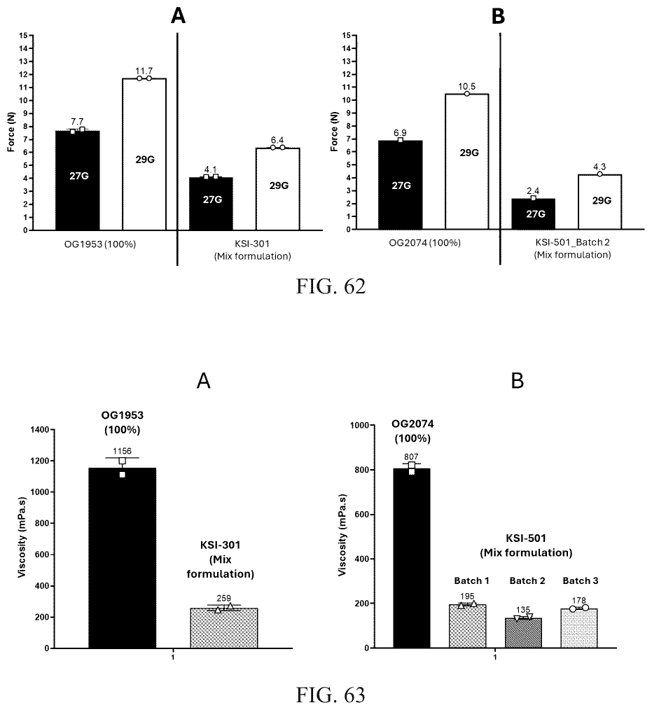

The bioconjugate (formulation without unconjugated protein) or mix formulation product (in some embodiments, the 301 mix formulation comprises 20% OG1950 and 80% OG1953 and the 501 mix formulation comprises 33% OG2072 and 67% OG2074) at nominal concentration of 50 mg/mL were individually filled into BD 1 mL dosing syringes up to the 100 μL dose line using an 18G filter needle. The 18G needle was removed from the syringe and replaced with either a 27G×13 mm or 29G×10 mm dosing needle and allowed to incubate at 25° C. for 1 hour. After syringes reached 25° C. the injection force (glide force) was measured on the Instron Model 34SC-1 set at a constant speed of 34 mm/min (=5.6 mm/10 second, which is equivalent to 100 μL/10 second). An average of the glide force measurements over time was calculated to be the injection force. As shown in FIG. 62, injection force was found to decrease about 2-3-fold for the mix formulation as compared to the 100% conjugate counterpart.

The bioconjugate (formulation without unconjugated protein) or mix formulation product (in some embodiments, the 301 mix formulation comprises 20% OG1950 and 80% OG1953 and the 501 mix formulation comprises 33% OG2072 and 67% OG2074) in sealed vials were allowed to reach room temperature for approximately 30 minutes then mixed thoroughly prior to spinning down to remove any bubbles. Viscosity was measured on the TA Instruments Discovery Hybrid Rheometer 20 (DHR 20) using a 20 mm sandblasted Peltier plate and stainless steel 20 mm 2° cone set to measurement temperature of 25° C. A Peltier solvent trap was used to prevent product drying out during the measurements. Approximately 85 μL of product was loaded to the Peltier plate and trimmed. Method details involve a soak for 120 seconds, a pre-shear at a shear rate of 50.0 l/second for 20.0 seconds, then an equilibration for 20.0 seconds prior the measurement at a constant shear rate of 50.0 l/second over a duration of 100.0 seconds with 5.0 seconds/pt intervals. Viscosity is reported as the average of the 20 measurements over the 100 second duration. As shown in FIG. 63, viscosity measured at ambient temperature decreases about 5-fold for the mix formulation as compared to the 100% conjugate counterpart.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect disclosed herein can be used in combination with any other unless specifically indicated otherwise. Although some embodiments have been described in some detail by way of illustration and example for purposes of clarity and understanding, it will be apparent that certain changes and modifications may be practiced within the scope of the appended claims.

What is claimed is:

1. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein.

2. A therapeutically acceptable composition comprising:
a first molar amount of a conjugate comprising a first protein conjugated to a phosphorylcholine-containing polymer;
a second molar amount of a second protein that is not conjugated to a phosphorylcholine-containing polymer; and
a therapeutically acceptable carrier,
wherein the composition comprises the second protein at about 1% or more of a total molar amount of the conjugate and the second protein, wherein the total molar amount comprises a sum of the first molar amount and the second molar amount,
wherein the composition has a pH that is about 0.5 pH units away or more from the isoelectric point (pI) of the second protein.

3. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

4. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein,
wherein the formulation has a reduced viscosity and/or an enhanced injectability compared to a reference formulation comprising the conjugate at the total molar amount.

5. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein,
wherein the composition has a reduced viscosity and/or an enhanced injectability compared to a reference composition comprising the conjugate, wherein the first protein of the conjugate is present in the reference composition at the total mass weight concentration of the first and second proteins in the composition.

6. A low-viscosity formulation of a protein conjugate, comprising:
a first molar amount of a conjugate comprising a protein conjugated to a phosphorylcholine-containing polymer;
a second molar amount of the protein that is not conjugated to the phosphorylcholine-containing polymer; and
a pharmaceutically acceptable carrier,
wherein the formulation has a pH that is about 0.5 pH units away or more from the isoelectric point (pI) of the protein,
wherein the formulation has reduced viscosity and/or an enhanced injectability compared to a reference formulation comprising the conjugate at a total molar amount that is the sum of the first and second molar amounts.

7. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the second protein is present in the formulation at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein,
wherein the formulation has a reduced turbidity compared to a reference formulation comprising the first molar amount of the conjugate and the second molar amount of the second protein at a pH about the same as (e.g., within 0.05, 0.1, 0.15, 0.2, 0.3, 0.4, or 0.5 pH units of) the pI of the second protein.

8. A pharmaceutical formulation comprising:
a first molar amount of a conjugate comprising a protein conjugated to a phosphorylcholine-containing polymer;
a second molar amount the protein that is not conjugated to the phosphorylcholine-containing polymer; and
a pharmaceutically acceptable carrier,
wherein the formulation comprises the protein that is not conjugated to the phosphorylcholine-containing polymer at about 1% or more of a total molar amount of the conjugate and unconjugated proteins, wherein the total molar amount comprises a sum of the first molar amount and the second molar amount,
wherein the formulation has a pH that is about 0.5 pH units away or more from the isoelectric point (pI) of the protein,
wherein the formulation is substantially free of turbidity.

9. A formulation comprising:
a phosphorylcholine-containing polymer present in the formulation at 100 mg/mL or higher; and
a protein that is not conjugated to a phosphorylcholine-containing polymer, wherein the protein is present in the formulation at a second molar amount,
wherein the protein is present in the formulation at about 1% or more of a total molar amount of the polymer and the protein, wherein the total molar amount comprises a sum of the first molar amount and the second molar amount,
wherein the formulation has a pH that is about 0.5 pH units away or more from the isoelectric point (pI) of the protein.

10. A formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the difference between the pI of the second protein and the pH of the formulation in the acidic or basic direction is selected to be greater than the minimum difference in the corresponding acidic or basic direction between the pI of the second protein and the pH for a reference formulation comprising:
a third molar amount of the conjugate comprising the first protein conjugated to the phosphorylcholine-containing polymer;
a fourth molar amount of the second protein that is not conjugated to the phosphorylcholine-containing polymer; and
the pharmaceutically acceptable carrier,
wherein a first total molar amount comprising a sum of the first molar amount and the second molar amount, and a second total molar amount comprising a sum of the third molar amount and the fourth molar amount are substantially the same, wherein the second molar amount is greater than the fourth molar amount,
wherein the reference formulation is substantially free of turbidity.

11. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the difference between the pI of the second protein and the pH of the formulation is selected to be greater than the minimum difference between the pI of the second protein and the pH for a reference formulation comprising:
the conjugate comprising the first protein conjugated to the phosphorylcholine-containing polymer;
the second protein that is not conjugated to the phosphorylcholine-containing polymer; and
the pharmaceutically acceptable carrier,
wherein the percent composition of the second protein relative to the total protein mass weight concentration of the first protein and the second protein in the composition is higher than the percent composition of the second protein relative to the total protein mass weight concentration of the first protein and the second protein in the reference composition,
wherein the reference composition is substantially free of turbidity.

12. A formulation comprising:
a first molar amount of a conjugate comprising a first protein conjugated to a polymer; and
a second molar amount of a second protein that is not conjugated to a polymer,
wherein the formulation comprises the second protein at about 1% or more of a total molar amount of the first protein and the second protein, wherein the total molar amount comprises a sum of the first molar amount and the second molar amount.

13. A therapeutically acceptable composition comprising:
a conjugate comprising a first protein conjugated to a phosphorylcholine-containing polymer;
a second protein that is not conjugated to a phosphorylcholine-containing polymer; and
a pharmaceutically acceptable carrier,
wherein the percent composition of the second protein relative to the total protein mass weight concentration of the first protein and the second protein in the composition is about 1% or more.

14. A therapeutically acceptable composition comprising:
a first molar amount of a conjugate comprising a first protein conjugated to a polymer; and
a second molar amount of a second protein that is not conjugated to a polymer,
wherein the composition comprises the second protein at about 1% or more of a total molar amount of the first protein and the second protein, wherein the total molar amount comprises a sum of the first molar amount and the second molar amount.

15. A formulation comprising:
a first molar amount of a first protein that is conjugated to a polymer; and
a second molar amount of a second protein that is not conjugated to a polymer,
the further improvement comprising: the formulation comprises the second protein at about 1% or more of a total molar amount of the conjugate and the second protein, wherein the total molar amount comprises a sum of the first molar amount and the second molar amount.

16. A formulation comprising:
a conjugate comprising a first protein conjugated to a polymer; and
a second protein that is not conjugated to a polymer,
wherein a first molar amount of the conjugate and a second molar amount of the second protein has been combined in the formulation such that the second molar amount is about 1% or more of a total molar amount of the conjugate and the second protein, wherein the total molar amount comprises a sum of the first molar amount and the second molar amount.

17. A therapeutically acceptable composition comprising:
a conjugate comprising a first protein conjugated to a polymer; and
a second protein that is not conjugated to a polymer,
wherein the second protein at a percent composition relative to the total protein mass weight concentration of the first protein and the second protein in the composition of about 1% or more has been combined with the conjugate, wherein the remainder of the total protein mass weight concentration comprises the first protein.

18. A formulation comprising:
a conjugate comprising a first protein conjugated to a phosphorylcholine-containing polymer, wherein the polymer has 9 arms and a molecular weight of between 600,000 and 1,000,000 Da, wherein the polymer is present in the formulation at about 100 mg/mL or more; and
a second protein that is not conjugated to a polymer, wherein the second protein is present in the formulation at 5-15 mg/mL.

19. An intraocular therapeutic composition comprising an anti-VEGF-A antibody at about 50 mg/mL of protein, the anti-VEGF-A antibody comprising:
a heavy chain comprising a complementarity
determining region 1
(CDR_{H}1):
(SEQ ID NO: 9)
GYDFTHYGMN,
CDR_{H}2:
(SEQ ID NO: 10)
WINTYTGEPTYAADFKR,
and
CDR_{H}3:
(SEQ ID NO: 11)
YPYYYGTSHWYFDV;
and
a light chain comprising
CDR_{L}1:
(SEQ ID NO: 12)
SASQDISNYLN,
CDR_{L}2:
(SEQ ID NO: 13)
FTSSLHS,
and
CDR_{L}3:
(SEQ ID NO: 14)
QQYSTVPWT,
wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody,
wherein the antibody conjugate comprises the anti-VEGF-A antibody conjugated to a phosphorylcholine-containing polymer at a non-native cysteine outside a variable region of the antibody, wherein the phosphorylcholine-containing polymer is present in the composition at about 100 mg/mL or more, wherein the phosphorylcholine-containing polymer has 9 arms and a molecular weight of between 600,000 and 1,000,000 Da,
wherein the pH of the composition is about 5.5 or lower.

20. An intraocular therapeutic composition comprising an anti-VEGF-A antibody at about 50 mg/mL of protein, the anti-VEGF-A antibody comprising:
a heavy chain comprising an amino acid sequence of SEQ ID NO: 1 (with or without the C-terminal lysine); and
a light chain comprising an amino acid sequence of SEQ ID NO: 2,
wherein the anti-VEGF-A antibody is present in the composition as either an antibody conjugate or unconjugated antibody, wherein the unconjugated antibody is present in the formulation at between about 10% to about 30% of a total molar amount of the antibody conjugate and the unconjugated antibody, wherein the total molar amount is the sum of the molar amount of the antibody conjugate and the molar amount of the unconjugated antibody,
wherein the antibody conjugate comprises the following structure: 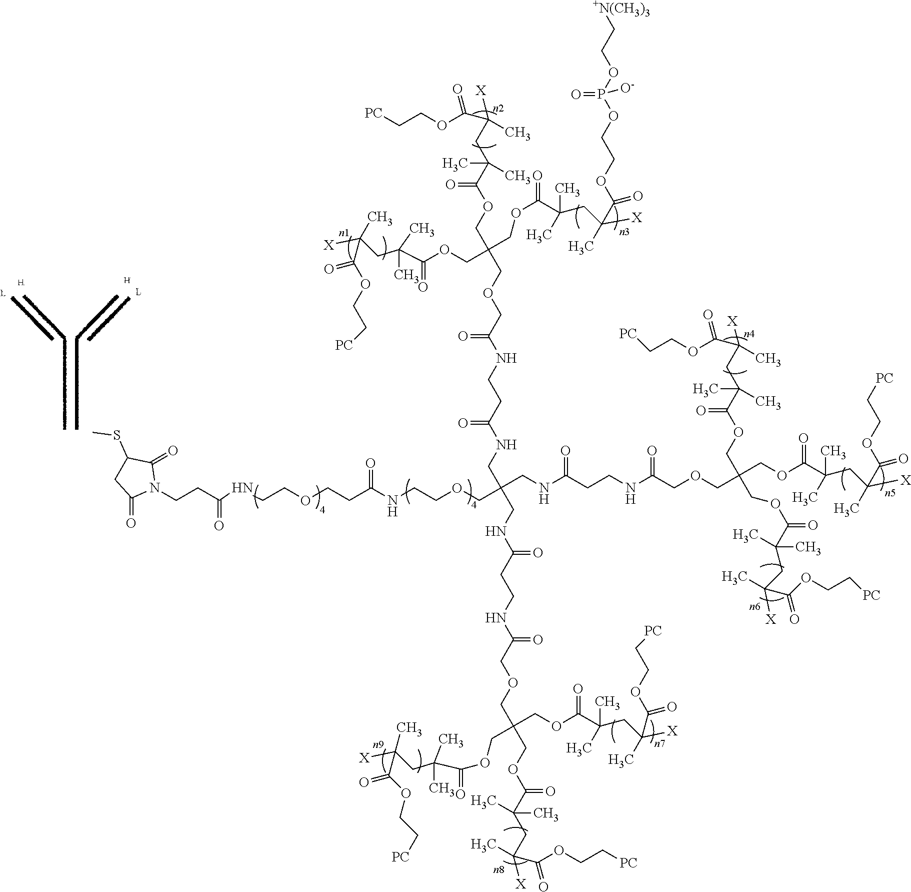
wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is 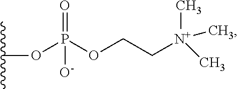 [SMILES: *OP(=O)([O-])OCC[N+](C)(C)C]
where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%,
wherein the phosphorylcholine-containing polymer is present in the composition at about 100 mg/mL or more,
wherein the pH of the composition is about 5.5 or lower.

21. An intraocular therapeutic composition comprising a fusion construct at about 53 mg/mL of protein, the fusion construct comprising a VEGF trap fused to an anti-IL-6 antibody, wherein the fusion construct comprises:
a heavy chain comprising an amino acid sequence of SEQ ID NO:105 (with or without the C-terminal lysine); and
a light chain comprising an amino acid sequence of SEQ ID NO:106, wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct,
wherein the conjugate comprises the fusion construct conjugated to a phosphorylcholine-containing polymer, wherein the phosphorylcholine-containing polymer is present in the composition at about 100 mg/mL or more, wherein the pH of the composition is about 5 or lower.

22. An intraocular therapeutic composition comprising a fusion construct at about 50 mg/mL of protein, the fusion construct comprising a VEGF trap fused to an anti-IL-6 antibody, wherein the fusion construct comprises:
a heavy chain comprising a complementarity
determining region 1
(CDR_{H}1):
(SEQ ID NO: 134)
PFAMH,
CDR_{H}2:
(SEQ ID NO: 135)
KISPGGSWTYYSDTVTD,
and
CDR_{H}3:
(SEQ ID NO: 136)
QAWGYYALDI;
and
a light chain comprising
CDR_{L}1:
(SEQ ID NO: 137)
SASISVSYLY,
CDR_{L}2:
(SEQ ID NO: 138)
DDSSLAS,
and
CDR_{L}3:
(SEQ ID NO: 139)
QQWSGYPYT,
wherein the fusion construct is present in the composition as either a conjugate or an unconjugated fusion construct, wherein the unconjugated fusion construct is present in the formulation at between about 20% to about 40% of a total molar amount of the conjugate and the unconjugated fusion construct, wherein the total molar amount is the sum of the molar amount of the conjugate and the molar amount of the unconjugated fusion construct,
wherein the conjugate comprises the following structure: 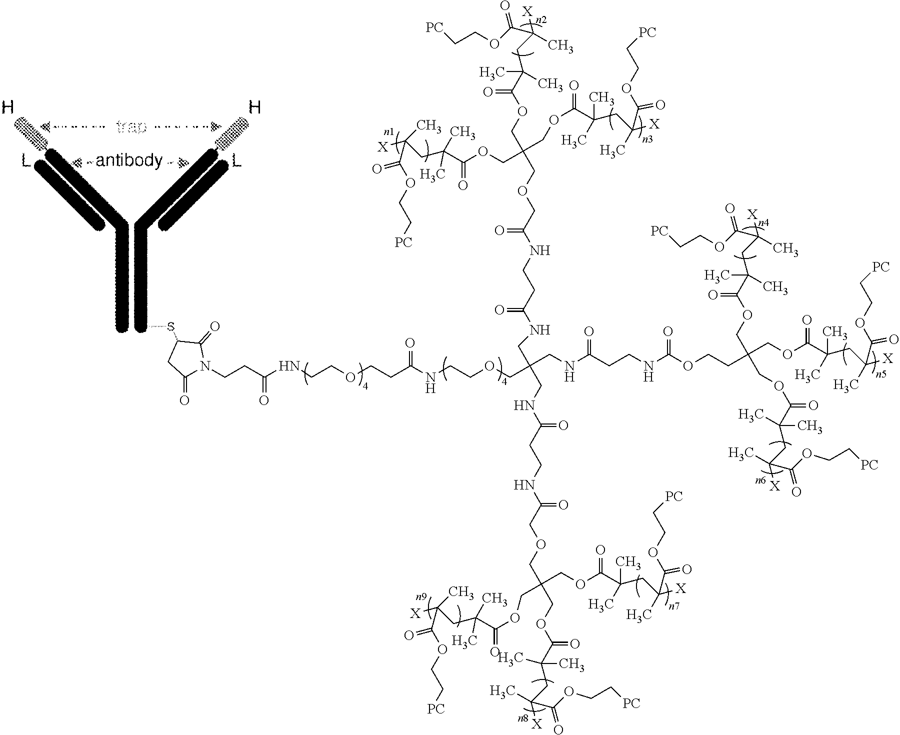
wherein:
each heavy chain of the conjugate is denoted by the letter H, and each light chain of the conjugate is denoted by the letter L;
the polymer is bonded to the heavy chain of the conjugate through the sulfhydryl of C443 (EU numbering), which bond is depicted on one of the heavy chains;
PC is 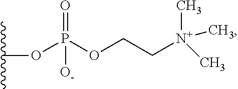
where the curvy line indicates the point of attachment to the rest of the polymer, where X is a) —OR where R is —H, methyl, ethyl, propyl, isopropyl, b) —H, c) any halogen, including —Br, —Cl, or —I, d) —SCN, or e) —NCS; and
n1, n2, n3, n4, n5, n6, n7, n8 and n9 are the same or different such that the sum of n1, n2, n3, n4, n5, n6, n7, n8 and n9 is 2500 plus or minus 15%, wherein the phosphorylcholine-containing polymer is present in the composition at about 100 mg/mL or more, wherein the pH of the composition is about 5 or lower.

23. A method of preparing a formulation, comprising combining in a formulation:
a first molar amount of a conjugate comprising a first protein conjugated to a phosphorylcholine-containing polymer; and
a second molar amount of a second protein that is not conjugated to a phosphorylcholine-containing polymer,
wherein the formulation comprises the second protein at about 1% or more of a total molar amount of the conjugate and the second protein, wherein the total molar amount comprises a sum of the first molar amount and the second molar amount,
wherein the formulation has a pH that is about 0.5 pH units away or more from the isoelectric point (pI) of the second protein.

24. A method of preparing a therapeutically acceptable composition, comprising combining in a therapeutically acceptable composition:
a first molar amount of a conjugate comprising a first protein conjugated to a phosphorylcholine-containing polymer; and
a second molar amount of a second protein that is not conjugated to a phosphorylcholine-containing polymer,
wherein the composition comprises the second protein at about 1% or more of a total molar amount of the conjugate and the second protein, wherein the total molar amount comprises a sum of the first molar amount and the second molar amount,
wherein the composition has a pH that is about 0.5 pH units away or more from the isoelectric point (pI) of the second protein.

25. A method of preparing a therapeutically acceptable composition, comprising combining in a therapeutically acceptable composition:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

26. A method of preparing a formulation, comprising adjusting the pH of a formulation to be about 0.5 pH units away or more from the isoelectric point (pI) of an unconjugated protein comprised in the formulation, wherein the formulation comprises:
a first molar amount of a conjugate comprising a first protein conjugated to a phosphorylcholine-containing polymer; and
a second molar amount of the unconjugated protein, wherein the unconjugated protein is not conjugated to a phosphorylcholine-containing polymer,
wherein the formulation comprises the unconjugated protein at about 1% or more of a total molar amount of the conjugate and the unconjugated protein, wherein the total molar amount comprises a sum of the first molar amount and the second molar amount.

27. A method of preparing a therapeutically acceptable composition, comprising adjusting the pH of a composition to be about 0.5 pH units away or more from the isoelectric point (pI) of an unconjugated protein comprised in the composition, wherein the composition comprises:
a first molar amount of a conjugate comprising a first protein conjugated to a phosphorylcholine-containing polymer; and
a second molar amount of the unconjugated protein, wherein the unconjugated protein is not conjugated to a phosphorylcholine-containing polymer,
wherein the composition comprises the unconjugated protein at about 0.1% or more of a total molar amount of the conjugate and the unconjugated protein, wherein the total molar amount comprises a sum of the first molar amount and the second molar amount.

28. A method of preparing a therapeutically acceptable composition, comprising adjusting the pH of a therapeutically acceptable composition to be about 0.5 pH units away or more from the isoelectric point (pI) of an unconjugated protein comprised in the composition, wherein the composition comprises:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more,
wherein the composition has a pH that is about 0.5 pH units away or more from the isoelectric point (pI) of the second protein.

29. A method of preparing a low-viscosity formulation of a protein conjugated to a phosphorylcholine-containing polymer, comprising combining in a formulation:
a first molar amount of a conjugate comprising a first protein conjugated to a phosphorylcholine-containing polymer; and
a second molar amount of the protein that is not conjugated to a phosphorylcholine-containing polymer,
wherein the formulation comprises the protein that is not conjugated to the phosphorylcholine-containing polymer at about 1% or more of a total molar amount of the conjugate unconjugated proteins, wherein the total molar amount comprises a sum of the first molar amount and the second molar amount,
wherein the formulation has a pH that is about 0.5 pH units away or more from the isoelectric point (pI) of the second protein,
wherein the formulation has reduced viscosity and/or an enhanced injectability compared to a reference formulation comprising the conjugate at the total molar amount.

30. A method of preparing a formulation, comprising combining in a formulation:
a first molar amount of a conjugate comprising a first protein conjugated to a phosphorylcholine-containing polymer; and
a second molar amount of a second protein that is not conjugated to a phosphorylcholine-containing polymer,
wherein the formulation comprises the second protein at about 1% or more of a total molar amount of the conjugate and the second protein, wherein the total molar amount comprises a sum of the first molar amount and the second molar amount.

31. A method of preparing a therapeutically acceptable composition, comprising combining in a therapeutically acceptable composition:
a conjugate comprising a first protein conjugated to a phosphorylcholine-containing polymer; and
a second protein that is not conjugated to a phosphorylcholine-containing polymer,
wherein the percent composition of the second protein relative to the total protein mass weight concentration of the first protein and the second protein in the composition is about 1% or more.

32. A method of treating a subject, comprising:
intraocularly administering a therapeutically effective amount of the formulation or composition of any one of claims 1-22 to a subject in need thereof.

33. A kit comprising:
a pre-filled syringe comprising a low-viscosity formulation comprising:
a conjugate comprising a first protein conjugated to a phosphorylcholine-containing polymer; and
a second protein that is not conjugated to a phosphorylcholine-containing polymer; and
a syringe needle for injection of the low-viscosity formulation, wherein the gauge of the needle is 27 or higher.

34. A formulation comprising about 40 to about 60 mM sodium acetate, about 0.01% to about 0.04% polysorbate 20, about 40 to about 60 mg/mL (total protein concentration) of a mixture of OG1950 and OG1953, the mixture containing about 15% to about 25% OG1950 and about 75% to about 85% OG1953 by molar amount, at pH about 4.5 to about 5.5.

35. A formulation comprising, consisting of, or consisting essentially of, about 50 mM sodium acetate, about 0.025% polysorbate 20, about 50 mg/mL (total protein concentration) of a mixture of OG1950 and OG1953, the mixture containing about 20% OG1950 and about 80% OG1953 by molar amount, at about pH 5.

36. A method of storing a protein, comprising maintaining a protein in a formulation for at least 2 months and up to 2 years, the formulation comprising:
a first molar amount of a conjugate comprising a first protein conjugated to a phosphorylcholine-containing polymer;
a second molar amount of a second protein that is not conjugated to a phosphorylcholine-containing polymer; and
a pharmaceutically acceptable carrier,
wherein the formulation comprises the second protein at about 1% or more of a total molar amount of the conjugate and the second protein, wherein the total molar amount comprises a sum of the first molar amount and the second molar amount, wherein the formulation has a pH that is about 0.5 pH units away or more from the isoelectric point (pI) of the second protein,
wherein the protein comprises an antibody or a fusion construct.


SEQUENCE LISTING (161 sequences)

SEQ ID NO: 1 - protein, 453 aa, synthetic construct
  REGION 1..453: note = Anti-VEGF-A heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 2 - protein, 214 aa, synthetic construct
  REGION 1..214: note = Anti-VEGF-A light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, synthetic construct
  REGION 1..453: note = Antigen binding protein components
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  REGION 1..214: note = Antigen binding protein components
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 231 aa, synthetic construct
  REGION 1..231: note = Antigen binding protein components
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 6 - protein, 214 aa, synthetic construct; identical to SEQ ID NO: 2
  REGION 1..214: note = Antigen binding protein components

SEQ ID NO: 7 - DNA, 235 bp, synthetic construct
  misc_feature 1..235: note = Heavy chain
  source 1..235: mol_type = other DNA
atgaaagctgtggtgctggccgtggctctggtcttcctgacagggagccaggctgaggtgcagctggtggaatccggcggaggcctggtccagcctggcggatccctgagactgtcctgtgccgcctccggctacgacttcacccattacggcatgaactgggtccgacaggcccctggcaagggcctggaatgggtcggatggatcaacacctacaccggcgagcccacctacg

SEQ ID NO: 8 - DNA, 702 bp, synthetic construct
  misc_feature 1..702: note = Light chain
  source 1..702: mol_type = other DNA
atgggatggagctgtatcatcctcttcttggtggcaacagctacaggcgtgcactccgacatccagctgacccagtccccctccagcctgtccgcctctgtgggcgacagagtgaccatcacctgttccgccagccaggacatctccaactacctgaactggtatcagcagaagcccggcaaggcccccaaggtgctgatctacttcacctcctccctgcactccggcgtgccctccagattctccggctctggctccggcaccgactttaccctgaccatctccagcctgcagcccgaggacttcgccacctactactgccagcagtactccaccgtgccctggaccttcggccagggcaccaaggtggaaatcaagcggaccgtggccgctccctccgtgttcatcttcccaccctccgacgagcagctgaagtccggaaccgcctccgtcgtgtgcctgctgaacaacttctacccccgcgaggccaaggtgcagtggaaggtggacaacgccctgcagagcggcaactcccaggaatccgtcaccgagcaggactccaaggacagcacctactccctgtccagcaccctgaccctgtccaaggccgactacgagaagcacaaggtgtacgcctgcgaagtgacccaccagggcctcagctccccagtgaccaagtccttcaaccggggcgagtgctag

SEQ ID NO: 9 - protein, 10 aa, synthetic construct
  REGION 1..10: note = Antigen binding protein components
GYDFTHYGMN

SEQ ID NO: 10 - protein, 17 aa, synthetic construct
  REGION 1..17: note = Antigen binding protein components
WINTYTGEPTYAADFKR

SEQ ID NO: 11 - protein, 14 aa, synthetic construct
  REGION 1..14: note = Antigen binding protein components
YPYYYGTSHWYFDV

SEQ ID NO: 12 - protein, 11 aa, synthetic construct
  REGION 1..11: note = Antigen binding protein components
SASQDISNYLN

SEQ ID NO: 13 - protein, 7 aa, synthetic construct
  REGION 1..7: note = Antigen binding protein components
FTSSLHS

SEQ ID NO: 14 - protein, 9 aa, synthetic construct
  REGION 1..9: note = Antigen binding protein components
QQYSTVPWT

SEQ ID NO: 15 - protein, 120 aa, synthetic construct
  source 1..120: note = KCD002 variable heavy chain
EVKLVESEGGLVQPGSSMKLSCTASGFTFSDYYMAWVRQVPEKGLEWVGNINYDGSSTYYLDSLKSRFIISRDSAKNILYLQMSSLKSEDTATYFCARGEDFYLYAMDYWGQGTSVTVSS

SEQ ID NO: 16 - protein, 116 aa, synthetic construct
  source 1..116: note = KCD003 variable heavy chain
EVQLQQSRPELVKPGASVKIFCKASGYTFTDYYMNWMRQRHGETLEWIGDINPNNGDPSYNQKFKDKATLTVDKSSSTASMELRSLTSDDSAVYYCAREGPSFAYWGQGTLVTVSA

SEQ ID NO: 17 - protein, 116 aa, synthetic construct
  source 1..116: note = KCD005 variable heavy chain
EVQLQQSGPELVKPGASVKISCKASGYTFTDHYMNWVKQSHGKSLEWIGDINPNNGGTSCNQKFKGKATLTVDKSSSTAYMELRSLTSGDSAVYYCTREGASFAFWGQGTLVTVSA

SEQ ID NO: 18 - protein, 116 aa, synthetic construct
  source 1..116: note = KCD009 variable heavy chain
QIQLVQSGPELKKPGETVKISCKASGYIFRNYGMNWVKQGPGKGLKWMGWINTYTGEPTYADDFKGRFAFSLETSASTAYLQISNLKNEDTATYFCVRDGPGFAYWGQGTLVTVSA

SEQ ID NO: 19 - protein, 114 aa, synthetic construct
  source 1..114: note = KCD010 variable heavy chain
EVLLQQSGPELVKPGASVKIPCKASGYTFTDYDMDWVKQSHGKSLEWIGHINPNNGGTIYNQKFKGKATLTVDKSSSTAYMELRSLTSEDTAVYYCGTGDFAYWGHGTLVTVSA

SEQ ID NO: 20 - protein, 114 aa, synthetic construct
  source 1..114: note = KCD023 variable heavy chain
EVLLQQSGPELVKPGASVKIPCKASGYTFTDYNIDWVKQSHGKSLEWIGDINPNNGGINYNQKFKGKATLTVDKSSSTAYMELRSLTSEDTAVYYCGTGDYAYWGQGTLVTVSA

SEQ ID NO: 21 - protein, 118 aa, synthetic construct
  source 1..118: note = KCD036 variable heavy chain
EVQLQQSGPELVKPGASMKISCKASGYSFTGYTMTWVKQSHGKNLEWIGLINPYNGGTNYNQKFKGKATFTVDKSSSTAYMELLSLTSEDSAVYYCARRHYGSSWDYWGQGTTLTVSS

SEQ ID NO: 22 - protein, 113 aa, synthetic construct
  source 1..113: note = KCD040 variable heavy chain
EVQLQQSGAELVKPGASVKLSCTASDFNIKDTYMHWVMQRPEQGLEWIGKIDPANGNTEFDPKFQGKATITADTSSNTAYLQLTSLTSEDTAVYYCTRAMDYWGQGTSVTVSS

SEQ ID NO: 23 - protein, 113 aa, synthetic construct
  source 1..113: note = KCD042 variable heavy chain
EVQLQQSGAELVKPGASVRLSCTASGFNIKHTYIHWVSQRPEQGLEWIGKIDPANGNTKYDPKFQGKATITADTSSNTAYLQLSSLTSEDTAVYYCVNAMEYWGQGTSVTVSS

SEQ ID NO: 24 - protein, 113 aa, synthetic construct
  source 1..113: note = KCD044 variable heavy chain
EVQLQQSGAELVKPGASVRLSCTASGFNIKHTYMHWVSQRPERGLEWIGKIDPANGNTKYDPKFQGKATITADTSSNTVYLQLSSLTSEDTAVYYCLNAMEYWGQGTSVTVSS

SEQ ID NO: 25 - protein, 113 aa, synthetic construct
  source 1..113: note = KCD047 variable heavy chain
EVQLQQSGAEFVKPGASVRLSCTASGFNIKDTYMHWVKQRPEQGLEWIGRIDPANGYTKDDPKFQGKATITADTSSNTAYLQLSSLTSEDTAVYYCASAMDYWGQGTSVTVSS

SEQ ID NO: 26 - protein, 113 aa, synthetic construct
  source 1..113: note = KCD048 variable heavy chain
EVQLQQSGADLVKPGASVKLSCTASGFNIKATYMHWVRQRPEKGLEWIGRIDPANGHTIYDPQFQGKATITSDTSSNTAYLQLNSLTSEDTAVYYCAEAMDYWGQGTSVTVSS

SEQ ID NO: 27 - protein, 118 aa, synthetic construct
  source 1..118: note = KCD070 variable heavy chain
EIQLQQTGPELVKPGASVKISCKASGYSFTDYIILWVKQSHGKSLEWIGNINPYYDYTSYNLKFKGKATLTVDKSSSTAYMQLNSLTSEDSAVYYCARSDGYYGGDYWGQGTSVTVSS

SEQ ID NO: 28 - protein, 118 aa, synthetic construct
  source 1..118: note = KCD101 variable heavy chain
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCGRNGYDGSMDYWGQGTSVTVSS

SEQ ID NO: 29 - protein, 118 aa, synthetic construct
  source 1..118: note = KCD102 variable heavy chain
QVQLQQPGAELVRPGTSVKLSCKASGYTFTSYWMHWVKQRPGQGLEWIGVIDPSDSYTNYNQKFKGKATLTVDTSSSTAYMQLSSLTSEDSAVYYCARNGYDGSMDYWGQGTSVTVSS

SEQ ID NO: 30 - protein, 118 aa, synthetic construct
  source 1..118: note = KCD103 variable heavy chain
QVQLQQPGAELVRPGTSVKLSCKASGYTFTSYWMHWVKQRPGQGLEWIGVIDPSDSYTKYNQKFKDKATLTVETSSSTAYMQLSSLTSEDSAVYYCAGNGYDGSMDYWGQGTSVTVSS

SEQ ID NO: 31 - protein, 118 aa, synthetic construct
  source 1..118: note = KCD104 variable heavy chain
QVQLQQPGAELVRPGTSVKLSCKASGYTFTSYWMHWVKQRPGQGLEWIGVIDPSDSYTYYNQKFKGKATLTVDTSSSTAYMQLSSLTSEDSAVYYCARNGYDGAMDYWGQGTSVTVSS

SEQ ID NO: 32 - protein, 116 aa, synthetic construct
  source 1..116: note = KCD118 variable heavy chain
EVQLQQSGPELVKPGASVKISCKAFGYTFTDYYKNWMRQRHGESLEWIGDINPNSGDANYNQKFKGKATLTVDKSSSTAYMELRSLTSEDSAVYYCAREGPSFAYWGHGTLVTVSA

SEQ ID NO: 33 - protein, 116 aa, synthetic construct
EVQLQQSGPELVKPGASVKISCKASGYTFTDYYTNWMRQRHGESLEWIGDINPNTGDTSYNQKFRVKATLTVDKSSGTAYMGLRSLTSEDSAVYYCTREGPSFAYWGQGTLVTVSA

SEQ ID NO: 34 - protein, 116 aa, synthetic construct
  source 1..116: note = KCD121 variable heavy chain
EVQLQQSGPELVKPGASVKISCKASGYTFTDYYKNWMRQRHGESLEWIGDINPNNGDTSYNQKFRGKATLTVDKSSSTAFMELRSLTSEDSAVYYCAREGPSFAYWGQGTLVTVSA

SEQ ID NO: 35 - protein, 116 aa, synthetic construct
  source 1..116: note = KCD122 variable heavy chain
EVQLQQSGPELVKPGASVKISCKASGYTFTDYYKNWMRQRHGESLEWIGDINPNNGDANYNQKFKGKATLTVDKSSSTAYMELRSLTSEDSAVYFCAREGPSFAYWGHGTLVTVSA

SEQ ID NO: 36 - protein, 116 aa, synthetic construct
  source 1..116: note = KCD123 variable heavy chain
EVHLQQSGPELVKPGASVKISCKASGYTFTDFYKNWMRQRHGESLEWIGDINPNNGGTNYNQKFKGKATLTVDKSSSTAYMELRSLTSEDSAVYYCAREGPSFAYWGQGTLVTVSA

SEQ ID NO: 37 - protein, 116 aa, synthetic construct
  source 1..116: note = KCD124 variable heavy chain
EVQLQQSGPELVKPGASVKISCKASGYTFTDHYMNWVKQSHGKSLEWIGDINPNNGGTSYNQKFKGKATLTVDKSSSTAYMELRSLTSGDSAVYYCTREGASFAFWGQGTLVTVSA

SEQ ID NO: 38 - protein, 122 aa, synthetic construct
  source 1..122: note = KCD131 variable heavy chain
QVQLQQSGPELVKPGASVKISCKASAYTFTDYYINWVKQRPGQGPEWIGWIFPGSNSTYSNEKFEVKATLTVDESSSTAYMLLSSLTSEDSAVYFCARLGYFGSSYHALDYWGQGTSVTVSS

SEQ ID NO: 39 - protein, 113 aa, synthetic construct
  source 1..113: note = KCD136 variable heavy chain
EVQLQQSVAELVRPGASVKLSCSASGFNIKNTYMHWVNQRPEQGLEWIGRIDPANGITKYAPNFQGKATITADTSSNTAYLQLSNLTSEDTAIYYCTRAMDYWGQGTSVTVSS

SEQ ID NO: 40 - protein, 116 aa, synthetic construct
  source 1..116: note = KCD200 variable heavy chain
EVQLQQSGPELVKPGASVKISCKASGYTFTSYYKNWMRQRHGESLEWIGDINPNSGDTAYNQKFKGKATLTVDRSSSTAYMELRSLTSEDSAVYYCAREGPSFAYWGQGTLVTVSA

SEQ ID NO: 41 - protein, 115 aa, synthetic construct
  source 1..115: note = KCD208 variable heavy chain
EVQLQQSVAELVRPGASVKLSCTVSGFNIKNTYMHWVKQRPEQGLEWIGRIDPANGDTTYAPKFQGKATITADTSSNSAYLHLSRLTSEDTAIYYCSLYDYDGYWGQGTTLTVSS

SEQ ID NO: 42 - protein, 115 aa, synthetic construct
  source 1..115: note = KCD214 variable heavy chain
EVQLQQSVAEFVRPGASVKLSCTASGFNIKNTYMHWVKQRPEQGLEWIGRIDPANGNTEYAPKFQGKATITADTSSNTAYLQLSSLTSEDTAIYYCALYDYDGYWGQGTTLTVSS

SEQ ID NO: 43 - protein, 119 aa, synthetic construct
  source 1..119: note = KCD220 variable heavy chain
QVQLQQSGAELMEPGASVKLSCKATGYTFTGYWIEWVKQRPGHGLEWIGETLPGSDSNNYNEKFKGKATFTADTSSNTAYMQLSSLTTEDSAIYYCARDYSNYWYFDVWGTGTTVTVSS

SEQ ID NO: 44 - protein, 119 aa, synthetic construct
  source 1..119: note = KCD224 variable heavy chain
QVQLKESGPGLVAPSQSLSITCTVSGFSLTSYGVDWIRQSPGKGLEWLGVIWGVGSTNYNSALKSRLSISKDNSKSQVFLKMNSLQTDDTAMYYCARSYDGSYWYFDVWGTGTTVTVSS

SEQ ID NO: 45 - protein, 116 aa, synthetic construct
  source 1..116: note = 119_TAF variable heavy chain
EVQLVESGGGLVQPGGSLRLSCAASGYTFTDYYMNWVRQAPGKGLEWIGDINPNTGDTSYNADFKRRFTFSLDTSKSTAYLQMNSLRAEDTAVYYCTREGPSFAYWGQGTLVTVSS

SEQ ID NO: 46 - protein, 117 aa, synthetic construct
  source 1..117: note = 119_TAF Germ variable heavy chain
EVQLVESGGGLVQPGGSLRLSCAASGYTFTDYYMSWVRQAPGKGLEWIGDINPNTGDTSYNADSVKGRFTISRDNSKNTLYLQMNSLRAEDTAVYYCTREGPSFAYWGQGTLVTVSS

SEQ ID NO: 47 - protein, 107 aa, synthetic construct
  source 1..107: note = 119_Human Germ variable heavy chain
EVKKPGASVKVSCKASGYTFTDYYMHWVRQAPGQGLEWIGDINPNTGDTSYNQKFQGRVTSTRDTSISTAYMELSRLRSDDTVVYYCTREGPSFAYWGQGTLVTVSS

SEQ ID NO: 48 - protein, 107 aa, synthetic construct
  source 1..107: note = KCD002 variable light chain
DIQMTQSPASLSASVGETVTITCRASENIHSYLAWYQQKQGKSPQLIVYNTKTLAEGVPSRFSGSGSGTQFSLKINSLQPEDFGSYYCQHHYGIPPTFGGGTKLEIK

SEQ ID NO: 49 - protein, 112 aa, synthetic construct
  source 1..112: note = KCD003 variable light chain
DVLMTQTPLSLPVSLGEQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 50 - protein, 112 aa, synthetic construct
  source 1..112: note = KCD005 variable light chain
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPVTFGAGTKLELK

SEQ ID NO: 51 - protein, 112 aa, synthetic construct
  source 1..112: note = KCD009 variable light chain
DVLMTQTPLSLPVSLGDQASISCRSSLIIEHSDGNTYLEWYLQKPGQSPKLLIYKVSNRFSGVPDRFSGSGSGTDFTLKISRVEADDLGVYYCFQGSHVPVTFGAGTNLELK

SEQ ID NO: 52 - protein, 108 aa, synthetic construct
  source 1..108: note = KCD010 variable light chain
QIVLTQSPAIMSVSPGEKVTLTCSASSSVSSSYLYWYQKKPGSSPKLWIYSTSNLASGVPARFSGSGSGTSYSLTISSMEAEDAASYFCHQWSSYPPTFGAGTKLELK

SEQ ID NO: 53 - protein, 108 aa, synthetic construct
  source 1..108: note = KCD023 variable light chain
QIVLTQSPAFMSASPGEKVTLTCSASSSVSSSYLYWYQQKPGSSPKLWIYSTSNLASGVPGRFSGSGSGTSYSLTISSMEAEDAASYFCHQWTSYPPTFGAGTKLELK

SEQ ID NO: 54 - protein, 107 aa, synthetic construct
  source 1..107: note = KCD036 variable light chain
DIKLTQSPSSMYASLGERVTITCKASQDINTYLSWFQQKPGKSPKTLIYRANRLVDGVPSRFSGSGSGQDYSLTISSLEYEEMGIYYCLQYDEFPYTFGGGTKLEIK

SEQ ID NO: 55 - protein, 106 aa, synthetic construct
  source 1..106: note = KCD040 variable light chain
QIVLTQSPAIMSASPGEKVTMTCSAGSSVSYMYWYQQKPGSSPRVLIYDTSNLASGVPVRFSGSGSGTSYSLTISRMEAEDAATYYCQQWSNYPYTFGGGTKLEIK

SEQ ID NO: 56 - protein, 106 aa, synthetic construct
  source 1..106: note = KCD042 variable light chain
QSVLTQSPAIMSASPGEKVTMTCSANSSVSDMYWFQQRPGSSPRLLIYDTSNLASGVPVRFSGSGSGTSYSLTISRMEAEDAATYYCQQWSTYPWTFGGGTKLEIK

SEQ ID NO: 57 - protein, 106 aa, synthetic construct
  source 1..106: note = KCD044 variable light chain
QSVLTQSPAIMSASPGEKVTMTCSANSSVSDMYWYQQRPGSSPRLLIYDTSNLASGVPVRFSGSGSGTSYSLTISRMEAEDAATYYCQQWSTYPWTFGGGTKLEIK

SEQ ID NO: 58 - protein, 106 aa, synthetic construct
  source 1..106: note = KCD047 variable light chain
QIVLTQSPAVMSASPGEKVAMTCSASSSVTYMYWYQQKPGSSPRLLIYDTSNLASGVPVRFSGSGSGTSYSLTISRMEAEDAATYYCQQWSTYPFPFGSGTKLEIK

SEQ ID NO: 59 - protein, 106 aa, synthetic construct
  source 1..106: note = KCD048 variable light chain
QIVLTQSPAIMSASPGEKVTLTCSATSSVSYMYWYQQKPGSSPRLLIYDTSNLASGVPVRFSGSGSGTSYSLTISRMEAEDDATYYCQQWSNYPFTFGGGTKLEIK

SEQ ID NO: 60 - protein, 107 aa, synthetic construct
  source 1..107: note = KCD070 variable light chain
DIVMTQSHKFMSTSVGDRVSITCKASQDVGTAVAWYQQKPGQSPKLLIYWASTRHTGVPDRFTGSGSGTDFTLTINNVQSEDLADYFCQQYSSYPWTFGGGTTLEIK

SEQ ID NO: 61 - protein, 107 aa, synthetic construct
  source 1..107: note = KCD101 variable light chain
DIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 62 - protein, 107 aa, synthetic construct
  source 1..107: note = KCD102 variable light chain
EIQMTQTTSSLSASLGDRVTISCRASQDISNYLNWYQQKPDGTVKLLIYYPSRLHSGVPSRFSGSGSGTDYSLTISNLEQEDFATYFCQQGNTLPYTFGGGTKLEIK

SEQ ID NO: 63 - protein, 107 aa, synthetic construct
  source 1..107: note = KCD103 variable light chain
DIQMTQTTSSLSASLGDRVTISCRASQDISNSLNWYQQKPDGTVKLLIYYTSRLHSRVPSRFSGSGSGTDYSLTISNLDQEDIATYFCQQANTLPYTFGGGTKLEIK

SEQ ID NO: 64 - protein, 107 aa, synthetic construct; identical to SEQ ID NO: 62
  source 1..107: note = KCD104 variable light chain

SEQ ID NO: 65 - protein, 112 aa, synthetic construct
  source 1..112: note = KCD118 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGIYYCFQGSHVPPTFGGGTKLEIK

SEQ ID NO: 66 - protein, 112 aa, synthetic construct
  source 1..112: note = KCD119 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTTLEIK

SEQ ID NO: 67 - protein, 112 aa, synthetic construct
  source 1..112: note = KCD121 variable light chain
DVLMTQTPLSLPVSLGDQASISCRSNQTIVHSNGDTYLEWYLQKPGQSPNLLIYKVSNRFSGVPDRFSGSGSGTDFTLRISRVEAEDLGVYYCFQGSHVPPTFGGGTKLEIK

SEQ ID NO: 68 - protein, 112 aa, synthetic construct
  source 1..112: note = KCD122 variable light chain
DVLMTQTPLSLPVSLGDQASISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 69 - protein, 112 aa, synthetic construct; identical to SEQ ID NO: 68
  source 1..112: note = KCD123 variable light chain

SEQ ID NO: 70 - protein, 112 aa, synthetic construct
  source 1..112: note = KCD124 variable light chain
DVLMTQTPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPLTFGAGTKLELK

SEQ ID NO: 71 - protein, 107 aa, synthetic construct
  source 1..107: note = KCD131 variable light chain
DIQMTQSPASLSVSVGETVTITCRASENIYSHLAWFQQKQGKSPRLLVYSATNLPDGVPSRFSGSGSGTQYSLKINILQSEDFGSYYCQHFWGTPWTFGGGTKLEIK

SEQ ID NO: 72 - protein, 106 aa, synthetic construct
  source 1..106: note = KCD136 variable light chain
QIVLTQSPAIMSASPGEKVTMTCSASSSVSYMYWYQQKPGSSPRLLIYDISNLASGVPVRFSGSGSGTSYSLTISRMEAEDAATYYCQQWDTYPWTFGGGTKLEIK

SEQ ID NO: 73 - protein, 112 aa, synthetic construct
  source 1..112: note = KCD200 variable light chain
DVLMTQTPLSLPVSLGDQVSISCRSSQTIVHSNGDTYLEWYLQKPGQSPNLLIYKVSNRFSGVPDRFSGSGSGTDFTLKISRVEAEDLGVYYCFQGSHVPPTFGGGTKLEIK

SEQ ID NO: 74 - protein, 108 aa, synthetic construct
  source 1..108: note = KCD208 variable light chain
EIVLTQSPALMAASPGEKVTITCSVSSSISSSSLHWYRQKSGTSPKPWIYGTSHLASGVPVRFSGSGSGTSYSLTISSMEAEDAATYYCQQWDTYPWTFGGGTKLEIK

SEQ ID NO: 75 - protein, 108 aa, synthetic construct
  source 1..108: note = KCD214 variable light chain
EIVLTQSPALMAASPGEKVTITCRVSSSISSSSLHWYQQKSGTSPKPWIYGTSNLASGVPVRFSGSRSGTSYSLTISSMEAEDAATYYCQQWSDYPWTFGGGTKLEIK

SEQ ID NO: 76 - protein, 106 aa, synthetic construct
  source 1..106: note = KCD220 variable light chain
DIQMTQTTSSLSASLGDRVTISCRASQDISNYLNWYQQKPDGTVKLLIYYTSNLHSGVPSRFSGSGSGTDYSLTISNLEQEDIATYFCQQDSKHRTFGGGTKLEIK

SEQ ID NO: 77 - protein, 107 aa, synthetic construct
  source 1..107: note = KCD224 variable light chain
DIQMTQTTSSLSASLGDRVTISCRASQVISNYLNWYQQKPDGTVKLLIYYTSRLHSGVPSRFSGSGSGTDYSLTISNLEPEDIATYYCQQYSKLPYTFGSGTKLEIK

SEQ ID NO: 78 - protein, 112 aa, synthetic construct
  source 1..112: note = 119_TAF variable light chain
DIQLTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 79 - protein, 112 aa, synthetic construct
  source 1..112: note = 119_TAF Germ variable light chain
DIQMTQSPSSLSASVGDRVTITCRSSQTIVHSNGDTYLEWYQQKPGKAPNLLIYKVSNRFSGVPSRFSGSGSGTDFTLTISSLQPEDFATYYCFQGSHVPPTFGQGTKVEIK

SEQ ID NO: 80 - protein, 112 aa, synthetic construct
  source 1..112: note = 119_Human Germ variable light chain
DVVMTQSPLSLPVTLGQPASISCRSSQTIVHSNGDTYLEWFQQRPGQSPNLLIYKVSNRFSGVPDRFSGSGSGTDFTLKISRVEAEDVGVYYCFQGSHVPPTFGGGTKVEIK

SEQ ID NO: 81 - protein, 118 aa, synthetic construct
  source 1..118: note = I IL-6 variable heavy chain
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVS

SEQ ID NO: 82 - protein, 119 aa, synthetic construct
  source 1..119: note = II IL-6 variable heavy chain
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSS

SEQ ID NO: 83 - protein, 119 aa, synthetic construct
  source 1..119: note = IIa IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSS

SEQ ID NO: 84 - protein, 119 aa, synthetic construct
  source 1..119: note = IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQSWGYYALDIWGQGTLVTVSS

SEQ ID NO: 85 - protein, 119 aa, synthetic construct
  source 1..119: note = IIc IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQGWGYYALDIWGQGTLVTVSS

SEQ ID NO: 86 - protein, 119 aa, synthetic construct
  source 1..119: note = IId IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQTWGYYALDIWGQGTLVTVSS

SEQ ID NO: 87 - protein, 119 aa, synthetic construct
  source 1..119: note = IIe Il-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQVWGYYALDIWGQGTLVTVSS

SEQ ID NO: 88 - protein, 119 aa, synthetic construct
  source 1..119: note = IIf IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQQWGYYALDIWGQGTLVTVSS

SEQ ID NO: 89 - protein, 119 aa, synthetic construct
  source 1..119: note = IIg IL-6 variable heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQKWGYYALDIWGQGTLVTVSS

SEQ ID NO: 90 - protein, 106 aa, synthetic construct
  source 1..106: note = III IL-6 variable light chain
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIK

SEQ ID NO: 91 - protein, 106 aa, synthetic construct
  source 1..106: note = IV IL-6 variable light chain
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIK

SEQ ID NO: 92 - protein, 106 aa, synthetic construct
  source 1..106: note = V IL-6 variable light chain
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIK

SEQ ID NO: 93 - protein, 664 aa, synthetic construct
  source 1..664: note = A heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 94 - protein, 213 aa, synthetic construct
  source 1..213: note = A light chain
DIQLTQSPSSLSASVGDRVTITCSASISVSYLYWYQQKPGKAPKLLIYDDSSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 664 aa, synthetic construct; identical to SEQ ID NO: 93
  source 1..664: note = B heavy chain

SEQ ID NO: 96 - protein, 213 aa, synthetic construct
  source 1..213: note = B light chain
DIQLTQSPSSLSASVGDRVTITCSASISVSYLYWYQQKPGKAPKLLIYDASSLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 664 aa, synthetic construct; identical to SEQ ID NO: 93
  source 1..664: note = C heavy chain

SEQ ID NO: 98 - protein, 213 aa, synthetic construct
  source 1..213: note = C light chain
DIQLTQSPSSLSASVGDRVTITCSASISVSYLYWYQQKPGKAPKLLIYDDSNLASGVPSRFSGSGSGTDFTLTISSLQPEDFATYYCQQWSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 664 aa, synthetic construct
  source 1..664: note = D heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 100 - protein, 213 aa, synthetic construct; identical to SEQ ID NO: 94
  source 1..213: note = D light chain

SEQ ID NO: 101 - protein, 664 aa, synthetic construct; identical to SEQ ID NO: 99
  source 1..664: note = E heavy chain

SEQ ID NO: 102 - protein, 213 aa, synthetic construct; identical to SEQ ID NO: 96
  source 1..213: note = E light chain

SEQ ID NO: 103 - protein, 664 aa, synthetic construct; identical to SEQ ID NO: 99
  source 1..664: note = F heavy chain

SEQ ID NO: 104 - protein, 213 aa, synthetic construct; identical to SEQ ID NO: 98
  source 1..213: note = F light chain

SEQ ID NO: 105 - protein, 664 aa, synthetic construct
  source 1..664: note = G heavy chain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 106 - protein, 213 aa, synthetic construct; identical to SEQ ID NO: 94
  source 1..213: note = G light chain

SEQ ID NO: 107 - protein, 664 aa, synthetic construct; identical to SEQ ID NO: 105
  source 1..664: note = H heavy chain

SEQ ID NO: 108 - protein, 213 aa, synthetic construct; identical to SEQ ID NO: 96
  source 1..213: note = H light chain

SEQ ID NO: 109 - protein, 664 aa, synthetic construct; identical to SEQ ID NO: 105
  source 1..664: note = I heavy chain

SEQ ID NO: 110 - protein, 213 aa, synthetic construct; identical to SEQ ID NO: 98
  source 1..213: note = I light chain

SEQ ID NO: 111 - protein, 664 aa, synthetic construct
  source 1..664: note = J heavy chain
EVQLVESGGGLVQPGGSLRLSCAASGFTFSPFAISWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 112 - protein, 213 aa, synthetic construct; identical to SEQ ID NO: 94
  source 1..213: note = J light chain

SEQ ID NO: 113 - protein, 664 aa, synthetic construct; identical to SEQ ID NO: 111
  source 1..664: note = K heavy chain

SEQ ID NO: 114 - protein, 213 aa, synthetic construct; identical to SEQ ID NO: 96
  source 1..213: note = K light chain

SEQ ID NO: 115 - protein, 664 aa, synthetic construct; identical to SEQ ID NO: 111
  source 1..664: note = L heavy chain

SEQ ID NO: 116 - protein, 213 aa, synthetic construct; identical to SEQ ID NO: 98
  source 1..213: note = L light chain

SEQ ID NO: 117 - protein, 664 aa, synthetic construct
  source 1..664: note = M heavy chain
EVQLVESGGGLVQPGGSLRLSCAASGFTFSPFAWSWVRQAPGKGLEWVAKISPGGSWTYYSDTVTDRFTFSLDTSKSTAYLQMNSLRAEDTAVYYCARQLWGYYALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 118 - protein, 213 aa, synthetic construct; identical to SEQ ID NO: 94
  source 1..213: note = M light chain

SEQ ID NO: 119 - protein, 664 aa, synthetic construct; identical to SEQ ID NO: 117
  source 1..664: note = N heavy chain

SEQ ID NO: 120 - protein, 213 aa, synthetic construct; identical to SEQ ID NO: 96
  source 1..213: note = N light chain

SEQ ID NO: 121 - protein, 664 aa, synthetic construct; identical to SEQ ID NO: 117
  source 1..664: note = O heavy chain

SEQ ID NO: 122 - protein, 213 aa, synthetic construct; identical to SEQ ID NO: 98
  source 1..213: note = O light chain

SEQ ID NO: 123 - protein, 664 aa, synthetic construct
  source 1..664: note = P heavy chain
EVQLVESGGGLVQPGGSLRLSCAASGFTFSPFAMHWVRQAPGKGLEWVAKISPGGSWTYYSDTVTDRFTFSLDTSKSTAYLQMNSLRAEDTAVYYCARQAWGYYALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 124 - protein, 213 aa, synthetic construct; identical to SEQ ID NO: 94
  source 1..213: note = P light chain

SEQ ID NO: 125 - protein, 664 aa, synthetic construct; identical to SEQ ID NO: 123
  source 1..664: note = Q heavy chain

SEQ ID NO: 126 - protein, 213 aa, synthetic construct; identical to SEQ ID NO: 96
  source 1..213: note = Q light chain

SEQ ID NO: 127 - protein, 664 aa, synthetic construct; identical to SEQ ID NO: 123
  source 1..664: note = R heavy chain

SEQ ID NO: 128 - protein, 213 aa, synthetic construct; identical to SEQ ID NO: 98
  source 1..213: note = R light chain

SEQ ID NO: 129 - protein, 446 aa, synthetic construct
  source 1..446: note = Exemplary human IgG1 heavy chain
EVQLVESGGGLVQPGGSLRLSCAASGYTFTDYYINWVRQAPGKGLEWIGDINPITGDTDYNADFKRRFTFSLDTSKSTAYLQMSSLRAEDTAVYYCTREGPSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 130 - protein, 219 aa, synthetic construct
  source 1..219: note = Exemplary human kappa light chain
DIQLTQSPSSLSASVGDRVTITCRSSQTIVHSNGDTYLEWYQQKPGKAPNLLIRKVSNRFSGVPSRFSGSGSGTDFTLTISSLQPEDFATYYCFQGSHVPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 745 aa, synthetic construct
  source 1..745: note = Synthetic polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 132 - protein, 214 aa, synthetic construct; identical to SEQ ID NO: 2
  source 1..214: note = Synthetic polypeptide

SEQ ID NO: 133 - protein, 205 aa, Synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 134 - protein, 5 aa, synthetic construct
PFAMH

SEQ ID NO: 135 - protein, 17 aa, synthetic construct
KISPGGSWTYYSDTVTD

SEQ ID NO: 136 - protein, 10 aa, synthetic construct
QAWGYYALDI

SEQ ID NO: 137 - protein, 10 aa, synthetic construct
SASISVSYLY

SEQ ID NO: 138 - protein, 7 aa, synthetic construct
DDSSLAS

SEQ ID NO: 139 - protein, 9 aa, synthetic construct
QQWSGYPYT

SEQ ID NO: 140 - protein, 5 aa, synthetic construct
FYHVH

SEQ ID NO: 141 - protein, 16 aa, synthetic construct
SIYTSGYTEYASALES

SEQ ID NO: 142 - protein, 11 aa, synthetic construct
EGLQRVGVLDA

SEQ ID NO: 143 - protein, 11 aa, synthetic construct
EGLQRVGVFDA

SEQ ID NO: 144 - protein, 11 aa, synthetic construct
EGLQRVGVMDA

SEQ ID NO: 145 - protein, 16 aa, synthetic construct
RSSQSLLDEAGETYLA

SEQ ID NO: 146 - protein, 7 aa, synthetic construct
EVSLLES

SEQ ID NO: 147 - protein, 9 aa, synthetic construct
QQATYFPYT

SEQ ID NO: 148 - protein, 8 aa, synthetic construct
GFSLTFYH

SEQ ID NO: 149 - protein, 7 aa, synthetic construct
IYTSGYT

SEQ ID NO: 150 - protein, 13 aa, synthetic construct
AREGLQRVGVFDA

SEQ ID NO: 151 - protein, 13 aa, synthetic construct
AREGLQRVGVMDA

SEQ ID NO: 152 - protein, 13 aa, synthetic construct
AREGLQRVGVLDA

SEQ ID NO: 153 - protein, 11 aa, synthetic construct
QSLLDEAGETY

SEQ ID NO: 154 - protein, 7 aa, synthetic construct
GFSLTFY

SEQ ID NO: 155 - protein, 5 aa, synthetic construct
YTSGY

SEQ ID NO: 156 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVFDAWGQGTLVTVSS

SEQ ID NO: 157 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVMDAWGQGTLVTVSS

SEQ ID NO: 158 - protein, 119 aa, synthetic construct
QVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVLDAWGQGTLVTVSS

SEQ ID NO: 159 - protein, 119 aa, synthetic construct
EVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVMDAWGQGTLVTVSS

SEQ ID NO: 160 - protein, 119 aa, synthetic construct
EVQLVESGGGVVQPGGSLRLSCAASGFSLTFYHVHWVRQAPGKGLEWVASIYTSGYTEYASALESRFTISRDTSKSTLYLQMNSLRAEDTAVYYCAREGLQRVGVLDAWGQGTLVTVSS

SEQ ID NO: 161 - protein, 112 aa, synthetic construct
DIQLTQSPSSLSASVGDRVTITCRSSQSLLDEAGETYLAWFQQKPGKAPKLLIYEVSLLESGVPSRFSGSGSGTDFTLTISSLQPEDFATYYCQQATYFPYTFGQGTKVEIK